(19) United States
(12) Patent Application Publication
(10) Pub. No.: US 2023/0227466 A1
(43) Pub. Date: Jul. 20, 2023

(54) SUBSTITUTED THIENOPYRIMIDINES THAT INTERACT WITH THE RAS SUPERFAMILY FOR THE TREATMENT OF CANCERS, INFLAMMATORY DISEASES, RASOPATHIES, AND FIBROTIC DISEASE

(71) Applicant: SHY Therapeutics, LLC, Harrison, NY (US)

(72) Inventors: Yaron R. Hadari, Harrison, NY (US); Michael Schmertzler, St. Petersburg, FL (US); Theresa M. Williams, Harleysville, PA (US); Luca Carta, Scarsdale, NY (US); Rebecca Hutcheson, Stamford, CT (US)

(21) Appl. No.: 18/010,697

(22) PCT Filed: Jun. 17, 2021
(86) PCT No.: PCT/US2021/037826

§ 371 Date: Dec. 15, 2022

Related U.S. Application Data
(60) Provisional application No. 63/040,916, filed on Jun. 18, 2020.
(60) Provisional application No. 63/211,126, filed on Jun. 16, 2021.

(51) Int. Cl. C07D 495/04; C07D 519/00; A61P 35/00

(52) U.S. Cl. CPC C07D 495/04; C07D 519/00; A61P 35/00

(57) ABSTRACT

Provided herein are methods and compositions for treating cancers, inflammatory diseases, rasopathies, and fibrotic disease involving aberrant Ras superfamily signaling through the binding of compounds to the GTP binding domain of Ras superfamily proteins including, in certain cases, K-Ras and mutants thereof, and a method for assaying such compositions.

### CROSS REFERENCE TO RELATED APPLICATIONS

This application claims the benefit under 35 U.S.C. § 119(e) of U.S. Provisional Application No. 63/211,126 filed on Jun. 16, 2021, and further claims the benefit of priority from U.S. Provisional Application No. 63/040,916, filed Jun. 18, 2020. Each of the foregoing related applications, in its entirety, is incorporated herein by reference.

### 1. FIELD

Provided herein are compositions and methods for treating cancers, inflammatory diseases, rasopathies, and fibrotic disease resulting from aberrant Ras signaling involving Ras, Rac, Rho, and Cdc42 members of the Ras superfamily of proteins through the binding of compounds to the GTP binding domain of these molecules.

### 2. BACKGROUND

Pathobiology of Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. (Roitt, I., Brostoff, J. and Kale, D., Immunology, 17.1-17.12 (3rd ed., Mosby, St. Louis, Mo., 1993))

Various stages of tumor development can be described generally as follows:

a) Tumor evolution commences when a cell within a normal population sustains a genetic mutation that expands its tendency to proliferate.

b) Such genetically altered cells and their offspring continue to appear normal, but they reproduce excessively and lead to a condition termed hyperplasia. The altered cells may also secrete signaling factors or other molecules that cause changes in their local cellular and extracellular environment, including without limitation, the response of the immune system to them. Such environmental effects may in turn affect the viability, proliferation, and further mutations of the altered cells. After some time (months or years) a very small fraction of these altered cells may sustain additional mutation with subsequent loss of control of cell growth and further potential effects on their environment.

c) The offspring of these cells not only proliferate excessively but also appear abnormal in shape and in orientation. The tissue is now said to exhibit a condition termed dysplasia. After some time, one or more additional mutations may further alter cell behavior and the effect of the cells on their environment.

d) The influenced and genetically altered cells turn still more abnormal in growth and appearance. If the tumor mass does not invade through any boundaries between tissues, it is termed an in situ tumor. This tumor may stay contained indefinitely, however, some cells may acquire still more mutations.

e) A malignant or invasive tumor results if the genetic changes allow the tumor mass to initiate invading underlying tissue and to cast off cells into the blood or lymph. The defector cells may install new tumors loci (metastases) throughout the body.

Metastases represent the end products of a multistep cell-biological process termed the invasion-metastasis cascade, which involves dissemination of cancer cells to anatomically distant organ sites and their subsequent adaptation to foreign tissue microenvironments. Each of these events is driven by the acquisition of genetic and/or epigenetic alterations within tumor cells and the co-option of non-neoplastic stromal cells, which together endow incipient metastatic cells with traits needed to generate macroscopic metastases. (Volastyan, S., et al., Cell, 2011, vol. 147, 275-292)

An enormous variety of cancers affect different tissues throughout the body, which are described in detail in the medical literature. Over 85% of human cancers are solid tumors, including carcinomas, sarcomas and lymphomas. Different types of solid tumors are named for the type of cells that form them. Examples include cancer of the lung, colon, rectum, pancreatic, prostate, breast, brain, and intestine. Other human tumors derive from cells involved in the formation of immune cells and other blood cells, including leukemias and myelomas.

The incidence of cancer continues to climb as the general population ages, as new cancers develop, and as susceptible populations grow. A tremendous demand therefore exists for new methods and compositions that can be used to treat subjects with cancer.

Methods of Treating Cancer

Current cancer therapy may involve surgery, chemotherapy, hormonal therapy, biological therapy, targeted therapy, immunotherapy and/or radiation treatment to eradicate neoplastic cells in a patient (see, e.g., Stockdale, 1998, Medicine, vol. 3, Rubenstein and Federman, eds., Chapter 12, Section IV; and Baudino T A “Targeted Cancer Therapy: The Next Generation of Cancer Treatment”, Curr Drug Discov Technol. 2015; 12(1):3-20).

Such therapies may be used independently or in combinations. Choices of therapy will depend on the history and nature of the cancer, the condition of the patient, and, under the circumstances, the anticipated efficacy and adverse effects of the therapeutic agents and methods considered.

With respect to chemotherapy, there are a variety of chemotherapeutic agents and methods of delivery of such agents available for the treatment of different cancers. Most first generation chemotherapeutic agents were not tumor specific, have broad systemic effects, are toxic, and may cause significant and often dangerous side effects, including severe nausea, bone marrow depression, and immunosuppression.

Additionally, even with administration of combinations of chemotherapeutic agents, many tumor cells are or become resistant to chemotherapeutic agents. In fact, cells resistant to the particular chemotherapeutic agents used in a treatment protocol often prove to be resistant to other drugs, even if those agents act by different mechanism from those of the drugs used in the specific treatment. This phenomenon is referred to as multidrug resistance. Because of drug resistance, many cancers prove refractory to standard chemotherapeutic treatment protocols.

Thus, there exists a significant need for alternative compounds, compositions and methods for treating, preventing and managing cancer.

Further, whereas surgical resection and adjuvant therapy can cure well-confined primary tumors, metastatic disease is largely incurable because of its systemic nature and the resistance of disseminated tumor cells to existing therapeutic agents. This explains why greater than 90% of mortality from cancer is attributable to metastases, not the primary tumors from which these malignant lesions arise.

Pathobiology of Inflammatory Disease

Inflammation is a complex protective biological response of body tissues to harmful stimuli, such as pathogens, damaged cells, or irritants, involving immune cells, blood vessels, and molecular mediators. The function of inflammation is to eliminate the initial cause of cell injury, clear out necrotic cells and tissues damaged from the original insult and the inflammatory process, and to initiate tissue repair. (Ferrero-Miliani L, Nielsen O H, Andersen P S, Girardin S E; Nielsen; Andersen; Girardin (February 2007) Clin. Exp. Immunol. 147)

Inflammation is classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue.

Prolonged inflammation, known as chronic inflammation, is characterized by simultaneous destruction and healing of the tissue from the inflammatory process. It leads to a progressive shift in the type of cells present at the site of inflammation, such as mononuclear cells, and increases in systemic concentrations of cytokines such as TNF-α, IL-6, and CRP. (Petersen, A. M.; Pedersen, B. K. (2005). J Appl Physiol. 98 (4): 1154-1162)

Many proteins are involved in inflammation. Any of them are susceptible to genetic mutation which may impair or otherwise dysregulate their normal function and expression.

Methods of Treating Inflammatory Disease

Both small molecules and biologics are used to treat inflammatory diseases. Most treatments, however, are largely palliative.

A clear unmet medical need remains to find treatments that can mechanistically reduce chronic inflammatory diseases.

Pathobiology of Fibrotic Disease

Fibrosis, or the accumulation of extracellular matrix molecules that constitute scar tissue, is a common result of tissue injury. Pulmonary fibrosis, renal fibrosis, and hepatic cirrhosis are among the common fibrotic diseases which altogether represent a large unmet medical need. (Friedman S L, Sheppard D, Duffield J S, Violette S. Sci Transl Med 2013 Jan. 9; 5(167): 167sr1).

Mechanisms of fibrogenesis include inflammation as well as other pathways and generally involve reorganization of the actin cytoskeleton of affected cells, including epithelial cells, fibroblasts, endothelial cells, and macrophages.

Actin filament assembly and actomyosin contraction are directed by the Rho-associated coiled-coil forming protein kinase (ROCK) family of serine/threonine kinases (ROCK1 and ROCK2) and thus Rho is associated with fibrogenesis.

Tissue fibrosis is a leading cause of morbidity and mortality. 45% of deaths in the United States are attributable to fibrotic disorders. (Wynn T A. “Fibrotic Disease and the TH1/TH2 Paradigm.” Nat Rev Immunol 2004 August: 4(8): 583-594.) Treatments are generally palliative.

Idiopathic pulmonary fibrosis (IPF) is characterized by progressive lung scarring, short median survival, and limited therapeutic options, creating great need for new pharmacologic therapies. It is thought to result from repetitive environmental injury to the lung epithelium.

Targeted Therapy of Cancer, Inflammatory, and Fibrotic Diseases

Targeted therapies are a cornerstone of what is also referred to as precision medicine, a form of medicine that uses information about a person's genes and proteins to prevent, diagnose, and treat disease. Such therapeutics are sometimes called “molecularly targeted drugs,” “molecularly targeted therapies,” or similar names. The process of discovering them is often referred to as “rational drug design.” This concept can also be referred to as “personalized medicine.”

A series of actions among molecules in a cell that leads to a certain end point or cell function is referred to as a molecular pathway.

Molecularly targeted drugs interact with a particular target molecule, or structurally related set of target molecules, in a pathway; thus modulating the endpoint effect of that pathway, such as a disease-related process; and, thus, yielding a therapeutic benefit.

Molecularly targeted drugs may be small molecules or biologics, usually antibodies. They may be useful alone or in combinations with other therapeutic agents and methods.

Because they target a particular molecule, or related set of molecules, and are usually designed to minimize their interactions with other molecules, targeted therapeutics may have fewer adverse side effects.

Targeted cancer drugs block the growth and spread of cancer by interacting with specific molecules or sets of structurally related molecules (altogether, “molecular targets”) that are involved, broadly speaking, in the growth, progression, lack of suppression or elimination, or spread of cancer. Such molecular targets may include proteins or genes involved in one or more cellular functions including, for example and without limitation, signal transduction, gene expression modulation, apoptosis induction or suppression, angiogenesis inhibition, or immune system modulation.

In some cases, the development of targeted cancer therapeutics involves identifying genes or proteins that are present in cancer cells but not normal cells or that are more abundant or more highly stimulated in cancer cells, especially if they are known to be involved in cancer processes, and then discovering agents that will interact with those targets and be associated with a desired therapeutic effect.

Targeted cancer therapies generally differ from standard cancer chemotherapy in several ways:
a. Targeted therapies are deliberately chosen or designed to interact with their target(s), whereas many standard chemotherapies were identified because they were found in general to kill cells.
b. Targeted therapies are intended to act on specific molecular targets that are associated with cancer, whereas most standard chemotherapies act on all rapidly dividing normal and cancerous cells. They may, however, also have known and sometime unknown interactions with other molecules, so-called off-target effects.
c. Most targeted therapies are cytostatic (that is, they block tumor cell proliferation), whereas standard chemotherapy agents are usually cytotoxic (that is, they kill tumor cells). However, some targeted therapies such as Antibody Drug Conjugates are cytotoxic.

Targeted therapy monoclonal antibodies (mAbs) and targeted small molecules are being used as treatments for inflammatory diseases (Kotsovilis S, Andreakos E., Methods Mol Biol. 2014; 1060:37-59). They are used either as a monotherapy or in combination with other conventional therapeutic modalities, particularly if the disease under treatment is refractory to therapy using solely conventional techniques.

Some treatments for fibrotic disorders, such as idiopathic pulmonary fibrosis, hepatic fibrosis, and systemic sclerosis, target inflammatory pathways.

The Ras GTPase Family

The Ras superfamily of proteins are small GTPases with substantial amino acid sequence homology that act as signal transducers between cell surface receptors and several intracellular signaling cascades. These molecules are involved in the regulation of such essential cellular functions as cell survival, proliferation, motility, and cytoskeletal organization (see Karnoub et al., Nat. Rev. Mol. Cell Biol., 9: 517-531 (2008)).

Research has defined a number of subfamilies of the Ras superfamily, based largely on amino acid sequence homologies. These subfamilies are often referred to in an abbreviated manner based on the most commonly studied member of the class.

The GTP binding domains of one subfamily of the Ras superfamily having substantial sequence homology is commonly referred to as the Ras family or Ras.

There are four isoforms of Ras proteins, expressed from three different genes: H-Ras (Harvey sarcoma viral oncogene), N-Ras (neuroblastoma oncogene), and the splice variants K-Ras4A and K-Ras4B (Kirsten sarcoma viral oncogene) (see Karnoub et al., supra).

The GTP binding domains of another subfamily of the Ras superfamily having substantial sequence homology is commonly referred to as the Rho family and includes proteins and groups of proteins referred to as Rho, Rac and Cdc42.

Ras Function and Pathways

All Ras isoforms share sequence identity in all of the regions that are responsible for GDP/GTP binding, GTPase activity, and effector interactions, suggesting a functional redundancy. However, studies clearly demonstrate that each Ras isoform can function in a unique, different way from the other Ras proteins in normal physiological processes as well as in pathogenesis (Quinlan et al., Future Oncol., 5: 105-116 (2009)).

Several cell surface receptors activate Ras, such as Receptor Tyrosine Kinases (RTKs), growth factor receptors, cytokine receptors and integrins.

Ras proteins cycle between ‘on’ and ‘off’ conformations that are conferred by the binding of GTP and GDP, respectively. Under physiological conditions, the transition between these two states is regulated by guanine nucleotide exchange factors (GEFs), such as Son of sevenless (Sos) (Bar-Sagi D, Trends Endocrin. Metab. 5, 165-169 (1994)), which promote the activation of Ras proteins by stimulating the exchange of GDP for GTP exchange, and by GTPase-activating proteins (GAPs), which accelerate Ras-mediated GTP hydrolysis to GDP.

The region of Sos functional for nucleotide exchange on Ras spans about 500 residues, and contains blocks of sequence that are conserved in Sos and other Ras-specific GEF's such as Cdc25, Sdc25 and Ras guanine-nucleotide-release factor (GRF) (Boguske et al, Nature 366, 643-654 (1993)).

Once activated, Ras initiates signaling of the “MAPK pathway” (also referred to as the Ras-RAF-MEK-MAPK/ERK1/2 pathway) that affects cell growth, differentiation, proliferation, apoptosis and migration. The MAPK pathway operates through a sequence of interactions among kinases. Activated by Ras in the “on”, GTP bound, state, a MAPK kinase kinase (MAPK3), such as Raf, MLK, or TAK, phosphorylates and activates a MAPK kinase, such as MEK, which then phosphorylates and increases the activity of one or more MAPKs, such as ERK1/2.

Ras activation also initiates signaling of the “Akt pathway” that affects cellular survival, proliferation, migration, anti-apoptotic and cell cycle regulation. Ras in the “on”, GTP bound, state, activates phosphoinositide 3-kinase (PI3K) which, in turn, induces the production of phosphatidylinositol (3,4,5) trisphosphates (PIP3). These lipids serve as plasma membrane docking sites for proteins that harbor pleckstrin-homology (PH) domains, including Akt (also known as protein kinase B or PKB) and its upstream activator PDK1. There are three highly related isoforms of Akt (Akt1, Akt2 and Akt3) that phosphorylate shared substrates, but isoform-specific Akt substrates have also been identified. At the membrane, Akt is phosphorylated and activated by PDK1, PDK2 and mTORC2. The Akt pathway can also be activated by receptor tyrosine kinases, integrins, B and T cell receptors, cytokine receptors and G-protein-coupled receptors that directly interact and activate PI3K.

Ras activation is also associated with signaling through other molecular pathways other than phosphoinositide 3-kinases (PI3Ks), such as Rac1 GEF and the Ral-guanine nucleotide dissociation stimulator (GDS). Regarding PI3K, that is part of the PI3K/AKT/mTOR pathway regulating intracellular signaling important for several cellular functions such as survival, anti-apoptotic and cell cycle regulation.

Ras Dysfunction is Causally Associated with Important Diseases and Disease Processes

Ras and its downstream pathways, including ERK1/2 and Akt, have been studied extensively. They are causally associated with a range of diseases, including certain cancers, inflammatory disorders, Ras-associated autoimmune leukoproliferative disorder, type II diabetes, and certain Rasopathies.

There is more than one distinct route to aberrant Ras activation including mutational activation of Ras itself, excessive activation of the wild-type protein through upstream signaling, and loss of a GAP function that is required to terminate activity of the protein.

One million deaths per year are attributed in the literature to mutations in K-Ras alone. (Frank McCormick. “K-Ras protein as a drug target.” Journal of Molecular Medicine (Berlin) 2016: 94: 253-258)

Ras is well documented in the literature as an oncogene. Ras oncogenes can initiate cancer in model organisms. Microinjection studies with antibodies that block Ras activity or block specific mutant alleles of Ras; ablation of K-Ras in mouse models of lung adenocarcinoma or pancreas cancer; and ablation of H-Ras all lead to tumor regression in mouse models.

About 30% (Prior IA, Lewis P D, Mattos C. Cancer Res. 2012 May 15; 72(10):2457-67) of human cancers have a mutated Ras protein with the most frequent mutations in residues G12, G13 and Q61. These oncogenic mutations result in impaired GTP hydrolysis and accumulation of Ras in the GTP-bound state leading to increased activation of Ras-dependent downstream effector pathways.

Table 1 summarizes recent data concerning the frequency of K-Ras and N-Ras mutations in an illustrative, but not exhaustive list, of human malignancies.

TABLE 1

| Mutation | Tumor Type | Frequency |
|---|---|---|
| K-Ras | Pancreas | 71% |
| K-Ras | Colon | 35% |
| K-Ras | Small intestine | 35% |
| K-Ras | Biliary tract | 28% |
| K-Ras | Endometrium | 22% |
| K-Ras | Lung | 20% |
| N-Ras | Skin (melanoma) | 20% |
| K-Ras | Cervix | 19% |
| K-Ras | Urinary tract | 16% |

Stephen AG, Esposito D, Bagni RK, McCormick F. Cancer Cell. 2014 Mar 17; 25(3):272-81.

Ras mutants, and in some cases Ras over-activation, are associated in the literature with a wide range of significant cancer associated processes including: cell proliferation, DNA checkpoint integrity, replicative stress related clonal selection, suppression of apoptosis, metabolic reprogramming, autophagy, microenvironment remodeling, immune response evasion, and metastatic processes. The detailed mechanisms, interdependencies, and frequency of these effects across different tumor types and stages of cancer development remain to be elucidated comprehensively.

Proliferative effects associated in the literature with oncogenic Ras include transcriptional upregulation of growth factors; upregulation of growth factor receptor expression; upregulation of integrins that promote proliferation and downregulation of those associated with cellular quiescence; upregulation of transcription factors required for cell cycle entry; acceleration through cell cycle transitions; downregulation of anti-proliferative TGFβ signaling; and the suppression of cyclin-dependent kinase inhibitors.

MAPK signaling has been shown to enhance programmed death-ligand 1 (PD-L1) expression in KRas mutant lung cancer cells. Thus, Ras mutations are associated with the suppression of immune responses to cancer. (Sumimoto et al., PLOS One 2016 Nov. 15; DOI:10.1371/journal.pone.0166626) Anti-PD-1 and anti-PD-L1 monoclonal antibodies have demonstrated clinical activity against tumors including non-small cell lung cancers.

Ras is also implicated through the ERK1/2 and Akt pathways as a cause of a range of pathological inflammatory conditions. In addition to ERK1/2 and Akt1, Akt2 and Akt3, the MAPKs ERK5, c-Jun N-terminal kinases (JNKs) and p38 isoforms have been implicated in inflammatory response. (Huang, et al. 2010, Protein Cell, 1(3), 218-226)

Ras is causally associated with inflammatory diseases including the following: rheumatoid arthritis (Abreu J R, de Launay D, Sanders M E, Grabiec A M, Sande van de MG, Tak P P, Reedquist K A: The Ras guanine nucleotide exchange factor RasGRF1 promotes matrix metalloproteinase-3 production in rheumatoid arthritis synovial tissue (Arthritis Res Ther. 2009, 11: R121-10.1186/ar2785), which is the most common cause of disability (Hootman J M, Brault M W, Helmick C G, Theis K A, Armour B S. Prevalence and most common causes of disability among adults-United States 2005, MMWR, 2009, 58(16):421-6); atherosclerosis (Fonarow G (2003), Cleve. Clin. J Med. 70: 431-434); inflammatory bowel disease (IBD), such as Crohn's disease (Ignacio C S, Sandvik A K, Bruland T, Andreu-Ballester J C, J. Crohns Colitis, 2017 Mar. 16. doi: 10); ulcerative colitis; spondyloarthropathies; idiopathic pulmonary fibrosis; juvenile arthritis; psoriasis; psoriatic arthritis; and others.

Ras has been causally associated with Ras-associated autoimmune leukoproliferative disorder, a nonmalignant clinical syndrome initially identified in a subset of putative autoimmune lymphoproliferative syndrome (ALPS) patients. (Katherin Calvo, et al. “JMML and RALD (Ras-associated autoimmune leukoproliferative disorder): common genetic etiology yet clinically distinct entities” Blood, 2015 Apr. 30; 125(18): 2753-2758)

Aberrant Ras signaling is causally implicated in the family of Rasopathies including neurofibromatosis type 1, Noonan's syndrome, and Costello syndrome.

Ras as a Therapeutic Molecular Target

Interference with Ras superfamily member signaling in cell based and animal models of the aforementioned diseases modulates disease processes.

Ras superfamily proteins, and particularly Ras and downstream pathway elements, have thus long been discussed as theoretical molecular targets for the development of targeted therapeutics. In theory, a molecule could serve as a therapeutic agent in diseases associated with aberrant Ras signaling if it could disrupt such Ras signaling.

In theory, it was recognized that a mechanism for downregulating aberrant Ras signaling could be to interfere with one or more steps in the Ras signaling process involving GTP binding in a manner that left Ras in other than an “on” configuration. In theory, a molecule could serve as therapeutic agent in diseases associated with abberant Ras signaling if it could disrupt such Ras signaling.

However, while this was a theoretical therapeutic strategy based on two widely accepted findings, it has also long been accepted by the scientific community that it would not be possible to achieve.

GTP and GDP had been found to bind to the GTP binding domain of Ras with single to double digit picomolar affinities.

The cellular concentration of GTP had been found to be substantially in excess of this range.

The widely accepted findings concerning the single to double digit picomolar range of affinities of GTP and GDP for the Ras GTP binding domain were determined by kinetic and filter binding measurements between Ras and radiolabeled GDP and GTP (Feuerstein J, Kalbitzer H R, John J, Goody R S, Wittinghofer A. Eur. J Biochem., 1987 Jan. 2, 162(1):49-55; and John J, Sohmen R, Feuerstein J, Linke R, Wittinghofer A, Goody R S. Biochemistry, 1990 Jun. 26, 29(25):6058-65).

Based on these findings, and often citing them, the GTP binding domain of Ras has widely been accepted and reported in preeminent journal editorials, reviews, and research papers to be “undruggable.” (Papke B, Der C J., Science, 2017 Mar. 17, 355(6330):1158-1163; Stephen A G, Esposito D, Bagni R K, McCormick F, Cancer Cell, 2014 Mar. 17, 25(3):272-81; and Ostrem J M, Shokat K M, Nat. Rev. DrugD iscov., 2016 Nov. 15(11):771-785)

Accordingly, research concerning targeted Ras therapeutics has focused on domains of the Ras protein other than the GTP binding site. These efforts have included, for example, seeking to develop farnesyltransferase inhibitors (FTIs) that prevent Ras attachment to the inner side of the plasma membrane, and to develop molecules that compete with the interaction of Ras with the exchange factor Sos or downstream effectors.

Thus, it has been thought that a molecule could not be developed to reversibly compete with GTP binding to the GTP binding domain of Ras. Compounds that do so, however, would fill a need in the field.

The Rho Family Function and Pathways

The Rho subfamily of the Ras superfamily currently includes approximately 22 proteins most of which scientists commonly divide into subgroups including those referred to as Cdc42, Rac, and Rho. (Boureux A, Vignal E, Faure S, Fort P (2007). “Evolution of the Rho family of ras-like GTPases in eukaryotes”. Mol Biol Evol 24 (1): 203-16).

The three most commonly studied members of the Rho subfamily have been Cdc42, Rac1, and RhoA.

The Cdc42 group includes Cdc42, TC10, TCL, Chip, and Wrch-1.

The Rac group includes Rac1, Rac2, Rac3, and RhoG.

The RhoA group includes RhoA, RhoB, and RhoC.

Other Rho subfamily GTPases not included in the Cdc42, Rac, or Rho groups include RhoE/Rnd3, RhoH/TTF, Rif, RhoBTB1, RhoBTB2, Miro-1, Miro-2, RhoD, Rnd1, and Rnd2.

Like other Ras superfamily proteins, the Rho subfamily GTPases cycle between ‘on’ and ‘off’ conformations that are conferred by the binding of GTP and GDP, respectively. Under physiological conditions, the transition between these two states is regulated by guanine nucleotide exchange factors (GEFs), which promote the activation of Rho subfamily proteins by stimulating the release of GDP and the binding of GTP, and by GTPase-activating proteins (GAPs), which accelerate Rho subfamily member-mediated GTP hydrolysis to GDP. Guanine nucleotide dissociation inhibitors (GDIs) proteins form a large complex with the Rho protein, helping to prevent diffusion within the membrane and into the cytosol and thus acting as an anchor and allowing tight spatial control of Rho activation.

The Rho subfamily members are intracellular proteins that affect a large number of downstream pathways broadly involving cytoskeleton organization, cell polarity, migration, transcription and proliferation, and, more particularly, membrane and vesicular trafficking, cell cycling, microtubule stability, actin membrane linkages, actin polymerization, myosin phosphorylation, API dependent gene expression, cell adhesion, cell contractility, cell adhesion, and MTOC orientation. (Martin Schwartz. “Rho Signalling at a Glance.” Journal of Cell Science. 2004: (117:pp. 5457-5458) and (Bustelo X R, Sauzeau V, Berenjeno I M (2007). “GTP-binding proteins of the Rho/Rac family: regulation, effectors and functions in vivo” BioEssays. 29 (4): 356-370).

Rho Family Dysfunction is Causally Associated with Important Diseases

Rho subfamily GTPases have been reported to contribute to most steps of cancer initiation and progression including the acquisition of unlimited proliferation potential, survival and evasion from apoptosis, angiogenesis, tissue invasion, motility, and the establishment of metastases. (Matteo Parri and Paolo Chiarugi. “Rac and Rho GTPases in Cancer Cell Motility Control.” Cell Communication and Signalling. 2010(8:23))

High Rho subfamily protein levels are frequently associated with human tumors. High RhoA levels have been associated with human liver, skin, colon, ovarian, bladder, gastric, esophageal squamous cell, testicular, and breast cancer. High Rho B, C, or H levels have been associated with breast, squamous cell, pancreatic, breast, liver, ovarian, head and neck, prostate, non-small cell lung, and gastric cancers and melanoma metastase. High Rac1 levels have been associated with human testicular, gastric, breast, and squamous cell cancers. High Rac2 or Rac3 have been associated with breast colon, head and neck, and squamous cell cancers. (Matteo Parri and Paolo Chiarugi. “Rac and Rho GTPases in Cancer Cell Motility Control.” Cell Communication and Signalling. 2010(8:23). Gain-of-function mutations such as P29S of Rac1 were detected in melanoma, breast, head and neck cancers (Alan J K, Lundquist E A. Mutationally activated Rho GTPases in cancer. Small GTPases. 2013 July-September; 4(3):159-63)

Unlike Ras proteins, which are frequently mutated in cancer (around 30%), Rho subfamily proteins themselves are generally not found to be mutated in cancer. Rather, aberrant activity of Rho subfamily proteins in cancer appears to occur by overexpression of these proteins or by aberrant regulation of molecules that control their activity, such as activation or overexpression of GEFs and inactivation or loss of GAPs or GDIs (Alan J K, Lundquist E A. Mutationally activated Rho GTPases in cancer. Small GTPases. 2013 July-September; 4(3):159-63).

Interactions between Rac and Rho proteins are believed to modulate certain forms of mesenchymal and amoeboid cell movement associated with cancer.

Rho subfamily associated kinases (ROCK1 and ROCK2) are implicated as mediators of multiple profibrotic processes including those associated with idiopathic pulmonary fibrosis. (Knipe R S, Tager E M, and Liao J K. “The Rho kinases: critical mediators of multiple profibrotic processes and rational targets for new therapies for pulmonary fibrosis.” Pharmacol Rev. 2015 67(1):103-17.)

Rho Family Members as Therapeutic Molecular Targets

Given their roles in disease processes, Rho subfamily members have been identified as potential Therapeutic Molecular Targets.

Rho subfamily members have been identified as potential Therapeutic Molecular Targets in cancer.

Rho subfamily members have been identified as potential Therapeutic Molecular Targets in fibrotic disease.

### 3. SUMMARY

As disclosed in International Application No. PCT/US2018/038613 and U.S. patent application Ser. No. 16/013,872, the affinity of GTP for the GTP binding domain of K-Ras utilizing a Scintillation Proximity Assay (SPA) and MicroScale Thermophoresis (MST) has been measured. These methods were not available when Wittinghofer and colleagues (referenced above) undertook their studies. The contents of International Application No. PCT/US2018/038613 and U.S. patent application Ser. No. 16/013,872 are incorporated by reference herein.

In SPA and MST studies, it was found and is disclosed herein that the affinity of GTP for the K-Ras GTP binding domain across wild type and mutant K-Ras is in the range of 100-465 nanomolar. This therefore led to the novel, unanticipated conclusion that compounds such as small molecules could be discovered that would bind to a Ras GTP binding domain and compete with the binding of GTP to Ras.

Provided herein is a cell-free assay for the identification of small molecules that bind to the GTP binding domain and compete with GTP binding to, for example, wild-type KRas, KRas G12D mutant, KRas G12C mutant, KRas Q61H mutant, Rac1 and RhoA proteins.

Provided herein is a cell-free assay for the identification of small molecules that bind to the GTP binding domain and compete with GTP binding to, for example, wild-type KRas, KRas G12D mutant, KRas G12C mutant, KRas Q61H mutant, HRas, Rac1 and RhoA proteins.

Utilizing this assay as a screening and analytical tool, over 80 small molecules are provided that bind competitively with GTP to a Ras GTP binding domain, thereby confirming the significance of the novel and unanticipated binding affinity findings. Provided herein is a method of testing the affinity of a compound for a Ras GTP binding domain comprising the cell-free assay.

Utilizing this assay, it was also demonstrated that over 80 small molecules provided herein also bind competitively with GTP to both Rac and Rho GTPase binding domains. Provided herein is a method of testing the affinity of a compound for Rac and Rho GTP binding domains comprising the cell-free assay.

It is also demonstrated herein in cell-based assays that certain of these molecules inhibit activation of the ERK1/2 pathway and downregulate the proliferation of different human tumor cell lines.

It is also demonstrated herein in cell-based assays that certain of these molecules inhibit activation of the AKT pathway and downregulate the proliferation of different human tumor cell lines.

It is also demonstrated herein in cell-based assays that certain of these molecules inhibit activation of the ERK1/2 pathway and AKT pathway and downregulate the proliferation of different human tumor cell lines.

It is further demonstrated herein in cell-based assays that certain of these molecules downregulate the secretion of inflammatory cytokines.

It has also been discovered and disclosed in International Application No. PCT/US2018/038613 and U.S. patent application Ser. No. 16/013,872 that certain amino acids in the Ras GTP binding domain enable the heretofore unanticipated GTP-competitive binding to that domain for compounds, such as small molecules. They include the following amino acids: Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147, and also Mg202 which is required for GTP binding.

It has also been discovered and disclosed in International Application No. PCT/US2018/038613 and U.S. patent application Ser. No. 16/013,872 that certain amino acids in the Rac1 GTP binding domain enable the heretofore unanticipated GTP-competitive binding to that domain for compounds, such as small molecules. They include the following amino acids: Gly12, Ala13, Gly15, Lys16, Thr17, Cys18, Leu19, Phe28, Ile33, Pro34, Val36, Ala59, Thr115, Lys116, Asp118, Leu119, Cys157, Ala159, and also Mg202 which is required for GTP binding.

It has also been discovered and disclosed in International Application No. PCT/US2018/038613 and U.S. patent application Ser. No. 16/013,872 that certain amino acids in the RhoA GTP binding domain enable the heretofore unanticipated GTP-competitive binding to that domain for compounds, such as small molecules. They include the following amino acids: Gly14, Ala15, Cys16, Gly17, Lys18, Thr19, Cys20, Phe30, Pro31, Glu32, Tyr34, Val35, Pro36, Thr37, Asp59, Lys118, Asp120, Lys162, and also Mg202 which is required for GTP binding.

Therefore, provided herein is a method of inhibiting the function of Ras, comprising administering to a subject a compound which competitively binds to a Ras GTP binding domain. In one embodiment, the compound for use in the method has a binding affinity (K_{d}) to a Ras GTP binding domain of less than 10 μM. In one embodiment, the compound for use in the method has a binding affinity (K_{d}) to a Ras GTP binding domain of less than 1 μM. In one embodiment, the compound for use in the method has a binding affinity (K_{d}) to a Ras GTP binding domain of less than 500 nM. In one embodiment, the compound for use in the method has a binding affinity (K_{d}) to a Ras GTP binding domain of less than 465 nM. In one embodiment, the compound for use in the method has a binding affinity (K_{d}) to a Ras GTP binding domain of less than 270 nM. In one embodiment, the compound for use in the method has a binding affinity (K_{d}) to a Ras GTP binding domain of less than 200 nM. In one embodiment, the compound for use in the method has a binding affinity (K_{d}) to a Ras GTP binding domain of less than 150 nM. In one embodiment, the compound for use in the method has a binding affinity (K_{d}) to a Ras GTP binding domain of less than 100 nM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain with a binding affinity (K_{d}) of less than 465 nM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain with a binding affinity (K_{d}) of less than 270 nM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a highly conserved Ras GTP binding domain with a binding affinity (K_{d}) of less than 10 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In an assay described herein, e.g., a cell-free assay, the compound for use in the method inhibits Ras. In one embodiment, the compound for use in the method inhibits Ras and has an IC₅₀ value of less than 10 μM. In one embodiment, the compound for use in the method inhibits Ras and has an IC₅₀ value of less than 1 μM. In one embodiment, the compound for use in the method inhibits Ras and has an IC₅₀ value of less than 500 nM. In one embodiment, the compound for use in the method inhibits Ras and has an IC₅₀ value of less than 465 nM. In one embodiment, the compound for use in the method inhibits Ras and has an IC₅₀ value of less than 270 nM. In one embodiment, the compound for use in the method inhibits Ras and has an IC₅₀ value of less than 200 nM. In one embodiment, the compound for use in the method inhibits Ras and has an IC₅₀ value of less than 150 nM. In one embodiment, the compound for use in the method inhibits Ras and has an IC₅₀ value of less than 100 nM. In one embodiment, the compound for use in the method inhibits Ras with greater than 15% inhibition at 20 μM. In one embodiment, the compound for use in the method inhibits Ras with greater than 25% inhibition at 20 μM. In one embodiment, the compound for use in the method inhibits Ras with greater than 50% inhibition at 20 μM. In one embodiment, the compound for use in the method inhibits Ras with greater than 75% inhibition at 20 μM. In one embodiment, the compound for use in the method inhibits Ras with greater than 80% inhibition at 20 μM. In one embodiment, the compound for use in the method inhibits Ras with greater than 85% inhibition at 20 μM. In one embodiment, the compound for use in the method inhibits Ras with greater than 90% inhibition at 20 μM. In one embodiment, the compound for use in the method inhibits Ras with greater than 95% inhibition at 20 μM. In one embodiment, the compound for use in the method inhibits Ras with greater than 99% inhibition at 20 μM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain and inhibits Ras with a corresponding IC₅₀ value of less than 465 nM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain and inhibits Ras with a corresponding IC₅₀ value of less than 270 nM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a highly conserved Ras GTP binding domain and inhibits Ras with a corresponding IC₅₀ value of less than 10 μM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a highly conserved Ras GTP binding domain and inhibits Ras with greater than 15% inhibition at 20 μM, such as greater than 25% inhibition at 20 μM. In one embodiment, the method of inhibiting the function of Ras, comprises administering to a subject a compound which binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a highly conserved Ras GTP binding domain and inhibits Ras with greater than 50% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In one embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

Also provided herein is a method of inhibiting the function of Rho, comprising administering to a subject a compound which competitively binds to a Rho GTP binding domain. In one embodiment, the compound for use in the method has a binding affinity (K_{d}) to a Rho GTP binding domain of less than 10 μM. In one embodiment, the compound for use in the method has a binding affinity (K_{d}) to a Rho GTP binding domain of less than 1 μM. In one embodiment, the compound for use in the method has a binding affinity (K_{d}) to a Rho GTP binding domain of less than 500 nM. In one embodiment, the compound for use in the method has a binding affinity (K_{d}) to a Rho GTP binding domain of less than 270 nM. In one embodiment, the compound for use in the method has a binding affinity (K_{d}) to a Rho GTP binding domain of less than 200 nM. In one embodiment, the compound for use in the method has a binding affinity (K_{d}) to a Rho GTP binding domain of less than 150 nM. In one embodiment, the compound for use in the method has a binding affinity (K_{d}) to a Rho GTP binding domain of less than 130 nM. In one embodiment, the compound for use in the method has a binding affinity (K_{d}) to a Rho GTP binding domain of less than 100 nM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain with a binding affinity (K_{d}) of less than 270 nM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain with a binding affinity (K_{d}) of less than 130 nM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a highly conserved Rho GTP binding domain with a binding affinity (K_{d}) of less than 10 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In an assay described herein, e.g., a cell-free assay, the compound for use in the method inhibits Rho. In one embodiment, the compound for use in the method inhibits Rho and has an IC₅₀ value of less than 10 μM. In one embodiment, the compound for use in the method inhibits Rho and has an IC₅₀ value of less than 1 μM. In one embodiment, the compound for use in the method inhibits Rho and has an IC₅₀ value of less than 500 nM. In one embodiment, the compound for use in the method inhibits Rho and has an IC₅₀ value of less than 270 nM. In one embodiment, the compound for use in the method inhibits Rho and has an IC₅₀ value of less than 200 nM. In one embodiment, the compound for use in the method inhibits Rho and has an IC₅₀ value of less than 150 nM. In one embodiment, the compound for use in the method inhibits Rho and has an IC₅₀ value of less than 130 nM. In one embodiment, the compound for use in the method inhibits Rho and has an IC₅₀ value of less than 100 nM. In one embodiment, the compound for use in the method inhibits Rho with greater than 15% inhibition at 20 μM. In one embodiment, the compound for use in the method inhibits Rho with greater than 25% inhibition at 20 μM. In one embodiment, the compound for use in the method inhibits Rho with greater than 50% inhibition at 20 μM. In one embodiment, the compound for use in the method inhibits Rho with greater than 75% inhibition at 20 μM. In one embodiment, the compound for use in the method inhibits Rho with greater than 80% inhibition at 20 μM. In one embodiment, the compound for use in the method inhibits Rho with greater than 85% inhibition at 20 μM. In one embodiment, the compound for use in the method inhibits Rho with greater than 90% inhibition at 20 μM. In one embodiment, the compound for use in the method inhibits Rho with greater than 95% inhibition at 20 μM. In one embodiment, the compound for use in the method inhibits Rho with greater than 99% inhibition at 20 μM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain and inhibits Rho with a corresponding IC₅₀ value of less than 270 nM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain and inhibits Rho with a corresponding IC₅₀ value of less than 130 nM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a highly conserved Rho GTP binding domain and inhibits Rho with a corresponding IC₅₀ value of less than 10 μM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a highly conserved Rho GTP binding domain and inhibits Rho with greater than 15% inhibition at 20 μM, such as greater than 25% inhibition at 20 μM. In one embodiment, the method of inhibiting the function of Rho, comprises administering to a subject a compound which binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a highly conserved Rho GTP binding domain and inhibits Rho with greater than 50% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

Also provided herein is a method of inhibiting the function of Rac, comprising administering to a subject a compound which competitively binds to a Rac GTP binding domain. In one embodiment, the compound for use in the method has a binding affinity (K_{d}) to a Rac GTP binding domain of less than 10 μM. In one embodiment, the compound for use in the method has a binding affinity (K_{d}) to a Rac GTP binding domain of less than 1 μM. In one embodiment, the compound for use in the method has a binding affinity (K_{d}) to a Rac GTP binding domain of less than 500 nM. In one embodiment, the compound for use in the method has a binding affinity (K_{d}) to a Rac GTP binding domain of less than 270 nM. In one embodiment, the compound for use in the method has a binding affinity (K_{d}) to a Rac GTP binding domain of less than 200 nM. In one embodiment, the compound for use in the method has a binding affinity (K_{d}) to a Rac GTP binding domain of less than 170 nM. In one embodiment, the compound for use in the method has a binding affinity (K_{d}) to a Rac GTP binding domain of less than 150 nM. In one embodiment, the compound for use in the method has a binding affinity (K_{d}) to a Rac GTP binding domain of less than 100 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain with a binding affinity (K_{d}) of less than 270 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain with a binding affinity (K_{d}) of less than 170 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rac GTP binding domain with a binding affinity (K_{d}) of less than 10 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In an assay described herein, e.g., a cell-free assay, the compound for use in the method inhibits Rac. In one embodiment, the compound for use in the method inhibits Rac and has an IC₅₀ value of less than 10 μM. In one embodiment, the compound for use in the method inhibits Rac and has an IC₅₀ value of less than 1 μM. In one embodiment, the compound for use in the method inhibits Rac and has an IC₅₀ value of less than 500 nM. In one embodiment, the compound for use in the method inhibits Rac and has an IC₅₀ value of less than 270 nM. In one embodiment, the compound for use in the method inhibits Rac and has an IC₅₀ value of less than 200 nM. In one embodiment, the compound for use in the method inhibits Rac and has an IC₅₀ value of less than 170 nM. In one embodiment, the compound for use in the method inhibits Rac and has an IC₅₀ value of less than 150 nM. In one embodiment, the compound for use in the method inhibits Rac and has an IC₅₀ value of less than 100 nM. In one embodiment, the compound for use in the method inhibits Rac with greater than 15% inhibition at 20 μM. In one embodiment, the compound for use in the method inhibits Rac with greater than 25% inhibition at 20 μM. In one embodiment, the compound for use in the method inhibits Rac with greater than 50% inhibition at 20 μM. In one embodiment, the compound for use in the method inhibits Rac with greater than 75% inhibition at 20 μM. In one embodiment, the compound for use in the method inhibits Rac with greater than 80% inhibition at 20 μM. In one embodiment, the compound for use in the method inhibits Rac with greater than 85% inhibition at 20 μM. In one embodiment, the compound for use in the method inhibits Rac with greater than 90% inhibition at 20 μM. In one embodiment, the compound for use in the method inhibits Rac with greater than 95% inhibition at 20 μM. In one embodiment, the compound for use in the method inhibits Rac with greater than 99% inhibition at 20 μM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain and inhibits Rac with a corresponding IC₅₀ value of less than 270 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain and inhibits Rac with a corresponding IC₅₀ value of less than 270 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain and inhibits Rac with a corresponding IC₅₀ value of less than 170 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain and inhibits Rac with a corresponding IC₅₀ value of less than 170 nM. In one embodiment, the method of inhibiting the function of Rac, comprises administering to a subject a compound which binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rac GTP binding domain and inhibits Rac with a corresponding IC₅₀ value of less than 10 μM. In one embodiment, the method of inhibiting the function of Rac, comprises administering to a subject a compound which binds to one or more of Gly12, Ala 13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rac GTP binding domain and inhibits Rac with greater than 15% inhibition at 20 μM, such as greater than 25% inhibition at 20 μM. In one embodiment, the method of inhibiting the function of Rac, comprises administering to a subject a compound which binds to one or more of Gly12, Ala 13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rac GTP binding domain and inhibits Rac with greater than 50% inhibition at 20 μM. In one embodiment, the method of inhibiting the function of Rac, comprises administering to a subject a compound which binds to one or more of Gly12, Ala 13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rac GTP binding domain and inhibits Rac with greater than 99% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is Rac. In one embodiment the Rac is RAC1; RAC2; RAC3 or RHOG. In one embodiment, the Rac is RAC1. In another embodiment, the Rac is a mutant form of a Rac described herein.

In one embodiment, provided herein is a method of treating or preventing cancer by administering a compound that inhibits one or more members of the Ras superfamily. In one embodiment, provided herein is a method of treating or preventing cancer by administering a compound that inhibits the binding of GTP to the GTP binding domain of one or more members of the Ras superfamily. In one embodiment, provided herein is a method of treating or preventing inflammation by administering a compound that inhibits one or more members of the Ras superfamily. In one embodiment, provided herein is a method of treating or preventing inflammation by administering a compound that inhibits the binding of GTP to the GTP binding domain of one or more members of the Ras superfamily. In one embodiment, provided herein is a method of treating or preventing a rasopathy by administering a compound that inhibits one or more members of the Ras superfamily. In one embodiment, provided herein is a method of treating or preventing a rasopathy by administering a compound that inhibits the binding of GTP to the GTP binding domain of one or more members of the Ras superfamily. In one embodiment, provided herein is a method of treating or preventing fibrotic disease by administering a compound that inhibits one or more members of the Ras superfamily. In one embodiment, provided herein is a method of treating or preventing fibrotic disease by administering a compound that inhibits the binding of GTP to the GTP binding domain of one or more members of the Ras superfamily. In one embodiment, provided herein is a method of treating or preventing cancer by administering a compound that inhibits the binding of GTP to a Ras GTP binding domain. In one embodiment, provided herein is a method of treating or preventing inflammation by administering a compound that inhibits the binding of GTP to a Ras GTP binding domain. In one embodiment, provided herein is a method of treating or preventing a rasopathy by administering a compound that inhibits the binding of GTP to a Ras GTP binding domain. In one embodiment, provided herein is a method of treating or preventing Ras-associated autoimmune leukoproliferative disorder by administering a compound that inhibits the binding of GTP to a Ras GTP binding domain. In one embodiment, provided herein is a method of treating or preventing fibrotic disease by administering a compound that inhibits the binding of GTP to a Ras GTP binding domain. In one embodiment, provided herein is a method of treating or preventing cancer by administering a compound that inhibits the binding of GTP to a Rho GTP binding domain. In one embodiment, provided herein is a method of treating or preventing inflammation by administering a compound that inhibits the binding of GTP to a Rho GTP binding domain. In one embodiment, provided herein is a method of treating or preventing a rasopathy by administering a compound that inhibits the binding of GTP to a Rho GTP binding domain. In one embodiment, provided herein is a method of treating or preventing fibrotic disease by administering a compound that inhibits the binding of GTP to a Rho GTP binding domain. In one embodiment, provided herein is a method of treating or preventing cancer by administering a compound that inhibits the binding of GTP to a Rac GTP binding domain. In one embodiment, provided herein is a method of treating or preventing inflammation by administering a compound that inhibits the binding of GTP to a Rac GTP binding domain. In one embodiment, provided herein is a method of treating or preventing a rasopathy by administering a compound that inhibits the binding of GTP to a Rac GTP binding domain. In one embodiment, provided herein is a method of treating or preventing fibrotic disease by administering a compound that inhibits the binding of GTP to a Rac GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

Provided herein are compounds which bind to a Ras GTP binding domain and compete with the binding of GTP to Ras. In one embodiment, the compounds also inhibit phosphorylation of MAPK, in particular ERK1/2, Akt (for example, Akt1, Akt2 and Akt3) cellular proliferation, secretion of IL-6 or TNF-α cytokines. The compounds provided herein are therefore useful in compositions and methods of treating cancer, inflammatory diseases, Ras-associated autoimmune leukoproliferative disorder and rasopathies.

Provided herein are compounds which bind to a Rac GTP binding domain and compete with the binding of GTP to Rac. In one embodiment, the compounds also inhibit the ERK1/2 and Akt signaling pathways. In one embodiment, the compounds also inhibit the ROCK signaling pathway. The compounds provided herein are therefore useful in compositions and methods of treating cancer, inflammatory diseases and fibrotic disease.

Provided herein are compounds which bind to a Rho GTP binding domain and ERK1/2 and Akt signaling pathways. In one embodiment, the compounds also inhibit the ROCK signaling pathway. The compounds provided herein are therefore useful in compositions and methods of treating cancer, inflammatory diseases and fibrotic disease.

In one embodiment, the compounds provided herein inhibit GTP binding to one or more members of the Ras superfamily. In one embodiment, the compounds provided herein inhibit GTP binding to Ras. In one embodiment, the compounds provided herein inhibit GTP binding to Rho. In one embodiment, the compounds provided herein inhibit GTP binding to Rac. In one embodiment, the compounds provided herein inhibit GTP binding to Ras and Rho. In one embodiment, the compounds provided herein inhibit GTP binding to Ras and Rac. In one embodiment, the compounds provided herein inhibit GTP binding to Rho and Rac. In one embodiment, the compounds provided herein inhibit GTP binding to Ras, Rho and Rac.

In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons.

### 4. DETAILED DESCRIPTION

### 4.1. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating cancer, inflammation or rasopathies.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

As used herein, the IC₅₀ refers to an amount, concentration or dosage of a particular test compound that achieves a 50% inhibition of a maximal response in an assay that measures such response.

As used herein, the K_{d} refers to the measured equilibrium dissociation constant between a compound (or ligand) and a protein (or binding domain of a protein).

As used herein, “Ras superfamily” means the protein superfamily of small guanosine triphosphatases (GTPases) which consists of the five main families Ras, Rho, Rab, Ran and Arf, or mutants thereof. Subfamilies of the five main families are also included, e.g., the Rac subfamily of the Rho main family.

As used herein, “Ras” or “Ras family” or “Ras subfamily” or “Ras group” means DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2, or mutants thereof.

As used herein, “Rho” or “Rho family” or “Rho subfamily” or “Rho group” means RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or mutants thereof.

As used herein, “Rac” or “Rac family” or “Rac subfamily” or “Rac group” means RAC1; RAC2; RAC3; RHOG, or mutants thereof.

As used herein, “GTP binding site” or “GTP binding domain” both mean the region of a protein which binds GTP, and the surrounding region of said protein in which another compound may bind, wherein such binding blocks the ability of GTP to bind to said protein.

As used herein, “GDP binding site” or “GDP binding domain” both mean the region of a protein which binds GDP, and the surrounding region of said protein in which another compound may bind, wherein such binding blocks the ability of GDP to bind to said protein.

As used herein, “guanosine binding region” means a region of a protein which is part of the GDP binding domain or GTP binding domain, that mediates interaction with the guanosine portion of GDP or GTP.

As used herein, “metal region” means a region of a protein which is part of the GDP binding domain or GTP binding domain, that is proximal to a magnesium (Mg202) binding site.

As used herein, “alternative Tyr32 conformation” means the conformation of the GTP or GDP binding domain in the region of Tyr 32 in KRas crystal structure PDB code:3gft in comparsion to the KRas crystal structure PDB code:4epr.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter enzymatic and biological activities of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as chiral reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included. For example, Formula A includes, but is not limited to, the three tautomeric structures below.

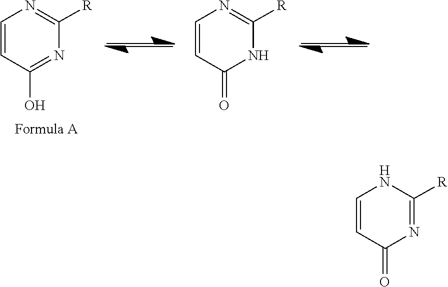

As used herein, the abbreviations for any protective groups, amino acids and other compounds, are, unless indicated otherwise, in accord with their common usage, recognized abbreviations, the IUPAC-IUB Commission on Biochemical Nomenclature (see, (1972) Biochem. 11:942-944), or the IUPAC Nomenclature of Organic Chemistry (see, Favre H A and Powell W H, Nomenclature of Organic Chemistry: IUPAC Recommendations and Preferred Names 2013, Cambridge, UK: The Royal Society of Chemistry, 2013: Print ISBN 978-0-85404-182-4, PDF eISBN 978-1-84973-306-9, DOI 10.1039/9781849733069; Nomenclature of Organic Chemistry, Sections A, B, C, D, E, F, and H, Pergamon Press, Oxford, 1979. Copyright 1979 IUPAC; and A Guide to IUPAC Nomenclature of Organic Compounds (Recommendations 1993), 1993, Blackwell Scientific publications, Copyright 1993 IUPAC).

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician. A therapeutically effective amount of a compound provided herein can be administered in one dose (i.e., a single dose administration) or divided and administered over time (i.e., continuous administration or multiple sub-dose administration). Single dose administration, continuous administration, or multiple sub-dose administration can be repeated, for example, to maintain the level of the compound in a biological molecule (e.g., a protein, enzyme, RNA, or DNA), cell, tissue, system, animal, or human.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 22nd ed.; Loyd et al., Eds.; The Pharmaceutical Press, 2012; Handbook of Pharmaceutical Excipients, 7th ed.; Rowe et al., Eds.; The Pharmaceutical Press, 2012; Handbook of Pharmaceutical Additives, 3rd ed.; Ash and Ash Eds.; Synapse Information Resources, Inc., 2007; Pharmaceutical Preformulation and Formulation, 2nd ed.; Gibson Ed.; CRC Press LLC, 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “percent by weight” or “% by weight” refers to the weight of a specified component (e.g., an active compound or excipient) in a composition (e.g., a pharmaceutical composition) as a percentage of the total weight of the composition. Thus, the sum of the weight percentages of all the components in a composition is 100%.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “racemate” is understood to refer to an equimolar mixture of a pair of enantiomers. It does not exhibit optical activity. The chemical name or formula of a racemate is distinguished from those of the enantiomers by the prefix (±)-, or rac- (or rac. or racem-) or by the symbols RS and SR. See IUPAC Recommendations 1996, Basic Terminology of Stereochemistry, Pure & Appl. Chem., Vol. 68, No. 12, pp. 2193-2222, 1996.

Racemic compounds disclosed herein that contain two asymmetric centers with known relative configuration are named using the configurational descriptors R,S or R,R, preceded by the prefix rac-. For example, Racemic Compound A below is named rac-(1R,3S)-1-bromo-3-chlorocyclohexane and is a 1:1 mixture of enantiomers (1R,3S)-1-bromo-3-chlorocyclohexane and (1S,3R)-1-bromo-3-chlorocyclohexane.

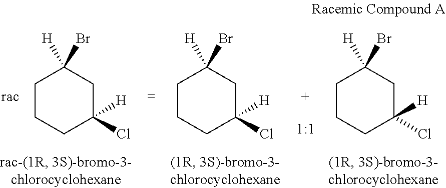 [SMILES: Cl[C@H]1CCC[C@@H](Br)C1 ; Cl[C@H]1CCC[C@@H](Br)C1 ; Cl[C@@H]1CCC[C@@H](Br)C1]

Lower case r/s stereo descriptors are used to describe pseudo-asymmetric centers, according to Cahn-Ingold-Prelog Rules (see R. S. Cahn, C. K. Ingold and V. Prelog, Angew. Chem. Internat. Ed. Eng. 5, 385-415, 511 (1966); and V. Prelog and G. Helmchen, Angew. Chem Internat. Ed. Eng. 21, 567-583 (1982)). For example, Compound B below is named (1s,4s)-1-bromo-4-chlorocyclohexane.

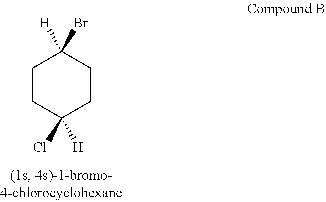

Compounds disclosed and named herein were named using the Structure>Name feature in ChemDraw® Professional version 20.0.0.38.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (14N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I) iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium. In some embodiments, a pharmaceutically acceptable derivative of a compound is an isotopic variant.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an isotopic variant thereof, or a pharmaceutically acceptable salt thereof; or a pharmaceutically acceptable solvate thereof” has the same meaning as the phrase “an isotopic variant of the compound referenced therein; or a pharmaceutically acceptable salt of the compound referenced therein; or a pharmaceutically acceptable salt of an isotopic variant of the compound referenced therein; or a pharmaceutically acceptable solvate of the compound referenced therein; or a pharmaceutically acceptable solvate of an isotopic variant of the compound referenced therein; or a pharmaceutically acceptable solvate of a pharmaceutically acceptable salt of the compound referenced therein; or a pharmaceutically acceptable solvate of a pharmaceutically acceptable salt of an isotopic variant of the compound referenced therein or its variant or its variant.”

### 4.2. RAS GTP Binding Domain

Data Concerning the Affinity of GTP to the Ras GTP Binding Domain

As disclosed in International Application No. PCT/US2018/038613 and U.S. patent application Ser. No. 16/013,872, the affinity of GTP for the GTP binding domain of K-Ras utilizing a Scintillation Proximity Assay (SPA) and MicroScale Thermophoresis (MST) has been measured. These methods were not available when Wittinghofer and colleagues undertook their studies.

In SPA and MST studies, it was found and is disclosed in International Application No. PCT/US2018/038613 and U.S. patent application Ser. No. 16/013,872 that the affinity of GTP for the K-Ras GTP binding domain across wild type and mutant K-Ras is in the range of 100-465 nanomolar (see results in Table 2). This therefore led to the novel, unanticipated conclusion that compounds such as small molecules could be discovered that would bind to a Ras GTP binding domain and compete with the binding of GTP to Ras. SPA and MST studies performed on the Rac-1 and Rho-A members of the Rho subfamily show that the affinity of GTP for the binding domain of these Rho subfamily members is in the range of 120-170 nanomolar (see results in Table 2).

TABLE 2
MST and SPA Results for GTP Affinity for K-Ras and
Rho Subfamily Binding Domains

| Protein | MST | SPA |
|---|---|---|
| K-Ras (wild type) | 463 ± 2 nM | 243 ± 15 nM |
| K-Ras (G12D) | 244 ± 12 nM | 270 ± 15 nM |
| K-Ras (G12C) | 207 ± 46 nM | 258 ± 18 nM |
| K-Ras (Q61H) | 157 ± 21 nM | 118 ± 11 nM |
| Rac-1 | 166 ± 10 nM | 151 ± 14 nM |
| Rho-A | 130 ± 5 nM | 129 ± 12 nM |

See Khawaja et al., “Scintillation proximity assay in lead discovery”, Expert Opin. Drug Discov., 2008 November; 3(11):1267-80 regarding SPA procedures. See the following references regarding MST technology: Wienken et al., Nature Communications (2010), Protein binding assays in biological liquids using MicroScale Thermophoresis; Jerabek-Willemsen et al., ASSAY and Drug Development Technologies (2011), Molecular interaction studies using MicroScale Thermophoresis; Lin et al., Cell (2012), Inhibition of basal FGF receptor signaling by dimeric Grb2; Seidel et al., Angewandte Chemie (2012), Label-Free MicroScale Thermophoresis discriminates sites and affinity of protein-ligand binding; Seidel et al., Methods (2012), MicroScale Thermophoresis quantifies biomolecular interactions under previously challenging conditions; Parker & Newstead, Nature (2014), Molecular basis of nitrate uptake by the plant nitrate transporter NRT1.1; and Jerabek-Willemsen et al., Journal of Molecular Structure (2014), MicroScale Thermophoresis: Interaction analysis and beyond.

The Discovery of Small Molecules that Bind to a Ras GTP Binding Domain in Competition with GTP

Provided herein is an assay for the identification of small molecules that bind to a Ras GTP binding domain in competition with GTP.

The useful approach for drug discovery described herein and in International Application No. PCT/US2018/038613 and U.S. patent application Ser. No. 16/013,872 is to identify small molecule inhibitors that will compete and block interactions between GTP and GTP-binding proteins. By interacting with the GDP/GTP-binding site of GTP-binding proteins small molecules so identified may induce a GDP-bound or other inactive conformation of the GTP-binding proteins and thus block signal transduction pathways downstream of the GTP-binding protein.

The assay disclosed herein measures and quantifies the ability of tested small molecules in a cell-free system to compete with GTP or GDP binding. The assay can be used in low volumes or for High Throughput Screening (HTS) to screen a compound library and to support medicinal chemistry Structure Activity Relationship (SAR) studies and optimization efforts.

This is a competitive binding assay. It involves the immobilization of a protein on solid phase, interaction with a small molecule drug candidate, and then competitive binding with a labeled native GTP or GDP ligand.

In one embodiment, provided herein is a method of testing the ability of one or more compounds to bind to the GTP binding domain and to compete for GTP binding of one or more members of the Ras superfamily comprising:

a) expressing a Ras superfamily protein or mutant thereof as a tagged protein;

b) contacting the one or more compounds to the tagged protein, followed by incubating the combination;

c) adding labeled-GTP or labeled-GDP to each protein-compound combination, followed by incubating the resulting mixture; and

d) measuring the amount of bound, labeled-GTP or bound, labeled-GDP.

In one embodiment, the method further comprises between step a) and step b): adding the tagged protein to one or more wells of a ligand-coated single or multi-well plate and incubating the tagged protein.

In one embodiment of the method, one or more members of the Ras superfamily is Ras. In one embodiment of the method, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In one embodiment of the method, the Ras is HRAS, KRAS, NRAS, or a mutant thereof. In one embodiment of the method, the Ras is HRAS or a mutant thereof. In one embodiment of the method, the Ras is KRAS or a mutant thereof. In one embodiment of the method, the Ras is NRAS or a mutant thereof.

In one embodiment of the method, the Ras superfamily protein is KRas G12D mutant protein. In one embodiment of the method, the Ras superfamily protein is KRas G12C mutant protein. In one embodiment of the method, the Ras superfamily protein is KRas wild type protein. In one embodiment of the method, the Ras superfamily protein is KRas Q61H mutant protein.

In one embodiment of the method, one or more members of the Ras superfamily is Rho. In one embodiment of the method, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof. In one embodiment of the method, the Ras superfamily protein is Rho-A protein.

In one embodiment of the method, one or more members of the Ras superfamily is Rac. In one embodiment of the method, the Rho is Rac or a mutant thereof. In one embodiment of the method, the Rac is RAC1; RAC2; RAC3; RHOG, or a mutant thereof. In one embodiment of the method, the Ras superfamily protein is Rac-1 protein.

In one embodiment of the method, the tagged protein is tagged with His. In one embodiment of the method, the ligand is nickel. In one embodiment of the method, the labeled-GTP is Cy3-GTP or Cy5-GTP. In one embodiment of the method, the buffer is Buffer-I which comprises 25 mM Tris (pH 7.4), 150 mM NaCl, 1 mM MgCl₂, and 1 mM DTT. In another embodiment of the method, the buffer is Buffer-II which comprises 50 mM Tris (pH 7.0), 1 mM MgCl₂, and 1 mM DTT.

The form of the assay involves the binding of His-tagged protein to nickel coated plates and a native form of GTP covalently labeled with Cy3 or Cy5 fluorescent probes.

In theory, the assay is suitable for use with any GTP or GDP binding protein. The Examples demonstrate that the assay can be utilized for Ras and Ras mutants, Rac-1 and Rho-A human proteins expressed and purified as recombinant proteins.

Different tag/ligand combinations can be used in the assay. The protein may be expressed as a fusion protein with a tag such as His, HA, Flag or GST; or, the protein can be labeled by a tag such as biotin via chemical reaction. The counter molecule (ligand or binder) interacting with the tag will bind or coat the solid phase. The solid phase could be a plate (96, 384 or 1536 wells plate) and column beads such as sepharose, agarose and cellulose. Binders could include metals such as nickel, copper or cobalt, and antibodies, glutathione and streptavidin. Examples of tag:ligand combinations include His (polyhistidine, at least 6 histidines): nickel, GST (Glutathione-S-transferase): glutathione, HA (amino acids 98-106 of human influenza hemagglutinin):anti-HA antibodies, Fc (constant region of human IgG):protein A, FLAG (the peptide DYDDDDK):Antibodies (M1. M2, 4E11), Myc (the peptide EQKLISEED derived the myc protein): Anti-myc antibodies, and biotin: streptavidin (or avidin).

Heretofore, attempts to measure small molecule competitors for GTP protein interactions have relied on the ability of the tested small molecules to prevent binding of labeled GTP to the GTP-binding protein. A component of this assay is the use of the highly sensitive Cy3 or Cy5 probes. Similar probes which might be used include other high sensitivity fluorophores that can be detected at concentrations below 1 micromolar in solution, and radioactive labeling.

The Identification of Amino Acids in the Ras GTP Binding Domain Enabling the Development and Function of Small Molecule Targeted Therapeutics

As noted in International Application No. PCT/US2018/038613 and U.S. patent application Ser. No. 16/013,872, it has also been discovered that amino acids in the Ras GTP binding domain, including Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 and Mg202, enable the competitive binding to that domain between compounds, such as small molecules, and GTP.

As described in International Application No. PCT/US2018/038613 and U.S. patent application Ser. No. 16/013,872, molecular modeling studies incorporating Ras superfamily protein structures from the RCSB PDB (www.rcsb.org) with either GDP, the GTP analog GNP (guanosine 5′-[β,γ-imido]triphosphate trisodium salt hydrate), or small molecules, were used to determine the amino acids in the Ras superfamily domain in close proximity to the GDP, GTP or small molecules when bound to the Ras superfamily protein.

As noted in International Application No. PCT/US2018/038613 and U.S. patent application Ser. No. 16/013,872, it has also been discovered that amino acids in the Rac1 GTP binding domain, including Gly12, Ala13, Gly15, Lys16, Thr17, Cys18, Leu19, Phe28, Ile33, Pro34, Val36, Ala59, Thr115, Lys116, Asp118, Leu119, Cys157, Ala159, and Mg202, enable the competitive binding to that domain between compounds, such as small molecules, and GTP.

As noted in International Application No. PCT/US2018/038613 and U.S. patent application Ser. No. 16/013,872, it has also been discovered that amino acids in the RhoA GTP binding domain, including Gly14, Ala15, Cys16, Gly17, Lys18, Thr19, Cys20, Phe30, Pro31, Glu32, Tyr34, Val35, Pro36, Thr37, Asp59, Lys118, Asp120, Lys162, and Mg202, enable the competitive binding to that domain between compounds, such as small molecules, and GTP.

### 4.3. Methods of Treatment

4.3.1 Cancer

In one embodiment, provided herein is a method of treating or preventing cancer, which comprises administering to a subject a compound that inhibits the function of one or more members of the Ras superfamily. In one embodiment, provided herein is a method of treating or preventing cancer, which comprises administering to a subject a compound that inhibits the function of one or more members of the Ras superfamily by binding to the GTP binding domain of one or more members of the Ras superfamily. In one embodiment, provided herein is a method of treating or preventing cancer, which comprises administering to a subject a compound that inhibits the function of Ras by binding to a Ras GTP binding domain. In another embodiment, provided herein is a method of treating or preventing cancer, which comprises administering to a subject a compound that binds to a Ras GTP binding domain with an IC₅₀ of less than 10 μM and a K_{d} of less than 10 μM. In another embodiment, provided herein is a method of treating or preventing cancer, which comprises administering to a subject a compound that binds to a Ras GTP binding domain with an IC₅₀ of less than 1 μM and a K_{d} of less than 1 μM. In another embodiment, provided herein is a method of treating or preventing cancer, which comprises administering to a subject a compound that binds to a Ras GTP binding domain with an IC₅₀ of less than 500 nM and a K_{d} of less than 500 nM. In another embodiment, provided herein is a method of treating or preventing cancer, which comprises administering to a subject a compound that binds to a Ras GTP binding domain with an IC₅₀ of less than 470 nM and a K_{d} of less than 470 nM. In another embodiment, provided herein is a method of treating or preventing cancer, which comprises administering to a subject a compound that binds to a Ras GTP binding domain with an IC₅₀ of less than 270 nM and a K_{d} of less than 270 nM. In another embodiment, provided herein is a method of treating or preventing cancer, which comprises administering to a subject a compound that binds to a Ras GTP binding domain with an IC₅₀ of less than 200 nM and a K_{d} of less than 200 nM. In another embodiment, provided herein is a method of treating or preventing cancer, which comprises administering to a subject a compound that binds to a Ras GTP binding domain with an IC₅₀ of less than 150 nM and a K_{d} of less than 150 nM. In another embodiment, provided herein is a method of treating or preventing cancer, which comprises administering to a subject a compound that binds to a Ras GTP binding domain with an IC₅₀ of less than 100 nM and a K_{d} of less than 100 nM. In another embodiment, provided herein is a method of treating or preventing cancer, which comprises administering to a subject a compound that binds to a Ras GTP binding domain with greater than 15% inhibition at 20 μM. In another embodiment, provided herein is a method of treating or preventing cancer, which comprises administering to a subject a compound that binds to a Ras GTP binding domain with greater than 25% inhibition at 20 μM. In another embodiment, provided herein is a method of treating or preventing cancer, which comprises administering to a subject a compound that binds to a Ras GTP binding domain with greater than 50% inhibition at 20 μM. In another embodiment, provided herein is a method of treating or preventing cancer, which comprises administering to a subject a compound that binds to a Ras GTP binding domain with greater than 75% inhibition at 20 μM. In another embodiment, provided herein is a method of treating or preventing cancer, which comprises administering to a subject a compound that binds to a Ras GTP binding domain with greater than 80% inhibition at 20 μM. In another embodiment, provided herein is a method of treating or preventing cancer, which comprises administering to a subject a compound that binds to a Ras GTP binding domain with greater than 85% inhibition at 20 μM. In another embodiment, provided herein is a method of treating or preventing cancer, which comprises administering to a subject a compound that binds to a Ras GTP binding domain with greater than 90% inhibition at 20 μM. In another embodiment, provided herein is a method of treating or preventing cancer, which comprises administering to a subject a compound that binds to a Ras GTP binding domain with greater than 95% inhibition at 20 μM. In another embodiment, provided herein is a method of treating or preventing cancer, which comprises administering to a subject a compound that binds to a Ras GTP binding domain with greater than 99% inhibition at 20 μM. In one embodiment, provided herein is a method of treating or preventing cancer, which comprises administering to a subject a compound that inhibits the function of Rho. In one embodiment, provided herein is a method of treating or preventing cancer, which comprises administering to a subject a compound that inhibits the function of Rho by binding to a Rho GTP binding domain. In another embodiment, provided herein is a method of treating or preventing cancer, which comprises administering to a subject a compound that binds to a Rho GTP binding domain with an IC₅₀ of less than 10 μM and a K_{d} of less than 10 μM. In another embodiment, provided herein is a method of treating or preventing cancer, which comprises administering to a subject a compound that binds to a Rho GTP binding domain with an IC₅₀ of less than 1 μM and a K_{d} of less than 1 μM. In another embodiment, provided herein is a method of treating or preventing cancer, which comprises administering to a subject a compound that binds to a Rho GTP binding domain with an IC₅₀ of less than 500 nM and a K_{d} of less than 500 nM. In another embodiment, provided herein is a method of treating or preventing cancer, which comprises administering to a subject a compound that binds to a Rho GTP binding domain with an IC₅₀ of less than 270 nM and a K_{d} of less than 270 nM. In another embodiment, provided herein is a method of treating or preventing cancer, which comprises administering to a subject a compound that binds to a Rho GTP binding domain with an IC₅₀ of less than 200 nM and a K_{d} of less than 200 nM. In another embodiment, provided herein is a method of treating or preventing cancer, which comprises administering to a subject a compound that binds to a Rho GTP binding domain with an IC₅₀ of less than 150 nM and a K_{d} of less than 150 nM. In another embodiment, provided herein is a method of treating or preventing cancer, which comprises administering to a subject a compound that binds to a Rho GTP binding domain with an IC₅₀ of less than 130 nM and a K_{d} of less than 130 nM. In another embodiment, provided herein is a method of treating or preventing cancer, which comprises administering to a subject a compound that binds to a Rho GTP binding domain with an IC₅₀ of less than 100 nM and a K_{d} of less than 100 nM. In another embodiment, provided herein is a method of treating or preventing cancer, which comprises administering to a subject a compound that binds to a Rho GTP binding domain with greater than 15% inhibition at 20 μM. In another embodiment, provided herein is a method of treating or preventing cancer, which comprises administering to a subject a compound that binds to a Rho GTP binding domain with greater than 25% inhibition at 20 μM. In another embodiment, provided herein is a method of treating or preventing cancer, which comprises administering to a subject a compound that binds to a Rho GTP binding domain with greater than 50% inhibition at 20 μM. In another embodiment, provided herein is a method of treating or preventing cancer, which comprises administering to a subject a compound that binds to a Rho GTP binding domain with greater than 75% inhibition at 20 μM. In another embodiment, provided herein is a method of treating or preventing cancer, which comprises administering to a subject a compound that binds to a Rho GTP binding domain with greater than 80% inhibition at 20 μM. In another embodiment, provided herein is a method of treating or preventing cancer, which comprises administering to a subject a compound that binds to a Rho GTP binding domain with greater than 85% inhibition at 20 μM. In another embodiment, provided herein is a method of treating or preventing cancer, which comprises administering to a subject a compound that binds to a Rho GTP binding domain with greater than 90% inhibition at 20 μM. In another embodiment, provided herein is a method of treating or preventing cancer, which comprises administering to a subject a compound that binds to a Rho GTP binding domain with greater than 95% inhibition at 20 μM. In another embodiment, provided herein is a method of treating or preventing cancer, which comprises administering to a subject a compound that binds to a Rho GTP binding domain with greater than 99% inhibition at 20 μM. In one embodiment, provided herein is a method of treating or preventing cancer, which comprises administering to a subject a compound that inhibits the function of Rac. In one embodiment, provided herein is a method of treating or preventing cancer, which comprises administering to a subject a compound that inhibits the function of Rac by binding to a Rac GTP binding domain. In another embodiment, provided herein is a method of treating or preventing cancer, which comprises administering to a subject a compound that binds to a Rac GTP binding domain with an IC₅₀ of less than 10 μM and a K_{d} of less than 10 μM. In another embodiment, provided herein is a method of treating or preventing cancer, which comprises administering to a subject a compound that binds to a Rac GTP binding domain with an IC₅₀ of less than 1 μM and a K_{d} of less than 1 μM. In another embodiment, provided herein is a method of treating or preventing cancer, which comprises administering to a subject a compound that binds to a Rac GTP binding domain with an IC₅₀ of less than 500 nM and a K_{d} of less than 500 nM. In another embodiment, provided herein is a method of treating or preventing cancer, which comprises administering to a subject a compound that binds to a Rac GTP binding domain with an IC₅₀ of less than 270 nM and a K_{d} of less than 270 nM. In another embodiment, provided herein is a method of treating or preventing cancer, which comprises administering to a subject a compound that binds to a Rac GTP binding domain with an IC₅₀ of less than 200 nM and a K_{d} of less than 200 nM. In another embodiment, provided herein is a method of treating or preventing cancer, which comprises administering to a subject a compound that binds to a Rac GTP binding domain with an IC₅₀ of less than 170 nM and a K_{d} of less than 170 nM. In another embodiment, provided herein is a method of treating or preventing cancer, which comprises administering to a subject a compound that binds to a Rac GTP binding domain with an IC₅₀ of less than 150 nM and a K_{d} of less than 150 nM. In another embodiment, provided herein is a method of treating or preventing cancer, which comprises administering to a subject a compound that binds to a Rac GTP binding domain with an IC₅₀ of less than 100 nM and a K_{d} of less than 100 nM. In another embodiment, provided herein is a method of treating or preventing cancer, which comprises administering to a subject a compound that binds to a Rac GTP binding domain with greater than 15% inhibition at 20 μM. In another embodiment, provided herein is a method of treating or preventing cancer, which comprises administering to a subject a compound that binds to a Rac GTP binding domain with greater than 25% inhibition at 20 μM. In another embodiment, provided herein is a method of treating or preventing cancer, which comprises administering to a subject a compound that binds to a Rac GTP binding domain with greater than 50% inhibition at 20 μM. In another embodiment, provided herein is a method of treating or preventing cancer, which comprises administering to a subject a compound that binds to a Rac GTP binding domain with greater than 75% inhibition at 20 μM. In another embodiment, provided herein is a method of treating or preventing cancer, which comprises administering to a subject a compound that binds to a Rac GTP binding domain with greater than 80% inhibition at 20 μM. In another embodiment, provided herein is a method of treating or preventing cancer, which comprises administering to a subject a compound that binds to a Rac GTP binding domain with greater than 85% inhibition at 20 μM. In another embodiment, provided herein is a method of treating or preventing cancer, which comprises administering to a subject a compound that binds to a Rac GTP binding domain with greater than 90% inhibition at 20 μM. In another embodiment, provided herein is a method of treating or preventing cancer, which comprises administering to a subject a compound that binds to a Rac GTP binding domain with greater than 95% inhibition at 20 μM. In another embodiment, provided herein is a method of treating or preventing cancer, which comprises administering to a subject a compound that binds to a Rac GTP binding domain with greater than 99% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In another embodiment, provided herein is a method of treating or preventing cancer, which comprises administering to a subject a compound that binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to four or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to five or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to six or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to seven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to eight or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to nine or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to ten or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to eleven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing cancer, which comprises administering to a subject a compound that binds to all of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In another embodiment, provided herein is a method of treating or preventing cancer, which comprises administering to a subject a compound that binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to two or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to three or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to four or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to five or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to six or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to seven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to eight or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to nine or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to ten or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to eleven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to twelve or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to thirteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to fourteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to fifteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to sixteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds to seventeen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing cancer, which comprises administering to a subject a compound that binds all of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

In one embodiment, provided herein is a method of treating or preventing cancer, which comprises administering to a subject a compound that binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to two or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rho GTP binding domain. In one embodiment, provided herein is a method of treating or preventing cancer, which comprises administering to a subject a compound that binds to three or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to four or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to five or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to six or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to seven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to eight or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to nine or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to ten or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to eleven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to twelve or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to thirteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to fourteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to fifteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to sixteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to seventeen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to eighteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing cancer, which comprises administering to a subject a compound that binds to all of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is Rac. In one embodiment the Rac is RAC1; RAC2; RAC3 or RHOG. In one embodiment, the Rac is RAC1. In another embodiment, the Rac is a mutant form of a Rac described herein.

In one embodiment, the compound for use in the methods and compositions provided herein inhibit GTP binding to one or more members of the Ras superfamily. In one embodiment, the compound for use in the methods and compositions provided herein inhibit GTP binding to Ras. In one embodiment, the compounds provided herein inhibit GTP binding to Rho. In one embodiment, the compound for use in the methods and compositions provided herein inhibit GTP binding to Rac. In one embodiment, the compound for use in the methods and compositions provided herein inhibit GTP binding to Ras and Rho. In one embodiment, the compound for use in the methods and compositions provided herein inhibit GTP binding to Ras and Rac. In one embodiment, the compound for use in the methods and compositions provided herein inhibit GTP binding to Rho and Rac. In one embodiment, the compound for use in the methods and compositions provided herein inhibit GTP binding to Ras, Rho and Rac. In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, provided herein is a method of treating or preventing cancer, which comprises administering to a subject a compound provided herein, or a derivative thereof. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In another embodiment, provided herein is method of managing cancer, which comprises administering to a subject a compound provided herein, or a derivative thereof. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

Also provided herein are methods of treating subjects who have been previously treated for cancer but are non-responsive to standard therapies, as well as those who have not previously been treated. Also provided are methods of treating subjects regardless of subject's age, although some diseases or disorders are more common in certain age groups. Also provided are methods of treating subjects who have undergone surgery in an attempt to treat the disease or condition at issue, as well as those who have not. Because subjects with cancer have heterogeneous clinical manifestations and varying clinical outcomes, the treatment given to a subject may vary, depending on his/her prognosis. The skilled clinician will be able to readily determine without undue experimentation specific secondary agents, types of surgery, and types of non-drug based standard therapy that can be effectively used to treat an individual subject with cancer.

As used herein, the term “cancer” includes, but is not limited to, solid tumors and blood borne tumors. The term “cancer” refers to disease of skin tissues, organs, blood, and vessels, including, but not limited to, cancers of the bladder, bone, blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

In certain embodiments, the cancer is a solid tumor. In certain embodiments, the solid tumor is metastatic. In certain embodiments, the solid tumor is drug-resistant. In certain embodiments, the solid tumor is hepatocellular carcinoma, prostate cancer, pancreatic cancer, lung cancer, breast cancer, ovarian cancer, colon cancer, small intestine cancer, biliary tract cancer, endometrium cancer, skin cancer (melanoma), cervix cancer, urinary tract cancer, or glioblastoma.

In certain embodiments, the cancer is a blood borne tumor. In certain embodiments, the blood borne tumor is metastatic. In certain embodiments, the blood borne tumor is drug resistant. In certain embodiments, the cancer is leukemia.

In one embodiment, methods provided herein encompass treating, preventing or managing various types of leukemias such as chronic lymphocytic leukemia (CLL), chronic myelocytic leukemia (CML), acute lymphoblastic leukemia (ALL), acute myeloid leukemia (AML), and acute myeloblastic leukemia (AML) by administering a therapeutically effective amount of a compound provided herein or a derivative thereof.

In some embodiments, the methods provided herein encompass treating, preventing or managing acute leukemia in a subject. In some embodiments, the acute leukemia is acute myeloid leukemia (AML), which includes, but is not limited to, undifferentiated AML (M0), myeloblastic leukemia (M1), myeloblastic leukemia (M2), promyelocytic leukemia (M3 or M3 variant (M3V)), myelomonocytic leukemia (M4 or M4 variant with eosinophilia (M4E)), monocytic leukemia (M5), erythroleukemia (M6), and megakaryoblastic leukemia (M7). In one embodiment, the acute myeloid leukemia is undifferentiated AML (M0). In one embodiment, the acute myeloid leukemia is myeloblastic leukemia (M1). In one embodiment, the acute myeloid leukemia is myeloblastic leukemia (M2). In one embodiment, the acute myeloid leukemia is promyelocytic leukemia (M3 or M3 variant (M3V)). In one embodiment, the acute myeloid leukemia is myelomonocytic leukemia (M4 or M4 variant with eosinophilia (M4E)). In one embodiment, the acute myeloid leukemia is monocytic leukemia (M5). In one embodiment, the acute myeloid leukemia is erythroleukemia (M6). In one embodiment, the acute myeloid leukemia is megakaryoblastic leukemia (M7). Thus, the methods of treating, preventing or managing acute myeloid leukemia in a subject comprise the step of administering to the subject an amount of a compound provided herein or a derivative thereof effective to treat, prevent or manage acute myeloid leukemia alone or in combination. In some embodiments, the methods comprise the step of administering to the subject a compound provided herein or a derivative thereof in combination with a second active agent in amounts effective to treat, prevent or manage acute myeloid leukemia.

In some embodiments, the methods provided herein encompass treating, preventing or managing acute lymphocytic leukemia (ALL) in a subject. In some embodiments, acute lymphocytic leukemia includes leukemia that originates in the blast cells of the bone marrow (B-cells), thymus (T-cells), and lymph nodes. The acute lymphocytic leukemia can be categorized according to the French-American-British (FAB) Morphological Classification Scheme as L1—Mature-appearing lymphoblasts (T cells or pre-B-cells), L2—Immature and pleomorphic (variously shaped) lymphoblasts (T-cells or pre-B-cells), and L3—Lymphoblasts (B-cells; Burkitt's cells). In one embodiment, the acute lymphocytic leukemia originates in the blast cells of the bone marrow (B-cells). In one embodiment, the acute lymphocytic leukemia originates in the thymus (T-cells). In one embodiment, the acute lymphocytic leukemia originates in the lymph nodes. In one embodiment, the acute lymphocytic leukemia is L1 type characterized by mature-appearing lymphoblasts (T-cells or pre-B-cells). In one embodiment, the acute lymphocytic leukemia is L2 type characterized by immature and pleomorphic (variously shaped) lymphoblasts (T-cells or pre-B-cells). In one embodiment, the acute lymphocytic leukemia is L3 type characterized by lymphoblasts (B-cells; Burkitt's cells). In certain embodiments, the acute lymphocytic leukemia is T cell leukemia. In one embodiment, the T-cell leukemia is peripheral T-cell leukemia. In another embodiment, the T-cell leukemia is T-cell lymphoblastic leukemia. In another embodiment, the T-cell leukemia is cutaneous T-cell leukemia. In another embodiment, the T-cell leukemia is adult T-cell leukemia. Thus, the methods of treating, preventing or managing acute lymphocytic leukemia in a subject comprise the step of administering to the subject an amount of a compound provided herein or a derivative thereof effective to treat, prevent or manage acute lymphocytic leukemia alone or in combination with a second active agent. In some embodiments, the methods comprise the step of administering to the subject a compound provided herein or a derivative thereof in combination with a second active agent in amounts effective to treat, prevent or manage acute lymphocytic leukemia.

In some embodiments, the methods provided herein encompass treating, preventing or managing chronic myelogenous leukemia (CML) in a subject. The methods comprise the step of administering to the subject an amount of a compound provided herein or a derivative thereof effective to treat, prevent or manage chronic myelogenous leukemia. In some embodiments, the methods comprise the step of administering to the subject a compound provided herein or a derivative thereof in combination with a second active agent in amounts effective to treat, prevent or manage chronic myelogenous leukemia.

In some embodiments, the methods provided herein encompass treating, preventing or managing chronic lymphocytic leukemia (CLL) in a subject. The methods comprise the step of administering to the subject an amount of a compound provided herein or a derivative thereof effective to treat, prevent or manage chronic lymphocytic leukemia. In some embodiments, the methods comprise the step of administering to the subject a compound provided herein or a derivative thereof in combination with a second active agent in amounts effective to treat, prevent or manage chronic lymphocytic leukemia.

In certain embodiments, provided herein are methods of treating, preventing, and/or managing disease in subjects with impaired renal function. In certain embodiments, provided herein are method of treating, preventing, and/or managing cancer in subjects with impaired renal function. In certain embodiments, provided herein are methods of providing appropriate dose adjustments for subjects with impaired renal function due to, but not limited to, disease, aging, or other subject factors.

In certain embodiments, provided herein are methods of treating, preventing, and/or managing lymphoma, including non-Hodgkin's lymphoma. In some embodiments, provided herein are methods for the treatment or management of non-Hodgkin's lymphoma (NHL), including but not limited to, diffuse large B-cell lymphoma (DLBCL), using prognostic factors.

In certain embodiments, provided herein are methods of treating, preventing, and/or managing multiple myeloma, including relapsed/refractory multiple myeloma in subjects with impaired renal function or a symptom thereof, comprising administering a therapeutically effective amount of a compound provided herein, or a derivative thereof to a subject having relapsed/refractory multiple myeloma with impaired renal function.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, e.g., Nair A B, Jacob S. A simple practice guide for dose conversion between animals and human. J Basic Clin Pharma 2016; 7:27-31). For example, a dose of 1 mg/kg/day for a 60 kg human is approximately equal to 37 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 500 μM, about 0.002 to about 200 μM, about 0.005 to about 100 PM, about 0.01 to about 50 μM, from about 1 to about 50 μM, about 0.02 to about 25 μM, from about 0.05 to about 20 μM, from about 0.1 to about 20 μM, from about 0.5 to about 20 PM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

In some embodiments, the compound administered is a compound as disclosed herein of Formula IA, IB, IC, ID, IE, IF, IIA, or IIB, or a pharmaceutically acceptable salt thereof. In some embodiments, the administered compound is a compound selected from Compounds 1-39, such as a compound selected from compounds 1-27 of Examples 1-27, or a pharmaceutically acceptable salt thereof.

In certain embodiments, the subject to be treated with one of the methods provided herein has not been treated with anticancer therapy prior to the administration of the compound provided herein, or a derivative thereof. In certain embodiments, the subject to be treated with one of the methods provided herein has been treated with anticancer therapy prior to the administration of the compound provided herein, or a derivative thereof. In certain embodiments, the subject to be treated with one of the methods provided herein has developed drug resistance to the anticancer therapy.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

Combination Therapy with a Second Active Agent

The compound provided herein, or a derivative thereof, can also be combined or used in combination with other therapeutic agents useful in the treatment and/or prevention of cancer described herein.

In one embodiment, provided herein is a method of treating, preventing, or managing cancer, comprising administering to a subject a compound provided herein, or a derivative thereof, in combination with one or more second active agents, and optionally in combination with radiation therapy, blood transfusions, or surgery. Examples of second active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent (an anticancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells and immunologically active poietic cells in vitro or in vivo. Other useful proteins stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four or six week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mcg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller subjects) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; the entireties of which are incorporated herein by reference.

Also provided for use in combination with a compound provided herein, or a derivative thereof, of are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with a compound provided herein, or a derivative thereof, include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. The compounds provided herein or an enantiomer or a mixture of enantiomers thereof, or a pharmaceutically acceptable salt, solvate, hydrate, co-crystal, clathrate, or polymorph thereof can also be combined with, or used in combination with, anti-TNF-α antibodies, and/or anti-EGFR antibodies, such as, for example, Erbitux® or panitumumab.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L. A., et al., Curr. Opinion Mol. Ther. 3(1):77-84 (2001).

Second active agents that are small molecules can also be used to alleviate adverse effects associated with the administration of a compound provided herein, or a derivative thereof. However, like some large molecules, many are believed to be capable of providing a synergistic effect when administered with (e.g., before, after or simultaneously) a compound provided herein, or a derivative thereof. Examples of small molecule second active agents include, but are not limited to, anti-cancer agents, antibiotics, immunosuppressive agents, and steroids.

In certain embodiments, the second agent is an HSP inhibitor, a proteasome inhibitor, a FLT3 inhibitor or a TOR kinase inhibitor.

Examples of anti-cancer agents to be used within the methods or compositions described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods or compositions include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6 benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents particularly useful in the methods or compositions include, but are not limited to, rituximab, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, Ara-C, doxetaxol, pacilitaxel, vinblastine, IL-2, GM CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

In certain embodiments of the methods provided herein, use of a second active agent in combination with a compound provided herein, or a derivative thereof, may be modified or delayed during or shortly following administration of a compound provided herein, or a derivative thereof, as deemed appropriate by the practitioner of skill in the art. In certain embodiments, subjects being administered a compound provided herein, or a derivative thereof, alone or in combination with other therapies may receive supportive care including antiemetics, myeloid growth factors, and transfusions of platelets, when appropriate. In some embodiments, subjects being administered a compound provided herein, or a derivative thereof, may be administered a growth factor as a second active agent according to the judgment of the practitioner of skill in the art. In some embodiments, provided is administration of a compound provided herein, or a derivative thereof, in combination with erythropoietin or darbepoetin (Aranesp).

In certain embodiments, a compound provided herein, or a derivative thereof, is administered with gemcitabine and cisplatinum to subjects with locally advanced or metastatic transitional cell bladder cancer.

In certain embodiments, a compound provided herein, or a derivative thereof, is administered in combination with a second active ingredient as follows: temozolomide to pediatric subjects with relapsed or progressive brain tumors or recurrent neuroblastoma; celecoxib, etoposide and cyclophosphamide for relapsed or progressive CNS cancer; temodar to subjects with recurrent or progressive meningioma, malignant meningioma, hemangiopericytoma, multiple brain metastases, relapsed brain tumors, or newly diagnosed glioblastoma multiforms; irinotecan to subjects with recurrent glioblastoma; carboplatin to pediatric subjects with brain stem glioma; procarbazine to pediatric subjects with progressive malignant gliomas; cyclophosphamide to subjects with poor prognosis malignant brain tumors, newly diagnosed or recurrent glioblastoma multiforms; Gliadel® for high grade recurrent malignant gliomas; temozolomide and tamoxifen for anaplastic astrocytoma; or topotecan for gliomas, glioblastoma, anaplastic astrocytoma or anaplastic oligodendroglioma.

In certain embodiments, a compound provided herein, or a derivative thereof, is administered with methotrexate, cyclophosphamide, taxane, abraxane, lapatinib, herceptin, aromatase inhibitors, selective estrogen modulators, estrogen receptor antagonists, and/or PLX3397 (Plexxikon) to subjects with metastatic breast cancer.

In certain embodiments, a compound provided herein, or a derivative thereof, is administered with temozolomide to subjects with neuroendocrine tumors.

In certain embodiments, a compound provided herein, or a derivative thereof, is administered with gemcitabine to subjects with recurrent or metastatic head or neck cancer.

In certain embodiments, a compound provided herein, or a derivative thereof, is administered with gemcitabine to subjects with pancreatic cancer.

In certain embodiments, a compound provided herein, or a derivative thereof, is administered to subjects with colon cancer in combination with ARISA®, avastatin, taxol, and/or taxotere.

In certain embodiments, a compound provided herein, or a derivative thereof, is administered with capecitabine and/or PLX4032 (Plexxikon) to subjects with refractory colorectal cancer or subjects who fail first line therapy or have poor performance in colon or rectal adenocarcinoma.

In certain embodiments, a compound provided herein, or a derivative thereof, is administered in combination with fluorouracil, leucovorin, and irinotecan to subjects with Dukes C & D colorectal cancer or to subjects who have been previously treated for metastatic colorectal cancer.

In certain embodiments, a compound provided herein, or a derivative thereof, is administered to subjects with refractory colorectal cancer in combination with capecitabine, xeloda, and/or CPT-11.

In certain embodiments, a compound provided herein, or a derivative thereof, is administered with capecitabine and irinotecan to subjects with refractory colorectal cancer or to subjects with unresectable or metastatic colorectal carcinoma.

In certain embodiments, a compound provided herein, or a derivative thereof, is administered alone or in combination with interferon alpha or capecitabine to subjects with unresectable or metastatic hepatocellular carcinoma; or with cisplatin and thiotepa to subjects with primary or metastatic liver cancer.

In certain embodiments, a compound provided herein, or a derivative thereof, is administered in combination with pegylated interferon alpha to subjects with Kaposi's sarcoma.

In certain embodiments, a compound provided herein, or a derivative thereof, is administered in combination with fludarabine, carboplatin, and/or topotecan to subjects with refractory or relapsed or high-risk acute myeloid leukemia.

In certain embodiments, a compound provided herein, or a derivative thereof, is administered in combination with liposomal daunorubicin, topotecan and/or cytarabine to subjects with unfavorable karotype acute myeloblastic leukemia.

In certain embodiments, a compound provided herein, or a derivative thereof, is administered in combination with gemcitabine, abraxane, erlotinib, geftinib, and/or irinotecan to subjects with non-small cell lung cancer.

In certain embodiments, a compound provided herein, or a derivative thereof, is administered in combination with carboplatin and irinotecan to subjects with non-small cell lung cancer.

In certain embodiments, a compound provided herein, or a derivative thereof, is administered with doxetaxol to subjects with non-small cell lung cancer who have been previously treated with carbo/VP 16 and radiotherapy.

In certain embodiments, a compound provided herein, or a derivative thereof, is administered in combination with carboplatin and/or taxotere, or in combination with carboplatin, pacilitaxel and/or thoracic radiotherapy to subjects with non-small cell lung cancer.

In certain embodiments, a compound provided herein, or a derivative thereof, is administered in combination with taxotere to subjects with stage IIIB or IV non-small cell lung cancer.

In certain embodiments, a compound provided herein, or a derivative thereof, is administered in combination with oblimersen (Genasense®) to subjects with small cell lung cancer.

In certain embodiments, a compound provided herein, or a derivative thereof, is administered in combination with ABT-737 (Abbott Laboratories) and/or obatoclax (GX15-070) to subjects with lymphoma and other blood cancers.

In certain embodiments, a compound provided herein, or a derivative thereof, is administered alone or in combination with a second active ingredient such as vinblastine or fludarabine to subjects with various types of lymphoma, including, but not limited to, Hodgkin's lymphoma, non-Hodgkin's lymphoma, cutaneous T-Cell lymphoma, cutaneous B-Cell lymphoma, diffuse large B-Cell lymphoma or relapsed or refractory low grade follicular lymphoma.

In certain embodiments, a compound provided herein, or a derivative thereof, is administered in combination with taxotere, IL-2, IFN, GM-CSF, PLX4032 (Plexxikon) and/or dacarbazine to subjects with various types or stages of melanoma.

In certain embodiments, a compound provided herein, or a derivative thereof, is administered alone or in combination with vinorelbine to subjects with malignant mesothelioma, or stage IIIB non-small cell lung cancer with pleural implants or malignant pleural effusion mesothelioma syndrome.

In certain embodiments, a compound provided herein, or a derivative thereof, is administered to subjects with various types or stages of multiple myeloma in combination with dexamethasone, zoledronic acid, palmitronate, GM-CSF, biaxin, vinblastine, melphalan, busulphan, cyclophosphamide, IFN, palmidronate, prednisone, bisphosphonate, celecoxib, arsenic trioxide, PEG INTRON-A, vincristine, or a combination thereof.

In certain embodiments, a compound provided herein, or a derivative thereof, is administered to subjects with relapsed or refractory multiple myeloma in combination with doxorubicin (Doxil®), vincristine and/or dexamethasone (Decadron®).

In certain embodiments, a compound provided herein, or a derivative thereof, is administered to subjects with various types or stages of ovarian cancer such as peritoneal carcinoma, papillary serous carcinoma, refractory ovarian cancer or recurrent ovarian cancer, in combination with taxol, carboplatin, doxorubicin, gemcitabine, cisplatin, xeloda, paclitaxel, dexamethasone, or a combination thereof.

In certain embodiments, a compound provided herein, or a derivative thereof, is administered to subjects with various types or stages of prostate cancer, in combination with xeloda, 5 FU/LV, gemcitabine, irinotecan plus gemcitabine, cyclophosphamide, vincristine, dexamethasone, GM-CSF, celecoxib, taxotere, ganciclovir, paclitaxel, adriamycin, docetaxel, estramustine, Emcyt, denderon or a combination thereof.

In certain embodiments, a compound provided herein, or a derivative thereof, is administered to subjects with various types or stages of renal cell cancer, in combination with capecitabine, IFN, tamoxifen, IL-2, GM-CSF, Celebrex®, or a combination thereof.

In certain embodiments, a compound provided herein, or a derivative thereof, is administered to subjects with various types or stages of gynecologic, uterus or soft tissue sarcoma cancer in combination with IFN, a COX-2 inhibitor such as Celebrex®, and/or sulindac.

In certain embodiments, a compound provided herein, or a derivative thereof, is administered to subjects with various types or stages of solid tumors in combination with celebrex, etoposide, cyclophosphamide, docetaxel, apecitabine, IFN, tamoxifen, IL-2, GM-CSF, or a combination thereof.

In certain embodiments, a compound provided herein, or a derivative thereof, is administered to subjects with scleroderma or cutaneous vasculitis in combination with celebrex, etoposide, cyclophosphamide, docetaxel, apecitabine, IFN, tamoxifen, IL-2, GM-CSF, or a combination thereof.

Also encompassed herein is a method of increasing the dosage of an anti-cancer drug or agent that can be safely and effectively administered to a subject, which comprises administering to the subject (e.g., a human) a compound provided herein, or a derivative thereof. Subjects that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of a compound provided herein, or a derivative thereof, alleviates or reduces adverse effects which are of such severity that it would otherwise limit the amount of anti-cancer drug.

In one embodiment, a compound provided herein, or a derivative thereof, is administered orally and daily in an amount ranging from about 0.1 to about 150 mg, from about 1 to about 50 mg, or from about 2 to about 25 mg, prior to, during, or after the occurrence of the adverse effect associated with the administration of an anti-cancer drug to a subject. In certain embodiments, a compound provided herein, or a derivative thereof, is administered in combination with specific agents such as heparin, aspirin, coumadin, or G CSF to avoid adverse effects that are associated with anti-cancer drugs such as but not limited to neutropenia or thrombocytopenia.

In one embodiment, a compound provided herein, or a derivative thereof, is administered to subjects with diseases and disorders associated with or characterized by, undesired angiogenesis in combination with additional active ingredients, including, but not limited to, anti-cancer drugs, anti-inflammatories, antihistamines, antibiotics, and steroids.

In another embodiment, encompassed herein is a method of treating, preventing and/or managing cancer, which comprises administering the compound provided herein, or a derivative thereof, in conjunction with (e.g. before, during, or after) conventional therapy including, but not limited to, surgery, immunotherapy, biological therapy, radiation therapy, or other non-drug based therapy presently used to treat, prevent or manage cancer. The combined use of the compound provided herein, or a derivative thereof, and conventional therapy may provide a unique treatment regimen that is unexpectedly effective in certain subjects. Without being limited by theory, it is believed that the compound provided herein, or a derivative thereof, may provide additive or synergistic effects when given concurrently with conventional therapy.

As discussed elsewhere herein, encompassed herein is a method of reducing, treating and/or preventing adverse or undesired effects associated with conventional therapy including, but not limited to, surgery, chemotherapy, radiation therapy, hormonal therapy, biological therapy and immunotherapy. A compound provided herein, or a derivative thereof, and other active ingredient can be administered to a subject prior to, during, or after the occurrence of the adverse effect associated with conventional therapy.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent disclosed herein, prior to, during, or after the use of conventional therapy.

In certain embodiments, a compound provided herein, or a derivative thereof, and doxetaxol are administered to subjects with non-small cell lung cancer who were previously treated with carbo/VP 16 and radiotherapy.

In certain embodiments, a compound provided herein, or a derivative thereof, is administered to subjects with various types or stages of cancer, in combination with an immune oncology drug or a combination of immune oncology drugs. In one embodiment, a compound provided herein, or a derivative thereof, is administered to subjects with various types or stages of cancer, in combination with Opdivo, Keytruda, Yervoy or a combination thereof.

4.3.2 Inflammation

As discussed herein, activation of MAPKs, in particular ERK1/2, is a component of the inflammatory response. Thus, the compounds provided herein, which are ERK1/2 inhibitors via inhibition of Ras and/or a Ras superfamily member, are useful in the treatment of inflammatory diseases.

As discussed herein, activation of Akt is a component of the inflammatory response. Thus, the compounds provided herein, which are Akt inhibitors via inhibition of Ras and/or a Ras superfamily member, are useful in the treatment of inflammatory diseases.

In one embodiment, provided herein is a method of treating or preventing inflammatory disease, which comprises administering to a subject a compound that inhibits the function of one or more members of the Ras superfamily. In one embodiment, provided herein is a method of treating or preventing inflammatory disease, which comprises administering to a subject a compound that inhibits the function of one or more members of the Ras superfamily by binding to the GTP binding domain or one or more members of the Ras superfamily. In one embodiment, provided herein is a method of treating or preventing inflammatory disease, which comprises administering to a subject a compound that inhibits the function of Ras by binding to a Ras GTP binding domain. In another embodiment, provided herein is a method of treating or preventing inflammatory disease, which comprises administering to a subject a compound that binds to a Ras GTP binding domain with an IC₅₀ of less than 10 μM and a K_{d} of less than 10 μM. In another embodiment, provided herein is a method of treating or preventing inflammatory disease, which comprises administering to a subject a compound that binds to a Ras GTP binding domain with an IC₅₀ of less than 1 μM and a K_{d} of less than 1 μM. In another embodiment, provided herein is a method of treating or preventing inflammatory disease, which comprises administering to a subject a compound that binds to a Ras GTP binding domain with an IC₅₀ of less than 500 nM and a K_{d} of less than 500 nM. In another embodiment, provided herein is a method of treating or preventing inflammatory disease, which comprises administering to a subject a compound that binds to a Ras GTP binding domain with an IC₅₀ of less than 470 nM and a K_{d} of less than 470 nM. In another embodiment, provided herein is a method of treating or preventing inflammatory disease, which comprises administering to a subject a compound that binds to a Ras GTP binding domain with an IC₅₀ of less than 270 nM and a K_{d} of less than 270 nM. In another embodiment, provided herein is a method of treating or preventing inflammatory disease, which comprises administering to a subject a compound that binds to a Ras GTP binding domain with an IC₅₀ of less than 200 nM and a K_{d} of less than 200 nM. In another embodiment, provided herein is a method of treating or preventing inflammatory disease, which comprises administering to a subject a compound that binds to a Ras GTP binding domain with an IC₅₀ of less than 150 nM and a K_{d} of less than 150 nM. In another embodiment, provided herein is a method of treating or preventing inflammatory disease, which comprises administering to a subject a compound that binds to a Ras GTP binding domain with an IC₅₀ of less than 100 nM and a K_{d} of less than 100 nM. In another embodiment, provided herein is a method of treating or preventing inflammatory disease, which comprises administering to a subject a compound that binds to a Ras GTP binding domain with greater than 15% inhibition at 20 μM. In another embodiment, provided herein is a method of treating or preventing inflammatory disease, which comprises administering to a subject a compound that binds to a Ras GTP binding domain with greater than 25% inhibition at 20 μM. In another embodiment, provided herein is a method of treating or preventing inflammatory disease, which comprises administering to a subject a compound that binds to a Ras GTP binding domain with greater than 50% inhibition at 20 μM. In another embodiment, provided herein is a method of treating or preventing inflammatory disease, which comprises administering to a subject a compound that binds to a Ras GTP binding domain with greater than 75% inhibition at 20 μM. In another embodiment, provided herein is a method of treating or preventing inflammatory disease, which comprises administering to a subject a compound that binds to a Ras GTP binding domain with greater than 80% inhibition at 20 μM. In another embodiment, provided herein is a method of treating or preventing inflammatory disease, which comprises administering to a subject a compound that binds to a Ras GTP binding domain with greater than 85% inhibition at 20 μM. In another embodiment, provided herein is a method of treating or preventing inflammatory disease, which comprises administering to a subject a compound that binds to a Ras GTP binding domain with greater than 90% inhibition at 20 μM. In another embodiment, provided herein is a method of treating or preventing inflammatory disease, which comprises administering to a subject a compound that binds to a Ras GTP binding domain with greater than 95% inhibition at 20 μM. In another embodiment, provided herein is a method of treating or preventing inflammatory disease, which comprises administering to a subject a compound that binds to a Ras GTP binding domain with greater than 99% inhibition at 20 μM. In one embodiment, provided herein is a method of treating or preventing inflammatory disease, which comprises administering to a subject a compound that inhibits the function of Rho. In one embodiment, provided herein is a method of treating or preventing inflammatory disease, which comprises administering to a subject a compound that inhibits the function of Rho by binding to a Rho GTP binding domain. In another embodiment, provided herein is a method of treating or preventing inflammatory disease, which comprises administering to a subject a compound that binds to a Rho GTP binding domain with an IC₅₀ of less than 10 μM and a K_{d} of less than 10 μM. In another embodiment, provided herein is a method of treating or preventing inflammatory disease, which comprises administering to a subject a compound that binds to a Rho GTP binding domain with an IC₅₀ of less than 1 μM and a K_{d} of less than 1 μM. In another embodiment, provided herein is a method of treating or preventing inflammatory disease, which comprises administering to a subject a compound that binds to a Rho GTP binding domain with an IC₅₀ of less than 500 nM and a K_{d} of less than 500 nM. In another embodiment, provided herein is a method of treating or preventing inflammatory disease, which comprises administering to a subject a compound that binds to a Rho GTP binding domain with an IC₅₀ of less than 270 nM and a K_{d} of less than 270 nM. In another embodiment, provided herein is a method of treating or preventing inflammatory disease, which comprises administering to a subject a compound that binds to a Rho GTP binding domain with an IC₅₀ of less than 200 nM and a K_{d} of less than 200 nM. In another embodiment, provided herein is a method of treating or preventing inflammatory disease, which comprises administering to a subject a compound that binds to a Rho GTP binding domain with an IC₅₀ of less than 150 nM and a K_{d} of less than 150 nM. In another embodiment, provided herein is a method of treating or preventing inflammatory disease, which comprises administering to a subject a compound that binds to a Rho GTP binding domain with an IC₅₀ of less than 130 nM and a K_{d} of less than 130 nM. In another embodiment, provided herein is a method of treating or preventing inflammatory disease, which comprises administering to a subject a compound that binds to a Rho GTP binding domain with an IC₅₀ of less than 100 nM and a K_{d} of less than 100 nM. In another embodiment, provided herein is a method of treating or preventing inflammatory disease, which comprises administering to a subject a compound that binds to a Rho GTP binding domain with greater than 15% inhibition at 20 μM. In another embodiment, provided herein is a method of treating or preventing inflammatory disease, which comprises administering to a subject a compound that binds to a Rho GTP binding domain with greater than 25% inhibition at 20 μM. In another embodiment, provided herein is a method of treating or preventing inflammatory disease, which comprises administering to a subject a compound that binds to a Rho GTP binding domain with greater than 50% inhibition at 20 μM. In another embodiment, provided herein is a method of treating or preventing inflammatory disease, which comprises administering to a subject a compound that binds to a Rho GTP binding domain with greater than 75% inhibition at 20 μM. In another embodiment, provided herein is a method of treating or preventing inflammatory disease, which comprises administering to a subject a compound that binds to a Rho GTP binding domain with greater than 80% inhibition at 20 μM. In another embodiment, provided herein is a method of treating or preventing inflammatory disease, which comprises administering to a subject a compound that binds to a Rho GTP binding domain with greater than 85% inhibition at 20 μM. In another embodiment, provided herein is a method of treating or preventing inflammatory disease, which comprises administering to a subject a compound that binds to a Rho GTP binding domain with greater than 90% inhibition at 20 μM. In another embodiment, provided herein is a method of treating or preventing inflammatory disease, which comprises administering to a subject a compound that binds to a Rho GTP binding domain with greater than 95% inhibition at 20 μM. In another embodiment, provided herein is a method of treating or preventing inflammatory disease, which comprises administering to a subject a compound that binds to a Rho GTP binding domain with greater than 99% inhibition at 20 μM. In one embodiment, provided herein is a method of treating or preventing inflammatory disease, which comprises administering to a subject a compound that inhibits the function of Rac. In one embodiment, provided herein is a method of treating or preventing inflammatory disease, which comprises administering to a subject a compound that inhibits the function of Rac by binding to a Rac GTP binding domain. In another embodiment, provided herein is a method of treating or preventing inflammatory disease, which comprises administering to a subject a compound that binds to a Rac GTP binding domain with an IC₅₀ of less than 10 μM and a K_{d} of less than 10 μM. In another embodiment, provided herein is a method of treating or preventing inflammatory disease, which comprises administering to a subject a compound that binds to a Rac GTP binding domain with an IC₅₀ of less than 1 μM and a K_{d} of less than 1 μM. In another embodiment, provided herein is a method of treating or preventing inflammatory disease, which comprises administering to a subject a compound that binds to a Rac GTP binding domain with an IC₅₀ of less than 500 nM and a K_{d} of less than 500 nM. In another embodiment, provided herein is a method of treating or preventing inflammatory disease, which comprises administering to a subject a compound that binds to a Rac GTP binding domain with an IC₅₀ of less than 270 nM and a K_{d} of less than 270 nM. In another embodiment, provided herein is a method of treating or preventing inflammatory disease, which comprises administering to a subject a compound that binds to a Rac GTP binding domain with an IC₅₀ of less than 200 nM and a K_{d} of less than 200 nM. In another embodiment, provided herein is a method of treating or preventing inflammatory disease, which comprises administering to a subject a compound that binds to a Rac GTP binding domain with an IC₅₀ of less than 170 nM and a K_{d} of less than 170 nM. In another embodiment, provided herein is a method of treating or preventing inflammatory disease, which comprises administering to a subject a compound that binds to a Rac GTP binding domain with an IC₅₀ of less than 150 nM and a K_{d} of less than 150 nM. In another embodiment, provided herein is a method of treating or preventing inflammatory disease, which comprises administering to a subject a compound that binds to a Rac GTP binding domain with an IC₅₀ of less than 100 nM and a K_{d} of less than 100 nM. In one embodiment, provided herein is a method of treating or preventing inflammatory disease, which comprises administering to a subject a compound that binds to a Rac GTP binding domain with greater than 15% inhibition at 20 μM. In another embodiment, provided herein is a method of treating or preventing inflammatory disease, which comprises administering to a subject a compound that binds to a Rac GTP binding domain with greater than 25% inhibition at 20 μM. In another embodiment, provided herein is a method of treating or preventing inflammatory disease, which comprises administering to a subject a compound that binds to a Rac GTP binding domain with greater than 50% inhibition at 20 μM. In another embodiment, provided herein is a method of treating or preventing inflammatory disease, which comprises administering to a subject a compound that binds to a Rac GTP binding domain with greater than 75% inhibition at 20 μM. In another embodiment, provided herein is a method of treating or preventing inflammatory disease, which comprises administering to a subject a compound that binds to a Rac GTP binding domain with greater than 80% inhibition at 20 μM. In another embodiment, provided herein is a method of treating or preventing inflammatory disease, which comprises administering to a subject a compound that binds to a Rac GTP binding domain with greater than 85% inhibition at 20 μM. In another embodiment, provided herein is a method of treating or preventing inflammatory disease, which comprises administering to a subject a compound that binds to a Rac GTP binding domain with greater than 90% inhibition at 20 μM. In another embodiment, provided herein is a method of treating or preventing inflammatory disease, which comprises administering to a subject a compound that binds to a Rac GTP binding domain with greater than 95% inhibition at 20 μM. In another embodiment, provided herein is a method of treating or preventing inflammatory disease, which comprises administering to a subject a compound that binds to a Rac GTP binding domain with greater than 99% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In another embodiment, provided herein is a method of treating or preventing inflammatory disease, which comprises administering to a subject a compound that binds to one or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to two or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to three or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to four or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to five or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to six or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to seven or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to eight or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to nine or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to ten or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to eleven or more of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing inflammatory disease, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing inflammatory disease, which comprises administering to a subject a compound that binds to all of Ala11, Gly12, Val14, Gly15, Lys16, Ser17, Ala 18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In another embodiment, provided herein is a method of treating or preventing inflammatory disease, which comprises administering to a subject a compound that binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to two or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to three or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to four or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to five or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to six or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to seven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to eight or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to nine or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to ten or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to eleven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to twelve or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to thirteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to fourteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to fifteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to sixteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds to seventeen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing inflammatory disease, which comprises administering to a subject a compound that binds all of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

In one embodiment, provided herein is a method of treating or preventing inflammatory disease, which comprises administering to a subject a compound that binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to two or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to three or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to four or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to five or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to six or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to seven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to eight or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to nine or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to ten or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to eleven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to twelve or more of Gly12, Ala 13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to thirteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to fourteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to fifteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to sixteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to seventeen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to eighteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing inflammatory disease, which comprises administering to a subject a compound that binds to all of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is Rac. In one embodiment the Rac is RAC1; RAC2; RAC3 or RHOG. In one embodiment, the Rac is RAC1. In another embodiment, the Rac is a mutant form of a Rac described herein.

In one embodiment, the compound for use in the methods and compositions provided herein inhibit GTP binding to one or more members of the Ras superfamily. In one embodiment, the compound for use in the methods and compositions provided herein inhibit GTP binding to Ras. In one embodiment, the compounds provided herein inhibit GTP binding to Rho. In one embodiment, the compound for use in the methods and compositions provided herein inhibit GTP binding to Rac. In one embodiment, the compound for use in the methods and compositions provided herein inhibit GTP binding to Ras and Rho. In one embodiment, the compound for use in the methods and compositions provided herein inhibit GTP binding to Ras and Rac. In one embodiment, the compound for use in the methods and compositions provided herein inhibit GTP binding to Rho and Rac. In one embodiment, the compound for use in the methods and compositions provided herein inhibit GTP binding to Ras, Rho and Rac.

In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the inflammatory disease is inflammation-associated cancer development. As disclosed here, the compounds provided herein are useful in treatment of cancer. It is well recognized that the immune inflammatory state serves as a key mediator of the middle stages of tumor development. It is also well known that chronic inflammation can predispose an individual to cancer. Chronic inflammation is caused by a variety of factors, including bacterial, viral, and parasitic infections. The longer the inflammation persists, the higher the risk of associated carcinogenesis. Anti-inflammatory cancer therapy prevents premalignant cells from turning fully cancerous or impedes existing tumors from spreading to distant sites in the body. Thus, in one embodiment, the compounds provided herein are useful in treating inflammatory cancers. Such cancers, and the chronic inflammatory conditions that predispose susceptible cells to neoplastic transformation, include gastric adenocarcinoma (gastritis), mucosa-associated lymphoid tissue (MALT) lymphoma (gastritis), bladder, liver and rectal carcinomas (schistosomiasis), cholangiocarcinoma and colon carcinoma (cholangitis), gall bladder cncer (chronic cholecystitis), ovarian and cervical carcinoma (pelvic inflammatory disease, chronic cervicitis), skin carcinoma (osteomyelitis), colorectal carcinoma (inflammatory bowel disease), esophageal carcinoma (reflux esophagitis, Barrett's esophagus), bladder cancer (bladder inflammation (cystitis)), mesothelioma and lung carcinoma (asbestosis, silicosis), oral squamous cell carcinoma (gingivitis, lichen planus), pancreatic carcinoma (pancreatitis, protease mutation), vulvar squamous cell carcinoma (lichen sclerosis), salivary gland carcinoma (slaladenitis), lung carcinoma (bronchitis) and MALT lymphoma (Sjogren syndrome, Hashimoto's thyroiditis). Shacter, et al., 2002, Oncology, 16(2), 217-26.

In certain embodiments, the compounds provided herein are useful in treating inflammatory diseases in the airways, such as nonspecific bronchial hyper-reactivity, chronic bronchitis, cystic fibrosis, and acute respiratory distress syndrome (ARDS).

In certain embodiments, the compounds provided herein are useful in treating asthma and idiopathic lung fibrosis or idiopathic pulmonary fibrosis (IPF), pulmonary fibrosis, and interstitial lung disease. As known to one of skill in the art, the differentiation of fibroblasts into cell types called myofibroblasts occurs during wound healing, when the cells contribute to the deposition of extracellular matrix (ECM) in the transient process of wound repair. In chronic inflammatory diseases such as asthma, pathological tissue remodeling often occurs, and is mediated by the functions of increased numbers of myofibroblasts in the diseased tissue, see Hinz, B. et al. Am J Pathol. 2007; 170: 1807-1816. In certain embodiments, the compounds provided herein prevent or reduce TGF-β-induced myofibroblast differentiation, as measured by the expression of alpha smooth muscle actin (α-SMA), a hallmark of myofibroblast differentiation (Serini, G. and Gabbiani, G. 1999; Exp. Cell Res. 250: 273-283).

In certain embodiments, the compounds provided herein are useful in treating psoriasis, chronic plaque psoriasis, psoriatic arthritis, acanthosis, atopic dermatitis, various forms of eczema, contact dermatitis (includes allergic dermatitis), systemic sclerosis (scleroderma), wound healing, and drug eruption.

In one embodiment, the disease is inflammation, arthritis, rheumatoid arthritis, spondylarthropathies, gouty arthritis, osteoarthritis, juvenile arthritis, and other arthritic conditions, systemic lupus erthematosus (SLE), skin-related conditions, eczema, Sjögren's syndrome, burns, dermatitis, neuroinflammation, allergy pain, autoimmune myositis, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, acute gout, pneumonitis, myocarditis, pericarditis, myositis, eczema, alopecia, vitiligo, bullous skin diseases, atherosclerosis, depression, retinitis, uveitis, scleritis, hepatitis, pancreatitis, primary biliary cirrhosis, sclerosing cholangitis, Addison's disease, hypophysitis, thyroiditis, type I diabetes, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement, acute rejection of transplanted organs. endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, postsurgical syndromes, sarcoidosis, Herxheimer reactions, encephalitis, myelitis, SIRS associated with viral infections such as influenza, herpes zoster, herpes simplex, coronavirus or dry eye syndrome (or keratoconjunctivitis sicca (KCS)).

In certain embodiments, the compounds provided herein are useful in treating neuropathic and nociceptive pain, chronic or acute, such as, without limitation, allodynia, inflammatory pain, inflammatory hyperalgesia, post herpetic neuralgia, neuropathies, neuralgia, diabetic neuropathy, HIV-related neuropathy, nerve injury, rheumatoid arthritic pain, osteoarthritic pain, burns, back pain, ocular pain, visceral pain, cancer pain, dental pain, headache, migraine, carpal tunnel syndrome, fibromyalgia, neuritis, sciatica, pelvic hypersensitivity, pelvic pain, post operative pain, post stroke pain, and menstrual pain.

In certain embodiments, the compounds provided herein are useful in treating Alzheimer's disease (AD), mild cognitive impairment (MCI), age-associated memory impairment (AAMI), multiple sclerosis, Parkinson's disease, vascular dementia, senile dementia, AIDS dementia, Pick's disease, dementia caused by cerebrovascular disorders, corticobasal degeneration, amyotrophic lateral sclerosis (ALS), Huntington's disease, diminished CNS function associated with traumatic brain injury.

In one embodiment, the compounds provided herein are useful in treating Alzheimer's disease (AD), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus, erythematous (SLE), nephritis, Parkinson's disease, ulcerative colitis.

When used for the treatment of inflammatory disease, the compounds provided herein may be administered in dosages, routes of administration and/or to achieve pK profiles as described herein for the treatment of cancer.

4.3.3 Rasopathies

As discussed herein, Ras signaling is causally implicated in rasopathies. Thus, the compounds provided herein, which inhibit the function of one or more members of the Ras superfamily, are useful in the treatment of rasopathies including neurofibromatosis type 1, Noonan's syndrome, and Costello syndrome.

In one embodiment, provided herein is a method of treating or preventing a rasopathy, which comprises administering to a subject a compound that inhibits the function of one or more members of the Ras superfamily. In one embodiment, provided herein is a method of treating or preventing a rasopathy, which comprises administering to a subject a compound that inhibits the function of one or more members of the Ras superfamily by binding to the GTP binding domain or one or more members of the Ras superfamily. In one embodiment, provided herein is a method of treating or preventing a rasopathy, which comprises administering to a subject a compound that inhibits the function of Ras by binding to a Ras GTP binding domain. In another embodiment, provided herein is a method of treating or preventing a rasopathy, which comprises administering to a subject a compound that binds to a Ras GTP binding domain with an IC₅₀ of less than 10 μM and K_{d} of less than 10 μM. In another embodiment, provided herein is a method of treating or preventing a rasopathy, which comprises administering to a subject a compound that binds to a Ras GTP binding domain with an IC₅₀ of less than 1 μM and a K_{d} of less than 1 μM. In another embodiment, provided herein is a method of treating or preventing a rasopathy, which comprises administering to a subject a compound that binds to a Ras GTP binding domain with an IC₅₀ of less than 500 nM and a K_{d} of less than 500 nM. In another embodiment, provided herein is a method of treating or preventing a rasopathy, which comprises administering to a subject a compound that binds to a Ras GTP binding domain with an IC₅₀ of less than 470 nM and a K_{d} of less than 470 nM. In another embodiment, provided herein is a method of treating or preventing a rasopathy, which comprises administering to a subject a compound that binds to a Ras GTP binding domain with an IC₅₀ of less than 270 nM and a K_{d} of less than 270 nM. In another embodiment, provided herein is a method of treating or preventing a rasopathy, which comprises administering to a subject a compound that binds to a Ras GTP binding domain with an IC₅₀ of less than 200 nM and a K_{d} of less than 200 nM. In another embodiment, provided herein is a method of treating or preventing a rasopathy, which comprises administering to a subject a compound that binds to a Ras GTP binding domain with an IC₅₀ of less than 150 nM and a K_{d} of less than 150 nM. In another embodiment, provided herein is a method of treating or preventing a rasopathy, which comprises administering to a subject a compound that binds to a Ras GTP binding domain with an IC₅₀ of less than 100 nM and a K_{d} of less than 100 nM. In another embodiment, provided herein is a method of treating or preventing a rasopathy, which comprises administering to a subject a compound that binds to a Ras GTP binding domain with greater than 15% inhibition at 20 μM. In another embodiment, provided herein is a method of treating or preventing a rasopathy, which comprises administering to a subject a compound that binds to a Ras GTP binding domain with greater than 25% inhibition at 20 μM. In another embodiment, provided herein is a method of treating or preventing a rasopathy, which comprises administering to a subject a compound that binds to a Ras GTP binding domain with greater than 50% inhibition at 20 μM. In another embodiment, provided herein is a method of treating or preventing a rasopathy, which comprises administering to a subject a compound that binds to a Ras GTP binding domain with greater than 75% inhibition at 20 μM. In another embodiment, provided herein is a method of treating or preventing a rasopathy, which comprises administering to a subject a compound that binds to a Ras GTP binding domain with greater than 80% inhibition at 20 μM. In another embodiment, provided herein is a method of treating or preventing a rasopathy, which comprises administering to a subject a compound that binds to a Ras GTP binding domain with greater than 85% inhibition at 20 μM. In another embodiment, provided herein is a method of treating or preventing a rasopathy, which comprises administering to a subject a compound that binds to a Ras GTP binding domain with greater than 90% inhibition at 20 μM. In another embodiment, provided herein is a method of treating or preventing a rasopathy, which comprises administering to a subject a compound that binds to a Ras GTP binding domain with greater than 95% inhibition at 20 μM. In another embodiment, provided herein is a method of treating or preventing a rasopathy, which comprises administering to a subject a compound that binds to a Ras GTP binding domain with greater than 99% inhibition at 20 μM. In one embodiment, provided herein is a method of treating or preventing a rasopathy, which comprises administering to a subject a compound that inhibits the function of Rho. In one embodiment, provided herein is a method of treating or preventing a rasopathy, which comprises administering to a subject a compound that inhibits the function of Rho by binding to a Rho GTP binding domain. In another embodiment, provided herein is a method of treating or preventing a rasopathy, which comprises administering to a subject a compound that binds to a Rho GTP binding domain with an IC₅₀ of less than 10 μM and a K_{d} of less than 10 μM. In another embodiment, provided herein is a method of treating or preventing a rasopathy, which comprises administering to a subject a compound that binds to a Rho GTP binding domain with an IC₅₀ of less than 1 μM and a K_{d} of less than 1 μM. In another embodiment, provided herein is a method of treating or preventing a rasopathy, which comprises administering to a subject a compound that binds to a Rho GTP binding domain with an IC₅₀ of less than 500 nM and a K_{d} of less than 500 nM. In another embodiment, provided herein is a method of treating or preventing a rasopathy, which comprises administering to a subject a compound that binds to a Rho GTP binding domain with an IC₅₀ of less than 270 nM and a K_{d} of less than 270 nM. In another embodiment, provided herein is a method of treating or preventing a rasopathy, which comprises administering to a subject a compound that binds to a Rho GTP binding domain with an IC₅₀ of less than 200 nM and a K_{d} of less than 200 nM. In another embodiment, provided herein is a method of treating or preventing a rasopathy, which comprises administering to a subject a compound that binds to a Rho GTP binding domain with an IC₅₀ of less than 150 nM and a K_{d} of less than 150 nM. In another embodiment, provided herein is a method of treating or preventing a rasopathy, which comprises administering to a subject a compound that binds to a Rho GTP binding domain with an IC₅₀ of less than 130 nM and a K_{d} of less than 130 nM. In another embodiment, provided herein is a method of treating or preventing a rasopathy, which comprises administering to a subject a compound that binds to a Rho GTP binding domain with an IC₅₀ of less than 100 nM and a K_{d} of less than 100 nM. In another embodiment, provided herein is a method of treating or preventing a rasopathy, which comprises administering to a subject a compound that binds to a Rho GTP binding domain with greater than 15% inhibition at 20 μM. In another embodiment, provided herein is a method of treating or preventing a rasopathy, which comprises administering to a subject a compound that binds to a Rho GTP binding domain with greater than 25% inhibition at 20 μM. In another embodiment, provided herein is a method of treating or preventing a rasopathy, which comprises administering to a subject a compound that binds to a Rho GTP binding domain with greater than 50% inhibition at 20 μM. In another embodiment, provided herein is a method of treating or preventing a rasopathy, which comprises administering to a subject a compound that binds to a Rho GTP binding domain with greater than 75% inhibition at 20 μM. In another embodiment, provided herein is a method of treating or preventing a rasopathy, which comprises administering to a subject a compound that binds to a Rho GTP binding domain with greater than 80% inhibition at 20 μM. In another embodiment, provided herein is a method of treating or preventing a rasopathy, which comprises administering to a subject a compound that binds to a Rho GTP binding domain with greater than 85% inhibition at 20 μM. In another embodiment, provided herein is a method of treating or preventing a rasopathy, which comprises administering to a subject a compound that binds to a Rho GTP binding domain with greater than 90% inhibition at 20 μM. In another embodiment, provided herein is a method of treating or preventing a rasopathy, which comprises administering to a subject a compound that binds to a Rho GTP binding domain with greater than 95% inhibition at 20 μM. In another embodiment, provided herein is a method of treating or preventing a rasopathy, which comprises administering to a subject a compound that binds to a Rho GTP binding domain with greater than 99% inhibition at 20 μM. In one embodiment, provided herein is a method of treating or preventing a rasopathy, which comprises administering to a subject a compound that inhibits the function of Rac In one embodiment, provided herein is a method of treating or preventing a rasopathy, which comprises administering to a subject a compound that inhibits the function of Rac by binding to a Rac GTP binding domain. In another embodiment, provided herein is a method of treating or preventing a rasopathy, which comprises administering to a subject a compound that binds to a Rac GTP binding domain with an IC₅₀ of less than 10 μM and a K_{d} of less than 10 μM. In another embodiment, provided herein is a method of treating or preventing a rasopathy, which comprises administering to a subject a compound that binds to a Rac GTP binding domain with an IC₅₀ of less than 1 μM and a K_{d} of less than 1 μM. In another embodiment, provided herein is a method of treating or preventing a rasopathy, which comprises administering to a subject a compound that binds to a Rac GTP binding domain with an IC₅₀ of less than 500 nM and a K_{d} of less than 500 nM. In another embodiment, provided herein is a method of treating or preventing a rasopathy, which comprises administering to a subject a compound that binds to a Rac GTP binding domain with an IC₅₀ of less than 270 nM and a K_{d} of less than 270 nM. In another embodiment, provided herein is a method of treating or preventing a rasopathy, which comprises administering to a subject a compound that binds to a Rac GTP binding domain with an IC₅₀ of less than 200 nM and a K_{d} of less than 200 nM. In another embodiment, provided herein is a method of treating or preventing a rasopathy, which comprises administering to a subject a compound that binds to a Rac GTP binding domain with an IC₅₀ of less than 170 nM and a K_{d} of less than 170 nM. In another embodiment, provided herein is a method of treating or preventing a rasopathy, which comprises administering to a subject a compound that binds to a Rac GTP binding domain with an IC₅₀ of less than 150 nM and a K_{d} of less than 150 nM. In another embodiment, provided herein is a method of treating or preventing a rasopathy, which comprises administering to a subject a compound that binds to a Rac GTP binding domain with an IC₅₀ of less than 100 nM and a K_{d} of less than 100 nM. In another embodiment, provided herein is a method of treating or preventing a rasopathy, which comprises administering to a subject a compound that binds to a Rac GTP binding domain with greater than 15% inhibition at 20 μM. In another embodiment, provided herein is a method of treating or preventing a rasopathy, which comprises administering to a subject a compound that binds to a Rac GTP binding domain with greater than 25% inhibition at 20 μM. In another embodiment, provided herein is a method of treating or preventing a rasopathy, which comprises administering to a subject a compound that binds to a Rac GTP binding domain with greater than 50% inhibition at 20 μM. In another embodiment, provided herein is a method of treating or preventing a rasopathy, which comprises administering to a subject a compound that binds to a Rac GTP binding domain with greater than 75% inhibition at 20 μM. In another embodiment, provided herein is a method of treating or preventing a rasopathy, which comprises administering to a subject a compound that binds to a Rac GTP binding domain with greater than 80% inhibition at 20 μM. In another embodiment, provided herein is a method of treating or preventing a rasopathy, which comprises administering to a subject a compound that binds to a Rac GTP binding domain with greater than 85% inhibition at 20 μM. In another embodiment, provided herein is a method of treating or preventing a rasopathy, which comprises administering to a subject a compound that binds to a Rac GTP binding domain with greater than 90% inhibition at 20 μM. In another embodiment, provided herein is a method of treating or preventing a rasopathy, which comprises administering to a subject a compound that binds to a Rac GTP binding domain with greater than 95% inhibition at 20 μM. In another embodiment, provided herein is a method of treating or preventing a rasopathy, which comprises administering to a subject a compound that binds to a Rac GTP binding domain with greater than 99% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In another embodiment, provided herein is a method of treating or preventing a rasopathy, which comprises administering to a subject a compound that binds to one or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to two or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to three or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to four or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to five or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to six or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to seven or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to eight or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to nine or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to ten or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to eleven or more of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing a rasopathy, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing a rasopathy, which comprises administering to a subject a compound that binds to all of Ala11, Gly12, Val14, Gly15, Lys16, Ser17, Ala18, Phe28, Val 29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In another embodiment, provided herein is a method of treating or preventing a rasopathy, which comprises administering to a subject a compound that binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to two or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to three or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to four or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to five or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to six or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to seven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to eight or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to nine or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to ten or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to eleven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to twelve or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to thirteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to fourteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to fifteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to sixteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds to seventeen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing a rasopathy, which comprises administering to a subject a compound that binds all of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

In one embodiment, provided herein is a method of treating or preventing a rasopathy, which comprises administering to a subject a compound that binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to two or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rho GTP binding domain. In one embodiment, provided herein is a method of treating or preventing a rasopathy, which comprises administering to a subject a compound that binds to three or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to four or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to five or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to six or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to seven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to eight or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to nine or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to ten or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to eleven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to twelve or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to thirteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to fourteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to fifteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to sixteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to seventeen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to eighteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing a rasopathy, which comprises administering to a subject a compound that binds to all of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is Rac. In one embodiment the Rac is RAC1; RAC2; RAC3 or RHOG. In one embodiment, the Rac is RAC1. In another embodiment, the Rac is a mutant form of a Rac described herein.

In one embodiment, the compound for use in the methods and compositions provided herein inhibit GTP binding to one or more members of the Ras superfamily. In one embodiment, the compound for use in the methods and compositions provided herein inhibit GTP binding to Ras. In one embodiment, the compounds provided herein inhibit GTP binding to Rho. In one embodiment, the compound for use in the methods and compositions provided herein inhibit GTP binding to Rac. In one embodiment, the compound for use in the methods and compositions provided herein inhibit GTP binding to Ras and Rho. In one embodiment, the compound for use in the methods and compositions provided herein inhibit GTP binding to Ras and Rac. In one embodiment, the compound for use in the methods and compositions provided herein inhibit GTP binding to Rho and Rac. In one embodiment, the compound for use in the methods and compositions provided herein inhibit GTP binding to Ras, Rho and Rac.

In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

4.3.4 Ras-Associated Autoimmune Leukoproliferative Disorder

As discussed herein, Ras has been causally associated with Ras-associated autoimmune leukoproliferative disorder. Thus, the compounds provided herein, which inhibit the function of Ras, are useful in the treatment of Ras-associated autoimmune leukoproliferative disorder.

In one embodiment, provided herein is a method of treating or preventing Ras-associated autoimmune leukoproliferative disorder, which comprises administering to a subject a compound that inhibits the function of Ras. In one embodiment, provided herein is a method of treating or preventing Ras-associated autoimmune leukoproliferative disorder, which comprises administering to a subject a compound that inhibits the function of Ras by binding to a Ras GTP binding domain. In another embodiment, provided herein is a method of treating or preventing Ras-associated autoimmune leukoproliferative disorder, which comprises administering to a subject a compound that binds to a Ras GTP binding domain with an IC₅₀ of less than 10 μM and K_{d} of less than 10 μM. In another embodiment, provided herein is a method of treating or preventing Ras-associated autoimmune leukoproliferative disorder, which comprises administering to a subject a compound that binds to a Ras GTP binding domain with an IC₅₀ of less than 1 μM and a K_{d} of less than 1 μM. In another embodiment, provided herein is a method of treating or preventing Ras-associated autoimmune leukoproliferative disorder, which comprises administering to a subject a compound that binds to a Ras GTP binding domain with an IC₅₀ of less than 500 nM and a K_{d} of less than 500 nM. In another embodiment, provided herein is a method of treating or preventing Ras-associated autoimmune leukoproliferative disorder, which comprises administering to a subject a compound that binds to a Ras GTP binding domain with an IC₅₀ of less than 470 nM and a K_{d} of less than 470 nM. In another embodiment, provided herein is a method of treating or preventing Ras-associated autoimmune leukoproliferative disorder, which comprises administering to a subject a compound that binds to a Ras GTP binding domain with an IC₅₀ of less than 270 nM and a K_{d} of less than 270 nM. In another embodiment, provided herein is a method of treating or preventing Ras-associated autoimmune leukoproliferative disorder, which comprises administering to a subject a compound that binds to a Ras GTP binding domain with an IC₅₀ of less than 200 nM and a K_{d} of less than 200 nM. In another embodiment, provided herein is a method of treating or preventing Ras-associated autoimmune leukoproliferative disorder, which comprises administering to a subject a compound that binds to a Ras GTP binding domain with an IC₅₀ of less than 150 nM and a K_{d} of less than 150 nM. In another embodiment, provided herein is a method of treating or preventing Ras-associated autoimmune leukoproliferative disorder, which comprises administering to a subject a compound that binds to a Ras GTP binding domain with an IC₅₀ of less than 100 nM and a K_{d} of less than 100 nM. In another embodiment, provided herein is a method of treating or preventing Ras-associated autoimmune leukoproliferative disorder, which comprises administering to a subject a compound that binds to a Ras GTP binding domain with greater than 15% inhibition at 20 μM. In another embodiment, provided herein is a method of treating or preventing Ras-associated autoimmune leukoproliferative disorder, which comprises administering to a subject a compound that binds to a Ras GTP binding domain with greater than 25% inhibition at 20 μM. In another embodiment, provided herein is a method of treating or preventing Ras-associated autoimmune leukoproliferative disorder, which comprises administering to a subject a compound that binds to a Ras GTP binding domain with greater than 50% inhibition at 20 μM. In another embodiment, provided herein is a method of treating or preventing Ras-associated autoimmune leukoproliferative disorder, which comprises administering to a subject a compound that binds to a Ras GTP binding domain with greater than 75% inhibition at 20 μM. In another embodiment, provided herein is a method of treating or preventing Ras-associated autoimmune leukoproliferative disorder, which comprises administering to a subject a compound that binds to a Ras GTP binding domain with greater than 80% inhibition at 20 μM. In another embodiment, provided herein is a method of treating or preventing Ras-associated autoimmune leukoproliferative disorder, which comprises administering to a subject a compound that binds to a Ras GTP binding domain with greater than 85% inhibition at 20 μM. In another embodiment, provided herein is a method of treating or preventing Ras-associated autoimmune leukoproliferative disorder, which comprises administering to a subject a compound that binds to a Ras GTP binding domain with greater than 90% inhibition at 20 μM. In another embodiment, provided herein is a method of treating or preventing Ras-associated autoimmune leukoproliferative disorder, which comprises administering to a subject a compound that binds to a Ras GTP binding domain with greater than 95% inhibition at 20 μM. In another embodiment, provided herein is a method of treating or preventing Ras-associated autoimmune leukoproliferative disorder, which comprises administering to a subject a compound that binds to a Ras GTP binding domain with greater than 99% inhibition at 20 μM. In some embodiments, the compound for use in the method, or the administered compound, is a compound as disclosed herein of Formula IA, IB, IC, ID, IE, IF, IIA, or IIB, or a pharmaceutically acceptable salt thereof. In some embodiments, the compound as disclosed herein for use in the method, or the administered compound, is a compound selected from Compounds 1-39, such as a compound selected from compounds 1-27 of Examples 1-27, or a pharmaceutically acceptable salt thereof.

In another embodiment, provided herein is a method of treating or preventing Ras-associated autoimmune leukoproliferative disorder, which comprises administering to a subject a compound that binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to four or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to five or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to six or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to seven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to eight or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to nine or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to ten or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to eleven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In another embodiment, provided herein is a method of treating or preventing Ras-associated autoimmune leukoproliferative disorder, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing Ras-associated autoimmune leukoproliferative disorder, which comprises administering to a subject a compound that binds to all of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146 and Lys147 or Mg202 in a Ras GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In one embodiment, the compound for use in the methods and compositions provided herein has a molecular weight less than 2000 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

4.3.5 Fibrotic Disease

As discussed herein, Ras superfamily members are potential targets in fibrotic disease treatment. In one embodiment, provided herein is a method of treating or preventing fibrotic disease, which comprises administering to a subject a compound that inhibits the function of one or more members of the Ras superfamily. In one embodiment, provided herein is a method of treating or preventing fibrotic disease, which comprises administering to a subject a compound that inhibits the function of one or more members of the Ras superfamily by binding to the GTP binding domain or one or more members of the Ras superfamily. In one embodiment, provided herein is a method of treating or preventing fibrotic disease, which comprises administering to a subject a compound that inhibits the function of Ras by binding to a Rho GTP binding domain. In another embodiment, provided herein is a method of treating or preventing fibrotic disease, which comprises administering to a subject a compound that binds to a Ras GTP binding domain with an IC₅₀ of less than 10 μM and K_{d} of less than 10 μM. In another embodiment, provided herein is a method of treating or preventing fibrotic disease, which comprises administering to a subject a compound that binds to a Ras GTP binding domain with an IC₅₀ of less than 1 μM and a K_{d} of less than 1 μM. In another embodiment, provided herein is a method of treating or preventing fibrotic disease, which comprises administering to a subject a compound that binds to a Ras GTP binding domain with an IC₅₀ of less than 500 nM and a K_{d} of less than 500 nM. In another embodiment, provided herein is a method of treating or preventing fibrotic disease, which comprises administering to a subject a compound that binds to a Ras GTP binding domain with an IC₅₀ of less than 470 nM and a K_{d} of less than 470 nM. In another embodiment, provided herein is a method of treating or preventing fibrotic disease, which comprises administering to a subject a compound that binds to a Ras GTP binding domain with an IC₅₀ of less than 270 nM and a K_{d} of less than 270 nM. In another embodiment, provided herein is a method of treating or preventing fibrotic disease, which comprises administering to a subject a compound that binds to a Ras GTP binding domain with an IC₅₀ of less than 200 nM and a K_{d} of less than 200 nM. In another embodiment, provided herein is a method of treating or preventing fibrotic disease, which comprises administering to a subject a compound that binds to a Ras GTP binding domain with an IC₅₀ of less than 150 nM and a K_{d} of less than 150 nM. In another embodiment, provided herein is a method of treating or preventing fibrotic disease, which comprises administering to a subject a compound that binds to a Ras GTP binding domain with an IC₅₀ of less than 100 nM and a K_{d} of less than 100 nM. In another embodiment, provided herein is a method of treating or preventing fibrotic disease, which comprises administering to a subject a compound that binds to a Ras GTP binding domain with greater than 15% inhibition at 20 μM. In another embodiment, provided herein is a method of treating or preventing fibrotic disease, which comprises administering to a subject a compound that binds to a Ras GTP binding domain with greater than 25% inhibition at 20 μM. In another embodiment, provided herein is a method of treating or preventing fibrotic disease, which comprises administering to a subject a compound that binds to a Ras GTP binding domain with greater than 50% inhibition at 20 μM. In another embodiment, provided herein is a method of treating or preventing fibrotic disease, which comprises administering to a subject a compound that binds to a Ras GTP binding domain with greater than 75% inhibition at 20 μM. In another embodiment, provided herein is a method of treating or preventing fibrotic disease, which comprises administering to a subject a compound that binds to a Ras GTP binding domain with greater than 80% inhibition at 20 μM. In another embodiment, provided herein is a method of treating or preventing fibrotic disease, which comprises administering to a subject a compound that binds to a Ras GTP binding domain with greater than 85% inhibition at 20 μM. In another embodiment, provided herein is a method of treating or preventing fibrotic disease, which comprises administering to a subject a compound that binds to a Ras GTP binding domain with greater than 90% inhibition at 20 μM. In another embodiment, provided herein is a method of treating or preventing fibrotic disease, which comprises administering to a subject a compound that binds to a Ras GTP binding domain with greater than 95% inhibition at 20 μM. In another embodiment, provided herein is a method of treating or preventing fibrotic disease, which comprises administering to a subject a compound that binds to a Ras GTP binding domain with greater than 99% inhibition at 20 μM. In one embodiment, provided herein is a method of treating or preventing fibrotic disease, which comprises administering to a subject a compound that inhibits the function of Rho. In one embodiment, provided herein is a method of treating or preventing fibrotic disease, which comprises administering to a subject a compound that inhibits the function of Rho by binding to a Rho GTP binding domain. In another embodiment, provided herein is a method of treating or preventing fibrotic disease, which comprises administering to a subject a compound that binds to a Rho GTP binding domain with an IC₅₀ of less than 10 μM and K_{d} of less than 10 μM. In another embodiment, provided herein is a method of treating or preventing fibrotic disease, which comprises administering to a subject a compound that binds to a Rho GTP binding domain with an IC₅₀ of less than 1 μM and a K_{d} of less than 1 μM. In another embodiment, provided herein is a method of treating or preventing fibrotic disease, which comprises administering to a subject a compound that binds to a Rho GTP binding domain with an IC₅₀ of less than 500 nM and a K_{d} of less than 500 nM. In another embodiment, provided herein is a method of treating or preventing fibrotic disease, which comprises administering to a subject a compound that binds to a Rho GTP binding domain with an IC₅₀ of less than 270 nM and a K_{d} of less than 270 nM. In another embodiment, provided herein is a method of treating or preventing fibrotic disease, which comprises administering to a subject a compound that binds to a Rho GTP binding domain with an IC₅₀ of less than 200 nM and a K_{d} of less than 200 nM. In another embodiment, provided herein is a method of treating or preventing fibrotic disease, which comprises administering to a subject a compound that binds to a Rho GTP binding domain with an IC₅₀ of less than 150 nM and a K_{d} of less than 150 nM. In another embodiment, provided herein is a method of treating or preventing fibrotic disease, which comprises administering to a subject a compound that binds to a Rho GTP binding domain with an IC₅₀ of less than 130 nM and a K_{d} of less than 130 nM. In another embodiment, provided herein is a method of treating or preventing fibrotic disease, which comprises administering to a subject a compound that binds to a Rho GTP binding domain with an IC₅₀ of less than 100 nM and a K_{d} of less than 100 nM. In another embodiment, provided herein is a method of treating or preventing fibrotic disease, which comprises administering to a subject a compound that binds to a Rho GTP binding domain with greater than 15% inhibition at 20 μM. In another embodiment, provided herein is a method of treating or preventing fibrotic disease, which comprises administering to a subject a compound that binds to a Rho GTP binding domain with greater than 25% inhibition at 20 μM. In another embodiment, provided herein is a method of treating or preventing fibrotic disease, which comprises administering to a subject a compound that binds to a Rho GTP binding domain with greater than 50% inhibition at 20 μM. In another embodiment, provided herein is a method of treating or preventing fibrotic disease, which comprises administering to a subject a compound that binds to a Rho GTP binding domain with greater than 75% inhibition at 20 μM. In another embodiment, provided herein is a method of treating or preventing fibrotic disease, which comprises administering to a subject a compound that binds to a Rho GTP binding domain with greater than 80% inhibition at 20 μM. In another embodiment, provided herein is a method of treating or preventing fibrotic disease, which comprises administering to a subject a compound that binds to a Rho GTP binding domain with greater than 85% inhibition at 20 μM. In another embodiment, provided herein is a method of treating or preventing fibrotic disease, which comprises administering to a subject a compound that binds to a Rho GTP binding domain with greater than 90% inhibition at 20 μM. In another embodiment, provided herein is a method of treating or preventing fibrotic disease, which comprises administering to a subject a compound that binds to a Rho GTP binding domain with greater than 95% inhibition at 20 μM. In another embodiment, provided herein is a method of treating or preventing fibrotic disease, which comprises administering to a subject a compound that binds to a Rho GTP binding domain with greater than 99% inhibition at 20 μM. In one embodiment, provided herein is a method of treating or preventing fibrotic disease, which comprises administering to a subject a compound that inhibits the function of Rac. In one embodiment, provided herein is a method of treating or preventing fibrotic disease, which comprises administering to a subject a compound that inhibits the function of Rac by binding to a Rac GTP binding domain. In another embodiment, provided herein is a method of treating or preventing fibrotic disease, which comprises administering to a subject a compound that binds to a Rac GTP binding domain with an IC₅₀ of less than 10 μM and K_{d} of less than 10 μM. In another embodiment, provided herein is a method of treating or preventing fibrotic disease, which comprises administering to a subject a compound that binds to a Rac GTP binding domain with an IC₅₀ of less than 1 μM and a K_{d} of less than 1 μM. In another embodiment, provided herein is a method of treating or preventing fibrotic disease, which comprises administering to a subject a compound that binds to a Rac GTP binding domain with an IC₅₀ of less than 500 nM and a K_{d} of less than 500 nM. In another embodiment, provided herein is a method of treating or preventing fibrotic disease, which comprises administering to a subject a compound that binds to a Rac GTP binding domain with an IC₅₀ of less than 270 nM and a K_{d} of less than 270 nM. In another embodiment, provided herein is a method of treating or preventing fibrotic disease, which comprises administering to a subject a compound that binds to a Rac GTP binding domain with an IC₅₀ of less than 200 nM and a K_{d} of less than 200 nM. In another embodiment, provided herein is a method of treating or preventing fibrotic disease, which comprises administering to a subject a compound that binds to a Rac GTP binding domain with an IC₅₀ of less than 170 nM and a K_{d} of less than 170 nM. In another embodiment, provided herein is a method of treating or preventing fibrotic disease, which comprises administering to a subject a compound that binds to a Rac GTP binding domain with an IC₅₀ of less than 150 nM and a K_{d} of less than 150 nM. In another embodiment, provided herein is a method of treating or preventing fibrotic disease, which comprises administering to a subject a compound that binds to a Rac GTP binding domain with an IC₅₀ of less than 100 nM and a K_{d} of less than 100 nM. In another embodiment, provided herein is a method of treating or preventing fibrotic disease, which comprises administering to a subject a compound that binds to a Rac GTP binding domain with greater than 15% inhibition at 20 μM. In another embodiment, provided herein is a method of treating or preventing fibrotic disease, which comprises administering to a subject a compound that binds to a Rac GTP binding domain with greater than 25% inhibition at 20 μM. In another embodiment, provided herein is a method of treating or preventing fibrotic disease, which comprises administering to a subject a compound that binds to a Rac GTP binding domain with greater than 50% inhibition at 20 μM. In another embodiment, provided herein is a method of treating or preventing fibrotic disease, which comprises administering to a subject a compound that binds to a Rac GTP binding domain with greater than 75% inhibition at 20 μM. In another embodiment, provided herein is a method of treating or preventing fibrotic disease, which comprises administering to a subject a compound that binds to a Rac GTP binding domain with greater than 80% inhibition at 20 μM. In another embodiment, provided herein is a method of treating or preventing fibrotic disease, which comprises administering to a subject a compound that binds to a Rac GTP binding domain with greater than 85% inhibition at 20 μM. In another embodiment, provided herein is a method of treating or preventing fibrotic disease, which comprises administering to a subject a compound that binds to a Rac GTP binding domain with greater than 90% inhibition at 20 μM. In another embodiment, provided herein is a method of treating or preventing fibrotic disease, which comprises administering to a subject a compound that binds to a Rac GTP binding domain with greater than 95% inhibition at 20 μM. In another embodiment, provided herein is a method of treating or preventing fibrotic disease, which comprises administering to a subject a compound that binds to a Rac GTP binding domain with greater than 99% inhibition at 20 μM. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In another embodiment, provided herein is a method of treating or preventing fibrotic disease, which comprises administering to a subject a compound that binds to one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to four or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to five or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to six or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to seven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to eight or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to nine or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to ten or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to eleven or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to twelve or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to thirteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to fourteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to fifteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to sixteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to seventeen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to eighteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to nineteen or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to twenty or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to twenty-one or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to twenty-two or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to twenty-three or more of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In another embodiment, provided herein is a method of treating or preventing fibrotic disease, which comprises administering to a subject a compound that binds to all of Ala11, Gly12, Val14, Gly15, Lys16, Ser17, Ala18, Phe28, Val29, Asp30, Glu31, Tyr32, Asp33, Pro34, Thr35, Ile36, Gly60, Gln61, Lys117, Asp119, Leu120, Ser145, Ala146, Lys147 or Mg202 in a Ras GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2. In another embodiment, the Ras is HRAS, KRAS or NRAS. In one embodiment, the Ras is HRAS. In one embodiment, the Ras is KRAS. In one embodiment, the Ras is NRAS. In another embodiment, the Ras is a mutant form of a Ras described herein.

In another embodiment, provided herein is a method of treating or preventing fibrotic disease, which comprises administering to a subject a compound that binds to one or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to two or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to three or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to four or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to five or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to six or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to seven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to eight or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to nine or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to ten or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to eleven or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to twelve or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to thirteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to fourteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to fifteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to sixteen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds to seventeen or more of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In another embodiment, provided herein is a method of treating or preventing fibrotic disease, which comprises administering to a subject a compound that binds all of Gly14, Ala15, Cys16, Gly17, Lys18, Thr19, Cys20, Phe30, Pro31, Glu32, Tyr34, Val35, Pro36, Thr37, Asp59, Lys118, Asp120, Lys162 or Mg202 in a Rho GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3 or CDC42. In one embodiment, the Rho is RHOA. In another embodiment, the Rho is a mutant form of a Rho described herein.

In one embodiment, provided herein is a method of treating or preventing fibrotic disease, which comprises administering to a subject a compound that binds to one or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to two or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a highly conserved Rho GTP binding domain. In one embodiment, provided herein is a method of treating or preventing fibrotic disease, which comprises administering to a subject a compound that binds to three or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to four or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to five or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to six or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to seven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to eight or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to nine or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to ten or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to eleven or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to twelve or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to thirteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to fourteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to fifteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to sixteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to seventeen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to eighteen or more of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In one embodiment, provided herein is a method of treating or preventing fibrotic disease, which comprises administering to a subject a compound that binds to all of Gly12, Ala13, Gly15, Lys16, Thr17, Cys18, Leu19, Phe28, Ile33, Pro34, Val36, Ala59, Thr115, Lys116, Asp118, Leu119, Cys157, Ala159, or Mg202 in a Rac GTP binding domain. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

In one embodiment, the Rho is Rac. In one embodiment the Rac is RAC1; RAC2; RAC3 or RHOG. In one embodiment, the Rac is RAC1. In another embodiment, the Rac is a mutant form of a Rac described herein.

In one embodiment, the compound for use in the methods and compositions provided herein inhibit GTP binding to one or more members of the Ras superfamily. In one embodiment, the compound for use in the methods and compositions provided herein inhibit GTP binding to Ras. In one embodiment, the compounds provided herein inhibit GTP binding to Rho. In one embodiment, the compound for use in the methods and compositions provided herein inhibit GTP binding to Rac. In one embodiment, the compound for use in the methods and compositions provided herein inhibit GTP binding to Ras and Rho. In one embodiment, the compound for use in the methods and compositions provided herein inhibit GTP binding to Ras and Rac. In one embodiment, the compound for use in the methods and compositions provided herein inhibit GTP binding to Rho and Rac. In one embodiment, the compound for use in the methods and compositions provided herein inhibit GTP binding to Ras, Rho and Rac.

In one embodiment, the compound for use in the methods and compositions provided herein has a molecular weight less than 2000 daltons. In one embodiment, the compound for use in the methods and compositions provided herein has a molecular weight less than 1750 daltons. In one embodiment, the compound for use in the methods and compositions provided herein has a molecular weight less than 1500 daltons. In one embodiment, the compound for use in the methods and compositions provided herein has a molecular weight less than 1250 daltons. In one embodiment, the compound for use in the methods and compositions provided herein has a molecular weight less than 1000 daltons. In one embodiment, the compound for use in the methods and compositions provided herein has a molecular weight less than 750 daltons. In one embodiment, the compound for use in the methods and compositions provided herein has a molecular weight less than 665 daltons. In one embodiment, the compound for use in the methods and compositions provided herein has a molecular weight less than 500 daltons. In some embodiments, the compound for use in the method is a compound as disclosed herein of Formula IA, IB, IC, ID, IE, IF, IIA, or IIB, or a pharmaceutically acceptable salt thereof. In some embodiments, the compound as disclosed herein for use in the method is a compound selected from Compounds 1-39, such as a compound selected from compounds 1-27 of Examples 1-27, or a pharmaceutically acceptable salt thereof.

### 4.4. Compounds for Use in Compositions and Methods

Provided herein are compounds which bind to the GTP binding domain of one or more Ras superfamily members and compete with the binding of GTP to one or more Ras superfamily members.

Provided herein are compounds which bind to a Ras GTP binding domain and compete with the binding of GTP to Ras. In one embodiment, the compounds also inhibit phosphorylation of ERK1/2, Akt (for example, Akt1, Akt2 and Akt3) cellular proliferation, secretion of IL-6 or TNF-α cytokines. The compounds provided herein are therefore useful in compositions and methods of treating cancer, inflammatory diseases, Ras-associated autoimmune leukoproliferative disorder and rasopathies.

Provided herein are compounds which bind to a Rac GTP binding domain and compete with the binding of GTP to Rac. In one embodiment, the compounds also inhibit the ERK1/2 and Akt signaling pathways. In one embodiment, the compounds also inhibit the ROCK signaling pathway. The compounds provided herein are therefore useful in compositions and methods of treating cancer, inflammatory diseases and fibrotic disease.

Provided herein are compounds which bind to a Rho GTP binding domain and compete with the binding of GTP to Rho. In one embodiment, the compounds also inhibit the ERK1/2 and Akt signaling pathways. In one embodiment, the compounds also inhibit the ROCK signaling pathway. The compounds provided herein are therefore useful in compositions and methods of treating cancer, inflammatory diseases and fibrotic disease.

In one embodiment, the compounds provided herein inhibit GTP binding to one or more members of the Ras superfamily. In one embodiment, the compounds provided herein inhibit GTP binding to Ras. In one embodiment, the compounds provided herein inhibit GTP binding to Rho. In one embodiment, the compounds provided herein inhibit GTP binding to Rac. In one embodiment, the compounds provided herein inhibit GTP binding to Ras and Rho. In one embodiment, the compounds provided herein inhibit GTP binding to Ras and Rac. In one embodiment, the compounds provided herein inhibit GTP binding to Rho and Rac. In one embodiment, the compounds provided herein inhibit GTP binding to Ras, Rho and Rac.

In one embodiment, the compounds provided herein inhibit activation of the MAPK pathway, in particular ERK1/2, and downregulate the proliferation of different human tumor cell lines. In one embodiment, the compounds provided herein inhibit activation of the AKT pathway and downregulate the proliferation of different human tumor cell lines. In one embodiment, the compounds provided herein inhibit activation of the MAPK pathway, in particular ERK1/2, and AKT pathway and downregulate the proliferation of different human tumor cell lines.

In certain embodiments, the compound for use in the compositions and methods provided herein is of Formula IA:

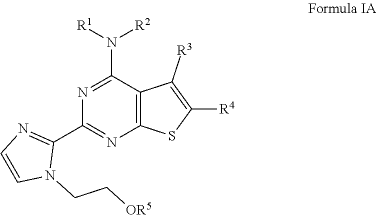

or a pharmaceutically acceptable derivative thereof, wherein:

—NR¹R² is

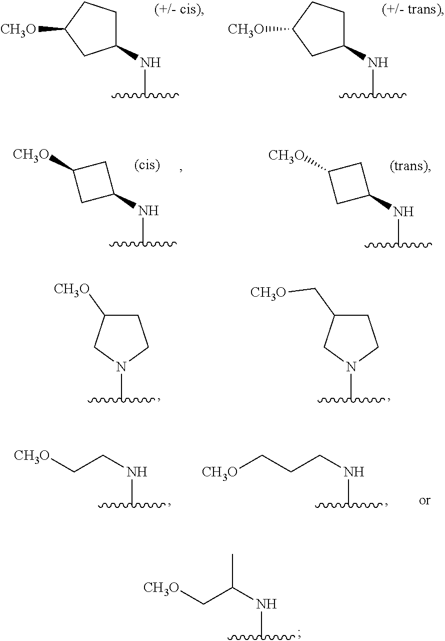

R³ is hydrogen, —CH₃, —CF₃, or phenyl;

R⁴ is hydrogen,

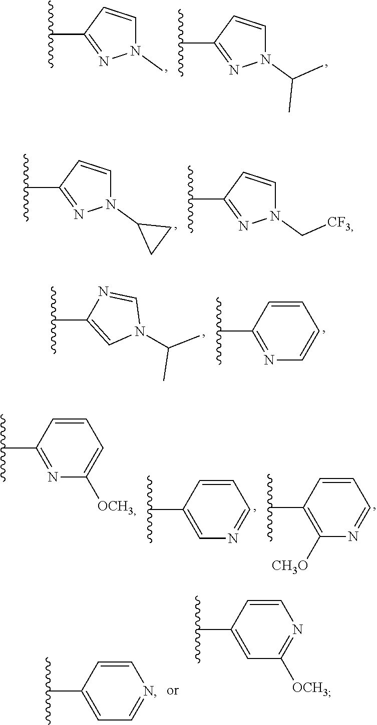

and

R⁵ is —CH₃ or —CH₂CF₃.

In some embodiments, the compound of Formula IA, wherein —NR¹R² is

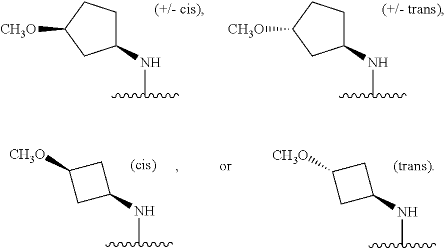

In some embodiments, the compound of Formula IA, wherein R³ is hydrogen.

In some embodiments, the compound of Formula IA, wherein R³ is —CH₃.

In some embodiments, the compound of Formula IA, wherein R³ is phenyl.

In certain embodiments, the compound for use in the compositions and methods provided herein is the compound of Formula IA, or a pharmaceutically acceptable derivative thereof, wherein:

—NR¹R² is

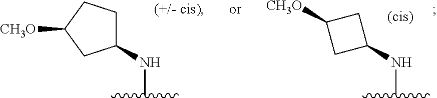

R³ is hydrogen or phenyl;

R⁴ is hydrogen of

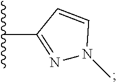 [SMILES: *c1ccn(C)n1]

and

R⁵ is —CH₃.

In some embodiments, the compound of Formula IA, wherein R³ is hydrogen.

In some embodiments, the compound of Formula IA, wherein R³ is phenyl.

In some embodiments, the compound of Formula IA, wherein R⁴ is hydrogen.

In some embodiments, the compound of Formula IA, wherein R⁴ is

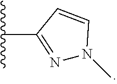 [SMILES: *c1ccn(C)n1]

In some embodiments, the compound of Formula IA is:

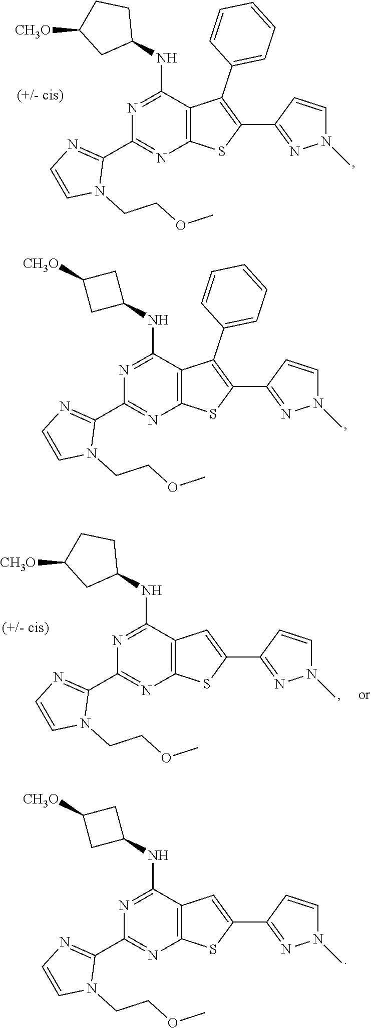

In certain embodiments, the pharmaceutically acceptable derivative of the compound of Formula IA is a pharmaceutically acceptable salt of the compound of Formula IA.

In certain embodiments, the compound for use in the compositions and methods provided herein is of Formula IB:

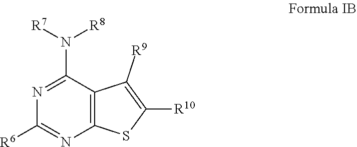

or a pharmaceutically acceptable derivative thereof, wherein:

R⁶ is

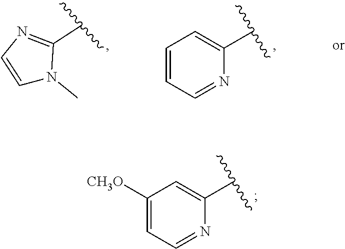

—NR⁷R⁸ is

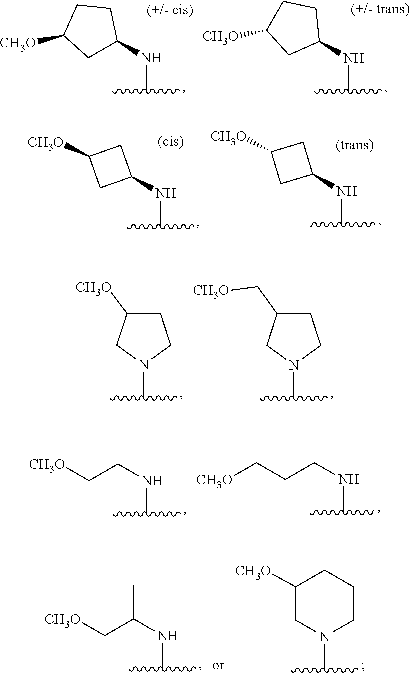

R⁹ is hydrogen, —CH₃, —CF₃, or phenyl; and

R¹⁰ is

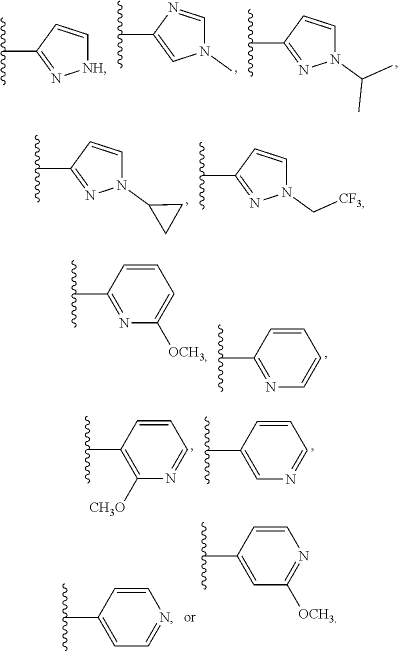

In some embodiments, the compound of Formula IB, wherein R⁶ is N

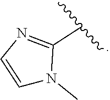 [SMILES: *c1nccn1C]

In some embodiments, the compound of Formula IB, wherein R⁶ is N

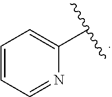 [SMILES: *c1ccccn1]

In some embodiments, the compound of Formula IB, wherein R⁶ is N

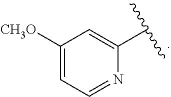

In some embodiments, the compound of Formula IB, wherein —NR⁷R⁸ is:

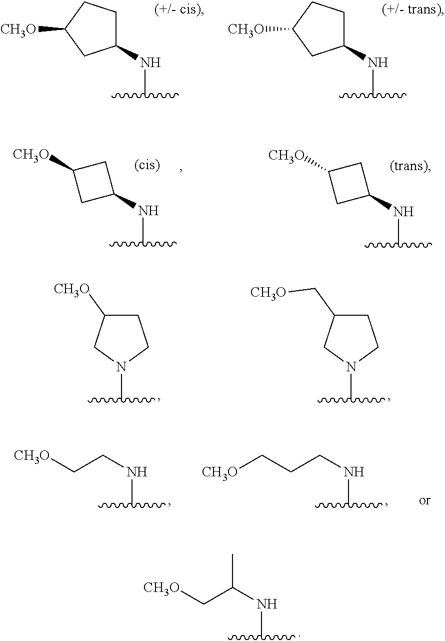

In some embodiments, the compound of Formula 1B, wherein —NR⁷R⁸ is

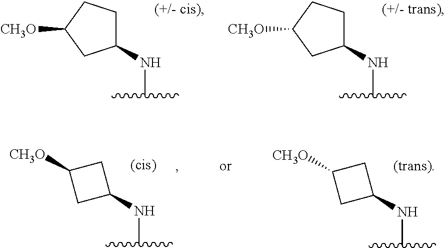

In some embodiments, the compound of Formula 1B, wherein —NR⁷R⁸ is

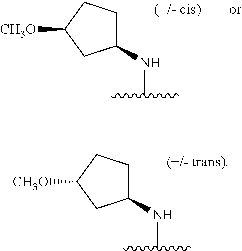

In some embodiments, the compound of Formula IB, wherein —NR⁷R⁸ is

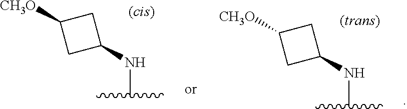

In some embodiments, the compound of Formula 1B, wherein —NR⁷R⁸ is

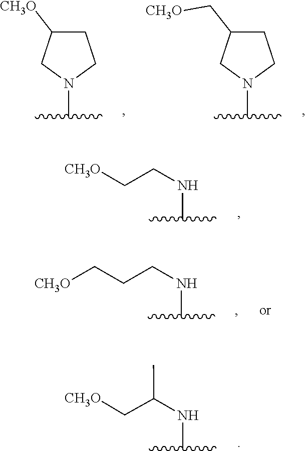

In some embodiments, the compound of Formula 11B, wherein —NR⁷R⁸ is

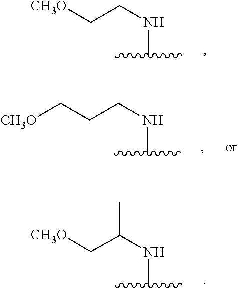

In some embodiments, the compound of Formula 1B, wherein —NR⁷R⁸ is

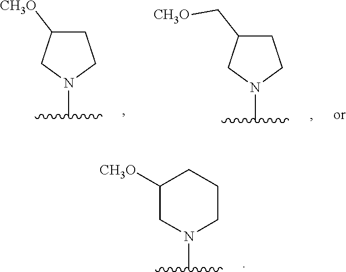

In some embodiments, the compound of Formula 1B, wherein —NR⁷R⁸ is

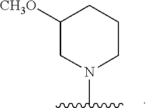

In some embodiments, the compound of Formula IB, wherein R⁹ is hydrogen.

In some embodiments, the compound of Formula IB, wherein R⁹ is —CH₃.

In some embodiments, the compound of Formula IB, wherein R⁹ is phenyl.

In some embodiments, the compound of Formula IB, wherein R¹⁰ is

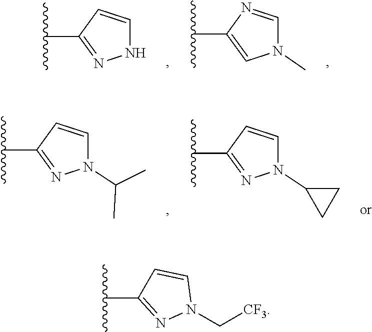

In some embodiments, the compound of Formula IB, wherein R¹⁰ is

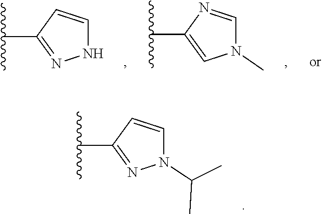

In some embodiments, the compound of Formula 1B, wherein R¹⁰ is

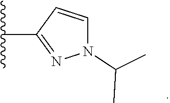 [SMILES: *c1ccn(C(C)C)n1]

In some embodiments, the compound of Formula IB, wherein R¹⁰ is

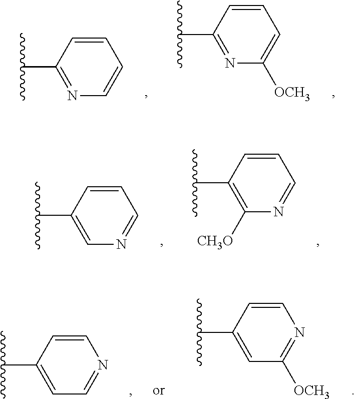

In some embodiments, the compound of Formula IB is:

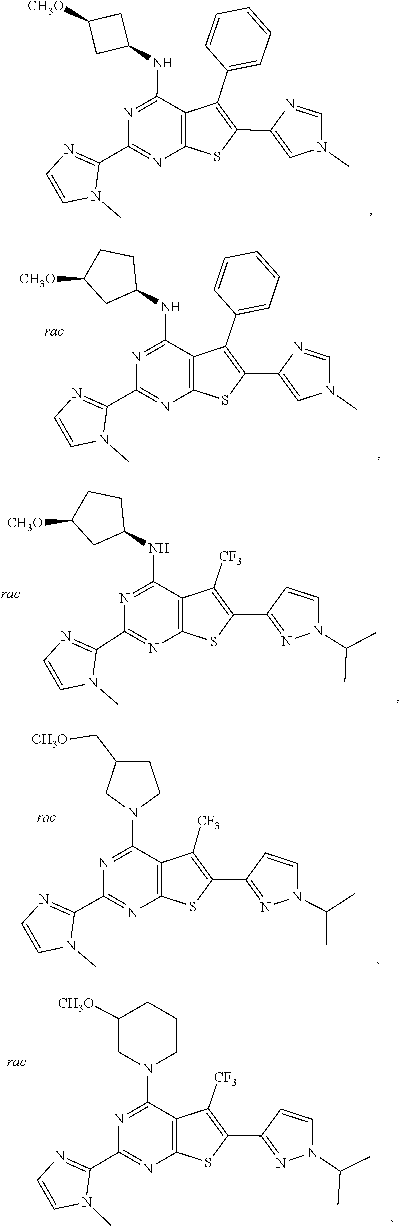

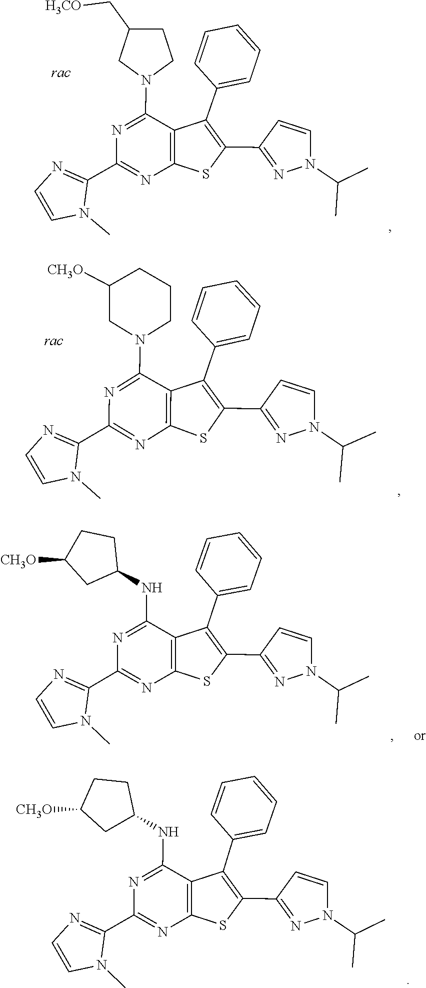

In some embodiments, the compound of Formula IB is:

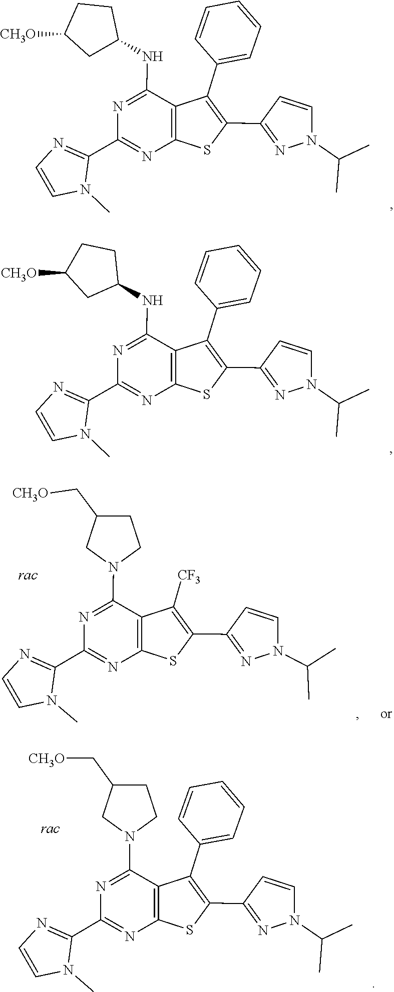

In some embodiments, the compound of Formula IB is

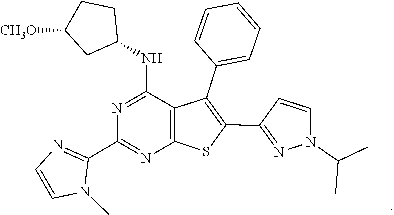

In certain embodiments, the compound for use in the compositions and methods provided herein is the compound of Formula IB, or a pharmaceutically acceptable derivative thereof, wherein:

R⁶ is

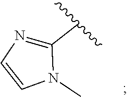 [SMILES: *c1nccn1C]

—NR⁷R⁸ is

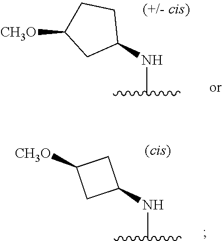

R⁹ is phenyl; and

R¹⁰ is

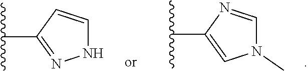

In some embodiments, the compound of Formula IB, wherein —NR⁷R⁸ is

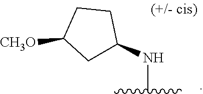

In some embodiments, the compound of Formula IB, wherein —NR⁷R⁸ is

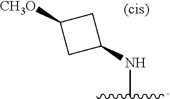

In some embodiments, the compound of Formula IB, wherein R¹⁰ is

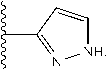 [SMILES: *c1cc[nH]n1]

In some embodiments, the compound of Formula IB, wherein R¹⁰ is

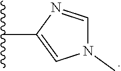 [SMILES: *c1cn(C)cn1]

In some embodiments, the compound of Formula IB is:

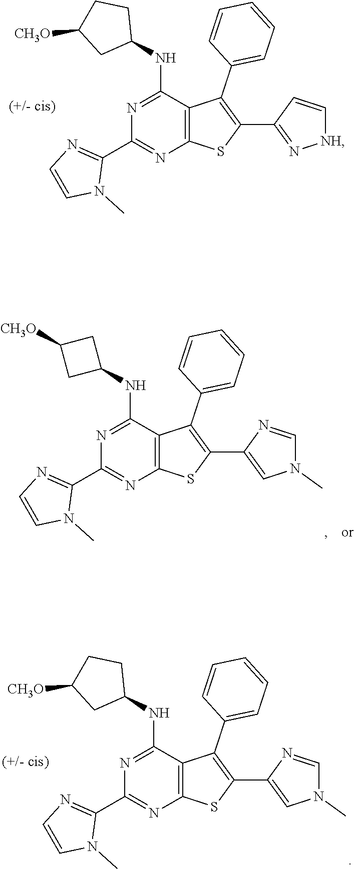

In certain embodiments, the pharmaceutically acceptable derivative of the compound of Formula IB is a pharmaceutically acceptable salt of the compound of Formula IB.

In certain embodiments, the compound for use in the compositions and methods provided herein is of Formula IC:

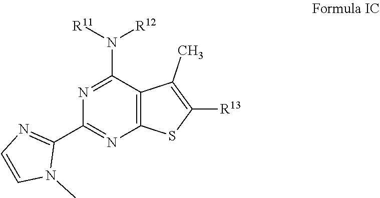

or a pharmaceutically acceptable derivative thereof, wherein:

—NR¹¹R¹² is

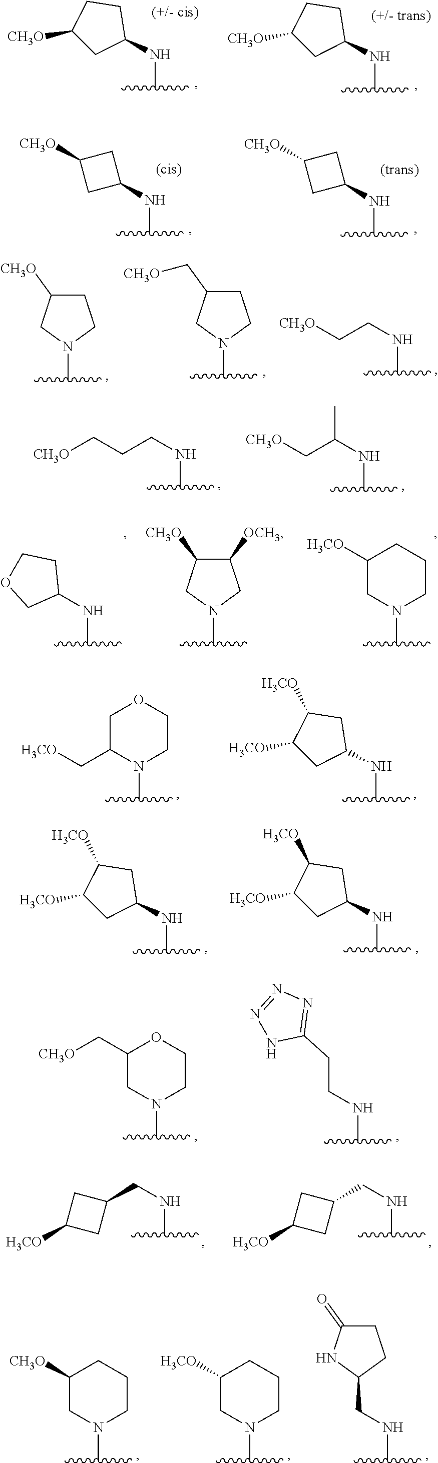

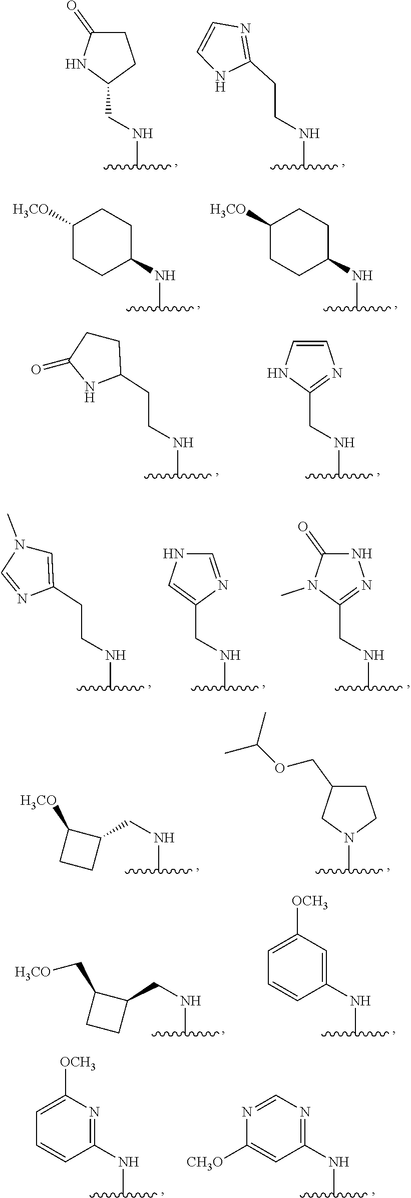

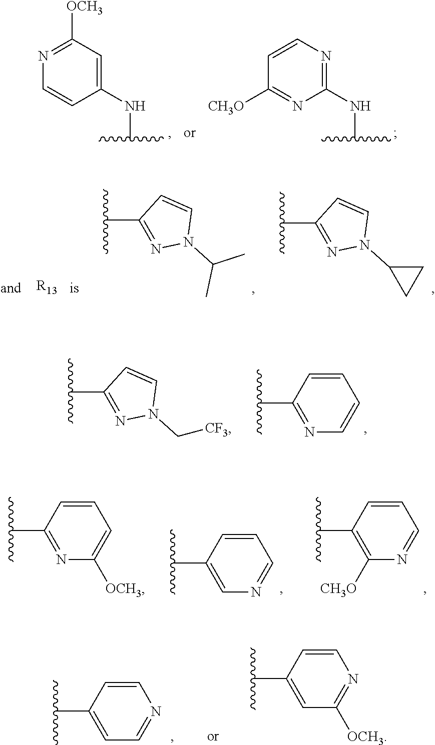

In some embodiments, the compound of Formula 1C, wherein R¹³ is

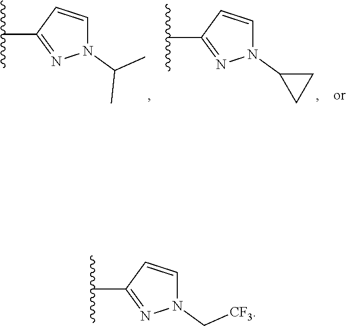

In some embodiments, the compound of Formula 1C, wherein R¹³ is

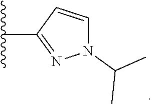 [SMILES: *c1ccn(C(C)C)n1]

In some embodiments, the compounds of Formula 1C, wherein R¹³ is

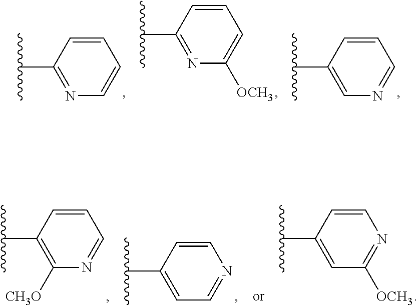

In some embodiments, the compound of Formula IC, wherein —NR¹¹R¹² is

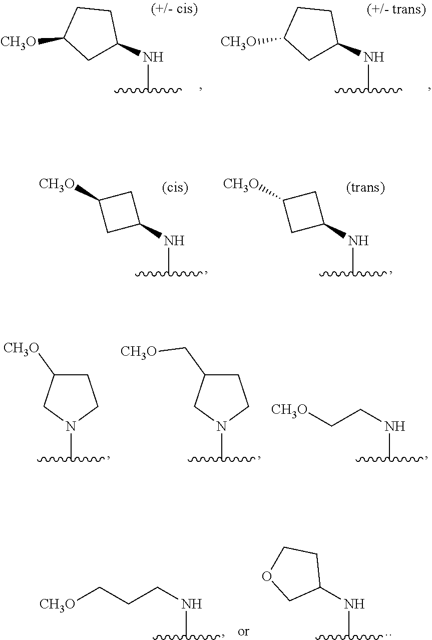

In some embodiments, the compound of Formula IC, wherein —NR¹¹R¹² is

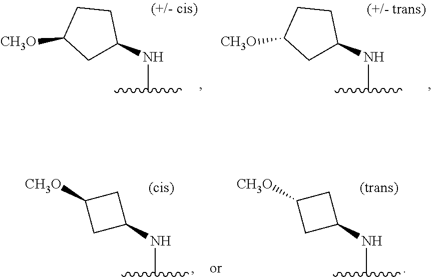

In some embodiments, the compound of Formula IC, wherein —NR¹¹R¹² is

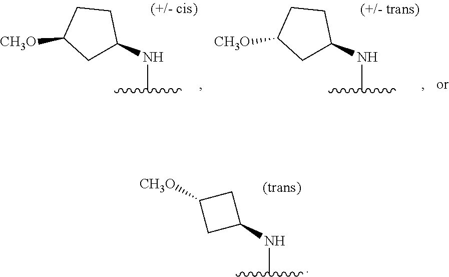

In some embodiments, the compound of Formula 1C, wherein —NR¹¹R¹² is

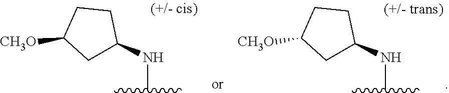

In some embodiments, the compound of Formula IC, wherein —NR¹¹R¹² is

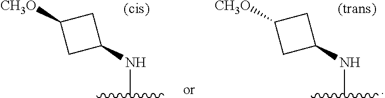

In some embodiments, the compound of Formula IC, wherein —NR¹¹R¹² is

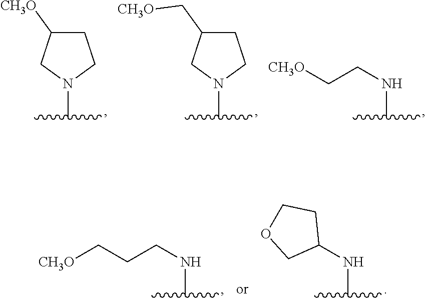

In some embodiments, the compound of Formula 1C, wherein —NR¹¹R¹² is

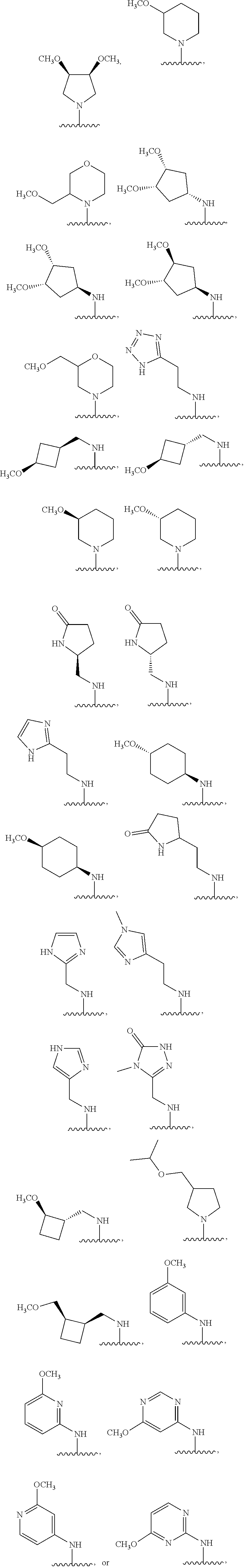

In some embodiments, the compound of Formula 1C, wherein —NR¹¹R¹² is

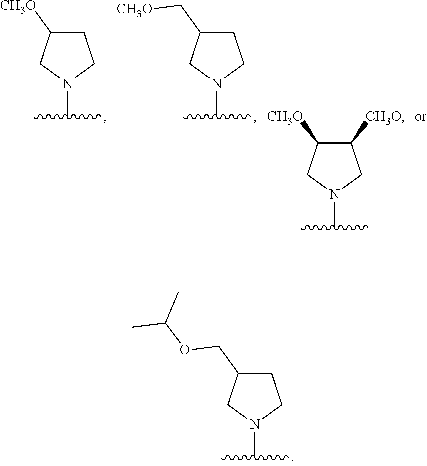

In some embodiments, the compound of Formula 1C, wherein —NR¹¹R¹² is

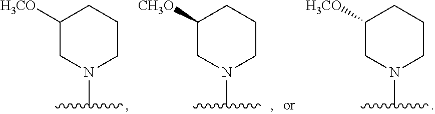

In some embodiments, the compound of Formula 1C, wherein —NR¹¹R¹² is

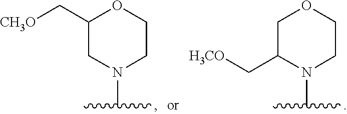

In some embodiments, the compound of Formula 1C, wherein —NR¹¹R¹² is

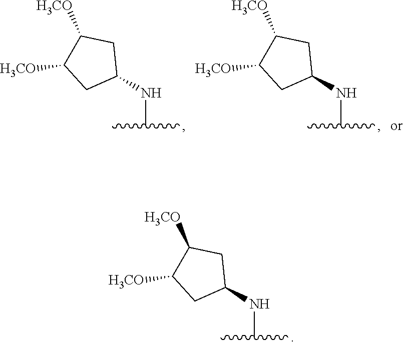

In some embodiments, the compound of Formula 1C, wherein —NR¹¹R¹² is

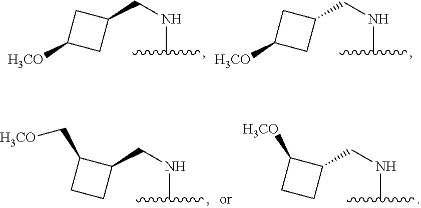 [SMILES: *NC[C@H]1C[C@@H](OC)C1 ; *NC[C@H]1C[C@H](OC)C1 ; *NC[C@H]1CC[C@H]1COC ; *NC[C@@H]1CC[C@H]1OC]

In some embodiments, the compound of Formula 1C, wherein —NR¹¹R¹² is

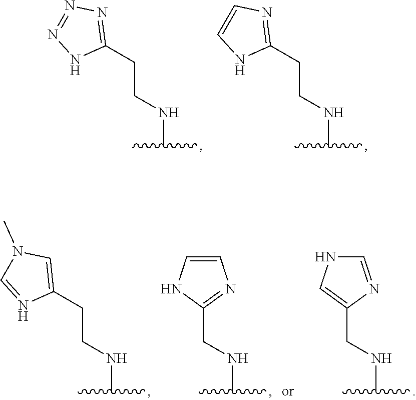

In some embodiments, the compound of Formula 1C, wherein —NR¹¹R¹² is:

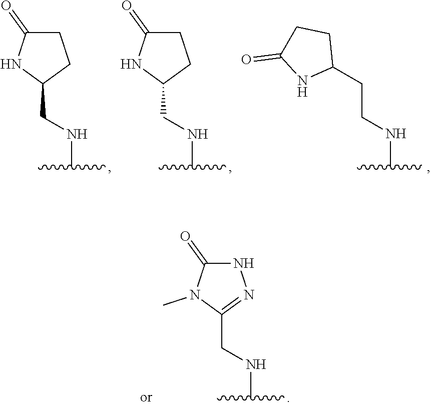

In some embodiments, the compound of Formula 1C, wherein —NR¹¹R¹² is:

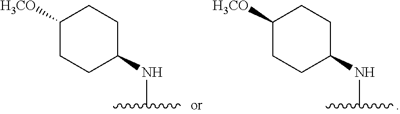 [SMILES: *N[C@H]1CC[C@H](OC)CC1 ; *N[C@H]1CC[C@@H](OC)CC1]

In some embodiments, the compound of Formula 1C, wherein —NR¹¹R¹² is:

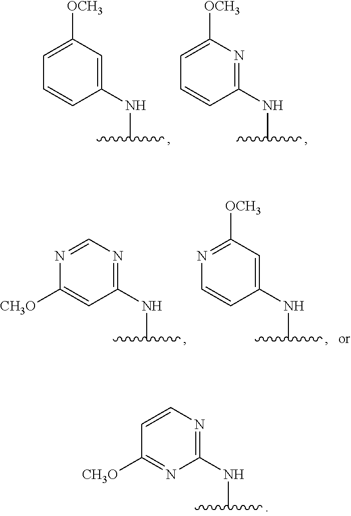

In some embodiments, the compound of Formula IC, wherein R¹³ is

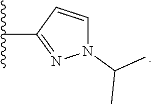 [SMILES: *c1ccn(C(C)C)n1]

In some embodiments, the compound of Formula IC, wherein R¹³ is

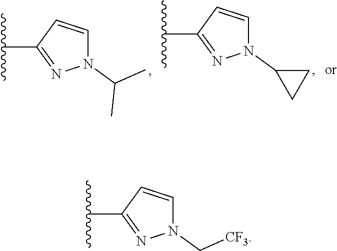

In some embodiments, the compound of Formula IC, wherein R¹³ is

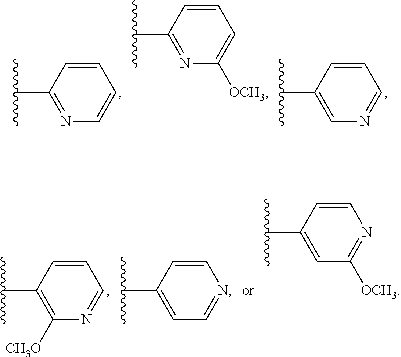

In certain embodiments, the compound for use in the compositions and methods provided herein is the compound of Formula IC, or a pharmaceutically acceptable derivative thereof, wherein:

—NR¹¹R¹² is

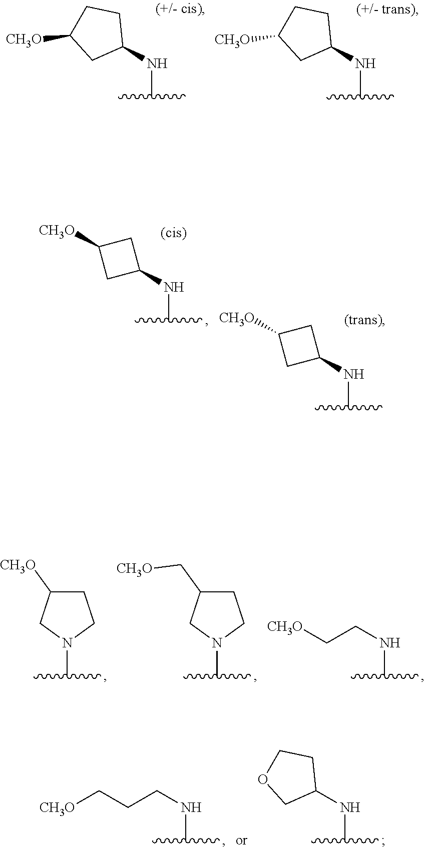

and

R¹³ is

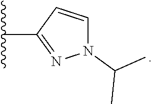 [SMILES: *c1ccn(C(C)C)n1]

In some embodiments, the compound of Formula IC is:

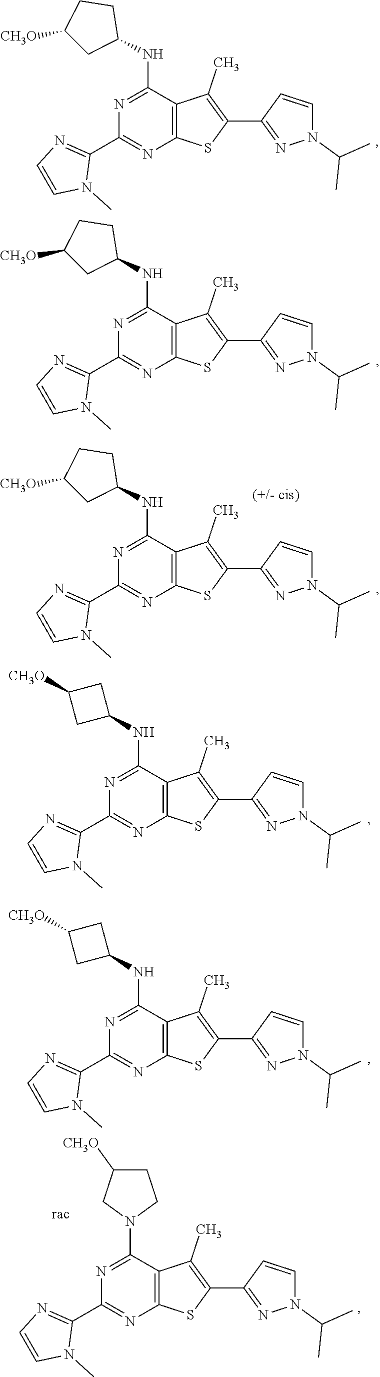

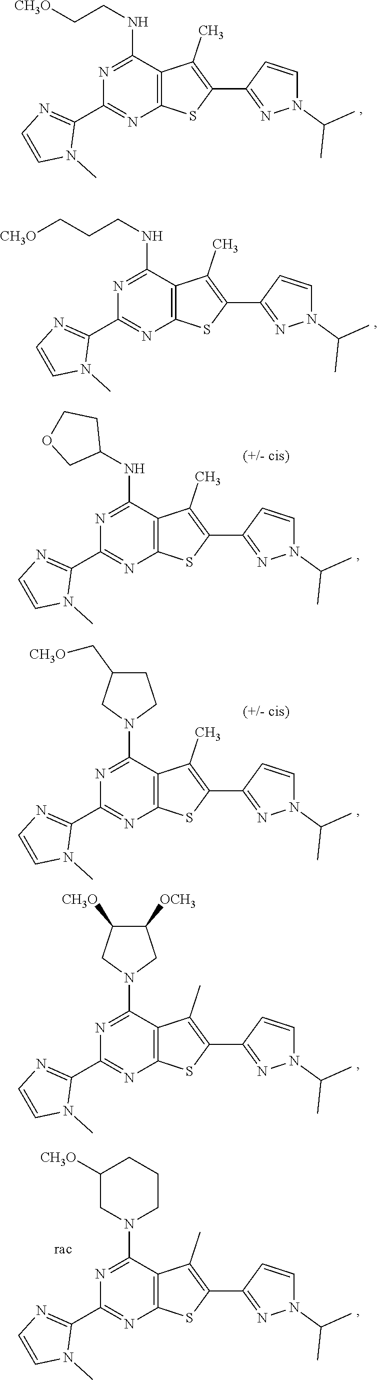

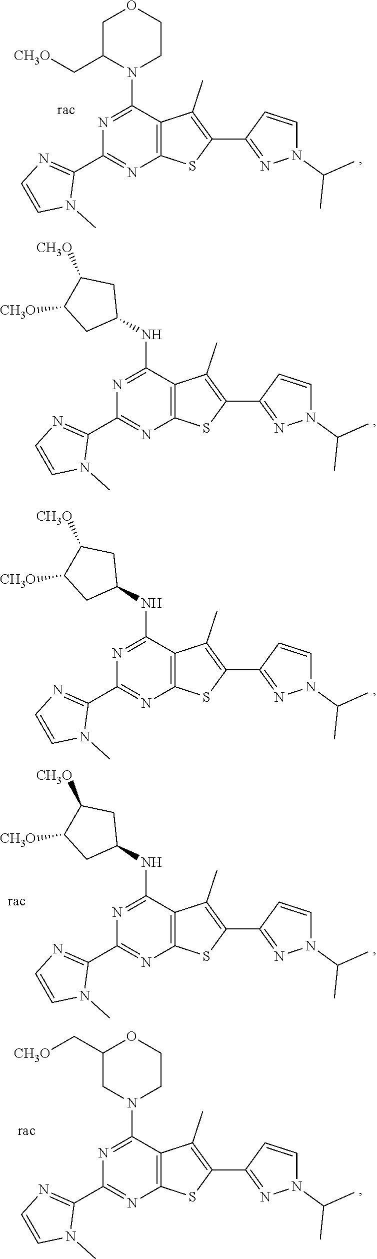

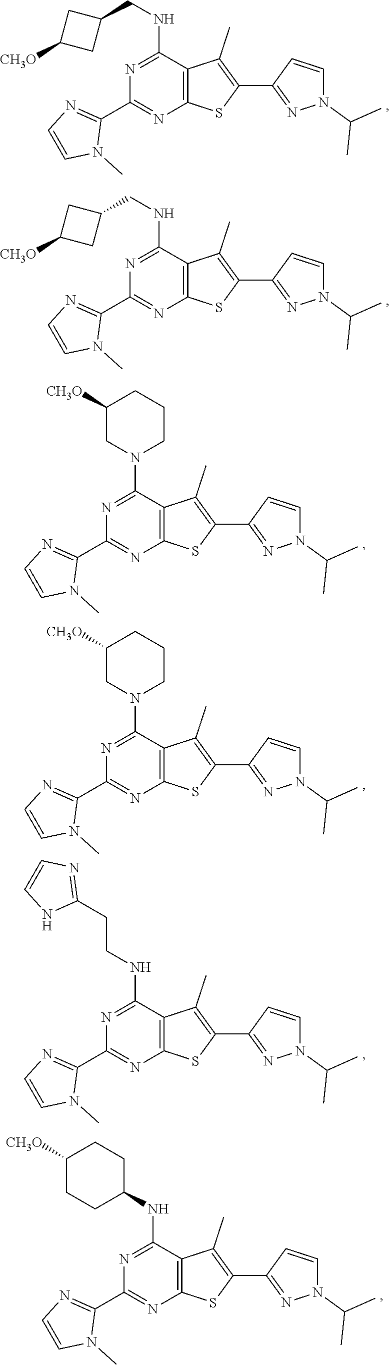

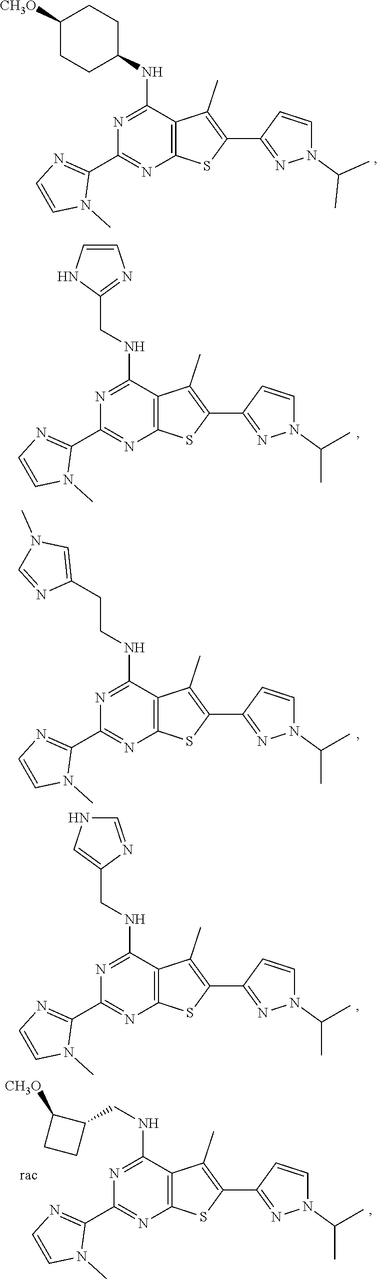

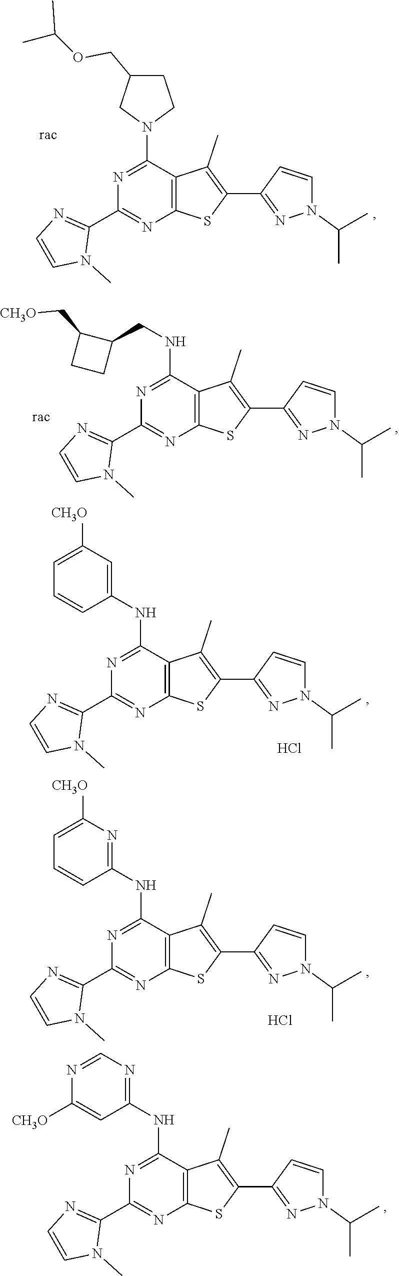

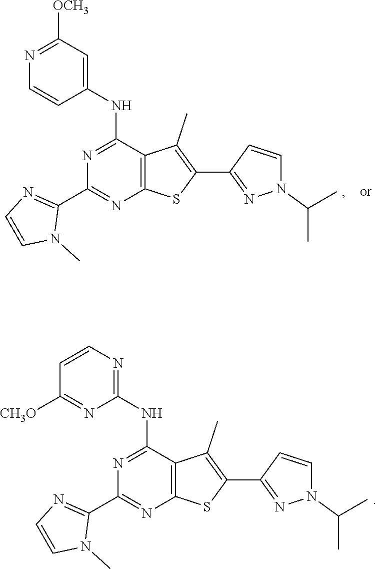

In some embodiments, the compound of Formula 1C is:

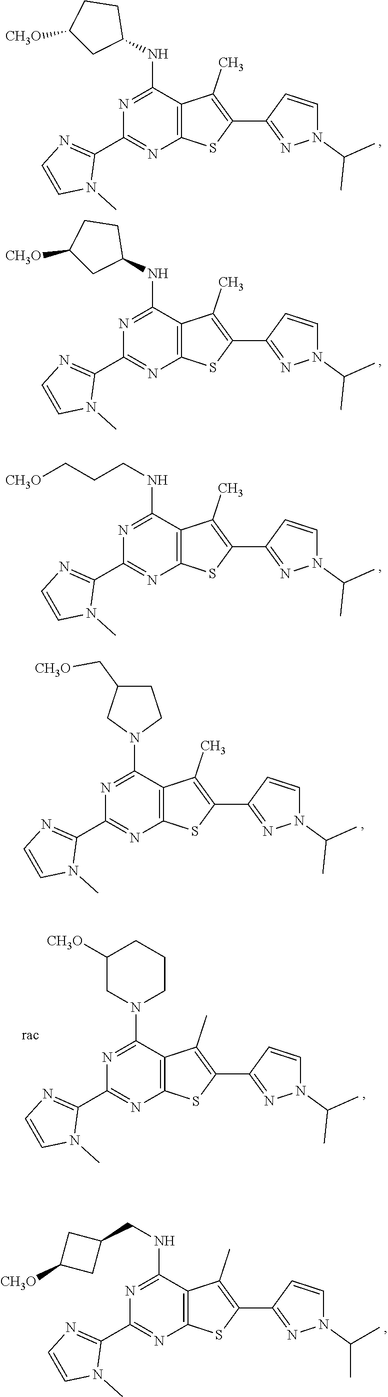

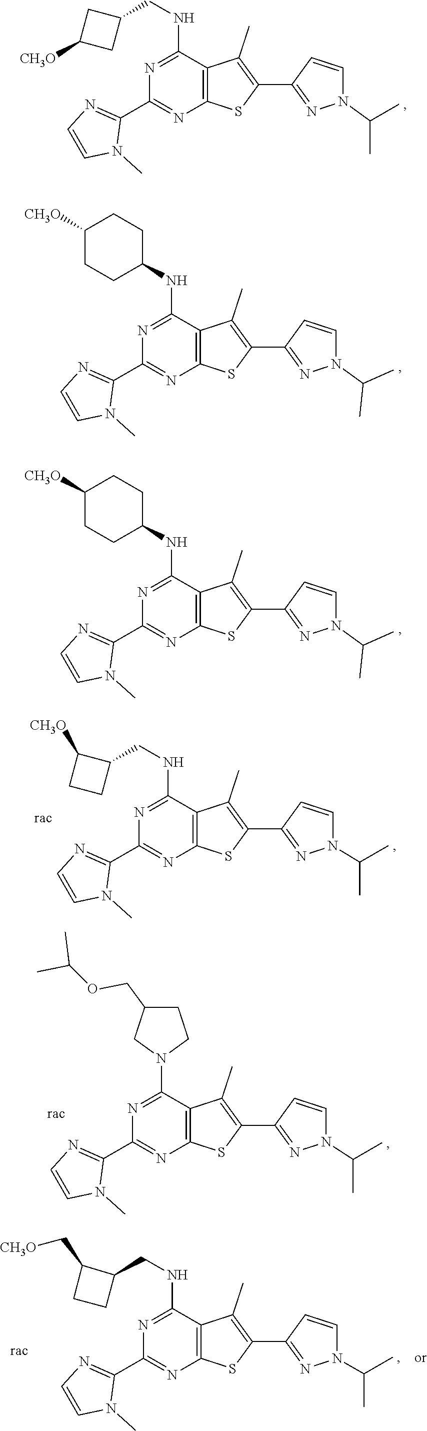

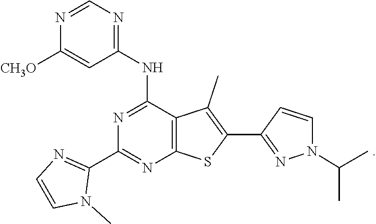

In certain embodiments, the pharmaceutically acceptable derivative of the compound of Formula IC is a pharmaceutically acceptable salt of the compound of Formula IC.

In certain embodiments, the compound for use in the compositions and methods provided herein is of Formula ID:

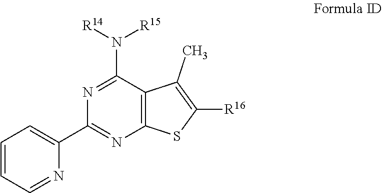

or a pharmaceutically acceptable derivative thereof, wherein:

—NR¹⁴R¹⁵ is

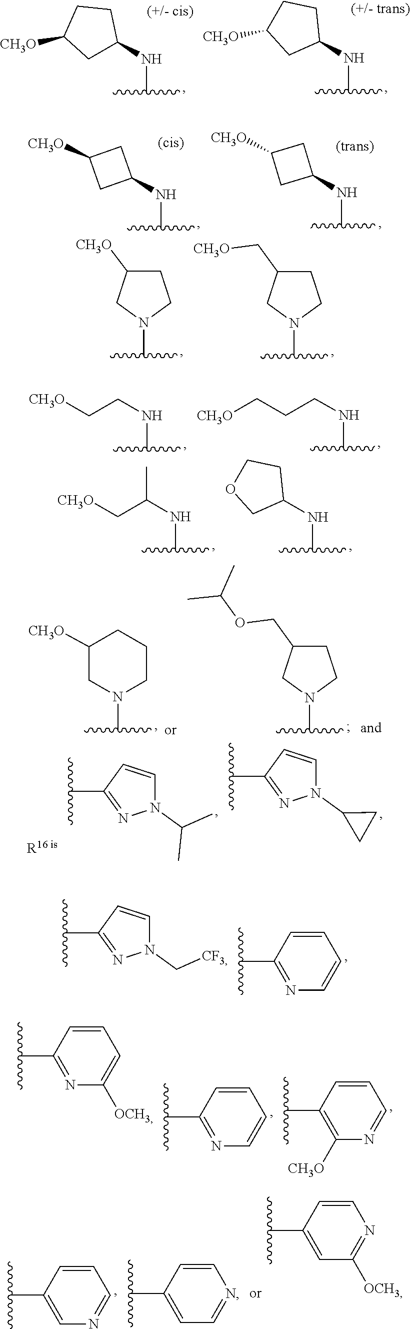

In some embodiments, the compound of Formula ID, wherein —NR¹⁴R¹⁵ is

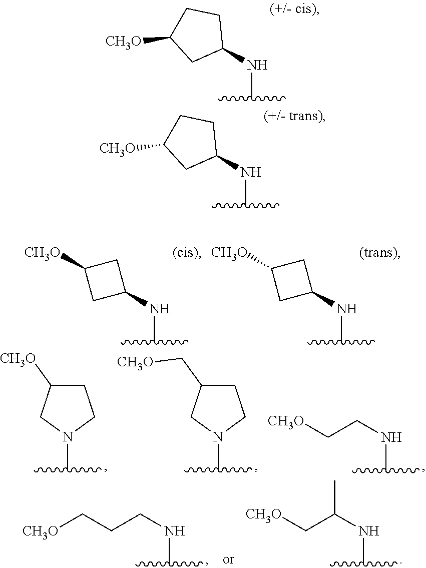

In some embodiments, the compound of Formula 1D, wherein —NR¹⁴R¹⁵ is

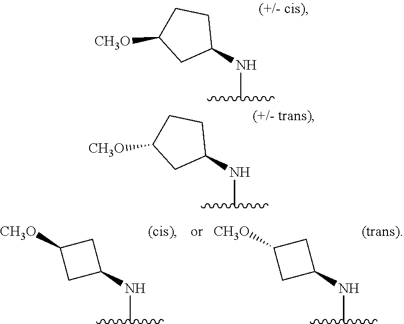

In some embodiments, the compound of Formula ID, wherein —NR¹⁴R¹⁵

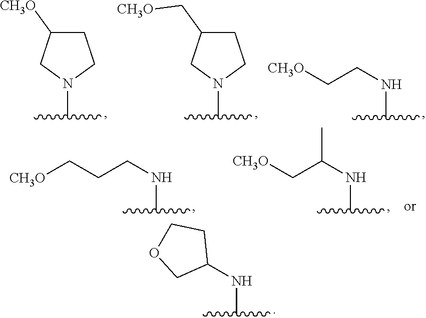

In some embodiments, the compound of Formula 1D, wherein —NR¹⁴R¹⁵ is

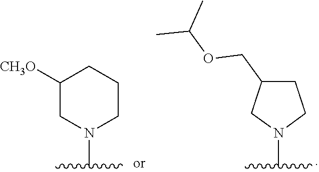

In some embodiments, the compound of Formula ID, wherein R¹⁶ is

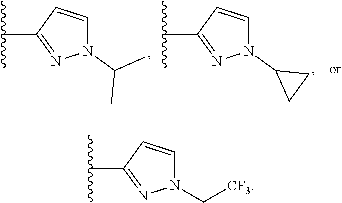

In some embodiments, the compound of Formula ID, wherein R¹⁶ is

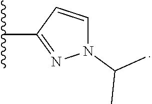 [SMILES: *c1ccn(C(C)C)n1]

In some embodiments, the compound of Formula ID, wherein R¹⁶ is

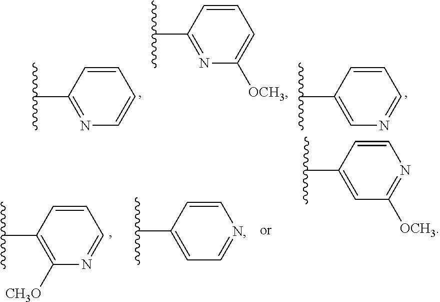

In certain embodiments, the compound for use in the compositions and methods provided herein is the compound of Formula ID, or a pharmaceutically acceptable derivative thereof, wherein:

—NR¹⁴R¹⁵ is

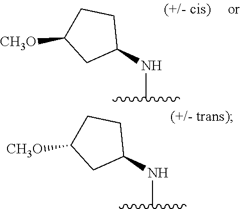

and

R¹⁶ is

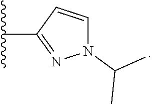 [SMILES: *c1ccn(C(C)C)n1]

In some embodiments, the compound of Formula ID, wherein —NR¹⁴R¹⁵ is

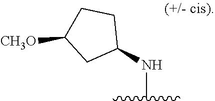

In some embodiments, the compound of Formula ID is:

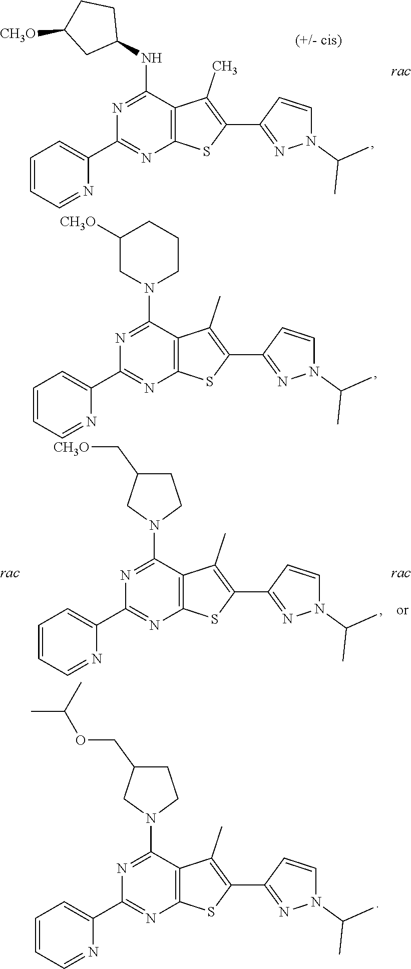

In certain embodiments, the pharmaceutically acceptable derivative of the compound of Formula ID is a pharmaceutically acceptable salt of the compound of Formula ID.

In certain embodiments, the compound for use in the compositions and methods provided herein is of Formula IE:

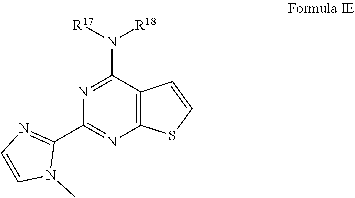

or a pharmaceutically acceptable derivative thereof, wherein:

—NR¹⁷R¹⁸ is

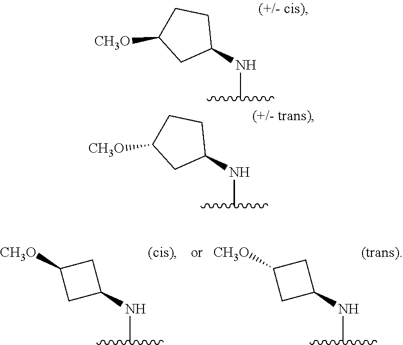

In some embodiments, the compound of Formula IE, wherein —NR¹⁷R¹⁸ is

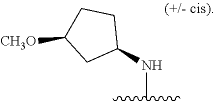

In some embodiments, the compound of Formula IE, wherein —NR¹⁷R¹⁸ is

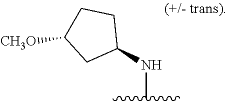

In some embodiments, the compound of Formula IE, wherein —NR¹⁷R¹⁸ is

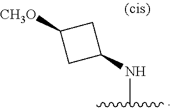

In some embodiments, the compound of Formula IE, wherein —NR¹⁷R¹⁸ is

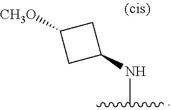

In some embodiments, the compound of Formula IE is:

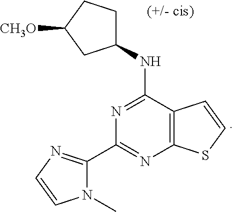

In certain embodiments, the pharmaceutically acceptable derivative of the compound of Formula IE is a pharmaceutically acceptable salt of the compound of Formula IE.

In certain embodiments, the compound for use in the compositions and methods provided herein is of Formula IF:

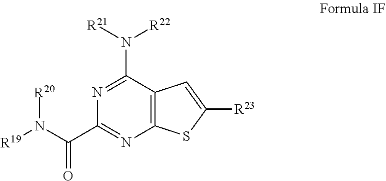

or a pharmaceutically acceptable derivative thereof, wherein:

—NR¹⁹R²⁰ is —NH₂,

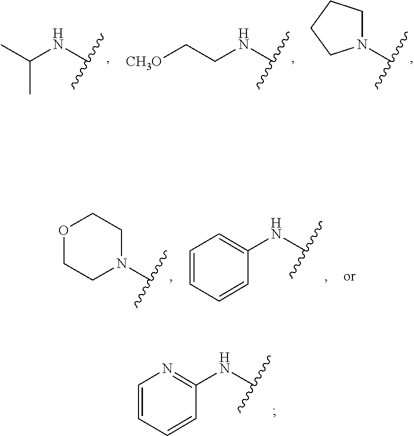

—NR²¹R²² is

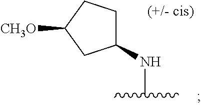

and

R²³ is hydrogen or

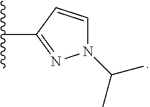 [SMILES: *c1ccn(C(C)C)n1]

In some embodiments, the compound of Formula IF, wherein R²³ is hydrogen.

In some embodiments, the compound of Formula IF, wherein R²³ is

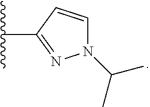 [SMILES: *c1ccn(C(C)C)n1]

In some embodiments, the compound of Formula IF is:

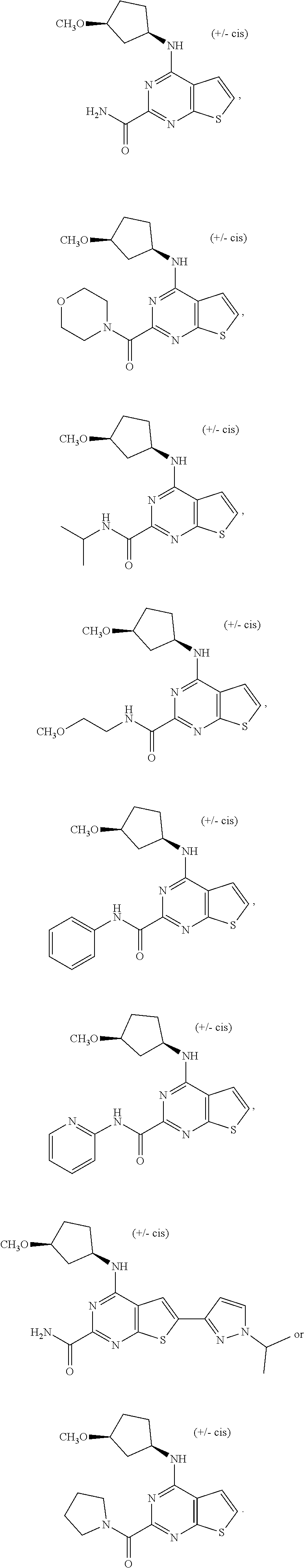

In certain embodiments, the pharmaceutically acceptable derivative of the compound of Formula IF is a pharmaceutically acceptable salt of the compound of Formula IF.

In certain embodiments, the compound for use in the compositions and methods provided herein is of Formula IIA:

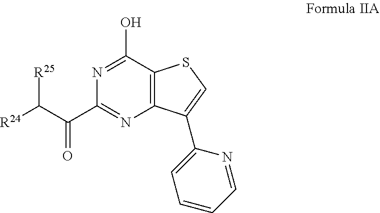

or a pharmaceutically acceptable derivative thereof, wherein:

—NR²⁴R²⁵ is —NH₂,

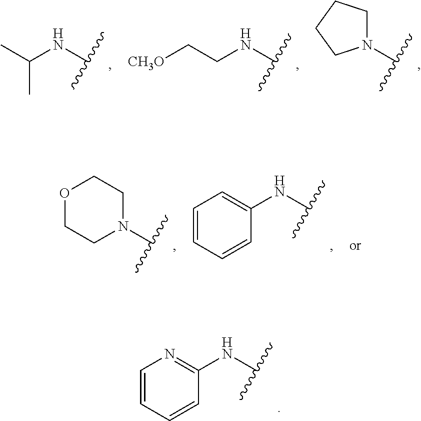

In some embodiments, the compound of Formula IIA is:

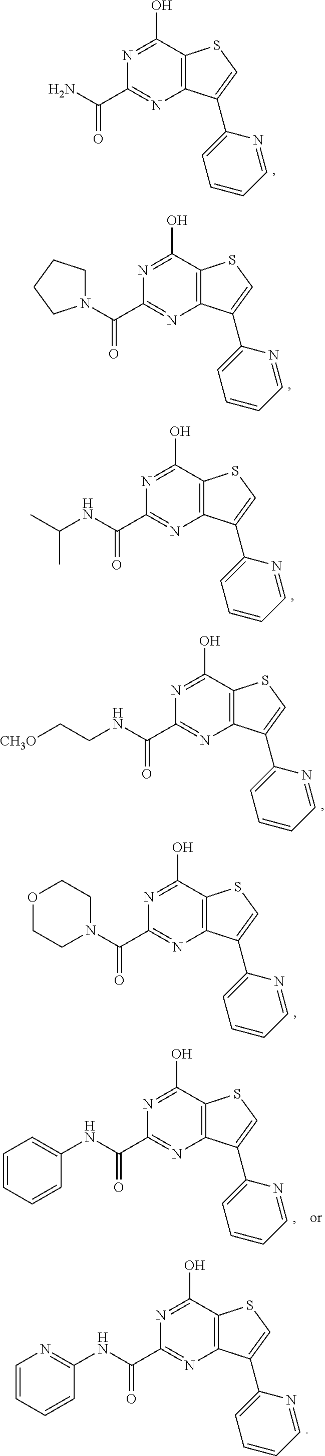

In certain embodiments, the pharmaceutically acceptable derivative of the compound of Formula IIA is a pharmaceutically acceptable salt of the compound of Formula IIA.

In certain embodiments, the compound for use in the compositions and methods provided herein is of Formula IIB:

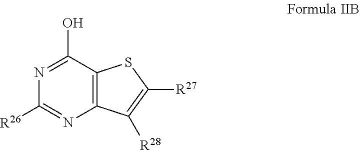

or a pharmaceutically acceptable derivative thereof, wherein:

R²⁶ is

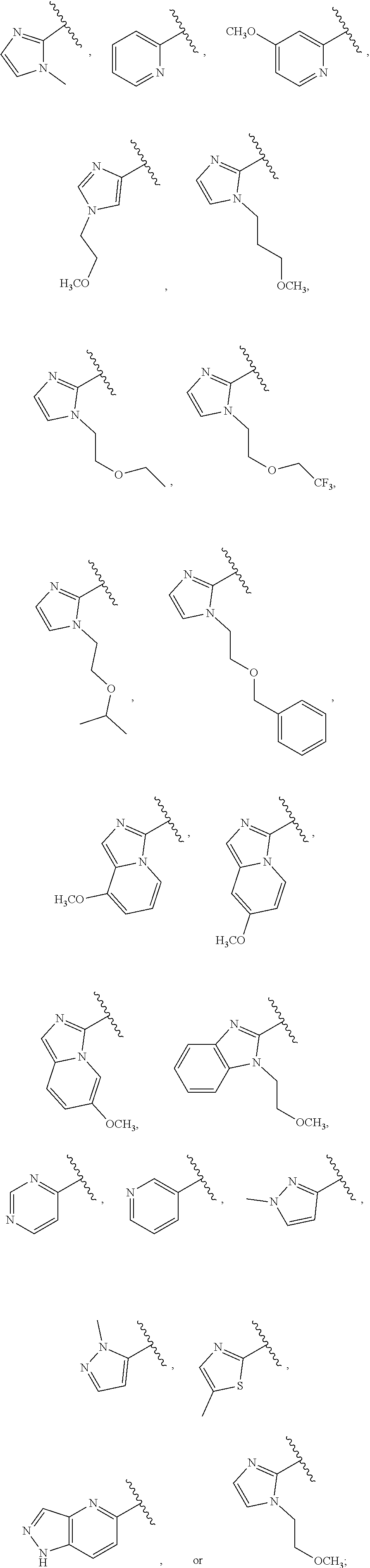

R²⁷ is hydrogen, —CH₃, or —CF₃; and

R²⁸ is

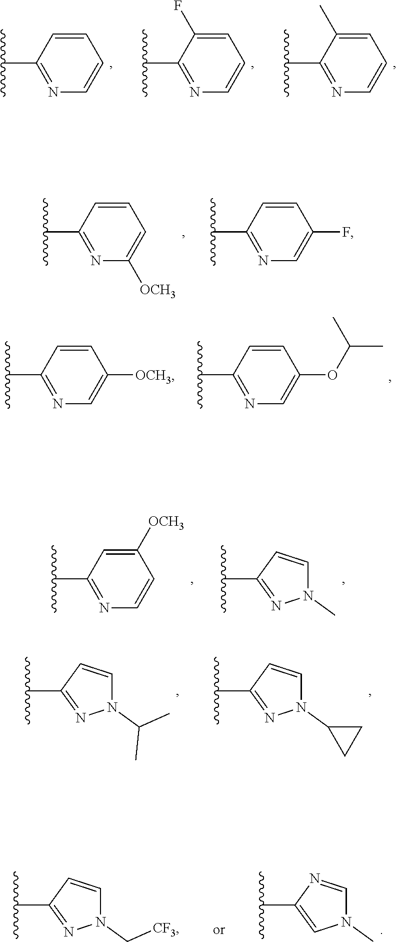

In some embodiments, the compound of Formula IIB, wherein R²⁶ is:

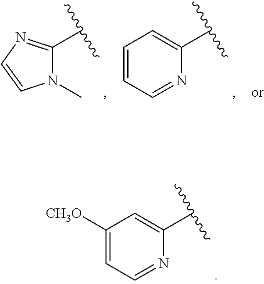

In some embodiments, the compound of Formula IB, wherein R²⁶ is

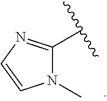 [SMILES: *c1nccn1C]

In some embodiments, the compound of Formula IIB, wherein R²⁶ is

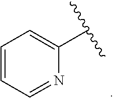 [SMILES: *c1ccccn1]

In some embodiments, the compound of Formula IIB, wherein R²⁶ is

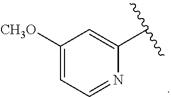

In some embodiments, the compound of Formula IIB, wherein R²⁶ is:

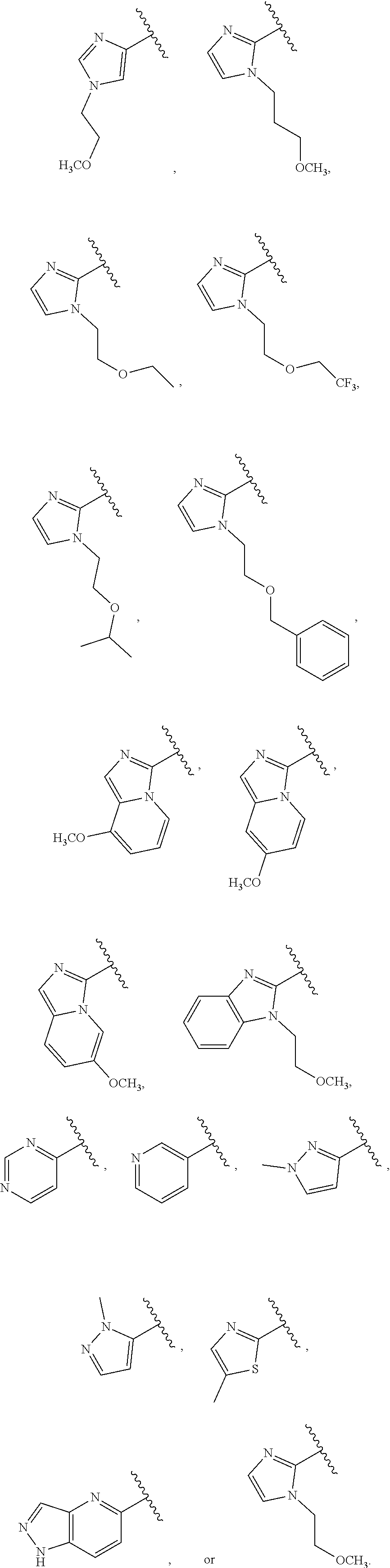

In some embodiments, the compound of Formula IIB, wherein R²⁶ is:

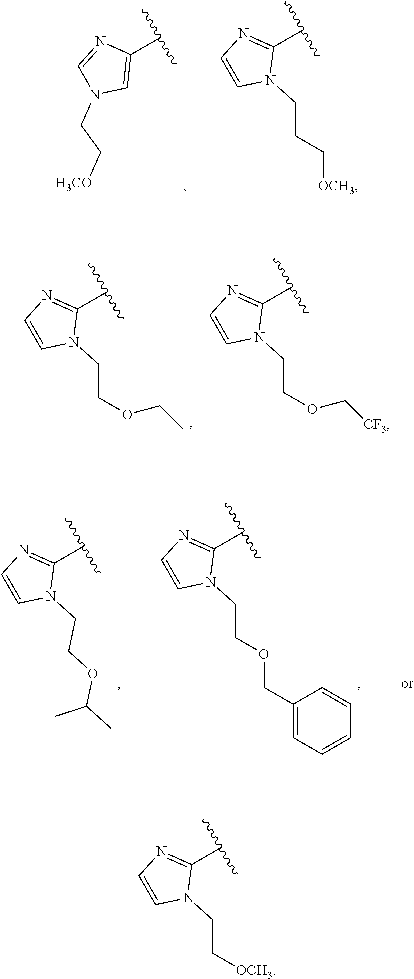

In some embodiments, the compound of Formula IIB, wherein R²⁶ is:

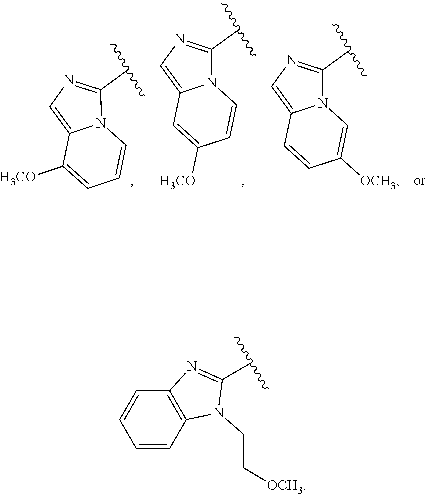

In some embodiments, the compound of Formula IIB, wherein R²⁶ is:

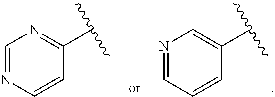 [SMILES: *c1ccncn1 ; *c1cccnc1]

In some embodiments, the compound of Formula IIB, wherein R²⁶ is:

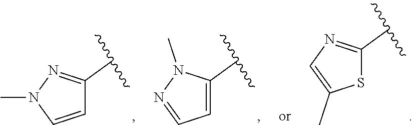

In some embodiments, the compound of Formula IIB, wherein R²⁶ is

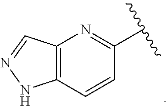 [SMILES: *c1ccc2[nH]ncc2n1]

In some embodiments, the compound of Formula IIB, wherein R²⁷ is hydrogen.

In some embodiments, the compound of Formula IIB, wherein R²⁷ is —CH₃.

In some embodiments, the compound of Formula IIB, wherein R²⁷ is —CF₃.

In some embodiments, the compound of Formula IIB, wherein R²⁸ is:

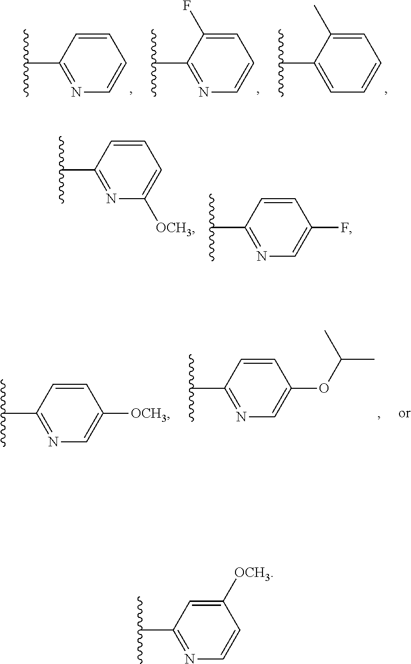

In some embodiments, the compound of Formula IIB, wherein R²⁸ is:

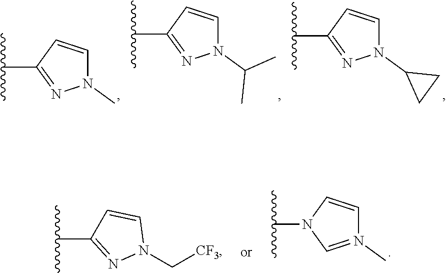

In certain embodiments, the compound for use in the compositions and methods provided herein is the compound of Formula IIB, or a pharmaceutically acceptable derivative thereof, wherein:

R²⁶ is

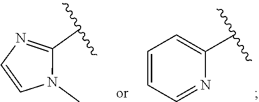

R²⁷ is hydrogen; and

R²⁸ is

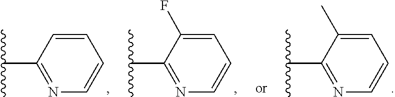

In some embodiments, the compound of Formula IIB, wherein R²⁶ is

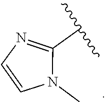 [SMILES: *c1nccn1C]

In some embodiments, the compound of Formula IIB, wherein R²⁶ is

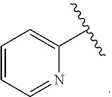 [SMILES: *c1ccccn1]

In some embodiments, the compound of Formula IIB, wherein R²⁸ is

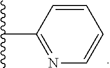 [SMILES: *c1ccccn1]

In some embodiments, the compound of Formula IIB, wherein R²⁸ is:

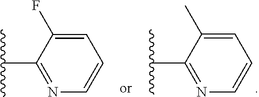

In some embodiments, the compound of Formula IIB, wherein with the proviso that when:

R²⁶ is

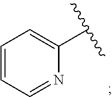 [SMILES: *c1ccccn1]

and R²⁷ is hydrogen; then R²⁸ is not

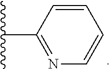 [SMILES: *c1ccccn1]

In some embodiments, the compound of Formula IIB is:

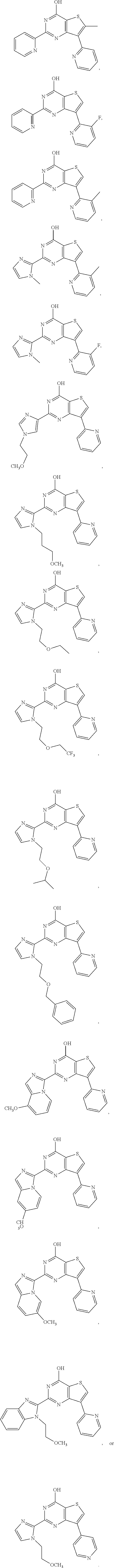

In some embodiments, the compound of Formula IIB is:

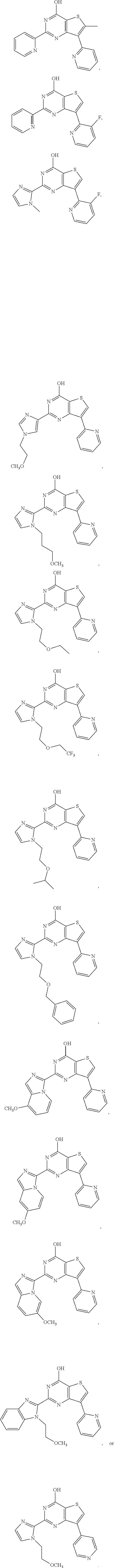

In some embodiments, the compound of Formula IIB is

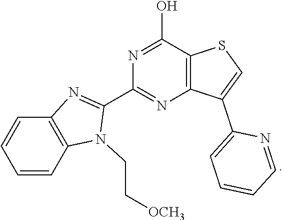 [SMILES: COCCn1c(-c2nc(O)c3scc(-c4ccccn4)c3n2)nc2ccccc21]

In certain embodiments, the pharmaceutically acceptable derivative of the compound of Formula IIB is a pharmaceutically acceptable salt of the compound of Formula IIB.

In some embodiments, the compound for use in the compositions and methods provided herein is:

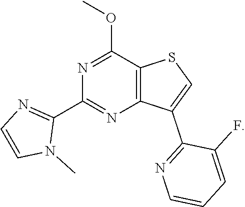 [SMILES: COc1nc(-c2nccn2C)nc2c(-c3ncccc3F)csc12]

In certain embodiments, the pharmaceutically acceptable derivative of the compound is a pharmaceutically acceptable salt.

### 4.5. Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MHLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonc.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% to 100% active ingredient, in certain embodiments, about 0.1 85% or about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461,6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof; or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### 4.6. Dosing

The compounds and pharmaceutical compositions provided herein may be dosed in certain therapeutically or prohylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, e.g., Nair A B, Jacob S. A simple practice guide for dose conversion between animals and human. J Basic Clin Pharma 2016; 7:27-31). For example, a dose of 1 mg/kg/day for a 60 kg human is approximately equal to 37 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 500 μM, about 0.002 to about 200 μM, about 0.005 to about 100 PM, about 0.01 to about 50 μM, from about 1 to about 50 μM, about 0.02 to about 25 μM, from about 0.05 to about 20 μM, from about 0.1 to about 20 μM, from about 0.5 to about 20 PM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 PM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

Combination Therapy with a Second Active Agent

The compound provided herein, or a derivative thereof, can also be combined or used in combination with other therapeutic agents useful in the treatment and/or prevention of cancers, inflammatory diseases, rasopathies, or fibrotic disease.

In one embodiment, provided herein is a method of treating, preventing, or managing cancers, inflammatory diseases, rasopathies, and fibrotic disease, comprising administering to a subject a compound provided herein, or a derivative thereof, in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

### 5. EXAMPLES

### Abbreviations

PdXphosG3: (2-Dicyclohexylphosphino-2′,4′,6′-triisopropyl-1,1′-biphenyl)[2-(2′-amino-1,1′-biphenyl)]palladium(II) methanesulfonate

Pd(dppf)Cl₂·CH₂Cl₂: [1,1′-Bis(diphenylphosphino)ferrocene]dichloropalladium(II), complex with dichloromethane

MTBE: Methyl tert-butyl ether

HATU: 1-[Bis(dimethylamino)methylene]-1H-1,2,3-triazolo[4,5-b]pyridinium 3-oxid hexafluorophosphate

DIPEA: N,N-Diisopropylethylamine

TCFH: N,N,N′,N′-tetramethylchloroformamidinium hexafluorophosphate

The following examples are offered to illustrate but not to limit the disclosure.

### Example 1

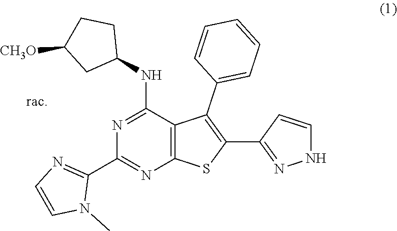

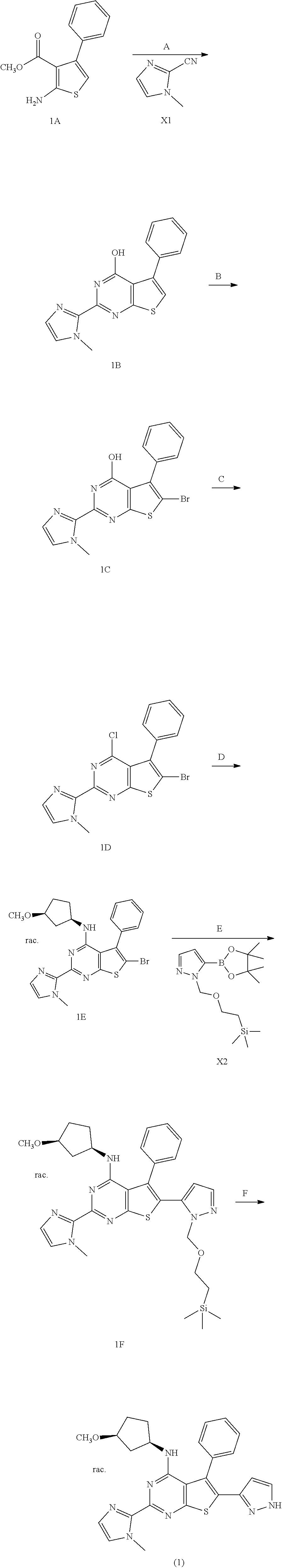

### Rac-N-((1R,3S)-3-methoxycyclopentyl)-2-(1-methyl-1H-imidazol-2-yl)-5-phenyl-6-(1H-pyrazol-3-yl)thieno[2,3-d]pyrimidin-4-amine (1)

### Step A: 2-(1-Methyl-1H-imidazol-2-yl)-5-phenylthieno[2,3-d]pyrimidin-4-ol 1B

Methyl 2-amino-4-phenylthiophene-3-carboxylate 1A (22.5 g, 96.45 mmol), 1-methyl-1H-imidazole-2-carbonitrile X1 (15.5 g, 144.67 mmol, 15.5 ml, 1.5 equiv), and potassium t-butoxide (86.58 g, 771.57 mmol) was dissolved in dry MeOH (220 mL) and stirred overnight at room temperature. The resulting mixture was evaporated and acetic acid was added to pH 5. Then it was extracted with EtOAc (250 mL), the organic layer was separated, dried over Na₂SO₄ and evaporated to give compound 1B, 2-(1-methyl-1H-imidazol-2-yl)-5-phenylthieno[2,3-d]pyrimidin-4-ol, (17.0 g, 55.13 mmol, 57.2% yield), which was used without additional purification in a further step.

### Step B: 6-Bromo-2-(1-methyl-1H-imidazol-2-yl)-5-phenylthieno[2,3-d]pyrimidin-4-ol 1C

Compound 1B, 2-(1-methyl-1H-imidazol-2-yl)-5-phenylthieno[2,3-d]pyrimidin-4-ol, (25.0 g, 81.07 mmol) was dissolved in DMF (250 mL), then N-bromosuccinimide (21.64 g, 121.61 mmol) was added at room temperature. Reaction mixture was heated at 65° C. overnight, cooled to room temperature and poured into ice (500 mL). The resulting precipitate was filtered and dried in air at 60° C. to give compound 1C, 6-bromo-2-(1-methyl-1H-imidazol-2-yl)-5-phenylthieno[2,3-d]pyrimidin-4-ol, (20.0 g, 80.0% purity, 41.32 mmol, 51% yield) as yellow solid.

### Step C: 6-Bromo-4-chloro-2-(1-methyl-1H-imidazol-2-yl)-5-phenylthieno[2,3-d]pyrimidine 1D

Compound 1C, 6-bromo-2-(1-methyl-1H-imidazol-2-yl)-5-phenylthieno[2,3-d]pyrimidin-4-ol, (23.0 g, 59.4 mmol) was suspended in P(O)C13 (91.08 g, 594.03 mmol, 55.37 mL, 10.0 eq) and N,N-diisopropylethylamine (23.03 g, 178.21 mmol, 31.04 mL, 3.0 equiv) was added at room temperature. The reaction mixture was refluxed for 16 h, the solution was cooled to room temperature, evaporated under reduced pressure, poured in ice, diluted with ice-cold ammonia (200 mL, 20-25% of ammonia). The product was extracted with chloroform (2×500 mL) and evaporated. Compound 1D, 6-bromo-4-chloro-2-(1-methyl-1H-imidazol-2-yl)-5-phenylthieno[2,3-d]pyrimidine, (22.0 g, 43.0% purity, 23.32 mmol, 39.3% yield) was obtained as yellow solid.

### Step D: rac-6-Bromo-N-((1R,3S)-3-methoxycyclopentyl)-2-(1-methyl-1H-imidazol-2-yl)-5-phenylthieno[2,3-d]pyrimidin-4-amine 1E

Compound 1D, 6-bromo-4-chloro-2-(1-methyl-1H-imidazol-2-yl)-5-phenylthieno[2,3-d]pyrimidine (4.46 g, 11.0 mmol) was dissolved in DMSO (50 mL) then N,N-diisopropylethylamine (3.55 g, 27.5 mmol, 4.79 ml, 2.5 equiv) and rac-(1R,3S)-3-methoxycyclopentan-1-amine (1.9 g, 16.5 mmol) was added in one portion. The mixture was heated at 100° C. overnight, cooled and purified by HPLC. Compound 1E, rac-6-bromo-N-((1R,3S)-3-methoxycyclopentyl)-2-(1-methyl-1H-imidazol-2-yl)-5-phenylthieno[2,3-d]pyrimidin-4-amine, was obtained as yellow viscous residue (1.25 g, 2.58 mmol, 23.5% yield).

### Step E: rac-N-((1R,3S)-3-methoxycyclopentyl)-2-(1-methyl-1H-imidazol-2-yl)-5-phenyl-6-(1-((2-(trimethylsilyl)ethoxy)methyl)-1H-pyrazol-5-yl)thieno[2,3-d]pyrimidin-4-amine 1F

Compound 1E, rac-6-bromo-N-((1R,3S)-3-methoxycyclopentyl)-2-(1-methyl-1H-imidazol-2-yl)-5-phenylthieno[2,3-d]pyrimidin-4-amine, (200.0 mg, 412.87 μmol), 5-(4,4,5,5-tetramethyl-1,3,2-dioxaborolan-2-yl)-1-[2-(trimethylsilyl)ethoxy]methyl-1H-pyrazole X2 (267.27 mg, 824.14 μmol), cesium carbonate (268.52 mg, 824.14 μmol) and PdXphosG3 (69.76 mg, 82.41 μmol) was mixed in dioxane/water (4 mL)/(0.2 mL). The reaction flask was filled with argon and the reaction mixture was stirred at 80° C. overnight, then cooled and filtered through a SiO₂ pad. The mother liquid was concentrated and purified by HPLC. Compound 1F, rac-N-((1R,3S)-3-methoxycyclopentyl)-2-(1-methyl-1H-imidazol-2-yl)-5-phenyl-6-(1-((2-(trimethylsilyl)ethoxy)methyl)-1H-pyrazol-5-yl)thieno[2,3-d]pyrimidin-4-amine was obtained and used in the next step (57.5 mg, 66.0% purity, 63.06 μmol, 15.3% yield).

### Step F: rac-N-((1R,3S)-3-methoxycyclopentyl)-2-(1-methyl-1H-imidazol-2-yl)-5-phenyl-6-(1H-pyrazol-3-yl)thieno[2,3-d]pyrimidin-4-amine (1)

To the solution of compound 1F, rac-N-((1R,3S)-3-methoxycyclopentyl)-2-(1-methyl-1H-imidazol-2-yl)-5-phenyl-6-(1-((2-(trimethylsilyl)ethoxy)methyl)-1H-pyrazol-5-yl)thieno[2,3-d]pyrimidin-4-amine, (57.5 mg, 95.54 μmol) in CH₂Cl₂ (0.5 mL), TFA (0.5 mL) was added dropwise. This mixture was stirred 16 h at room temperature, concentrated and purified by HPLC. The Example compound (1), rac-N-((1R,3S)-3-methoxycyclopentyl)-2-(1-methyl-1H-imidazol-2-yl)-5-phenyl-6-(1H-pyrazol-3-yl)thieno[2,3-d]pyrimidin-4-amine (6.1 mg, 92.0% purity, 11.9 μmol, 12.4% yield) was obtained.

### Example 2

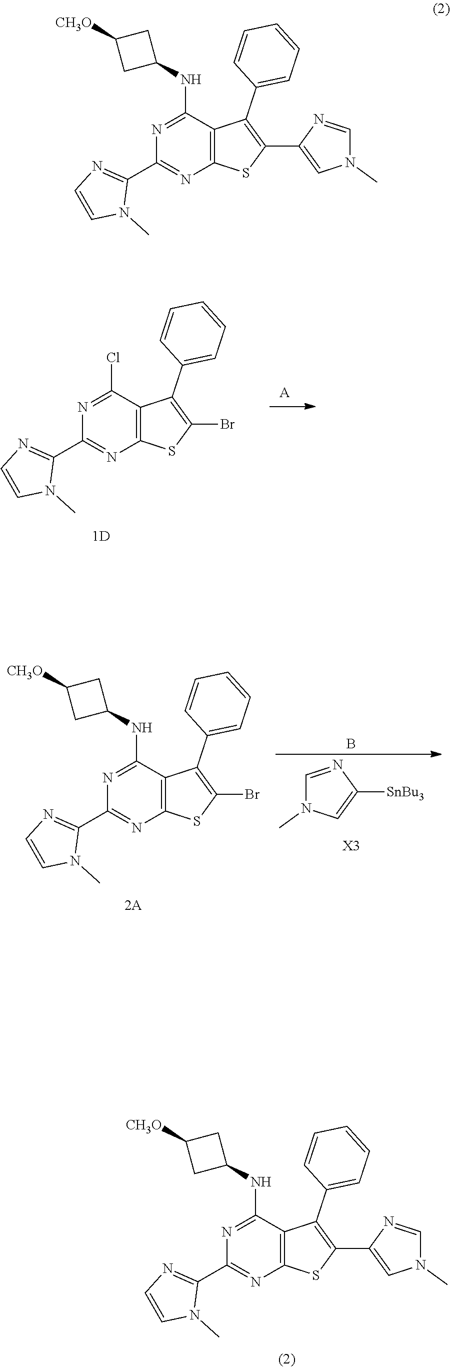

### N-((1s,3s)-3-methoxycyclobutyl)-2-(1-methyl-1H-imidazol-2-yl)-6-(1-methyl-1H-imidazol-4-yl)-5-phenylthieno[2,3-d]pyrimidin-4-amine (2)

### Step A: 6-Bromo-N-((1s,3s)-3-methoxycyclobutyl)-2-(1-methyl-1H-imidazol-2-yl)-5-phenylthieno[2,3-d]pyrimidin-4-amine 2A

Compound 1D, 6-bromo-4-chloro-2-(1-methyl-1H-imidazol-2-yl)-5-phenylthieno[2,3-d]pyrimidine, (13.76 g, 33.92 mmol) was dissolved in DMSO (140 mL) then N,N-diisopropylethylamine (10.96 g, 84.8 mmol, 14.77 mL, 2.5 equiv) and (1s,3s)-3-methoxycyclobutan-1-amine hydrochloride (7.0 g, 50.88 mmol) was added in one portion. The mixture was heated at 100° C. overnight, cooled and purified by HPLC. Compound 2A, 6-bromo-N-((1s,3s)-3-methoxycyclobutyl)-2-(1-methyl-1H-imidazol-2-yl)-5-phenylthieno[2,3-d]pyrimidin-4-amine, (2.64 g, 91.0% purity, 5.11 mmol, 15.1% yield) was obtained as yellow viscous residue.

### Step B: N-((1s,3s)-3-methoxycyclobutyl)-2-(1-methyl-1H-imidazol-2-yl)-6-(1-methyl-1H-imidazol-4-yl)-5-phenylthieno[2,3-d]pyrimidin-4-amine (2)

Compound 2A, 6-bromo-N-((1s,3s)-3-methoxycyclobutyl)-2-(1-methyl-1H-imidazol-2-yl)-5-phenylthieno[2,3-d]pyrimidin-4-amine, (300.0 mg, 637.77 μmol), 1-methyl-4-(tributylstannyl)-1H-imidazole X3 (526.44 mg, 1.42 mmol), Pd(dppf)Cl₂ CH₂Cl₂ (52.12 mg, 63.83 μmol), CuI (243.12 mg, 1.28 mmol), were dissolved in dioxane (5 mL). The reaction flask was filled with argon and the reaction mixture was stirred at 80° C. overnight. The resulting material was purified by HPLC without pre-workup. The Example compound, N-((1s,3s)-3-methoxycyclobutyl)-2-(1-methyl-1H-imidazol-2-yl)-6-(1-methyl-1H-imidazol-4-yl)-5-phenylthieno[2,3-d]pyrimidin-4-amine (2), (6.6 mg, 14.0 μmol, 2.2% yield) was obtained.

### Example 3

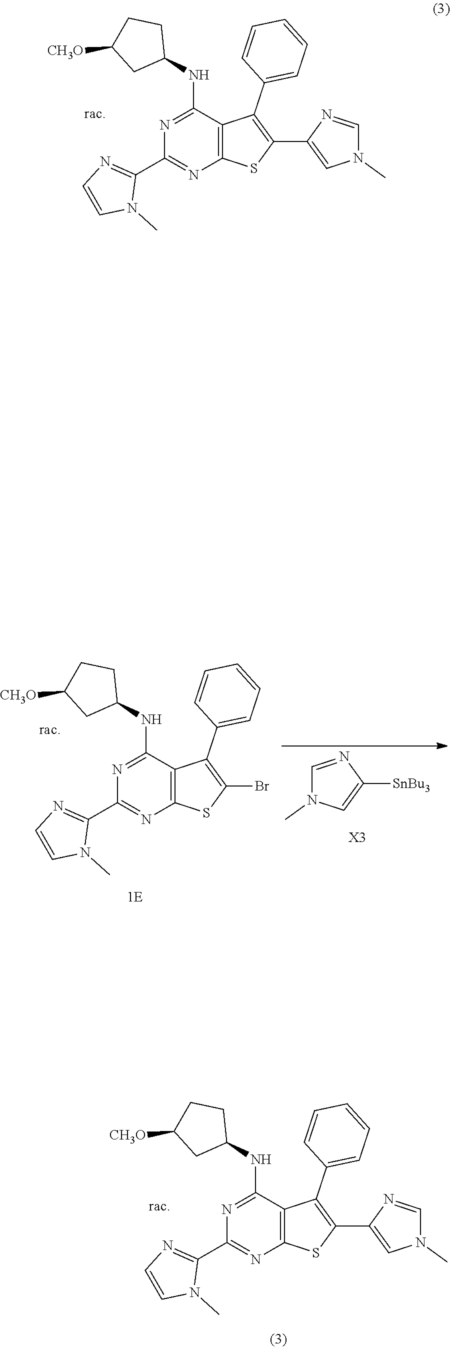

### Rac-N-((1R,3S)-3-methoxycyclopentyl)-2-(1-methyl-1H-imidazol-2-yl)-6-(1-methyl-1H-imidazol-4-yl)-5-phenylthieno[2,3-d]pyrimidin-4-amine (3)

Compound 1E, rac-6-bromo-N-((1R,3S)-3-methoxycyclopentyl)-2-(1-methyl-1H-imidazol-2-yl)-5-phenylthieno[2,3-d]pyrimidin-4-amine (316.75 mg, 673.39 μmol), 1-methyl-4-(tributylstannyl)-1H-imidazole X3 (300.0 mg, 808.3 μmol), Pd(dppf)Cl₂ CH₂Cl₂ (54.99 mg, 67.34 μmol), CuI (25.65 mg, 134.68 μmol), were dissolved in dioxane (3 mL). The reaction flask was filled with argon and the reaction mixture was stirred at 80° C. overnight. The resulting material was purified by HPLC without pre-workup. The Example compound, rac-N-((1R,3S)-3-methoxycyclopentyl)-2-(1-methyl-1H-imidazol-2-yl)-6-(1-methyl-1H-imidazol-4-yl)-5-phenylthieno[2,3-d]pyrimidin-4-amine (3), (6.1 mg, 92.0% purity, 11.56 μmol, 1.7% yield) was obtained.

### Example 4

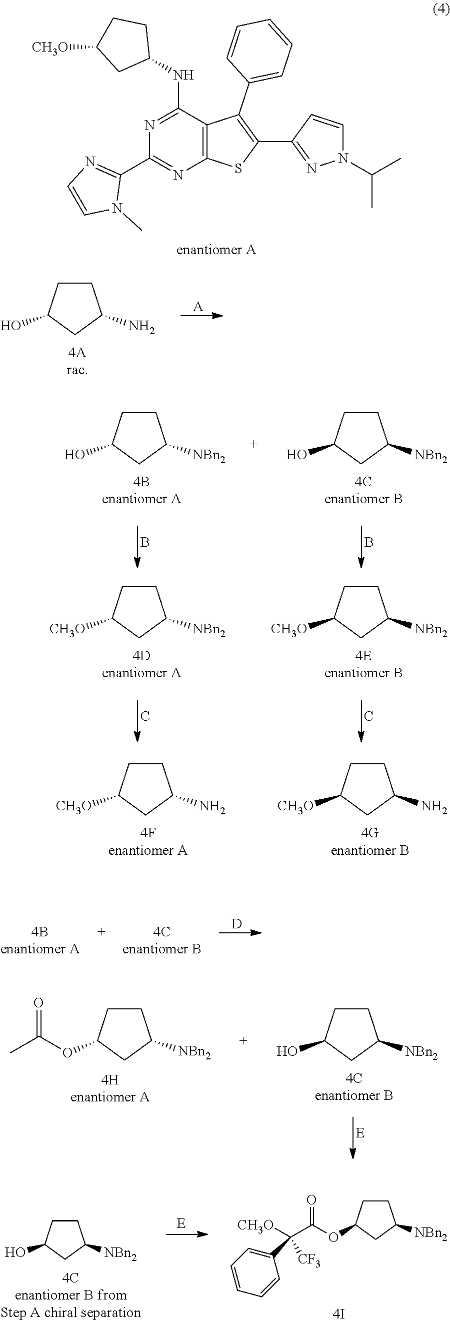

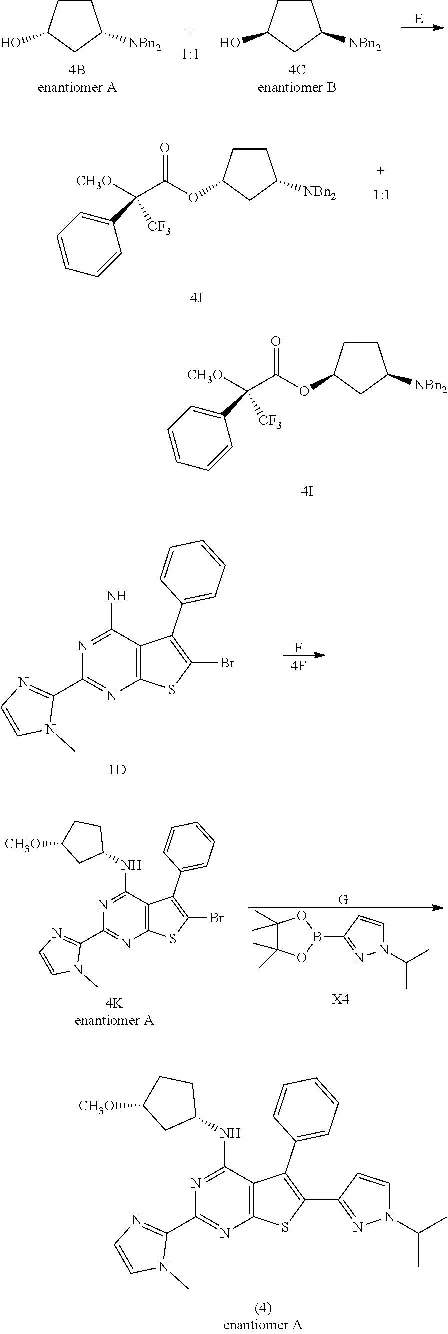

### 6-(1-isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine (4)

### Step A: (1R,3S)-3-(Dibenzylamino)cyclopentan-1-ol 4B and (1S,3R)-3-(dibenzylamino)cyclopentan-1-ol 4C

Sodium hydride (10.16 g, 423.56 mmol) was suspended in 100 mL of dry THF. The solution of rac-(1R,3S)-3-aminocyclopentan-1-ol (20.4 g, 201.7 mmol) in 200 mL of dry THE was added dropwise with water bath cooling. The resulting mixture was stirred until gas evolution ceased and then cooled to 0° C. (Bromomethyl)benzene (75.89 g, 443.73 mmol, 52.78 mL, 2.2 equiv) was added dropwise at that temperature, the resulting mixture was warmed to room temperature and then stirred overnight. After consumption of the starting material (¹H-NMR control) the reaction mixture was poured into saturated aq. ammonium chloride solution (250 mL H₂O+17.5 g ammonium chloride). The resulting mixture was extracted twice with 250 mL of CH₂Cl₂. Organic phases were combined, dried over sodium sulphate and evaporated to obtain a 1:1 mixture of (1R,3S)-3-(dibenzylamino)cyclopentan-1-ol 4B and (1S,3R)-3-(dibenzylamino)cyclopentan-1-ol 4C (35.0 g, 85.0% purity, 105.72 mmol, 52.4% yield). The product was purified by column chromatography to obtain 25 g (100% by LCMS) of a 1:1 mixture of (1R,3S)-3-(dibenzylamino)cyclopentan-1-ol 4B and (1S,3R)-3-(dibenzylamino)cyclopentan-1-ol 4C.

Preparative Separation of Enantiomers 4B and 4C:

Instrument: Agilent Technologies 1200: G1361A PrepPump, G2260A Prep ALS, G1315B DAD, G1364B Prer FC

Column: Chiralcel OD-H (250*30 mm*5 m); Mobile phase: Hexane-IPA-MeOH, 95-2.5-2.5 Flow Rate: 12 mL/min; Column Temperature: 24° C.; Wavelength: 205 nm, 254 nm),

RetTime (4B)=18.76 min; RetTime 4C)=34.49 min. The assignment of (1R,3S)-3-(dibenzylamino)cyclopentan-1-ol 4B and (1S,3R)-3-(dibenzylamino)cyclopentan-1-ol 4C absolute configuration was accomplished using Kazlauskas' Rule (see Step D and Step E below).

### Step B: (1S,3R)-N,N-dibenzyl-3-methoxycyclopentan-1-amine 4D and (1R,3S)-N,N-dibenzyl-3-methoxycyclopentan-1-amine 4E

Sodium hydride (68.24 mg, 2.84 mmol, 2.0 equiv) was suspended in 5 mL of dry THF. The solution of (1S,3R)-3-(dibenzylamino)cyclopentan-1-ol 4C (400.0 mg, 1.42 mmol) in 4 mL of dry THE was added dropwise with water bath cooling. The resulting mixture was stirred until gas evolution ceased and then cooled to 0° C. Iodomethane (403.59 mg, 2.84 mmol) was added dropwise at that temperature, the resulting mixture was warmed to room temperature and then stirred overnight. After consumption of the starting material (¹H-NMR control) the reaction mixture was poured into saturated aq. ammonium chloride solution (25 mL H₂O+1.5 g ammonium chloride). The resulting mixture was extracted twice with 25 mL of CH₂Cl₂. Organic phases were combined, dried over sodium sulphate and evaporated to obtain (1R,3S)-N,N-dibenzyl-3-methoxycyclopentan-1-amine 4E (390.0 mg, 91.0% purity, 1.2 mmol, 84.5% yield). The product was used on the next step without additional purification. Using the same procedure but starting with (1R,3S)-3-(dibenzylamino)cyclopentan-1-ol 4B, gave (1S,3R)-N,N-dibenzyl-3-methoxycyclopentan-1-amine 4D (85% yield).

### Step C: (1S,3R)-3-methoxycyclopentan-1-amine 4F and (1R,3S)-3-methoxycyclopentan-1-amine 4G

To a solution of (1R,3S)-N,N-dibenzyl-3-methoxycyclopentan-1-amine 4E (389.88 mg, 1.32 mmol) in MeOH (30 mL) was added palladium (70.0 mg, 657.77 μmol). The suspension was degassed and hydrogenated at 40° C. and ambient pressure for 7 days. Upon completion the mixture was cooled down to room temperature, the catalyst was removed off, and the filtrate was evaporated to dried to afford (1R,3S)-3-methoxycyclopentan-1-amine 4G (120.0 mg, 96.0% purity, 1.0 mmol, 75.8% yield). Using the same procedure but starting with (1S,3R)-N,N-dibenzyl-3-methoxycyclopentan-1-amine 4D gave (1S,3R)-3-methoxycyclopentan-1-amine 4F (77% yield)

### Step D: Enantiomeric resolution of (1R,3S)-3-(dibenzylamino)cyclopentan-1-ol 4B and (1S,3R)-3-(dibenzylamino)cyclopentan-1-ol 4C

To the product of Step A prior to chiral separation (1:1 mixture of 4B and 4C) (502.0 mg, 1.78 mmol) and prop-1-en-2-yl acetate (536.31 mg, 5.36 mmol, 590.0 μl, 3.0 equiv) in 5 mL MTBE 0.5 g (10% mass %) of Lipase PS “Amano” IM (CAS [9001-62-1]) was added in one portion. Solution was stirred 72 h at room temperature. Solution was filtered off and concentrated. Residue was purified by column chromatography, eluted by Hex:EtOAc 10:1. Recovered alcohol 4C (270.0 mg, 959.52 μmol, 53.8% yield) was isolated as light-yellow oil. The stereochemical assignment of the acylated product (1R,3S)-3-(dibenzylamino)cyclopentyl acetate 4H (not isolated) was made using Kazlauskas' rule, “A rule to predict which enantiomer of a secondary alcohol reacts faster in reactions catalyzed by cholesterol esterase, lipase from Pseudomonas cepacia, and lipase from Candida rugosa” R. J. Kazlauskas, A. N. E. Weissfloch, A. T. Rappaport, and L. A. Cuccia, J. Org. Chem. 1991, 56, 2656-2665. Therefore 4C is (1S,3R)-3-(dibenzylamino)cyclopentan-1-ol.

### Step E: Comparison of ¹⁹F NMR Measurements of R-Mosher's Acid Derivatives

The ¹⁹F NMR (in CDCl₃) of the R-Mosher's ester prepared from the alcohol 4C isolated in Step D (compound 41, (1S,3R)-3-(dibenzylamino)cyclopentyl (R)-3,3,3-trifluoro-2-methoxy-2-phenylpropanoate, −72.06 ppm) matched the ¹⁹F NMR of the R-Mosher's ester prepared from alcohol 4C isolated after chiral chromatography in Step A (−72.04 ppm). The ¹⁹F NMR of a 1:1 mixture of R-Mosher's esters 4J (1R,3S)-3-(dibenzylamino)cyclopentyl (R)-3,3,3-trifluoro-2-methoxy-2-phenylpropanoate and 41 (1S,3R)-3-(dibenzylamino)cyclopentyl (R)-3,3,3-trifluoro-2-methoxy-2-phenylpropanoate (prepared from the 1:1 mixture of 4B and 4C from Step A prior to chiral separation) had peaks at −71.95 ppm (4J) and −72.05 ppm (4I). For the preparation and use of Mosher esters see “Methoxy-α-trifluoromethylphenylacetic acid, a versatile reagent for the determination of enantiomeric composition of alcohols and amines” J. A. Dale, D. L. Dull, H. S. Mosher, J. Org. Chem., 1969, 34(9), 2543-2549.

### Step F: 6-Bromo-N-((1S,3R)-3-methoxycyclopentyl)-2-(1-methyl-1H-imidazol-2-yl)-5-phenylthieno[2,3-d]pyrimidin-4-amine 4K

6-Bromo-4-chloro-2-(1-methyl-1H-imidazol-2-yl)-5-phenylthieno[2,3-d]pyrimidine 1D (279.34 mg, 688.55 μmol), (1S,3R)-3-methoxycyclopentan-1-amine 4F (198.26 mg, 1.72 mmol) and N,N-diisopropylethylamine (267.12 mg, 2.07 mmol, 360.0 μL, 3.0 equiv) were mixed in DMSO (5 mL). The resulting reaction mixture was stirred at 100° C. for 15 h and then allowed to cool to room temperature. The mixture was diluted with water (10 mL) and extracted several times with EtOAc (15 mL*3). The combined extracts were washed with brine (10 mL), dried over anhydrous sodium sulfate, and filtered. The filtrate was concentrated and the residue was purified by HPLC to afford the 6-bromo-N-((1S,3R)-3-methoxycyclopentyl)-2-(1-methyl-1H-imidazol-2-yl)-5-phenylthieno[2,3-d]pyrimidin-4-amine 4K (119.0 mg, 245.66 μmol, 37.5% yield).

### Step G: 6-(1-Isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine (4)

6-Bromo-N-((1S,3R)-3-methoxycyclopentyl)-2-(1-methyl-1H-imidazol-2-yl)-5-phenylthieno[2,3-d]pyrimidin-4-amine 4K (119.77 mg, 247.25 μmol), 1-isopropyl-3-(4,4,5,5-tetramethyl-1,3,2-dioxaborolan-2-yl)-1H-pyrazole X4 (70.06 mg, 296.7 μmol), cesium carbonate (161.12 mg, 494.49 μmol) and PdXphosG3 (20.93 mg, 24.72 μmol) was dissolved in degassed dioxane/H₂O (2 mL:0.1 mL). The mixture was refluxed overnight. The mixture was cooled, filtered off, concentrated and purified by HPLC. The Example compound, 6-(1-isopropyl-1H-pyrazol-3-yl)-N-((1S,3R)-3-methoxycyclopentyl)-2-(1-methyl-1H-imidazol-2-yl)-5-phenylthieno[2,3-d]pyrimidin-4-amine (4) (6.7 mg, 13.04 μmol, 5.3% yield) was obtained.

### Example 5

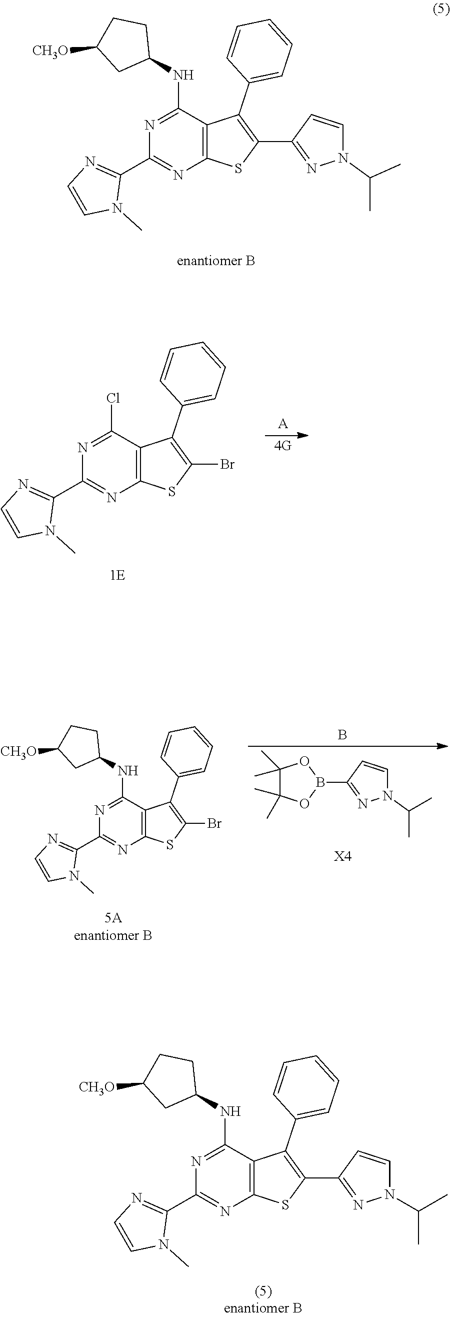

### 6-(1-Isopropyl-1H-pyrazol-3-yl)-N-((1R,3S)-3-methoxycyclopentyl)-2-(1-methyl-1H-imidazol-2-yl)-5-phenylthieno[2,3-d]pyrimidin-4-amine (5)

### Step A: 6-Bromo-N-((1R,3S)-3-methoxycyclopentyl)-2-(1-methyl-1H-imidazol-2-yl)-5-phenylthieno[2,3-d]pyrimidin-4-amine 5A

6-Bromo-4-chloro-2-(1-methyl-1H-imidazol-2-yl)-5-phenylthieno[2,3-d]pyrimidine 1E (352.48 mg, 868.83 μmol), (1R,3S)-3-methoxycyclopentan-1-amine 4G (125.0 mg, 1.09 mmol) and N,N-diisopropylethylamine (336.87 mg, 2.61 mmol, 450.0 μL, 3.0 equiv) were mixed in DMSO (5 mL). The resulting reaction mixture was stirred at 100° C. for 15 h and then allowed to cool to room temperature. The mixture was diluted with water (10 mL) and extracted several times with EtOAc (15 mL*3). The combined extracts were washed with brine (10 mL), dried over anhydrous sodium sulfate, and filtered. The filtrate was concentrated and the residue was purified by HPLC to afford the 6-bromo-N-((1R,3S)-3-methoxycyclopentyl)-2-(1-methyl-1H-imidazol-2-yl)-5-phenylthieno[2,3-d]pyrimidin-4-amine 5A (120.0 mg, 247.72 μmol, 30% yield).

### Step B: 6-(1-Isopropyl-1H-pyrazol-3-yl)-N-((1R,3S)-3-methoxycyclopentyl)-2-(1-methyl-1H-imidazol-2-yl)-5-phenylthieno[2,3-d]pyrimidin-4-amine (5)

6-Bromo-N-((1R,3S)-3-methoxycyclopentyl)-2-(1-methyl-1H-imidazol-2-yl)-5-phenylthieno[2,3-d]pyrimidin-4-amine 5A (122.77 mg, 247.35 μmol), 1-isopropyl-3-(4,4,5,5-tetramethyl-1,3,2-dioxaborolan-2-yl)-1H-pyrazole X4 (70.06 mg, 296.7 μmol), cesium carbonate (161.12 mg, 494.49 μmol) and PdXphosG3 (20.93 mg, 24.72 μmol) was dissolved in degassed dioxane:H₂O (2 mL:0.1 mL). Mixture was refluxed overnight. Mixture was cooled, filtered off, concentrated and purified by HPLC. The Example compound (5), 6-(1-isopropyl-1H-pyrazol-3-yl)-N-((1R,3S)-3-methoxycyclopentyl)-2-(1-methyl-1H-imidazol-2-yl)-5-phenylthieno[2,3-d]pyrimidin-4-amine (29.0 mg, 95.0% purity, 53.64 μmol, 21.7% yield) was obtained.

### Example 6

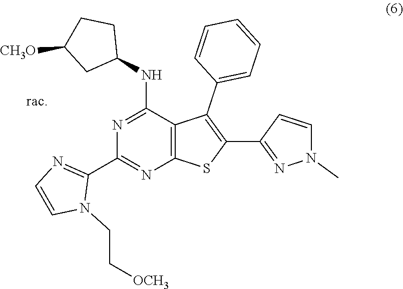

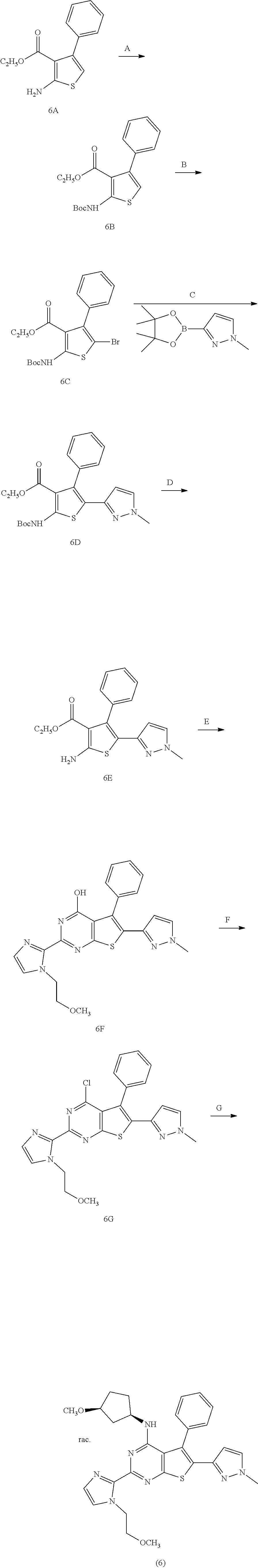

### Rac-N-((1R,3S)-3-methoxycyclopentyl)-2-(1-(2-methoxyethyl)-1H-imidazol-2-yl)-6-(1-methyl-1H-pyrazol-3-yl)-5-phenylthieno[2,3-d]pyrimidin-4-amine (6)

### Step A: Ethyl 2-((tert-butoxycarbonyl)amino)-4-phenylthiophene-3-carboxylate 6B

To a solution of ethyl 2-amino-4-phenylthiophene-3-carboxylate 6A (16.0 g, 64.7 mmol) and di-tert-butyl dicarbonate (16.94 g, 77.63 mmol) in dioxane (210 mL) N,N-dimethylpyridin-4-amine (790.37 mg, 6.47 mmol) was added. The mixture was stirred at 80° C. overnight. Then it was cooled, concentrated and purified by column chromatography. Eluent Hexane/EtOAc—10:1. Ethyl 2-[(tert-butoxy)carbonyl]amino-4-phenylthiophene-3-carboxylate 6B (15.0 g, 95.0% purity, 41.02 mmol, 63.4% yield) was obtained as white powder.

### Step B: Ethyl 5-bromo-2-[(tert-butoxy)carbonyl]amino-4-phenylthiophene-3-carboxylate 6C

To a stirred solution of ethyl 2-[(tert-butoxy)carbonyl]amino-4-phenylthiophene-3-carboxylate 6B (15.0 g, 43.17 mmol) in DMF (30 mL) N-bromosuccinimide (9.22 g, 51.81 mmol) was added in one portion. The mixture was stirred at room temperature overnight. Then it was poured in ice-water (150 mL) and diluted with EtOAc (250 mL). Organic phase was washed with water (5×50 mL), dried over Na₂SO₄, concentrated and purified by column chromatography. Eluent Hex/EtOAc—7:1. Ethyl 5-bromo-2-[(tert-butoxy)carbonyl]amino-4-phenylthiophene-3-carboxylate 6C (10.5 g, 95.0% purity, 23.4 mmol, 54.2% yield) was obtained as light-yellow powder.

### Step C: Ethyl 2-[(tert-butoxy)carbonyl]amino-5-(1-methyl-1H-pyrazol-3-yl)-4-phenylthiophene-3-carboxylate 6D

To a stirred suspension of ethyl 5-bromo-2-[(tert-butoxy)carbonyl]amino-4-phenylthiophene-3-carboxylate 6C (3.7 g, 8.68 mmol), 1-methyl-3-(tetramethyl-1,3,2-dioxaborolan-2-yl)-1H-pyrazole X5 (2.17 g, 10.41 mmol) and Cs₂CO₃ (5.65 g, 17.36 mmol) in dioxane/H₂O (74 mL)/(3.7 mL) Pd(dppf)Cl₂·CH₂Cl₂ (634.98 mg, 867.8 μmol) was added in one portion under Ar. The mixture was stirred at 60° C. overnight. After cooling to room temperature, insolubles were filtered off and washed once with EtOAc (25 mL). The filtrate was concentrated and purified by column chromatography. Eluent Hex/EtOAc—100:1 to 10:1. Ethyl 2-[(tert-butoxy)carbonyl]amino-5-(1-methyl-1H-pyrazol-3-yl)-4-phenylthiophene-3-carboxylate 6D (1.9 g, 95.0% purity, 4.22 mmol, 48.7% yield) was obtained as yellow powder.

### Step D: Ethyl 2-amino-5-(1-methyl-1H-pyrazol-3-yl)-4-phenylthiophene-3-carboxylate 6E

To a solution of ethyl 2-[(tert-butoxy)carbonyl]amino-5-(1-methyl-1H-pyrazol-3-yl)-4-phenylthiophene-3-carboxylate 6D (1.9 g, 4.44 mmol) in CH₂Cl₂ (20 mL) dioxane*HCl was added (7 mL 8M). Mixture was stirred overnight at room temperature. Ethyl 2-amino-5-(1-methyl-1H-pyrazol-3-yl)-4-phenylthiophene-3-carboxylate 6E (1.1 g, 3.36 mmol, 75.6% yield) was obtained as black oil.

### Step E: 2-(1-(2-Methoxyethyl)-1H-imidazol-2-yl)-6-(1-methyl-1H-pyrazol-3-yl)-5-phenylthieno[2,3-d]pyrimidin-4-ol 6F

Ethyl 2-amino-5-(1-methyl-1H-pyrazol-3-yl)-4-phenylthiophene-3-carboxylate 6E (3.45 g, 11.02 mmol), 1-(2-methoxyethyl)-1H-imidazole-2-carbonitrile (2.0 g, 13.23 mmol), potassium t-butoxide (3.71 g, 33.07 mmol) was dissolved in dry MeOH (10 mL) and stirred overnight at room temperature. The residue was evaporated and acetic acid was added to pH 5. The resulted mixture was extracted with EtOAc (25 mL), organic layer was dried over Na₂SO₄, evaporated to give: 2-(1-(2-methoxyethyl)-1H-imidazol-2-yl)-6-(1-methyl-1H-pyrazol-3-yl)-5-phenylthieno[2,3-d]pyrimidin-4-ol 6F (4.2 g, 43.0% purity, 4.18 mmol, 37.9% yield) which was used without additional purification on the next step.

### Step F: 4-Chloro-2-(1-(2-methoxyethyl)-1H-imidazol-2-yl)-6-(1-methyl-1H-pyrazol-3-yl)-5-phenylthieno[2,3-d]pyrimidine 6G

To the suspension of 2-[1-(2-methoxyethyl)-1H-imidazol-2-yl]-6-(1-methyl-1H-pyrazol-3-yl)-5-phenylthieno[2,3-d]pyrimidin-4-ol 6F (123.72 mg, 286.07 μmol) in phosphoroyl trichloride (438.63 mg, 2.86 mmol, 270.0 μL, 10.0 equiv) N,N-diisopropylethylamine (111.3 mg, 861.17 μmol, 150.0 μL, 3.0 equiv) was added at room temperature. The reaction mixture was refluxed for 16 h, cooled to room temperature and evaporated under reduced pressure. The residue was poured in ice and diluted with ice-cold ammonia (20 mL, 20-25% of ammonia), the product was extracted with CHCl₃ (2×50 mL) and evaporated. 4-Chloro-2-(1-(2-methoxyethyl)-1H-imidazol-2-yl)-6-(1-methyl-1H-pyrazol-3-yl)-5-phenylthieno[2,3-d]pyrimidine 6G (125.0 mg, 75.4% purity, 209.01 μmol, 73.1% yield) was obtained as yellow solid.

### Step G: rac-N-((1R,3S)-3-Methoxycyclopentyl)-2-(1-(2-methoxyethyl)-1H-imidazol-2-yl)-6-(1-methyl-1H-pyrazol-3-yl)-5-phenylthieno[2,3-d]pyrimidin-4-amine (6)

To the solution of 4-chloro-2-(1-(2-methoxyethyl)-1H-imidazol-2-yl)-6-(1-methyl-1H-pyrazol-3-yl)-5-phenylthieno[2,3-d]pyrimidine 6G (146.44 mg, 324.74 μmol) in DMSO (5 mL), N,N-diisopropylethylamine (126.14 mg, 975.99 μmol, 170.0 μl, 3.0 equiv) and rac-(1R,3S)-3-methoxycyclopentan-1-amine (56.1 mg, 487.11 μmol) were added in one portion. The mixture was heated at 100° C. overnight, cooled and purified by HPLC. The Example compound (6), rac-N-((1R,3S)-3-methoxycyclopentyl)-2-(1-(2-methoxyethyl)-1H-imidazol-2-yl)-6-(1-methyl-1H-pyrazol-3-yl)-5-phenylthieno[2,3-d]pyrimidin-4-amine (7.2 mg, 13.59 μmol, 4.2% yield) was obtained as yellow viscous residue.

### Example 7

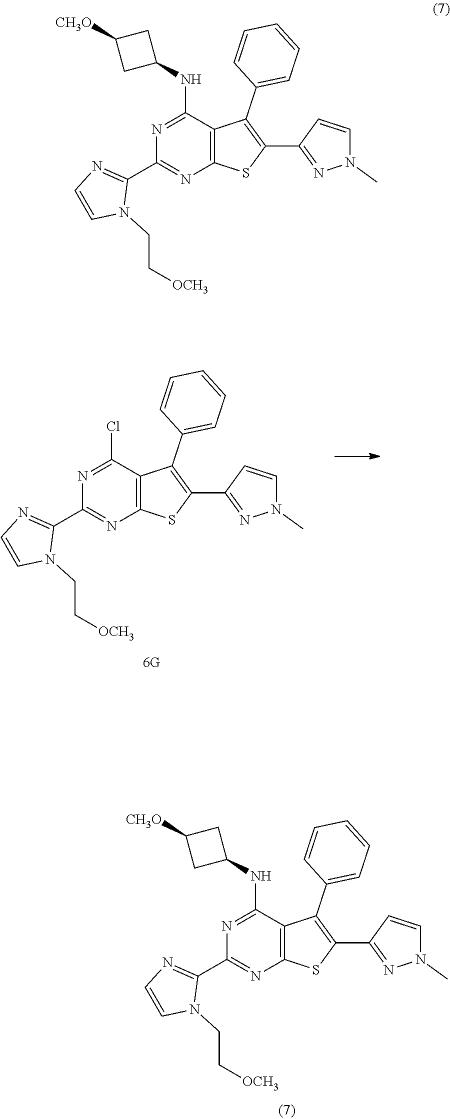

### N-((1s,3s)-3-Methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine (7)

To the solution of 4-chloro-2-(1-(2-methoxyethyl)-1H-imidazol-2-yl)-6-(1-methyl-1H-pyrazol-3-yl)-5-phenylthieno[2,3-d]pyrimidine 6G (138.18 mg, 306.42 μmol) in DMSO (5 mL), N,N-diisopropylethylamine (118.72 mg, 918.58 μmol, 160.0 μL, 3.0 equiv) with (1s,3s)-3-methoxycyclobutan-1-amine hydrochloride (46.49 mg, 459.63 μmol) were added in one portion. The mixture was heated at 100° C. overnight, cooled and purified by HPLC. The Example compound (7), N-((1s,3s)-3-methoxycyclobutyl)-2-(1-(2-methoxyethyl)-1H-imidazol-2-yl)-6-(1-methyl-1H-pyrazol-3-yl)-5-phenylthieno[2,3-d]pyrimidin-4-amine (25.0 mg, 48.48 μmol, 15.8% yield) was obtained as yellow viscous residue.

### Example 8

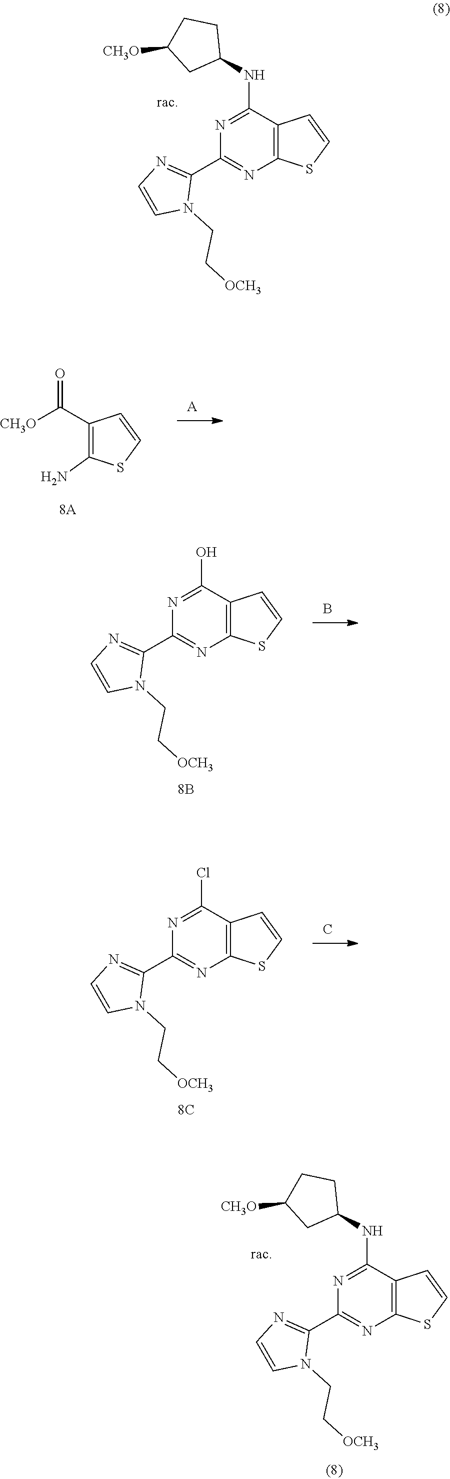

### Rac-N-((1R,3S)-3-methoxycyclopentyl)-2-(1-(2-methoxyethyl)-1H-imidazol-2-yl)thieno[2,3-d]pyrimidin-4-amine (8)

### Step A: 2-(1-(2-Methoxyethyl)-1H-imidazol-2-yl)thieno[2,3-d]pyrimidin-4-ol 8B

Methyl 2-aminothiophene-3-carboxylate 8A (415.85 mg, 2.65 mmol), 1-(2-methoxyethyl)-1H-imidazole-2-carbonitrile (600.0 mg, 3.97 mmol), potassium t-butoxide (890.58 mg, 7.94 mmol) was dissolved in dry MeOH (6 mL) and stirred overnight at room temperature. The reaction mixture was evaporated and acetic acid was added to pH 5. The resulted mixture was extracted with EtOAc (15 ml), organic layer was separated, dried over Na₂SO₄ and evaporated to give 2-(1-(2-methoxyethyl)-1H-imidazol-2-yl)thieno[2,3-d]pyrimidin-4-ol 8B (850.0 mg, 73.8% purity, 2.27 mmol, 85.8% yield) which was used without additional purification on a further step.

### Step B. 4-Chloro-2-(1-(2-methoxyethyl)-1H-imidazol-2-yl)thieno[2,3-d]pyrimidine 8C

To a suspension of 2-(1-(2-methoxyethyl)-1H-imidazol-2-yl)thieno[2,3-d]pyrimidin-4-ol 8B (999.29 mg, 3.62 mmol) in phosphoroyl trichloride (5.55 g, 36.17 mmol, 3.37 mL, 10.0 equiv) N,N-diisopropylethylamine (1.4 g, 10.85 mmol, 1.89 mL, 3.0 equiv) was added at room temperature. The reaction mixture was refluxed for 16 h, cooled to room temperature and evaporated under reduced pressure. The residue was poured in ice and diluted with ice-cold ammonia (20 mL, 20-25% of ammonia), the product was extracted with CHCl₃ (2×50 mL) and evaporated. 4-Chloro-2-(1-(2-methoxyethyl)-1H-imidazol-2-yl)thieno[2,3-d]pyrimidine 8C (1.0 g, 88.4% purity, 3.0 mmol, 82.9% yield) was obtained as yellow solid.

### Step C: rac-N-((1R,3S)-3-Methoxycyclopentyl)-2-(1-(2-methoxyethyl)-1H-imidazol-2-yl)thieno[2,3-d]pyrimidin-4-amine (8)

To the solution of 4-chloro-2-(1-(2-methoxyethyl)-1H-imidazol-2-yl)thieno[2,3-d]pyrimidine 8C (801.08 mg, 2.72 mmol) in DMSO (5 mL), N,N-diisopropylethylamine (1.05 g, 8.15 mmol, 1.42 mL, 3.0 equiv) and rac-(1R,3S)-3-methoxycyclopentan-1-amine (469.52 mg, 4.08 mmol) were added in one portion. The mixture was heated at 100° C. overnight, cooled and purified by HPLC. The Example compound, rac-N-((1R,3S)-3-methoxycyclopentyl)-2-(1-(2-methoxyethyl)-1H-imidazol-2-yl)thieno[2,3-d]pyrimidin-4-amine (8) (50.0 mg, 96.0% purity, 128.52 μmol, 4.7% yield) was obtained as yellow viscous residue.

### Example 9

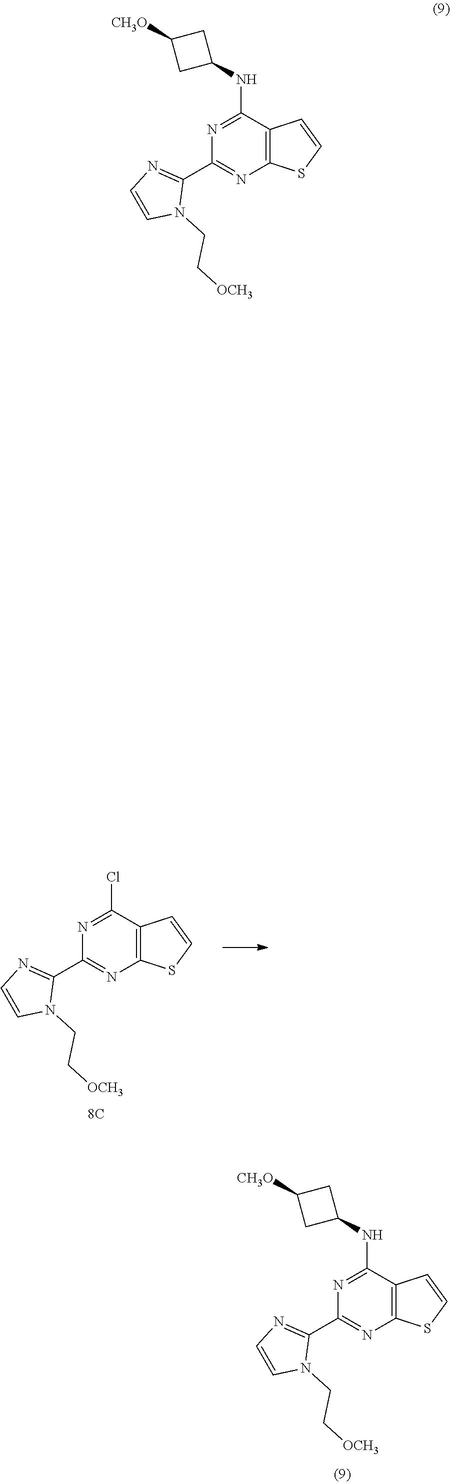

### N-((1s,3s)-3-Methoxycyclobutyl)-2-(1-(2-methoxyethyl)-1H-imidazol-2-yl)thieno[2,3-d]pyrimidin-4-amine (9)

The 4-chloro-2-(1-(2-methoxyethyl)-1H-imidazol-2-yl)thieno[2,3-d]pyrimidine 8C (801.18 mg, 2.72 mmol) was dissolved in DMSO (5 mL) and N,N-diisopropylethylamine (1.05 g, 8.15 mmol, 1.42 mL, 3.0 equiv) with corresponding amine cis-3-methoxycyclobutan-1-amine hydrochloride (561.04 mg, 4.08 mmol) was added. The mixture was heated at 100° C. overnight, cooled and purified by HPLC. The Example compound, (9) N-((1s,3s)-3-methoxycyclobutyl)-2-(1-(2-methoxyethyl)-1H-imidazol-2-yl)thieno[2,3-d]pyrimidin-4-amine (60.0 mg, 166.92 μmol, 6.1% yield) was obtained as yellow viscous residue.

### Example 10

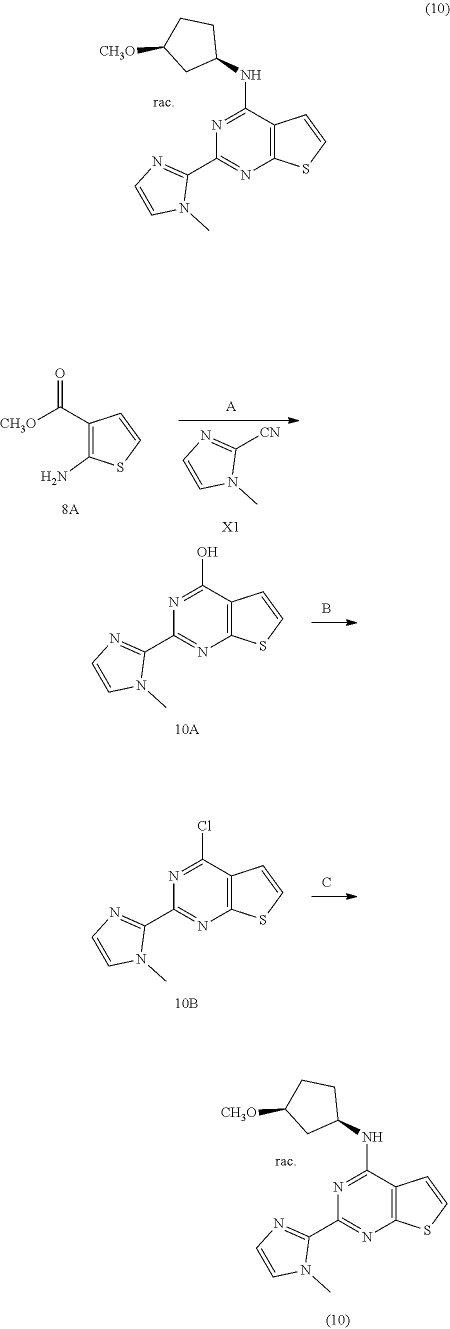

### Rac-N-((1R,3S)-3-methoxycyclopentyl)-2-(1-methyl-1H-imidazol-2-yl)thieno[2,3-d]pyrimidin-4-amine (10)

### Step A: 2-(1-Methyl-1H-imidazol-2-yl)thieno[2,3-d]pyrimidin-4-ol 10A

Methyl 2-aminothiophene-3-carboxylate 8A (1.0 g, 6.36 mmol), 1-methyl-1H-imidazole-2-carbonitrile X1 (1.02 g, 9.55 mmol), potassium t-butoxide (2.14 g, 19.09 mmol) was dissolved in dry MeOH (10 mL) and stirred overnight at room temperature. The reaction mixture was evaporated and acetic acid was added to pH 5. The resulted mixture was extracted with EtOAc (25 mL), organic layer was separated, dried over Na₂SO₄ and evaporated to give 2-(1-methyl-1H-imidazol-2-yl)thieno[2,3-d]pyrimidin-4-ol 10A (1.5 g, 92.0% purity, 5.94 mmol, 93.4% yield) which was used without additional purification on a further step.

### Step B: 4-Chloro-2-(1-methyl-1H-imidazol-2-yl)thieno[2,3-d]pyrimidine 10B

To the suspension of 2-(1-methyl-1H-imidazol-2-yl)thieno[2,3-d]pyrimidin-4-ol 10A (1.75 g, 7.54 mmol) in phosphoroyl trichloride (11.56 g, 75.39 mmol, 7.03 mL, 10.0 equiv) N,N-diisopropylethylamine (1.4 g, 10.85 mmol, 1.89 mL, 3.0 equiv) was added at room temperature. The reaction mixture was refluxed for 16 h, cooled to room temperature. and evaporated under reduced pressure. The residue was poured in ice and diluted with ice-cold ammonia (20 mL, 20-25% of ammonia), the product was extracted with CHCl₃ (2×50 mL) and evaporated. 4-Chloro-2-(1-methyl-1H-imidazol-2-yl)thieno[2,3-d]pyrimidine 10B (1.85 g, 86.7% purity, 6.4 mmol, 84.9% yield) was obtained as yellow solid.

### Step C: rac-N-((1R,3S)-3-Methoxycyclopentyl)-2-(1-methyl-1H-imidazol-2-yl)thieno[2,3-d]pyrimidin-4-amine (10)

To the solution of 4-chloro-2-(1-methyl-1H-imidazol-2-yl)thieno[2,3-d]pyrimidine 10B (1.3 g, 5.19 mmol) in DMSO (5 mL) N,N-diisopropylethylamine (1.01 g, 7.78 mmol, 1.35 mL, 1.5 equiv) and rac-(1R,3S)-3-methoxycyclopentan-1-amine (895.74 mg, 7.78 mmol) were added in one portion. The mixture was heated at 100° C. overnight, cooled and purified by HPLC. The Example compound, rac-N-((1R,3S)-3-methoxycyclopentyl)-2-(1-methyl-1H-imidazol-2-yl)thieno[2,3-d]pyrimidin-4-amine (10) (90.0 mg, 273.21 μmol, 5.3% yield) was obtained as yellow viscous residue.

### Example 11

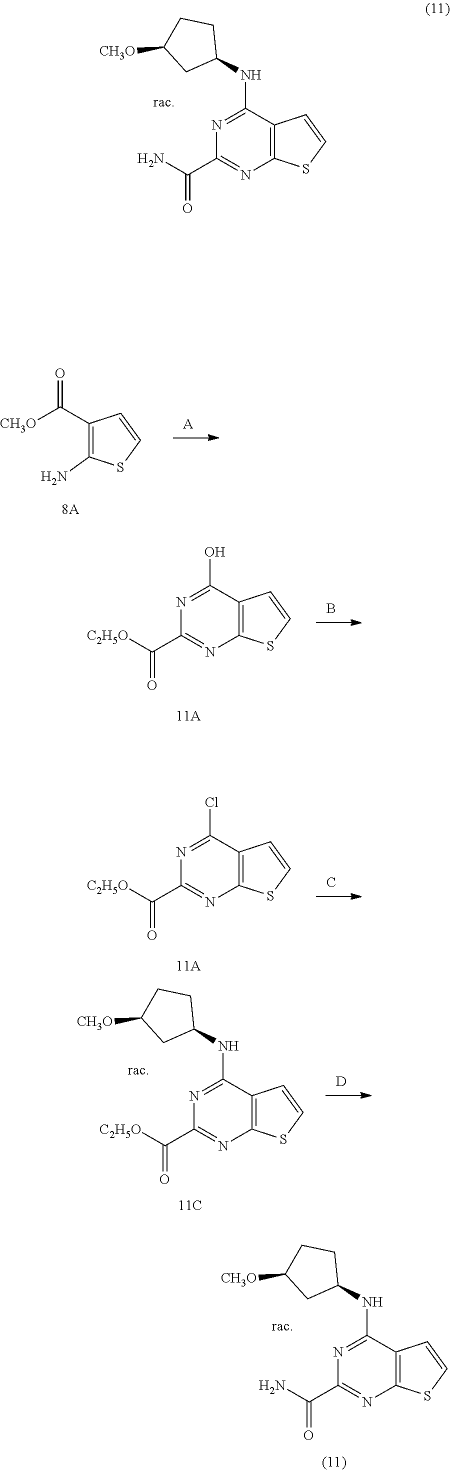

Rac-4-(((1R,3S)-3-methoxycyclopentyl)amino)thieno[2,3-d]pyrimidine-2-carboxamide (11).

### Step A: Ethyl 4-hydroxythieno[2,3-d]pyrimidine-2-carboxylate 11A

Methyl 2-aminothiophene-3-carboxylate 8A (24.99 g, 159.01 mmol) and ethyl cyanoformate (23.63 g, 238.52 mmol, 23.4 mL, 1.5 equiv) were dissolved in dioxane*HCl (8 M solution, 350 mL) and resulted mixture was refluxed overnight. Then dioxane*HCl 250 mL (8 M solution) was added to the reaction mixture and refluxed 8 h. The reaction mixture was filtered and the product washed with CH₃CN (350 mL) and MTBE (500 mL) to give ethyl 4-hydroxythieno[2,3-d]pyrimidine-2-carboxylate 11A (25.0 g, 82.0% purity, 91.42 mmol, 57.5% yield) as brown solid.

### Step B: Ethyl 4-chlorothieno[2,3-d]pyrimidine-2-carboxylate 11B

To the suspension of ethyl 4-hydroxythieno[2,3-d]pyrimidine-2-carboxylate 11A (11.5 g, 51.29 mmol) in phosphoroyl trichloride (62.91 g, 410.28 mmol, 38.24 mL, 8.0 equiv) N,N-diispropylethylamine (19.88 g, 153.86 mmol, 26.8 mL, 3.0 equiv) was added at room temperature. The reaction mixture was refluxed for 16 h, cooled to room temperature and evaporated under reduced pressure. The residue was poured in ice and diluted with ice-cold ammonia (150 mL, 20-25% of ammonia) to pH >7.5, the product was extracted with CHCl₃ (2×250 mL). Organic phase was dried over Na₂SO₄ and evaporated. Ethyl 4-chlorothieno[2,3-d]pyrimidine-2-carboxylate 11B (12.0 g, 90.0% purity, 44.5 mmol, 86.8% yield) was obtained as brown solid.

### Step C: rac-Ethyl 4-(((1R,3S)-3-methoxycyclopentyl)amino)thieno[2,3-d]pyrimidine-2-carboxylate 11C

To a solution of 4-chlorothieno[2,3-d]pyrimidine-2-carboxylate 11B (5.75 g, 23.69 mmol) in DMSO (5 mL), N,N-diispropylethylamine (4.59 g, 35.54 mmol, 6.19 mL, 1.5 equiv) and rac-(1R,3S)-3-methoxycyclopentan-1-amine (3.0 g, 26.06 mmol) were added in one portion. The mixture was heated at 100° C. overnight, cooled and purified by HPLC. rac-Ethyl 4-(((1R,3S)-3-methoxycyclopentyl)amino)thieno[2,3-d]pyrimidine-2-carboxylate 11C (1.5 g, 84.0% purity, 3.92 mmol, 16.5% yield) was obtained as yellow viscous residue.

### Step D: rac-4-(((1R,3S)-3-Methoxycyclopentyl)amino)thieno[2,3-d]pyrimidine-2-carboxamide (11)

General procedure: rac-Ethyl 4-(((1R,3S)-3-methoxycyclopentyl)amino)thieno[2,3-d]pyrimidine-2-carboxylate 11C (1 eq) (0.2 g, 0.622 mmol) was suspended in the appropriate corresponding amine (5 eq), (and stirred 24-72 h at room temperature. The reaction mixture was concentrated and purified by HPLC. Using NH₄OH 25% as the amine produced the Example compound (11), rac-4-(((1R,3S)-3-methoxycyclopentyl)amino)thieno[2,3-d]pyrimidine-2-carboxamide (0.050 g, 29.9% yield).

### Example 12

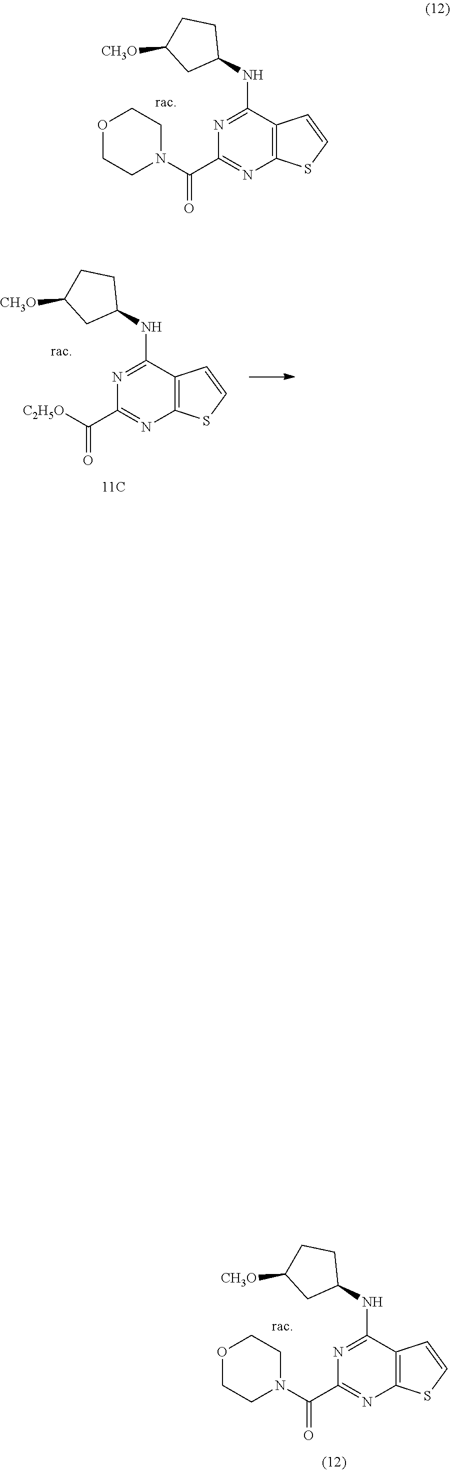

### Rac-(4-(((1R,3S)-3-methoxycyclopentyl)amino)thieno[2,3-d]pyrimidin-2-yl)(morpholino)methanone (12)

Following the general procedure described in Example 11, Step D using rac-ethyl 4-(((1R,3S)-3-methoxycyclopentyl)amino)thieno[2,3-d]pyrimidine-2-carboxylate 11C (1 eq) (0.2 g, 0.622 mmol) and morpholine as the amine produced the Example compound (12), rac-(4-(((1R,3S)-3-methoxycyclopentyl)amino)thieno[2,3-d]pyrimidin-2-yl)(morpholino)methanone (0.009 g, 4.3% yield).

### Example 13

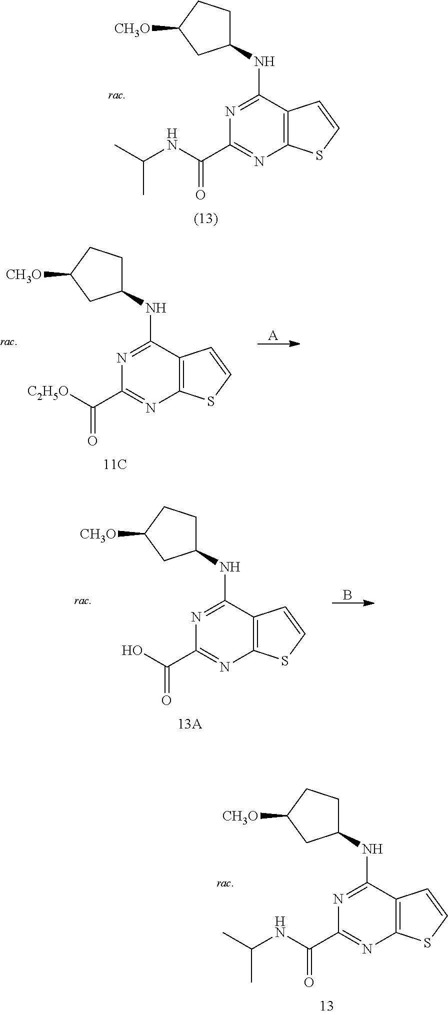

### Rac-N-isopropyl-4-(((1R,3S)-3-methoxycyclopentyl)amino)thieno[2,3-d]pyrimidine-2-carboxamide (13)

### Step A: rac-4-(((1R,3S)-3-Methoxycyclopentyl)amino)thieno[2,3-d]pyrimidine-2-carboxylic acid 13A

To a solution of rac-ethyl 4-(((1R,3S)-3-methoxycyclopentyl)amino)thieno[2,3-d]pyrimidine-2-carboxylate 11C (600.0 mg, 1.87 mmol) in THE (6 mL) and H₂O (1 mL) LiOH×H₂O (235.12 mg, 5.6 mmol) was added in one portion. The reaction mixture was stirred for 16 h at room temperature. Then it was diluted with water (5 mL), acidified by HCl (1N) to pH-2 and extracted by EtOAc (2×15 mL). The organic phase was dried over Na₂SO₄ and concentrated. rac-4-(((1R,3S)-3-Methoxycyclopentyl)amino)thieno[2,3-d]pyrimidine-2-carboxylic acid 13A (200.0 mg, 681.8 μmol, 39.8% yield) was obtained as white powder.

### Step B: rac-N-Isopropyl-4-(((1R,3S)-3-methoxycyclopentyl)amino)thieno[2,3-d]pyrimidine-2-carboxamide (13)

General procedure: To a solution of rac-4-(((1R,3S)-3-methoxycyclopentyl)amino)thieno[2,3-d]pyrimidine-2-carboxylic acid 13A (0.1 g, 0.343 mmol, 1 eq) and HATU (0.156 g, 0.411 mmol, 1.2 eq) in DMF (20 mL/g), DIPEA (0.18 mL, 1 mmol, 3 eq) was added. The resulted mixture was stirred 30 min at room temperature and the appropriate corresponding amine (1.5 eq) was added in one portion. The final product was isolated by HPLC, without pre-workup. Using isopropylamine as the corresponding amine produced Example compound (13), rac-N-isopropyl-4-(((1R,3S)-3-methoxycyclopentyl)amino)thieno[2,3-d]pyrimidine-2-carboxamide (0.0131 g, 12.2% yield).

### Example 14-Example 16

Following the general procedure described in Example 13, Step B, rac-4-(((1R,3S)-3-methoxycyclopentyl)amino)thieno[2,3-d]pyrimidine-2-carboxylic acid 13A (0.1 g, 0.343 mmol, 1 eq) was treated with the appropriate corresponding amine (1.5 eq) to produce the Example compounds (14)-(16) shown in Table A.

TABLE A

| | | | Amount, |
|---|---|---|---|
| Ex. | Structure | Name | Yield |
| 14 | 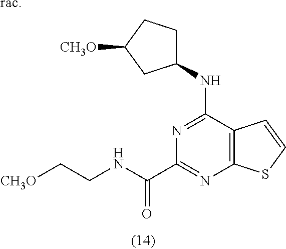 | rac-4-(((1R,3S)-3- Methoxycyclopentyl)amino)-N- (2-methoxyethyl)thieno[2,3- d]pyrimidine-2-carboxamide | 0.0477 g, 42.6% |
| 15 | 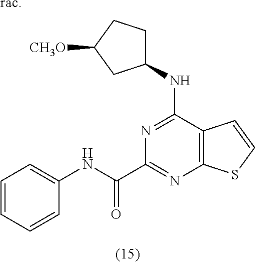 | rac-4-(((1R,3S)-3- Methoxycyclopentyl)amino)-N- phenylthieno[2,3-d]pyrimidine- 2-carboxamide | 0.0992 g, 84.8% |
| 16 | 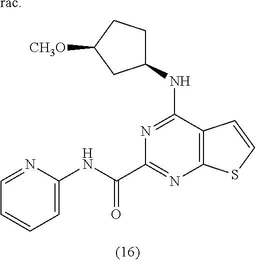 | rac-4-(((1R,3S)-3- Methoxycyclopentyl)amino)-N- (pyridin-2-yl)thieno[2,3- d]pyrimidine-2-carboxamide | 0.015 g, 12.7% |

### Example 17

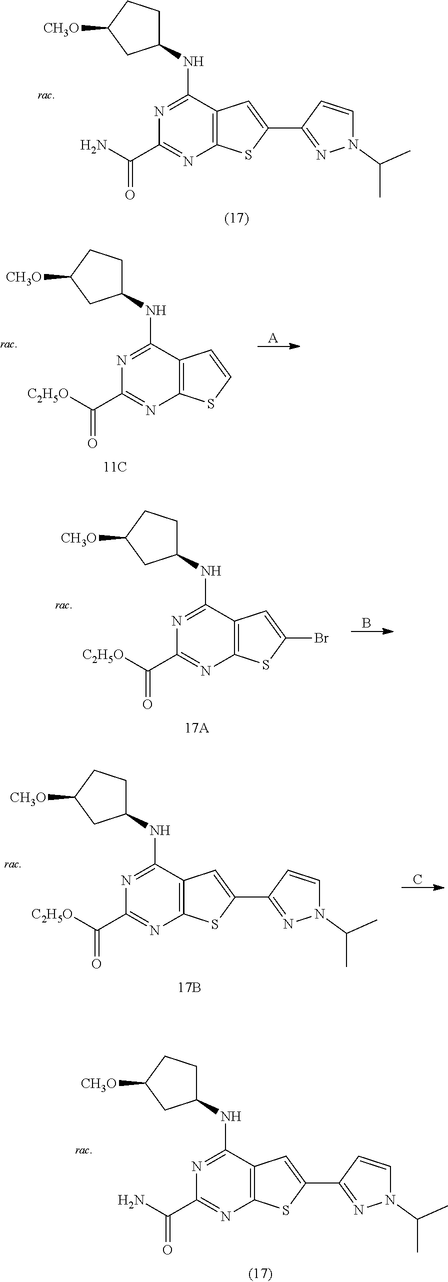

### Rac-6-(1-isopropyl-1H-pyrazol-3-yl)-4-(((1R,3S)-3-methoxycyclopentyl)amino)thieno[2,3-d]pyrimidine-2-carboxamide (17)

### Step A: rac-Ethyl 6-bromo-4-(((1R,3S)-3-methoxycyclopentyl)amino)thieno[2,3-d]pyrimidine-2-carboxylate 17A

To a solution of rac-ethyl 4-(((1R,3S)-3-methoxycyclopentyl)amino)thieno[2,3-d]pyrimidine-2-carboxylate 11C (300.0 mg, 749.46 μmol) in DMF (5 mL), N-bromosuccinimide (160.07 mg, 899.36 μmol) was added in one portion. The resulted solution was stirred overnight at room temperature and then it was poured in ice water (10 mL). The product-precipitate formed was filtered and dried on air overnight. rac-Ethyl 6-bromo-4-(((1R,3S)-3-methoxycyclopentyl)amino)thieno[2,3-d]pyrimidine-2-carboxylate 17A (200.0 mg, 78.0% purity, 389.72 μmol, 55.3% yield) was obtained as dark brown powder.

### Step B: rac-Ethyl 6-(1-isopropyl-1H-pyrazol-3-yl)-4-(((1R,3S)-3-methoxycyclopentyl)amino)thieno[2,3-d]pyrimidine-2-carboxylate 17B

rac-Ethyl 6-bromo-4-(((1R,3S)-3-methoxycyclopentyl)amino)thieno[2,3-d]pyrimidine-2-carboxylate 17A (200.39 mg, 500.6 μmol), 5-(4,4,5,5-tetramethyl-1,3,2-dioxaborolan-2-yl)-1-[2-(trimethylsilyl)ethoxy]methyl-1H-pyrazole (267.27 mg, 824.14 μmol), cesium carbonate (326.21 mg, 1.0 mmol) and PdXPhosG3 (42.37 mg, 50.06 μmol) was mixed in dioxane/H₂O (4 mL)/(0.2 mL). The reaction flask was filled by argon and the reaction mixture was stirred at 80° C. overnight, then cooled, filtered through SiO₂ pad (10 mL) and washed by EtOAc (2×15 mL). This solution was concentrated and used on next step without further purification. rac-Ethyl 6-(1-isopropyl-1H-pyrazol-3-yl)-4-(((1R,3S)-3-methoxycyclopentyl)amino)thieno[2,3-d]pyrimidine-2-carboxylate 17B (200.0 mg, 64.0% purity, 298.0 μmol, 63.1% yield) was obtained as brown oil.

### Step C: rac-6-(1-Isopropyl-1H-pyrazol-3-yl)-4-(((1R,3S)-3-methoxycyclopentyl)amino)thieno[2,3-d]pyrimidine-2-carboxamide (17)

rac-Ethyl 6-(1-isopropyl-1H-pyrazol-3-yl)-4-(((1R,3S)-3-methoxycyclopentyl)amino)thieno[2,3-d]pyrimidine-2-carboxylate 17B (200.0 mg, 465.62 μmol) was suspended in NH₄OH (2 mL 25% w/w). This mixture was stirred 24 h at room temperature, then concentrated and purified by HPLC. The Example compound (17), rac-6-(1-isopropyl-1H-pyrazol-3-yl)-4-(((1R,3S)-3-methoxycyclopentyl)amino)thieno[2,3-d]pyrimidine-2-carboxamide (28.0 mg, 95.0% purity, 66.42 μmol, 14.3% yield) was obtained as yellow solid.

### Example 18

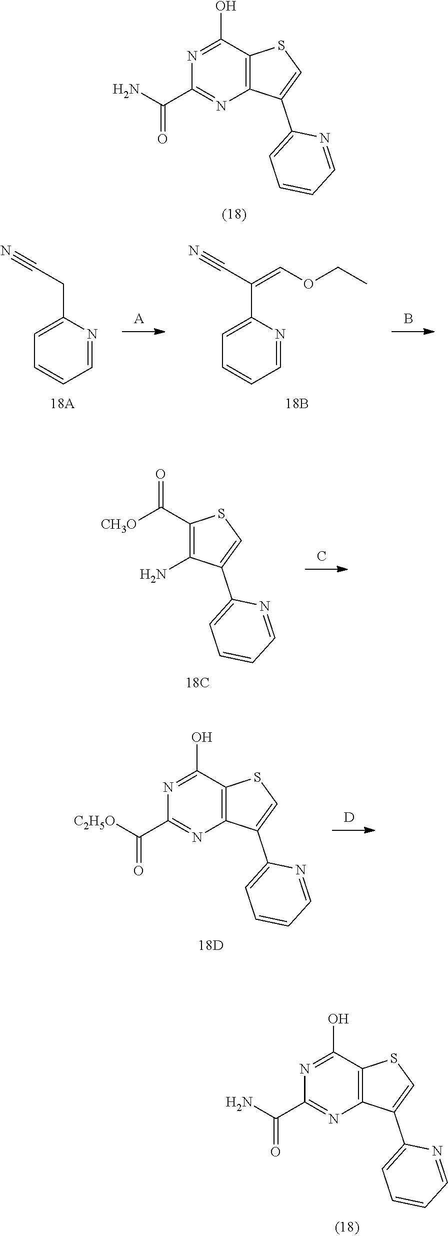

### 4-Hydroxy-7-(pyridin-2-yl)thieno[3,2-d]pyrimidine-2-carboxamide (18)

### Step A: (E)-3-Ethoxy-2-(pyridin-2-yl)acrylonitrile 18B

A mixture of 2-(pyridin-2-yl)acetonitrile 18A (21.0 g, 177.76 mmol), triethoxymethane (39.52 g, 266.64 mmol, 44.4 mL, 1.5 equiv) and acetic anhydride (181.47 g, 1.78 mol, 168.03 mL, 10.0 equiv) was refluxed for 4 hours. Then it was evaporated and purified by flash chromatography to give (E)-3-ethoxy-2-(pyridin-2-yl)acrylonitrile 18B (27.0 g, 65.0% purity, 100.75 mmol, 56.7% yield).

### Step B: Methyl 3-amino-4-(pyridin-2-yl)thiophene-2-carboxylate 18C

To a stirred solution of methyl 2-sulfanylacetate (98.11 g, 924.27 mmol, 82.65 mL, 1.15 eq) in dry MeOH (500 mL), sodium methanolate (78.16 g, 1.45 mol) was added at 0° C. After 5 min to the resulted mixture a solution of (E)-3-ethoxy-2-(pyridin-2-yl)acrylonitrile 18B (140.01 g, 803.72 mmol) in MeOH (200 mL) was added dropwise. This mixture was heated at 65° C. overnight. Then the solvent was evaporated and distributed in water/EtOAc (700 mL/700 mL). Organic phase was separated, dried over Na₂SO₄ and evaporated. Methyl 3-amino-4-(pyridin-2-yl)thiophene-2-carboxylate 18C (20.0 g, 85.37 mmol, 10.6% yield) was obtained by column chromatography.

### Step C: Ethyl 4-hydroxy-7-(pyridin-2-yl)thieno[3,2-d]pyrimidine-2-carboxylate 18D

Methyl 3-amino-4-(pyridin-2-yl)thiophene-2-carboxylate 18C (4.5 g, 19.22 mmol) and ethyl cyanoformate (3.33 g, 33.64 mmol, 3.3 mL, 1.75 eq) were mixed in dioxane*HCl (8 M solution, 75 mL) and refluxed overnight. Then to the reaction mixture dioxane*HCl (8 M solution) 50 mL was added and refluxed for 8 hours. The resulted mixture was cooled to room temperature. The precipitate formed was filtered and washed with CH₃CN (50 mL), MTBE (100 mL) and dried on open air to give ethyl 4-hydroxy-7-(pyridin-2-yl)thieno[3,2-d]pyrimidine-2-carboxylate 18D (5.25 g, 83.5% purity, 14.55 mmol, 75.7% yield) as brown solid.

### Step D: 4-Hydroxy-7-(pyridin-2-yl)thieno[3,2-d]pyrimidine-2-carboxamide (18)

General procedure: Ethyl 4-hydroxy-7-(pyridin-2-yl)thieno[3,2-d]pyrimidine-2-carboxylate 18D (0.3 g (995.62 μmol) was suspended in 5 mL of appropriate corresponding amine and the resulted mixture was stirred for 24 h at room temperature. After that, the reaction mixture was concentrated and purified by HPLC. Using 25% NH₄OH as the amine produced the Example compound (18), 4-hydroxy-7-(pyridin-2-yl)thieno[3,2-d]pyrimidine-2-carboxamide (0.0267 g, 9.9% yield).

### Example 19

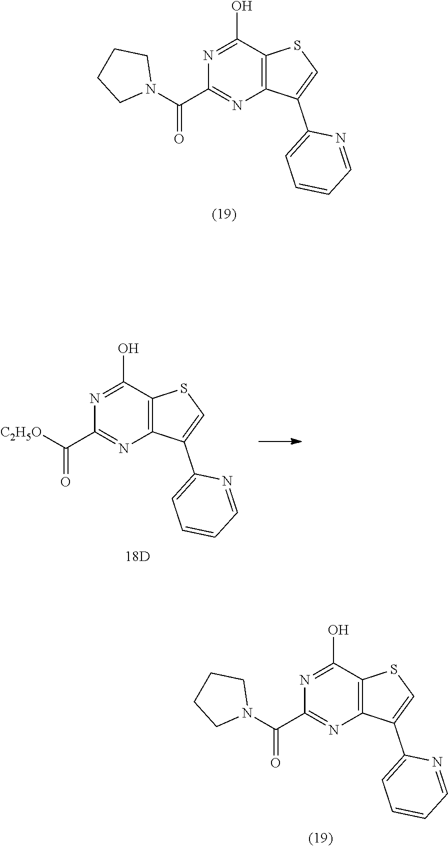

### (4-Hydroxy-7-(pyridin-2-yl)thieno[3,2-d]pyrimidin-2-yl)(pyrrolidin-1-yl)methanone (19)

Using the general procedure described in Example 18, Step D, ethyl 4-hydroxy-7-(pyridin-2-yl)thieno[3,2-d]pyrimidine-2-carboxylate 18D (0.3 g, 995.62 μmol) and pyrrolidine produced the Example compound (19), (4-hydroxy-7-(pyridin-2-yl)thieno[3,2-d]pyrimidin-2-yl)(pyrrolidin-1-yl)methanone (0.0131 g, 4% yield).

### Example 20

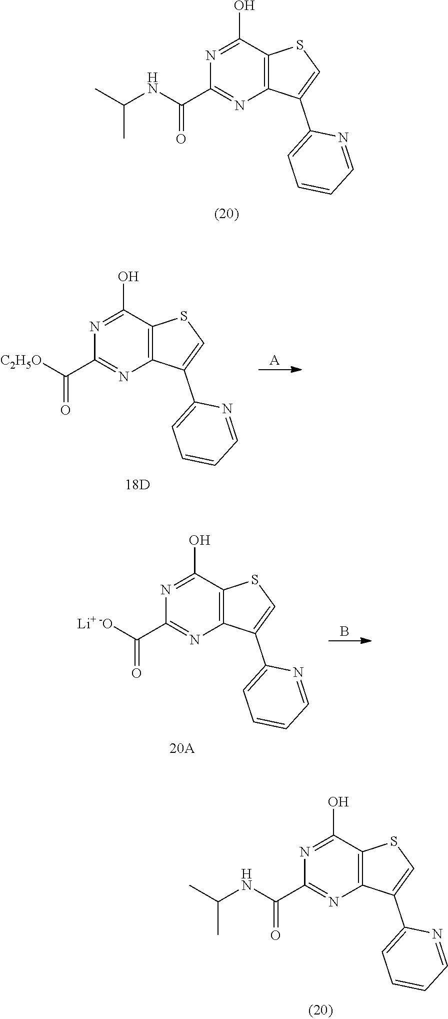

### 4-Hydroxy-N-isopropyl-7-(pyridin-2-yl)thieno[3,2-d]pyrimidine-2-carboxamide (20)

### Step A: 4-Hydroxy-7-(pyridin-2-yl)thieno[3,2-d]pyrimidine-2-carboxylic acid 20A (Li⁺ Salt)

To a solution of ethyl 4-hydroxy-7-(pyridin-2-yl)thieno[3,2-d]pyrimidine-2-carboxylate 18D (10.5 g, 34.85 mmol) in THE (100 mL) and H₂O (20 mL) LiOH×H₂O (3.66 g, 87.11 mmol) was added in one portion. The resulted mixture was stirred for 16 h at room temperature. The precipitate of desired product was filtered, washed by H₂O (2×3 mL) and by THE (3 mL) and dried in open air. 4-Hydroxy-7-(pyridin-2-yl)thieno[3,2-d]pyrimidine-2-carboxylic acid 20A (Li⁺ salt) (6.6 g, 24.15 mmol, 74.4% yield) was obtained as white powder.

### Step B: 4-Hydroxy-N-isopropyl-7-(pyridin-2-yl)thieno[3,2-d]pyrimidine-2-carboxamide (20)

General procedure: Hydroxy-7-(pyridin-2-yl)thieno[3,2-d]pyrimidine-2-carboxylic acid (Li⁺ salt) 20A (400.04 mg, 1.46 mmol) was dissolved in 6 M Dioxane*HCl (10 mL/g) and stirred for 15 min and evaporated. The resulted residue was dissolved in DMF (20 mL/g) then HATU (834.94 mg, 2.2 mmol) and 6 eq of DIPEA (8.78 mmol, 1.53 mL, 6.0 equiv) were added subsequently. This mixture was stirred for 30 min at room temperature then the appropriate corresponding amine (1.5 eq) was added and the reaction mixture was stirred 16 h at room temperature. The final product was purified by NPLC without pre-workup. Using isopropyl amine produced the Example compound (20), 4-hydroxy-N-isopropyl-7-(pyridin-2-yl)thieno[3,2-d]pyrimidine-2-carboxamide (0.0375 g, 6.50 yield).

### Example 21-Example 23

Following the general procedure described in Example 20, Step B, hydroxy-7-(pyridin-2-yl)thieno[3,2-d]pyrimidine-2-carboxylic acid (Li⁺ salt) 20A (400.04 mg, 1.46 mmol) and the appropriate corresponding amines (1.5 eq) produced the Example compounds (21)-(23) shown in Table B.

TABLE B

| | | | Amount, |
|---|---|---|---|
| Ex. | Structure | Name | Yield |
| 21 | 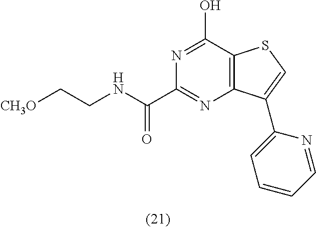 | 4-Hydroxy-N-(2- methoxyethyl)-7-(pyridin-2- yl)thieno[3,2-d]pyrimidine- 2-carboxamide | 0.0575 g, 15.8% |
| 22 | 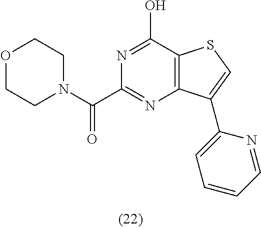 [SMILES: O=C(c1nc(O)c2scc(-c3ccccn3)c2n1)N1CCOCC1] | (4-Hydroxy-7-(pyridin-2- yl)thieno[3,2-d]pyrimidin-2- yl)(morpholino)methanone | 0.0456 g, 9.2% |
| 23 | 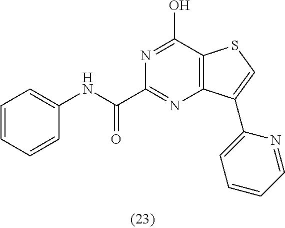 [SMILES: O=C(Nc1ccccc1)c1nc(O)c2scc(-c3ccccn3)c2n1] | 4-Hydroxy-N-phenyl-7- (pyridin-2-yl)thieno[3,2- d]pyrimidine-2- carboxamide | 0.0113 g, 2.2% |

### Example 24

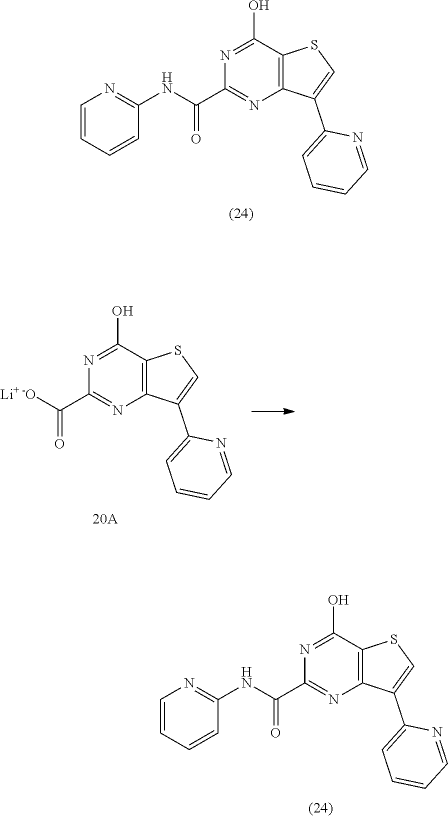

### 4-Hydroxy-N,7-di(pyridin-2-yl)thieno[3,2-d]pyrimidine-2-carboxamide (24)

To a solution of 4-hydroxy-7-(pyridin-2-yl)thieno[3,2-d]pyrimidine-2-carboxylic acid (Li⁺ salt) 20A (297.23 mg, 1.09 mmol) and TCFH (457.77 mg, 1.63 mmol) in DMF, 1-methyl-1H-imidazole (267.8 mg, 3.26 mmol, 260.0 μL, 3.0 equiv) was added in one portion and the mixture was stirred 30 min at room temperature, then to the stirred mixture pyridin-2-amine (204.73 mg, 2.18 mmol) was added. After that, the reaction mixture was stirred for 16 h at room temperature and the final product was purified by HPLC without pre-workup. The Example compound (24), 4-hydroxy-N,7-di(pyridin-2-yl)thieno[3,2-d]pyrimidine-2-carboxamide (6.4 mg, 18.32 μmol, 1.7% yield) was obtained.

### Example 25

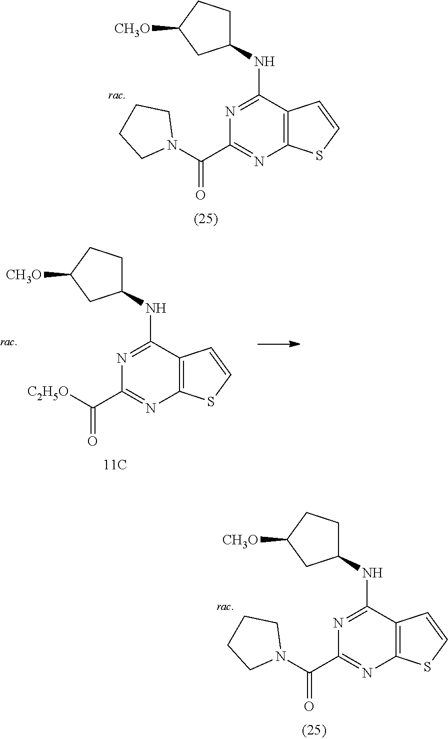

### Rac-(4-(((1R,3S)-3-Methoxycyclopentyl)amino)thieno[2,3-d]pyrimidin-2-yl)(pyrrolidin-1-yl)methanone (25)

Following the general procedure described in Example 11, Step D using rac-ethyl 4-(((1R,3S)-3-methoxycyclopentyl)amino)thieno[2,3-d]pyrimidine-2-carboxylate 11C (1 eq) (0.2 g, 0.622 mmol) and pyrrolidine as the amine produced the Example compound (25), rac-(4-(((1R,3S)-3-methoxycyclopentyl)amino)thieno[2,3-d]pyrimidin-2-yl)(pyrrolidin-1-yl)methanone (0.0297 g, 14.8% yield).

### Example 26

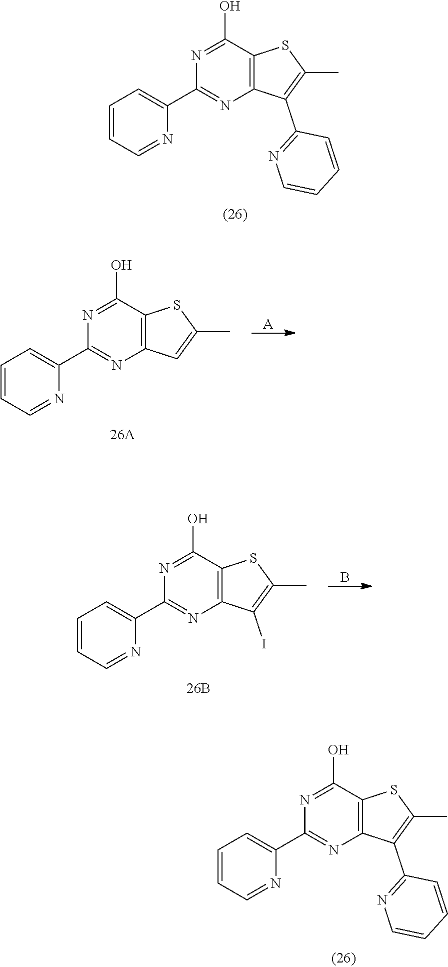

### 6-Methyl-2,7-di(pyridin-2-yl)thieno[3,2-d]pyrimidin-4-ol (26)

### Step A: 7-Iodo-6-methyl-2-(pyridin-2-yl)thieno[3,2-d]pyrimidin-4-ol 26B

A solution of 6-methyl-2-(pyridin-2-yl)thieno[3,2-d]pyrimidin-4-ol 26A (500.17 mg, 2.06 mmol) (for preparation see Hadari, Y. et al., WO 2018237084, Scheme 10) and N-bromosuccinimide (384.21 mg, 2.16 mmol) in acetonitrile (10 mL) was heated at reflux for 12 h. The solvent was evaporated under vacuum. The residue was diluted with water and extracted with EtOAc (6 mL). The organic extract was dried over Na₂SO₄, and evaporated in vacuo to afford the 7-iodo-6-methyl-2-(pyridin-2-yl)thieno[3,2-d]pyrimidin-4-ol 26B (400.0 mg, 90.0% purity, 975.13 μmol, 47.4% yield).

### Step B: 6-Methyl-2,7-di(pyridin-2-yl)thieno[3,2-d]pyrimidin-4-ol (26)

To a stirred solution of 7-iodo-6-methyl-2-(pyridin-2-yl)thieno[3,2-d]pyrimidin-4-ol 26B (383.74 mg, 1.04 mmol) in toluene (6 mL) were added copper(I) iodide (237.55 mg, 1.25 mmol), 2-(tributylstannyl)pyridine (420.69 mg, 1.14 mmol, 370.0 μL, 1.1 equiv) under Argon atmosphere and tetrakis(triphenylphosphine)palladium(0) (60.27 mg, 51.97 μmol) was added under inert atmosphere. The mixture was heated at 100° C. for 16 h, concentrated under reduced pressure and purified by HPLC to afford the Example compound (26), 6-methyl-2,7-di(pyridin-2-yl)thieno[3,2-d]pyrimidin-4-ol (8.8 mg, 27.47 μmol, 2.6% yield).

### Example 27

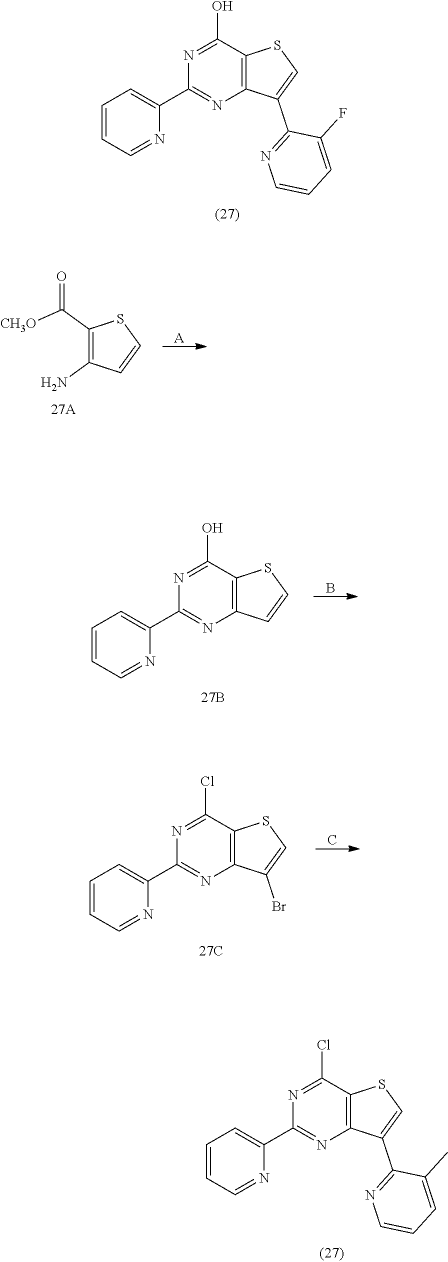

### 7-(3-Fluoropyridin-2-yl)-2-(pyridin-2-yl)thieno[3,2-d]pyrimidin-4-ol (27)

### Step A: 2-(Pyridin-2-yl)thieno[3,2-d]pyrimidin-4-ol 27B

Methyl 3-aminothiophene-2-carboxylate (10.0 g, 63.62 mmol) was dissolved in) Dioxane*HCl (500 mL) and pyridine-2-carbonitrile (9.93 g, 95.43 mmol) was added. The resulting mixture was stirred under reflux for 12 h. Then, the mixture was evaporated under reduced pressure. The residue was diluted with acetonitrile (100 mL) and filtered. The obtained solid was dried at 50° C. to afford 2-(Pyridin-2-yl)thieno[3,2-d]pyrimidin-4-ol 27B (9.0 g, 39.26 mmol, 61.7% yield).

### Step B: 7-Bromo-4-chloro-2-(pyridin-2-yl)thieno[3,2-d]pyrimidine 27C

To a solution 2-(pyridin-2-yl)thieno[3,2-d]pyrimidin-4-ol 27B (9 g, 0.0393 mol) in 90 mL of acetic acid, 10.0 mL of bromine was added at room temperature. The reaction mixture was stirred under reflux for 48 h. Then, the resulting mixture was allowed to cool to room temperature and the volatiles were evaporated under reduced pressure. The residue was diluted with water (100 mL). The resulting precipitate was filtered, washed with water (100 mL*2) and dried to give 7-bromo-4-chloro-2-(pyridin-2-yl)thieno[3,2-d]pyrimidine 27C (7.3 g, mol, 65% yield).

### Step C: 7-(3-Fluoropyridin-2-yl)-2-(pyridin-2-yl)thieno[3,2-d]pyrimidin-4-ol (27)

7-Bromo-4-chloro-2-(pyridin-2-yl)thieno[3,2-d]pyrimidine 27C (1.2 g, 3.89 mmol), 3-fluoro-2-(tributylstannyl)pyridine (1.8 g, 4.67 mmol), cesium fluoride (887.31 mg, 5.84 mmol) and copper(I) iodide (74.16 mg, 389.42 μmol) were mixed together in DMF (10 mL). The resulting mixture was degassed and Pd(PPh₃)₄ (225.78 mg, 194.71 μmol) was added. The reaction mixture was heated at 100° C. overnight. The mixture was extracted with ethyl acetate (25 mL)/Brine (15 mL). The organic phase was washed with water (20 mL), dried over MgSO₄ and concentrated in vacuo. The crude material was purified via HPLC (2-10 min; 55-65% water+FA-acetonitrile+FA; Flow rate 30 mL/min; loading pump 4 mL/min acetonitrile; column SunFire 19*100 mm) to provide 7-(3-fluoropyridin-2-yl)-2-(pyridin-2-yl)thieno[3,2-d]pyrimidin-4-ol.

### Examples 28-29

The following compounds are offered to illustrate but not to limit the disclosure, and may be for use in the compositions and methods provided herein:

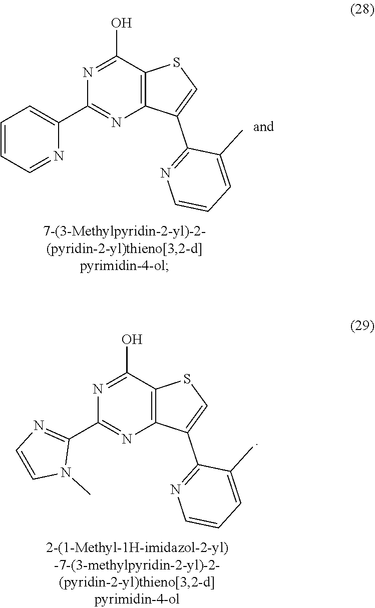 [SMILES: Cc1cccnc1-c1csc2c(O)nc(-c3ccccn3)nc12 ; Cc1cccnc1-c1csc2c(O)nc(-c3nccn3C)nc12]

### Example 30

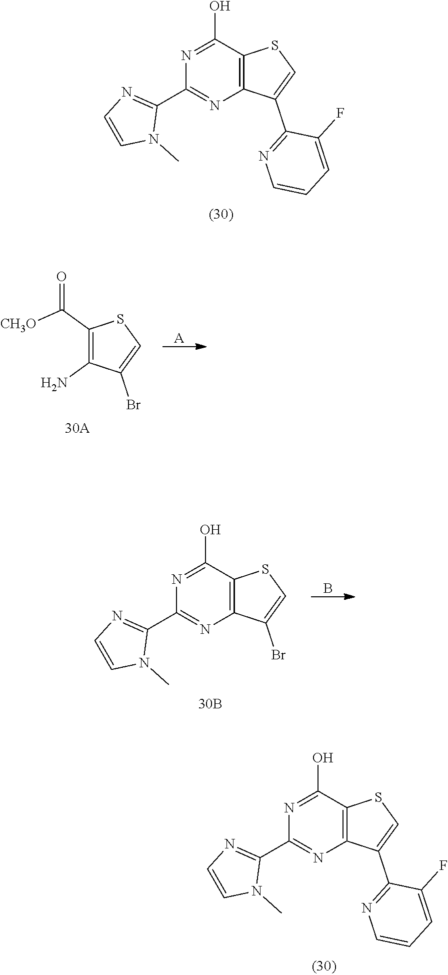

### 7-(3-Fluoropyridin-2-yl)-2-(1-methyl-1H-imidazol-2-yl)thieno[3,2-d]pyrimidin-4-ol (30)

### Step A: 7-Bromo-2-(1-methyl-1H-imidazol-2-yl)thieno[3,2-d]pyrimidin-4-ol 30B

Sodium hydride (201.6 mg, 8.4 mmol) was dissolved in DMF (5 mL) and methyl 3-amino-4-bromothiophene-2-carboxylate 30A (991.67 mg, 4.2 mmol) was added. The resulting mixture was stirred at room temperature for 2 h following by the addition of 1-methyl-1H-imidazole-2-carbonitrile (450.0 mg, 4.2 mmol, 450.0 μl, 1.0 equiv). The reaction mixture was heated at 80° C. for 16 h. Then, the mixture was evaporated under reduced pressure, diluted with water (10 mL) and extracted with EtOAc (15 mL*3). The organic layer dried over Na₂SO₄ and evaporated to give 7-bromo-2-(1-methyl-1H-imidazol-2-yl)thieno[3,2-d]pyrimidin-4-ol 30B (300.0 mg, 964.14 μmol, 23% yield).

### Step B: 7-(3-Fluoropyridin-2-yl)-2-(1-methyl-1H-imidazol-2-yl)thieno[3,2-d]pyrimidin-4-ol (30)

7-Bromo-2-(1-methyl-1H-imidazol-2-yl)thieno[3,2-d]pyrimidin-4-ol 30B (350.0 mg, 1.12 mmol), 2-methyl-6-(tributylstannyl)pyridine (472.61 mg, 1.24 mmol) and copper(I) iodide (21.41 mg, 112.42 μmol) were mixed together in DMF (5 mL). The mixture was degassed and Pd(PPh₃)₄ (130.36 mg, 112.42 μmol) was added. The reaction mixture was heated at 100° C. overnight. The mixture was extracted with ethyl acetate (25 mL)/brine (15 mL). The organic phase was washed with water (20 mL), dried over MgSO₄ and concentrated in vacuo. The crude material was purified via HPLC (2-10 min; 10-50% MeCN/Water+FA; Flow rate 30 mL/min (loading pump 4 mL MeCN); column: SunFire 100*19 mm, 5 microM) to provide 7-(3-fluoropyridin-2-yl)-2-(1-methyl-1H-imidazol-2-yl)thieno[3,2-d]pyrimidin-4-ol (30) (3.0 mg, 91.4% purity, 8.71 μmol, 0.8% yield).

### Example 31

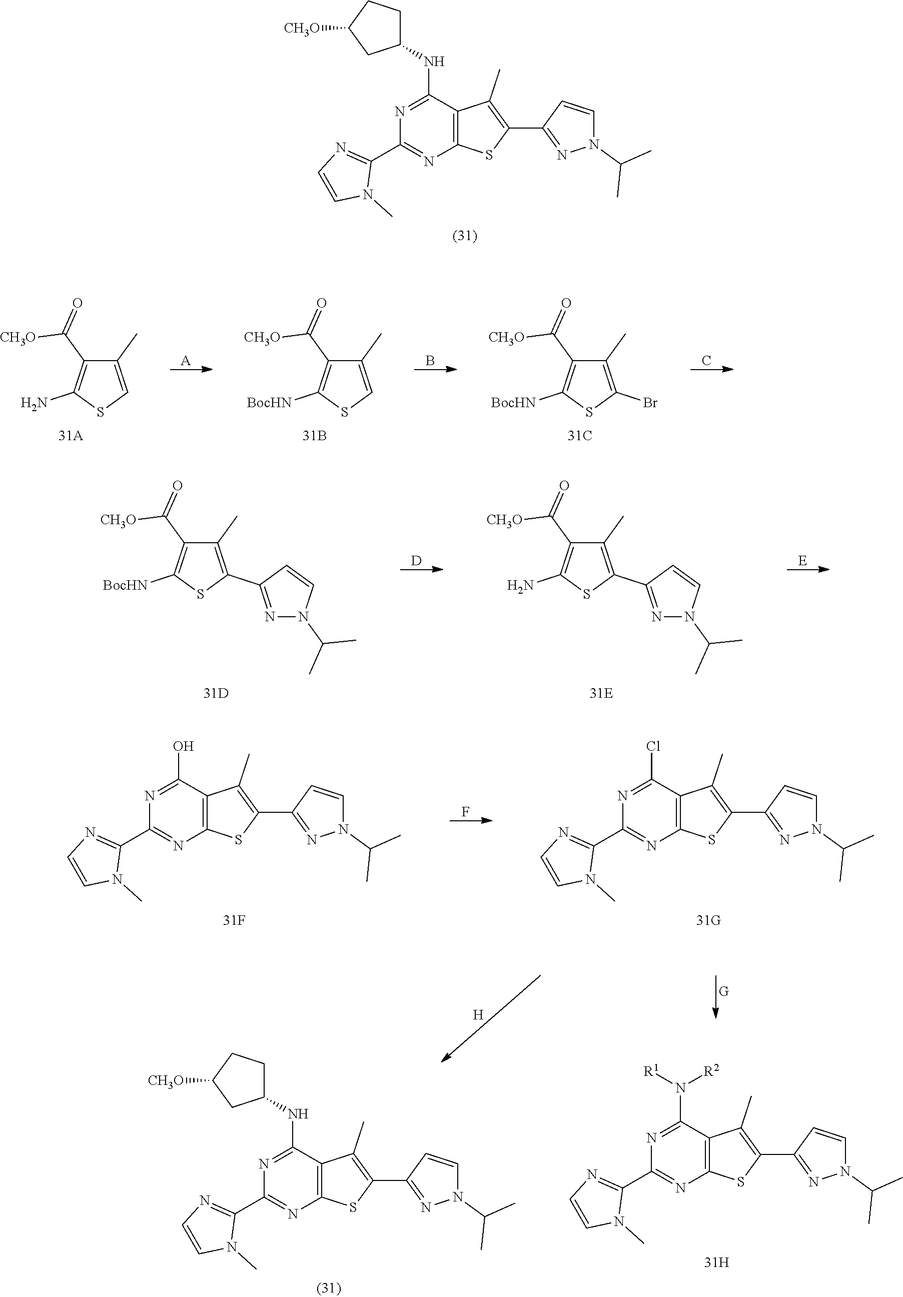

### 6-(1-Isopropyl-1H-pyrazol-3-yl)-N-((1S,3R)-3-methoxycyclopentyl)-5-methyl-2-(1-methyl-1H-imidazol-2-yl)thieno[2,3-d]pyrimidin-4-amine (31)

### Step A: Methyl 2-[(tert-butoxy)carbonyl]amino-4-methylthiophene-3-carboxylate 31B

DMAP (642.19 mg, 5.26 mmol) was added to a stirred solution of methyl 2-amino-4-methylthiophene-3-carboxylate 31A (9.0 g, 52.56 mmol) and Boc₂O (13.77 g, 63.08 mmol) in 108 mL of dioxane. The mixture was refluxed overnight. After 16 h reaction was complete (monitored by NMR). The mixture was concentrated under reduced pressure and purified by column chromatography (Eluted by Hex:EtOAc 10:1). Methyl 2-[(tert-butoxy)carbonyl]amino-4-methylthiophene-3-carboxylate 31B (8.1 g, 29.85 mmol, 56.8% yield) was obtained as white solid.

### Step B: Methyl 5-bromo-2-[(tert-butoxy)carbonyl]amino-4-methylthiophene-3-carboxylate 31C

To a solution of methyl 2-[(tert-butoxy)carbonyl]amino-4-methylthiophene-3-carboxylate 31B (8.1 g, 29.85 mmol) in 160 mL of DMF, N-bromosuccinimide (6.38 g, 35.82 mmol) was added in one portion. The resulting mixture was stirred at room temperature overnight. Then, the mixture was allowed to cool to room temperature and poured in ice water (350 mL). The obtained precipitate was filtered, washed 3 times with water and dried on air. Methyl 5-bromo-2-[(tert-butoxy)carbonyl]amino-4-methylthiophene-3-carboxylate 31C (9.2 g, 26.27 mmol, 88% yield) was obtained as light-yellow powder.

### Step C: Methyl 2-((tert-butoxycarbonyl)amino)-5-(1-isopropyl-1H-pyrazol-3-yl)-4-methylthiophene-3-carboxylate 31D

Methyl 5-bromo-2-[(tert-butoxy)carbonyl]amino-4-methylthiophene-3-carboxylate 31C (2.0 g, 5.71 mmol), 1-(propan-2-yl)-3-(tetramethyl-1,3,2-dioxaborolan-2-yl)-1H-pyrazole (1.62 g, 6.85 mmol), cesium carbonate (3.72 g, 11.42 mmol) and (2-dicyclohexylphosphino-2′,4′,6′-triisopropyl-1,1′-biphenyl)[2-(2′-amino-1,1′-biphenyl)]palladium(II) methanesulfonate (966.74 mg, 1.14 mmol) were mixed together in degassed dioxane:H₂O (40 mL:2 mL) and refluxed overnight. Then, the mixture was allowed to cool to room temperature, filtered and concentrated under reduced pressure. The residue was purified by column chromatography (SiO₂; Elute by Hexane:EtOAc 10:1 to 1:10). Methyl 2-((tert-butoxycarbonyl)amino)-5-(1-isopropyl-1H-pyrazol-3-yl)-4-methylthiophene-3-carboxylate 31D (1.2 g, 90.0% purity, 2.85 mmol, 49.8% yield) was obtained as orange powder.

### Step D: Methyl 2-amino-5-(1-isopropyl-1H-pyrazol-3-yl)-4-methylthiophene-3-carboxylate 31E

Methyl 2-((tert-butoxycarbonyl)amino)-5-(1-isopropyl-1H-pyrazol-3-yl)-4-methylthiophene-3-carboxylate 31D (1.2 g, 3.16 mmol) was dissolved in 5 mL of dioxane and 6M dioxane*HCl was added dropwise at 0° C. The reaction mixture was stirred overnight at room temperature. Then, the volatiles were removed in vacuo and the residue was purified by flash chromatography (SiO₂; Eluted by EtOAc). Methyl 2-amino-5-(1-isopropyl-1H-pyrazol-3-yl)-4-methylthiophene-3-carboxylate 31E (530.0 mg, 90.0% purity, 1.71 mmol, 57.3% yield) was obtained as dark-grey gum.

### Step E: 6-(1-Isopropyl-1H-pyrazol-3-yl)-5-methyl-2-(1-methyl-1H-imidazol-2-yl)thieno[2,3-d]pyrimidin-4-ol 31F

Methyl 2-amino-5-(1-isopropyl-1H-pyrazol-3-yl)-4-methylthiophene-3-carboxylate 31E (430.35 mg, 1.54 mmol), 1-methyl-1H-imidazole-2-carbonitrile (247.51 mg, 2.31 mmol) and potassium 2-methylpropan-2-olate (691.45 mg, 6.16 mmol) were dissolved in dry MeOH (5 mL). The reaction mixture was stirred overnight at room temperature. The residue was evaporated under reduced pressure and HOAc (conc) was added to pH 5. Then, the solution was mixed with EtOAc (15 mL) and extracted. The organic phase was dried over Na₂SO₄ and concentrated under reduced pressure to afford 6-(1-isopropyl-1H-pyrazol-3-yl)-5-methyl-2-(1-methyl-1H-imidazol-2-yl)thieno[2,3-d]pyrimidin-4-ol 31F (400.0 mg, 80.0% purity, 902.86 μmol, 58.6% yield).

### Step F: 4-Chloro-6-(1-isopropyl-1H-pyrazol-3-yl)-5-methyl-2-(1-methyl-1H-imidazol-2-yl)thieno[2,3-d]pyrimidine 31G

The crude material from the previous step (6-(1-isopropyl-1H-pyrazol-3-yl)-5-methyl-2-(1-methyl-1H-imidazol-2-yl)thieno[2,3-d]pyrimidin-4-ol 31F) (100.0 mg, 282.14 μmol)) was suspended in POCl₃ (2 mL) and diisopropylethylamine (0.6 mL) was added at room temperature. The reaction mixture was stirred under reflux for 16 h. The mixture was allowed to cool to the room temperature and evaporated under reduced pressure. The residue was poured into ice and basified with liquid ammonia (20 mL, 20-25% of ammonia) and filtered on to afford 4-chloro-6-(1-isopropyl-1H-pyrazol-3-yl)-5-methyl-2-(1-methyl-1H-imidazol-2-yl)thieno[2,3-d]pyrimidine 31G (100.0 mg, 55.0% purity, 147.5 μmol, 52.4% yield) as brown liquid.

### Step G: General Procedure: 4-Chloro-6-(1-isopropyl-1H-pyrazol-3-yl)-5-methyl-2-(1-methyl-1H-imidazol-2-yl)thieno[2,3-d]pyrimidine 31G

(100.0 mg, 268.19 μmol, 1.0 equiv), corresponding amine (1.5 equiv) and ethylbis(propan-2-yl)amine (4 equiv) were mixed in DMSO (2 mL). The resulting mixture was stirred at 100° C. for 12 hours and then allowed to cool to room temperature. The mixture was diluted with water (10 mL) and extracted several times with EtOAc (3*15 mL). The combined extracts were washed with brine (10 mL), dried over anhydrous sodium sulfate, and filtered. The filtrate was evaporated. The residue was subjected for HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-8 min 50-75% MeOH-water; flow 30 mL/min (loading pump 4 mL/min MeOH)).

### Step H: 6-(1-Isopropyl-1H-pyrazol-3-yl)-N-((1S,3R)-3-methoxycyclopentyl)-5-methyl-2-(1-methyl-1H-imidazol-2-yl)thieno[2,3-d]pyrimidin-4-amine (31)

4-Chloro-6-(1-isopropyl-1H-pyrazol-3-yl)-5-methyl-2-(1-methyl-1H-imidazol-2-yl)thieno[2,3-d]pyrimidine 31G (1.88 g, 4.33 mmol) was dissolved in DMSO (18 mL) and ethylbis(propan-2-yl)amine (2.24 g, 17.34 mmol, 3.02 ml, 4.0 equiv) was added. The resulting mixture was stirred for 5 min at room temperature following by the addition of (1S,3R)-3-methoxycyclopentan-1-amine 4F (prepared as described in Example 4, Step C) (499.12 mg, 4.33 mmol). The reaction mixture was stirred at 100° C. overnight. Then, the mixture was allowed to cool to the room temperature and subjected to HPLC (2-10 min; 65-80% water-MeOH; Flow rate 30 mL/min; loading pump 4 mL/min methanol; column: SunFire 100*19 mm, 5 microM) purification to afford 6-(1-isopropyl-1H-pyrazol-3-yl)-N-((1S,3R)-3-methoxycyclopentyl)-5-methyl-2-(1-methyl-1H-imidazol-2-yl)thieno[2,3-d]pyrimidin-4-amine (31). (390.0 mg, 95.0% purity, 721.3 μmol, 16.6% yield) as yellow gum.

### Example 32

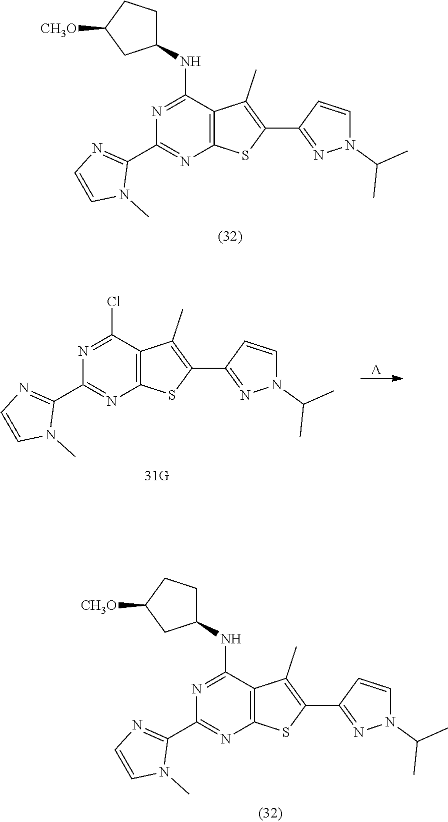

### 6-(1-Isopropyl-1H-pyrazol-3-yl)-N-((1R,3S)-3-methoxycyclopentyl)-5-methyl-2-(1-methyl-1H-imidazol-2-yl)thieno[2,3-d]pyrimidin-4-amine (32)

4-Chloro-6-(1-isopropyl-1H-pyrazol-3-yl)-5-methyl-2-(1-methyl-1H-imidazol-2-yl)thieno[2,3-d]pyrimidine 31G (428.54 mg, 1.15 mmol) was dissolved in DMSO (18 mL) and ethylbis(propan-2-yl)amine (593.6 mg, 4.59 mmol, 800.0 μl, 4.0 equiv) was added. The resulting mixture was stirred for 5 min at room temperature following by the addition of (1R,3S)-3-methoxycyclopentan-1-amine 4G (prepared as described in Example 4, Step C) (145.6 mg, 1.26 mmol). The reaction mixture was stirred at 100° C. overnight. Then, the mixture was allowed to cool to the room temperature and subjected to HPLC (2-8 min; 0-75% water-MeOH; Flow rate 30 mL/min; loading pump 4 mL/min MeOH; column SunFire 19*100 mm) to afford 6-(1-isopropyl-1H-pyrazol-3-yl)-N-((1R,3S)-3-methoxycyclopentyl)-5-methyl-2-(1-methyl-1H-imidazol-2-yl)thieno[2,3-d]pyrimidin-4-amine (32) (83.0 mg, 95.0% purity, 174.61 μmol, 15.2% yield) as yellow gum.

### Examples 33-63

Following the general procedure described in Example 31, Step G, 4-chloro-6-(1-isopropyl-1H-pyrazol-3-yl)-5-methyl-2-(1-methyl-1H-imidazol-2-yl)thieno[2,3-d]pyrimidine 31G (100.0 mg, 268.19 μmol, 1.0 equiv) was treated with the appropriate corresponding amine (1.5 eq) to produce the Example compounds (33)-(63) shown in Table C.

TABLE C

| | | | Amount |
|---|---|---|---|
| Example | Structure | Name | (Yield) |
| 33 | 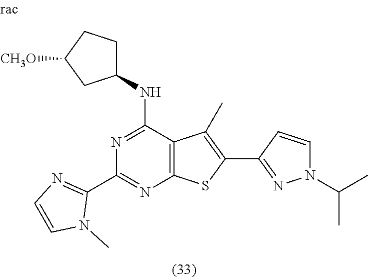 | rac-6-(1-Isopropyl- 1H-pyrazol-3-yl)-N- ((1R,3R)-3- methoxycyclopentyl)- 5-methyl-2-(1- methyl-1H-imidazol- 2-yl)thieno[2,3- d]pyrimidin-4-amine | 62.0 mg, (16.2%) |
| 34 | 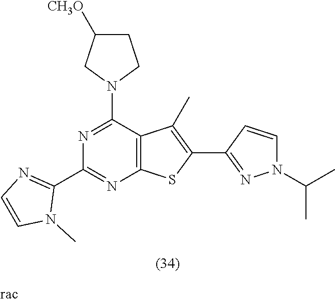 | rac-6-(1-Isopropyl- 1H-pyrazol-3-yl)-4- (3- methoxypyrrolidin-1- yl)-5-methyl-2-(1- methyl-1H-imidazol- 2-yl)thieno[2,3- d]pyrimidine | 58.0 mg (15.7%) |
| 35 | 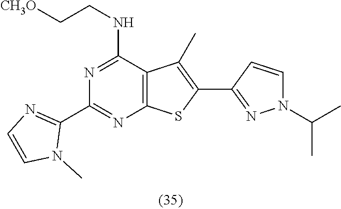 | 6-(1-Isopropyl-1H- pyrazol-3-yl)-N-(2- methoxyethyl)-5- methyl-2-(1-methyl- 1H-imidazol-2- yl)thieno[2,3- d]pyrimidin-4-amine | 160.0 mg (17.8%) |
| 36 | 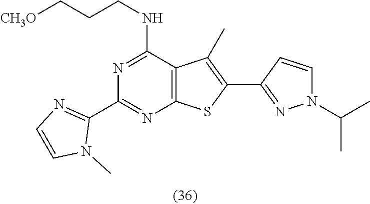 | 6-(1-Isopropyl-1H- pyrazol-3-yl)-N-(3- methoxypropyl)-5- methyl-2-(1-methyl- 1H-imidazol-2- yl)thieno[2,3- d]pyrimidin-4-amine | 150.0 mg (14.5%) |
| 37 | 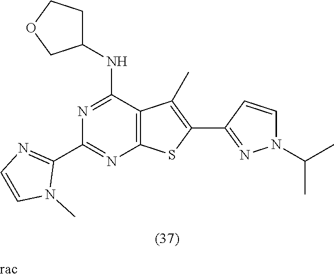 [SMILES: Cc1c(-c2ccn(C(C)C)n2)sc2nc(-c3nccn3C)nc(NC3CCOC3)c12] | rac-6-(1-Isopropyl- 1H-pyrazol-3-yl)-5- methyl-2-(1-methyl- 1H-imidazol-2-yl)-N- (tetrahydrofuran-3- yl)thieno[2,3- d]pyrimidin-4-amine | 62.7 mg (18.4%) |
| 38 | 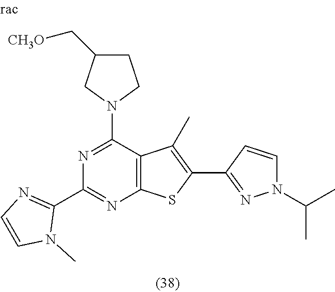 | rac-6-(1-Isopropyl- 1H-pyrazol-3-yl)-4- (3- (methoxymethyl) pyrrolidin-1-yl)-5- methyl-2-(1-methyl- 1H-imidazol-2- yl)thieno[2,3- d]pyrimidine | 65.3 mg (17.1%) |
| 39 | 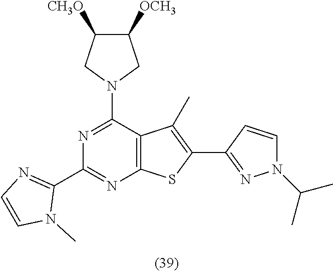 | rac-4-((3S,4R)-3,4- Dimethoxypyrrolidin- 1-yl)-6-(1-isopropyl- 1H-pyrazol-3-yl)-5- methyl-2-(1-methyl- 1H-imidazol-2- yl)thieno[2,3- d]pyrimidine | 14.6 mg (11.7%) |
| 40 | 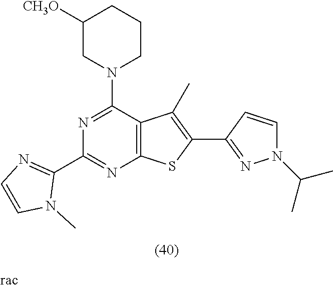 | rac-6-(1-Isopropyl- 1H-pyrazol-3-yl)-4- (3-methoxypiperidin- 1-yl)-5-methyl-2-(1- methyl-1H-imidazol- 2-yl)thieno[2,3- d]pyrimidine | 19.6 mg (14.7%) |
| 41 | 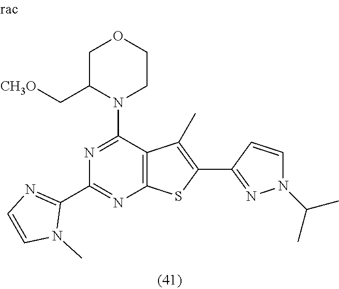 | rac-4-(6-(1- Isopropyl-1H- pyrazol-3-yl)-5- methyl-2-(1-methyl- 1H-imidazol-2- yl)thieno[2,3- d]pyrimidin-4-yl)-3- (methoxymethyl) morpholine | 15.8 mg (12.8%) |
| 42 | 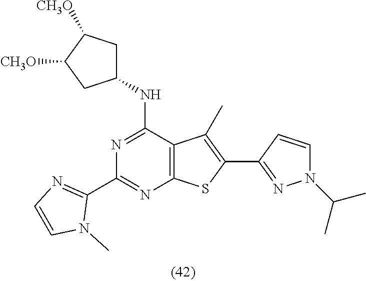 | N-((1r,3R,4S)-3,4- Dimethoxycyclopentyl)- 6-(1-isopropyl- 1H-pyrazol-3-yl)-5- methyl-2-(1-methyl- 1H-imidazol-2- yl)thieno[2,3- d]pyrimidin-4-amine | 19.4 mg (16.3%) |
| 43 | 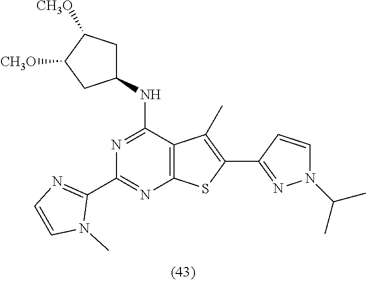 | N-((1s,3R,4S)-3,4- Dimethoxycyclopentyl)- 6-(1-isopropyl- 1H-pyrazol-3-yl)-5- methyl-2-(1-methyl- 1H-imidazol-2- yl)thieno[2,3- d]pyrimidin-4-amine | 9.0 mg (8.1%) |
| 44 | 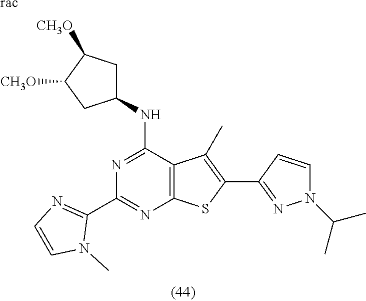 | rac-N-((3S,4S)-3,4- Dimethoxycyclopentyl)- 6-(1-isopropyl- 1H-pyrazol-3-yl)-5- methyl-2-(1-methyl- 1H-imidazol-2- yl)thieno[2,3- d]pyrimidin-4-amine | 15.4 mg (12.2%) |
| 45 | 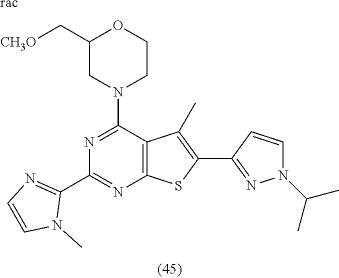 | rac-4-(6-(1- Isopropyl-1H- pyrazol-3-yl)-5- methyl-2-(1-methyl- 1H-imidazol-2- yl)thieno[2,3- d]pyrimidin-4-yl)-2- (methoxymethyl) morpholine | 36.2 mg (28.8%) |
| 46 | 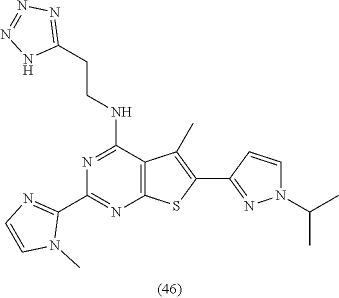 [SMILES: Cc1c(-c2ccn(C(C)C)n2)sc2nc(-c3nccn3C)nc(NCCc3nnn[nH]3)c12] | N-(2-(1H-Tetrazol-5- yl)ethyl)-6-(1- isopropyl-1H- pyrazol-3-yl)-5- methyl-2-(1-methyl- 1H-imidazol-2- yl)thieno[2,3- d]pyrimidin-4-amine | 10.3 mg (8.2%) |
| 47 | 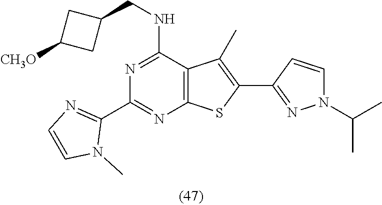 | 6-(1-Isopropyl-1H- pyrazol-3-yl)-N- (((1s,3s)-3- methoxycyclobutyl) methyl)-5-methyl-2- (1-methyl-1H- imidazol-2- yl)thieno[2,3- d]pyrimidin-4-amine | 21.7 mg (16.6%) |
| 48 | 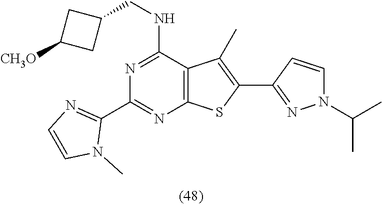 | 6-(1-Isopropyl-1H- pyrazol-3-yl)-N- (((1r,3r)-3- methoxycyclobutyl) methyl)-5-methyl-2- (1-methyl-1H- imidazol-2- yl)thieno[2,3- d]pyrimidin-4-amine | 24.8 mg (19.8%) |
| 49 | 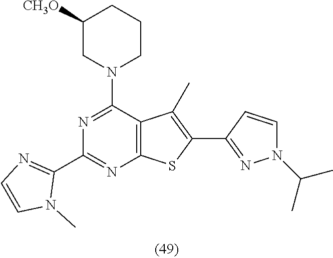 | (S)-6-(1-Isopropyl- 1H-pyrazol-3-yl)-4- (3-methoxypiperidin- 1-yl)-5-methyl-2-(1- methyl-1H-imidazol- 2-yl)thieno[2,3- d]pyrimidine | 16.3 mg (13.8%) |
| 50 | 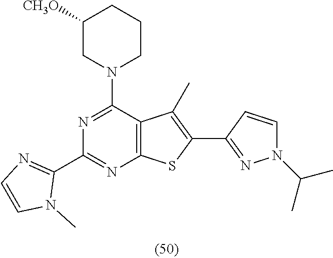 | (R)-6-(1-Isopropyl- 1H-pyrazol-3-yl)-4- (3-methoxypiperidin- 1-yl)-5-methyl-2-(1- methyl-1H-imidazol- 2-yl)thieno[2,3- d]pyrimidine | 18.6 mg (14.3%) |
| 51 | 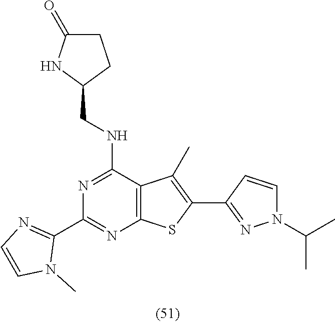 [SMILES: Cc1c(-c2ccn(C(C)C)n2)sc2nc(-c3nccn3C)nc(NC[C@@H]3CCC(=O)N3)c12] | (S)-5-((6-(1- Isopropyl-1H- pyrazol-3-yl)-5- methyl-2-(1-methyl- 1H-imidazol-2- yl)thieno[2,3- d]pyrimidin-4- yl)amino)methyl) pyrrolidin-2-one | 18.0 mg (14.4%) |
| 52 | 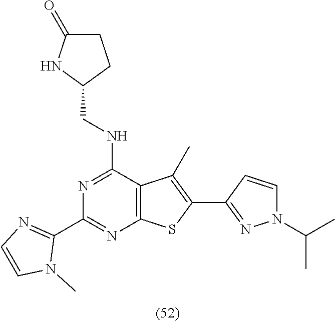 [SMILES: Cc1c(-c2ccn(C(C)C)n2)sc2nc(-c3nccn3C)nc(NC[C@H]3CCC(=O)N3)c12] | (R)-5-(((6-(1- Isopropyl-1H- pyrazol-3-yl)-5- methyl-2-(1-methyl- 1H-imidazol-2- yl)thieno[2,3- d]pyrimidin-4- yl)amino)methyl) pyrrolidin-2-one | 14.4 mg (11.2%) |
| 53 | 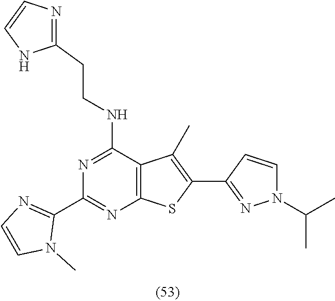 [SMILES: Cc1c(-c2ccn(C(C)C)n2)sc2nc(-c3nccn3C)nc(NCCc3ncc[nH]3)c12] | N-(2-(1H-Imidazol-2- yl)ethyl)-6-(1- isopropyl-1H- pyrazol-3-yl)-5- methyl-2-(1-methyl- 1H-imidazol-2- yl)thieno[2,3- d]pyrimidin-4-amine | 11.9 mg (9.5%) |
| 54 | 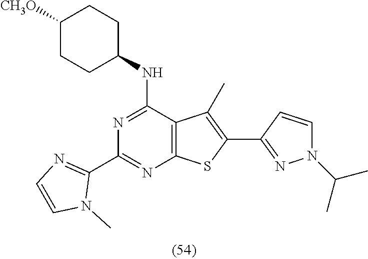 | 6-(1-isopropyl-1H- pyrazol-3-yl)-5- methyl-2-(1-methyl- 1H-imidazol-2-yl)-4- (((1r,4r)-4-(oxo-λ⁶- methyl)cyclohexyl) amino)thieno[2,3- d]pyrimidine | 13.4 mg (10.2%) |
| 55 | 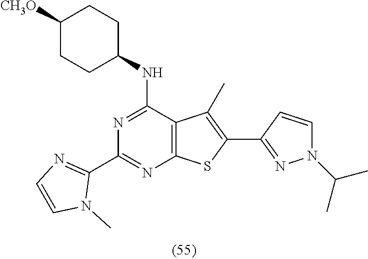 | 6-(1-isopropyl-1H- pyrazol-3-yl)-5- methyl-2-(1-methyl- 1H-imidazol-2-yl)-4- (((1s,4s)-4-(oxo-λ⁶- methyl)cyclohexyl) amino)thieno[2,3- d]pyrimidine | 7.7 mg (9.2%) |
| 56 | 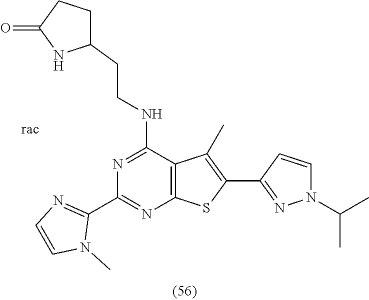 [SMILES: Cc1c(-c2ccn(C(C)C)n2)sc2nc(-c3nccn3C)nc(NCCC3CCC(=O)N3)c12 ; "rac"] | rac-5-(2-((6-(1- Isopropyl-1H- pyrazol-3-yl)-5- methyl-2-(1-methyl- 1H-imidazol-2- yl)thieno[2,3- d]pyrimidin-4- yl)amino)ethyl) pyrrolidin-2-one | 5.7 mg (4.6%) |
| 57 | 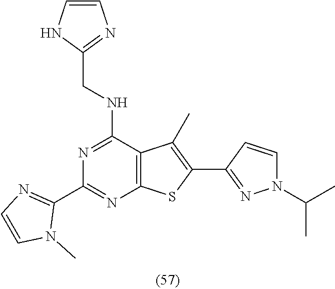 [SMILES: Cc1c(-c2ccn(C(C)C)n2)sc2nc(-c3nccn3C)nc(NCc3ncc[nH]3)c12] | N-((1H-Imidazol-2- yl)methyl)-6-(1- isopropyl-1H- pyrazol-3-yl)-5- methyl-2-(1-methyl- 1H-imidazol-2- yl)thieno[2,3- d]pyrimidin-4-amine | 17.4 mg (13.8%) |
| 58 | 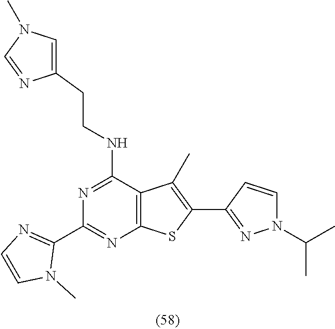 [SMILES: Cc1c(-c2ccn(C(C)C)n2)sc2nc(-c3nccn3C)nc(NCCc3cn(C)cn3)c12] | 6-(1-Isopropyl-1H- pyrazol-3-yl)-5- methyl-2-(1-methyl- 1H-imidazol-2-yl)-N- (2-(1-methyl-1H- imidazol-4- yl)ethyl)thieno[2,3- d]pyrimidin-4-amine | 18.7 mg (14.5%) |
| 59 | 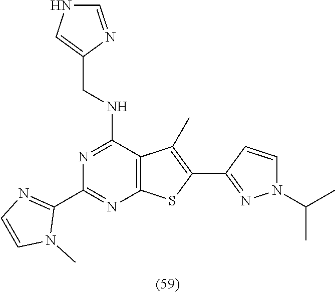 [SMILES: Cc1c(-c2ccn(C(C)C)n2)sc2nc(-c3nccn3C)nc(NCc3c[nH]cn3)c12] | N-((1H-Imidazol-4- yl)methyl)-6-(1- isopropyl-1H- pyrazol-3-yl)-5- methyl-2-(1-methyl- 1H-imidazol-2- yl)thieno[2,3- d]pyrimidin-4-amine | 3.3 mg (2.6%) |
| 60 | 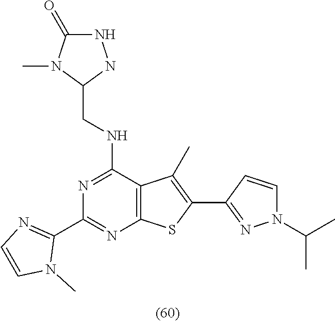 [SMILES: Cc1c(-c2ccn(C(C)C)n2)sc2nc(-c3nccn3C)nc(NCC3NNC(=O)N3C)c12] | 5-(((6-(1-Isopropyl- 1H-pyrazol-3-yl)-5- methyl-2-(1-methyl- 1H-imidazol-2- yl)thieno[2,3- d]pyrimidin-4- yl)amino)methyl)-4- methyl-2,4-dihydro- 3H-1,2,4-triazol-3- one | 11.2 mg (9.0%) |
| 61 | 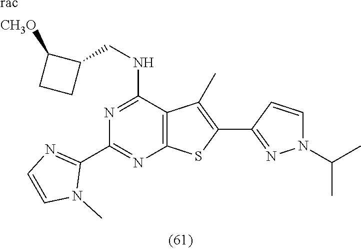 | rac-6-(1-Isopropyl- 1H-pyrazol-3-yl)-N- (((1S,2R)-2- methoxycyclobutyl) methyl)-5-methyl-2- (1-methyl-1H- imidazol-2- yl)thieno[2,3- d]pyrimidin-4-amine | 5.3 mg (4.2%) |
| 62 | 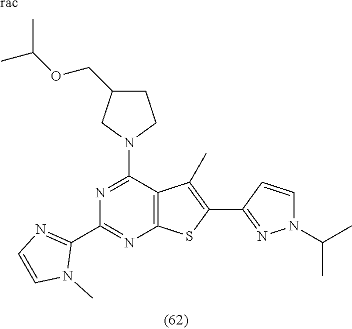 | rac-4-(3- (Isopropoxymethyl) pyrrolidin-1-yl)-6-(1- isopropyl-1H- pyrazol-3-yl)-5- methyl-2-(1-methyl- 1H-imidazol-2- yl)thieno[2,3- d]pyrimidine | 9.3 mg (7.2%) |
| 63 | 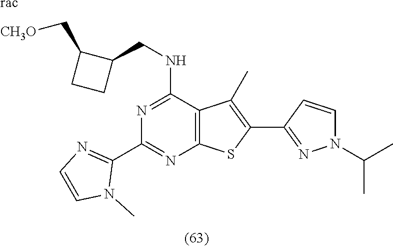 | rac-6-(1-Isopropyl- 1H-pyrazol-3-y)-N- (((1S,2R)-2- (methoxymethyl)cyclo- butyl)methyl)-5- methyl-2-(1-methyl- 1H-imidazol-2- yl)thieno[2,3- d]pyrimidin-4-amine | 20.1 mg (7.5%) |

### Example 64

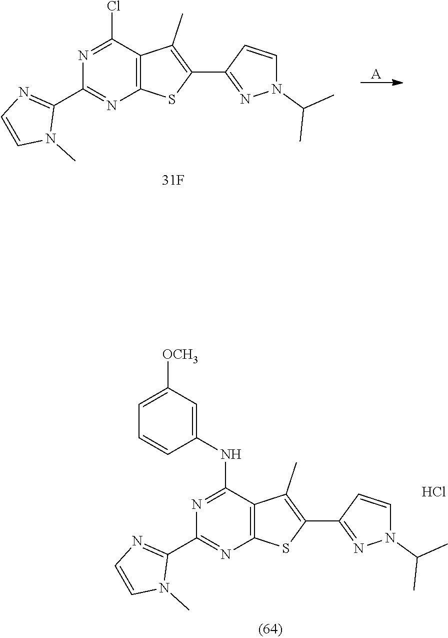

### 6-(1-Isopropyl-1H-pyrazol-3-yl)-N-(3-methoxyphenyl)-5-methyl-2-(1-methyl-1H-imidazol-2-yl)thieno[2,3-d]pyrimidin-4-amine (64)

General Procedure: To a solution of 4-chloro-6-(1-isopropyl-1H-pyrazol-3-yl)-5-methyl-2-(1-methyl-1H-imidazol-2-yl)thieno[2,3-d]pyrimidine 31F (prepared as described in Example 31, Step E) (100.0 mg, 268.19 μmol, 1 equiv) and the appropriate corresponding amine (1.5 equiv) in dry DMF (1 mL) sodium hydride (13.42 mg, 559.11 μmol, 1.5 eq) was added under an Ar atmosphere at 0° C. The reaction mixture was stirred at 50° C. for 18 h. Then, the mixture was allowed to cool to room temperature, diluted with water (2 mL) and concentrated to dryness under reduced pressure. The residue was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-8 min 50-75% MeOH-water; flow 30 mL/min (loading pump 4 mL/min MeOH)) to afford pure product. Using 3-methoxyaniline as the corresponding amine produced 6-(1-isopropyl-1H-pyrazol-3-yl)-N-(3-methoxyphenyl)-5-methyl-2-(1-methyl-1H-imidazol-2-yl)thieno[2,3-d]pyrimidin-4-amine (64) (17.2 mg, 16.7% yield).

### Examples 65-68

Following the general procedure described in Example 65, Step A, 4-chloro-6-(1-isopropyl-1H-pyrazol-3-yl)-5-methyl-2-(1-methyl-pH-imidazol-2-yl)thieno[2,3-d]pyrimidine 31F (100.0 mg, 268.19 μmol, 1.0 equiv) (100.0 mg, 268.19 μmol, 1 equiv) and the appropriate corresponding amine (1.5 equiv), produced the Example compounds (65)-(68) shown in Table D.

TABLE D

| | | | Amount |
|---|---|---|---|
| Example | Structure | Name | (Yield) |
| 65 | 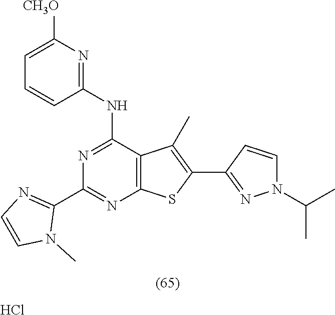 | 6-(1-Isopropyl-1H- pyrazol-3-yl)-N-(6- methoxypyridin-2- yl)-5-methyl-2-(1- methyl-1H-imidazol- 2-yl)thieno[2,3- d]pyrimidin-4-amine hydrochloride salt | 6.6 mg (6.4%) |
| 66 | 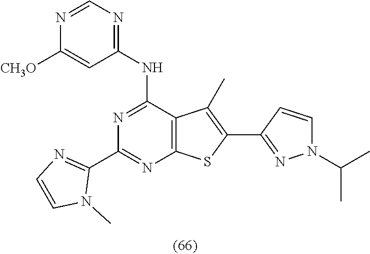 | 6-(1-Isopropyl-1H- pyrazol-3-yl)-N-(6- methoxypyrimidin-4- yl)-5-methyl-2-(1- methyl-1H-imidazol- 2-yl)thieno[2,3- d]pyrimidin-4-amine | 29.0 mg (7.8%) |
| 67 | 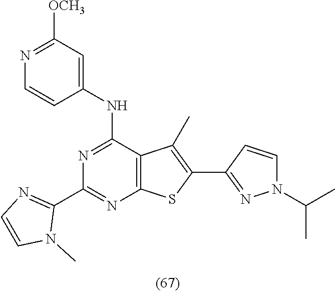 [SMILES: COc1cc(Nc2nc(-c3nccn3C)nc3sc(-c4ccn(C(C)C)n4)c(C)c23)ccn1] | 6-(1-Isopropyl-1H- pyrazol-3-yl)-N-(2- methoxypyridin-4- yl)-5-methyl-2-(1- methyl-1H-imidazol- 2-yl)thieno[2,3- d]pyrimidin-4-amine | 30.5 mg (23.1%) |
| 68 | 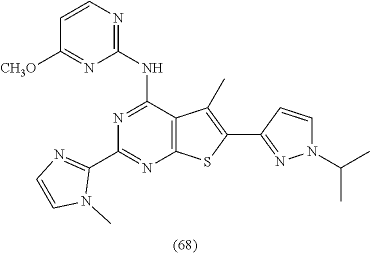 | 6-(1-Isopropyl-1H- pyrazol-3-yl)-N-(4- methoxypyrimidin-2- yl)-5-methyl-2-(1- methyl-1H-imidazol- 2-yl)thieno[2,3- d]pyrimidin-4-amine | 32.6 mg (15.3%) |

### Example 69

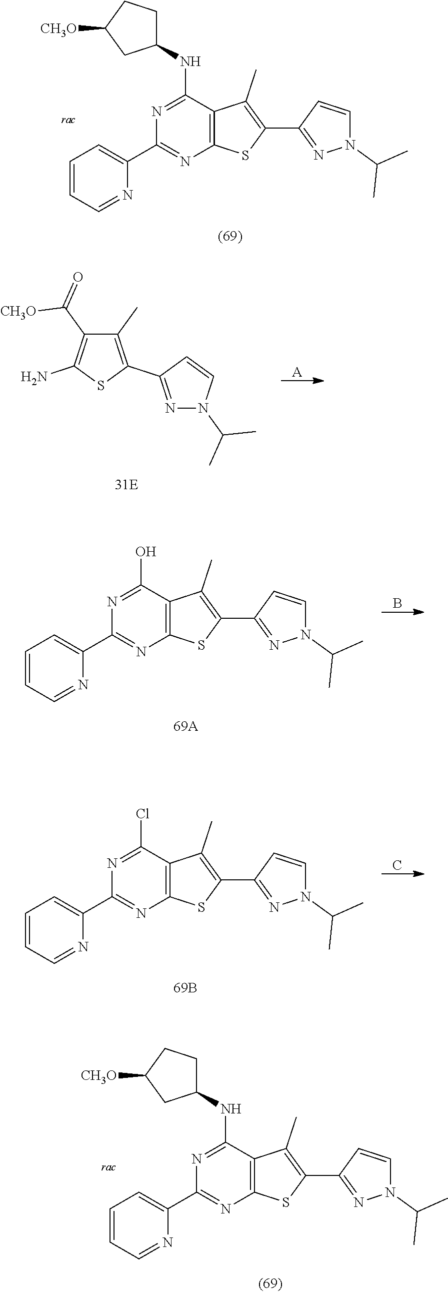

### Rac-6-(1-isopropyl-1H-pyrazol-3-yl)-N-((1R,3S)-3-methoxycyclopentyl)-5-methyl-2-(pyridine-2-yl)thieno[2,3-d]pyrimidin-4-amine (69)

### Step A: 6-(1-Isopropyl-1H-pyrazol-3-yl)-5-methyl-2-(pyridin-2-yl)thieno[2,3-d]pyrimidin-4-ol 69A

Pyridine-2-carbonitrile (532.8 mg, 5.12 mmol) was dissolved in DMF (10 mL) and sodium hydride (558.24 mg, 23.26 mmol) was added. The resulting mixture was stirred at room temperature for 2 h following by the addition methyl 2-amino-5-(1-isopropyl-1H-pyrazol-3-yl)-4-methylthiophene-3-carboxylate 31E (prepared as described in Example 31, Step D) (1.3 g, 4.65 mmol). The reaction mixture was stirred at 80° C. for 16 h. Then the volatiles were removed in vacuo. The residue was diluted with water (10.0 mL) and extracted with EtOAc (15.0 mL*3). The organic layer was concentrated under reduced pressure to afford 6-(1-isopropyl-1H-pyrazol-3-yl)-5-methyl-2-(pyridin-2-yl)thieno[2,3-d]pyrimidin-4-ol 69A (1.0 g, 2.85 mmol, 61.2% yield).

### Step B: 4-Chloro-6-(1-isopropyl-1H-pyrazol-3-yl)-5-methyl-2-(pyridin-2-yl)thieno[2,3-d]pyrimidine 69B

6-(1-Isopropyl-1H-pyrazol-3-yl)-5-methyl-2-(pyridin-2-yl)thieno[2,3-d]pyrimidin-4-ol 69A (1.0 g, 2.85 mmol) was suspended in phosphoroyl trichloride (3.5 g, 22.82 mmol, 2.13 mL, 8.0 equiv) and ethylbis(propan-2-yl)amine (1.11 g, 8.55 mmol, 1.49 mL, 3.0 equiv) was added at room temperature. The reaction mixture was stirred under reflux for 16 h. Then, the resulting mixture was allowed to cool to the room temperature and concentrated under reduced pressure. The residue was poured in ice and diluted with ice-cold ammonia (20 mL, 20-25% of ammonia). The product was extracted with chloroform (50.0 mL*2) and evaporated in vacuo to afford 4-Chloro-6-(1-isopropyl-1H-pyrazol-3-yl)-5-methyl-2-(pyridin-2-yl)thieno[2,3-d]pyrimidine 69B (800.0 mg, 2.16 mmol, 75.8% yield) as brown slurry.

### Step C: rac-6-(1-Isopropyl-1H-pyrazol-3-yl)-N-((1R,3S)-3-methoxycyclopentyl)-5-methyl-2-(pyridin-2-yl)thieno[2,3-d]pyrimidin-4-amine (69)

rac trans-3-Methoxycyclopentan-1-amine (60.64 mg, 526.51 μmol), ethylbis(propan-2-yl)amine (185.5 mg, 1.44 mmol, 250.0 μl, 3.0 equiv) and 4-chloro-6-(1-isopropyl-1H-pyrazol-3-yl)-5-methyl-2-(pyridin-2-yl)thieno[2,3-d]pyrimidine 69B (178.0 mg, 478.65 μmol) were mixed in DMSO (2.0 mL). The resulting reaction mixture was stirred at 100° C. for 15 hours and then allowed to cool to room temperature. The mixture was diluted with water (10 mL) and extracted three times with EtOAc (15 mL). The combined extracts were washed with brine (10 mL), dried over anhydrous sodium sulfate and filtered. The filtrate was concentrated under reduced pressure and the residue was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min, 0-60% MeOH—H₂O flow 30 mL/min (loading pump 4 mL/min MeOH)) to afford rac-6-(1-isopropyl-1H-pyrazol-3-yl)-N-((1R,3S)-3-methoxycyclopentyl)-5-methyl-2-(pyridin-2-yl)thieno[2,3-d]pyrimidin-4-amine (69) (10.0 mg, 95.0% purity, 21.18 μmol, 4.6% yield).

### Example 70

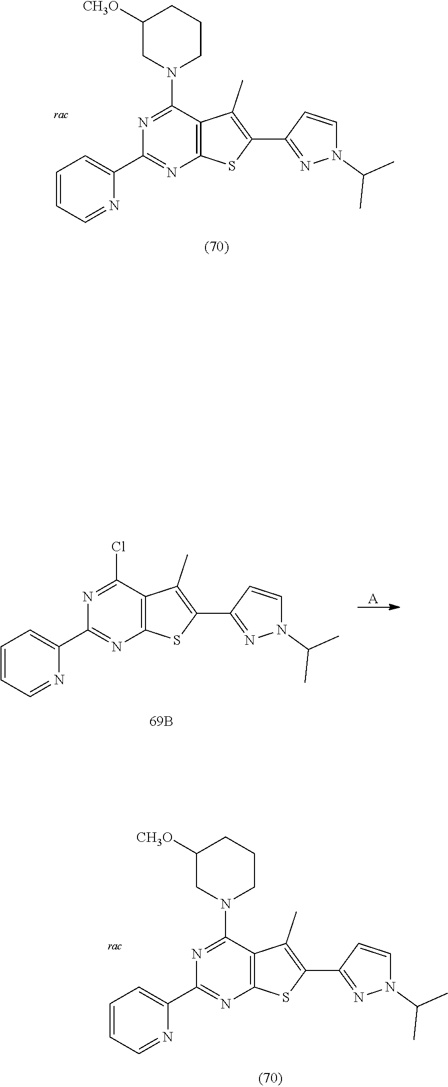

### Rac-6-(1-Isopropyl-1H-pyrazol-3-yl)-4-(3-methoxypiperidin-1-yl)-5-methyl-2-(pyridin-2-yl)thieno[2,3-d]pyrimidine (70)

4-Chloro-6-(1-isopropyl-1H-pyrazol-3-yl)-5-methyl-2-(pyridin-2-yl)thieno[2,3-d]pyrimidine 69B (prepared as described in Example 69, Step C) (249.83 mg, 675.46 μmol), rac 3-methoxypiperidine hydrochloride (153.63 mg, 1.01 mmol) and ethylbis(propan-2-yl)amine (348.74 mg, 2.7 mmol, 470.0 μL, 4.0 equiv) were mixed in DMSO (5 mL). The resulting reaction mixture is stirred at 100° C. for 12 hours and then allowed to cool to room temperature. The mixture is diluted with water (20 ml) and extracted several times with EtOAc (3*25 mL). The combined extracts were washed with brine (25 mL), dried over anhydrous sodium sulfate, and filtered. The filtrate was concentrated under reduced pressure. The residue was subjected for HPLC purification (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 mL/min (loading pump 4 mL/min MeOH)) to afford rac-6-(1-isopropyl-1H-pyrazol-3-yl)-4-(3-methoxypiperidin-1-yl)-5-methyl-2-(pyridin-2-yl)thieno[2,3-d]pyrimidine (70) (85.0 mg, 189.49 μmol, 28.1% yield).

### Example 71

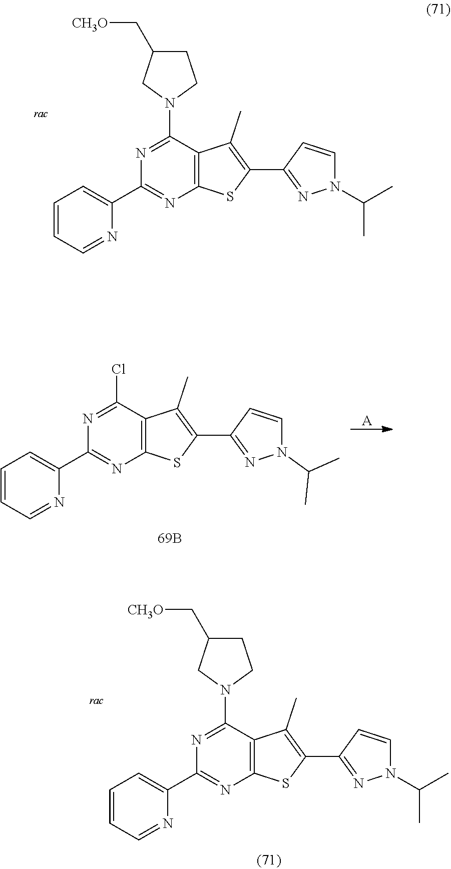

### Rac-6-(1-Isopropyl-1H-pyrazol-3-yl)-4-(3-(methoxymethyl)pyrrolidin-1-yl)-5-methyl-2-(pyridin-2-yl)thieno[2,3-d]pyrimidine (71)

4-Chloro-6-(1-isopropyl-1H-pyrazol-3-yl)-5-methyl-2-(pyridin-2-yl)thieno[2,3-d]pyrimidine 69B (prepared as described in Example 69, Step B) (148.42 mg, 401.26 μmol), rac 3-(methoxymethyl)pyrrolidine (69.32 mg, 601.89 μmol) and ethylbis(propan-2-yl)amine (155.82 mg, 1.21 mmol, 210.0 μL, 3.0 equiv) were mixed in DMSO (5 mL). The resulting reaction mixture is stirred at 100° C. for 12 hours and then allowed to cool to room temperature. The mixture is diluted with water (20 mL) and extracted several times with EtOAc (3*25 mL). The combined extracts were washed with brine (25 mL), dried over anhydrous sodium sulfate, and filtered. The filtrate was concentrated under reduced pressure. The residue was subjected for HPLC purification (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-80% MeCN-water; flow 30 mL/min (loading pump 4 mL/min MeCN)) to afford rac-6-(1-isopropyl-1H-pyrazol-3-yl)-4-(3-(methoxymethyl)pyrrolidin-1-yl)-5-methyl-2-(pyridin-2-yl)thieno[2,3-d]pyrimidine (71) (59.0 mg, 95.0% purity, 124.95 μmol, 31.1% yield).

### Example 72

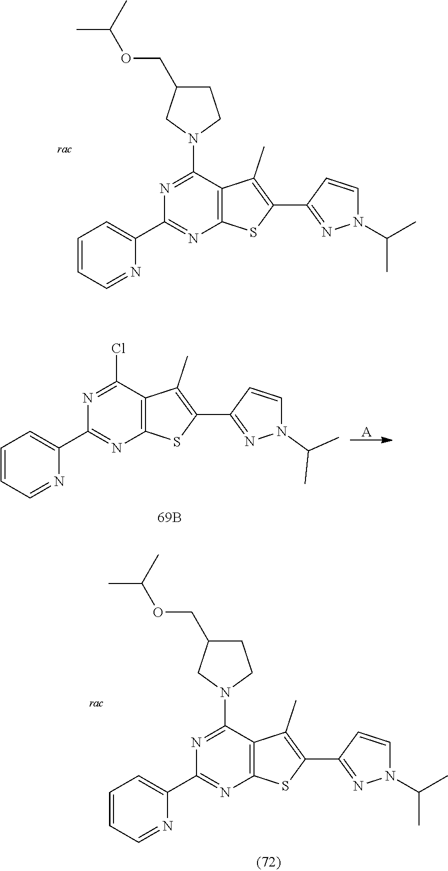

### 4-(3-(Isopropoxymethyl)pyrrolidin-1-yl)-6-(1-isopropyl-1H-pyrazol-3-yl)-5-methyl-2-(pyridin-2-yl)thieno[2,3-d]pyrimidine (72)

4-Chloro-6-(1-isopropyl-1H-pyrazol-3-yl)-5-methyl-2-(pyridin-2-yl)thieno[2,3-d]pyrimidine 69B (prepared as described in Example 69, Step B) (175.38 mg, 474.16 μmol), 3-[(propan-2-yloxy)methyl]pyrrolidine (101.87 mg, 711.23 μmol) and ethylbis(propan-2-yl)amine (244.86 mg, 1.89 mmol, 330.0 μL, 4.0 equiv) were mixed in DMSO (5 mL). The resulting reaction mixture is stirred at 100° C. for 12 hours and then allowed to cool to room temperature. The mixture was diluted with water (20 mL) and extracted several times with EtOAc (3*25 mL). The combined extracts were washed with brine (25 mL), dried over anhydrous sodium sulfate, and filtered. The solvent was removed in vacuo. The residue was subjected for HPLC purification (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 35-65% MeCN-water+TFA; flow 30 mL/min (loading pump 4 mL/min MeCN)) to afford 4-(3-(isopropoxymethyl)pyrrolidin-1-yl)-6-(1-isopropyl-1H-pyrazol-3-yl)-5-methyl-2-(pyridin-2-yl)thieno[2,3-d]pyrimidine (72) (7.6 mg, 95.0% purity, 15.15 μmol, 3.2% yield).

### Example 73

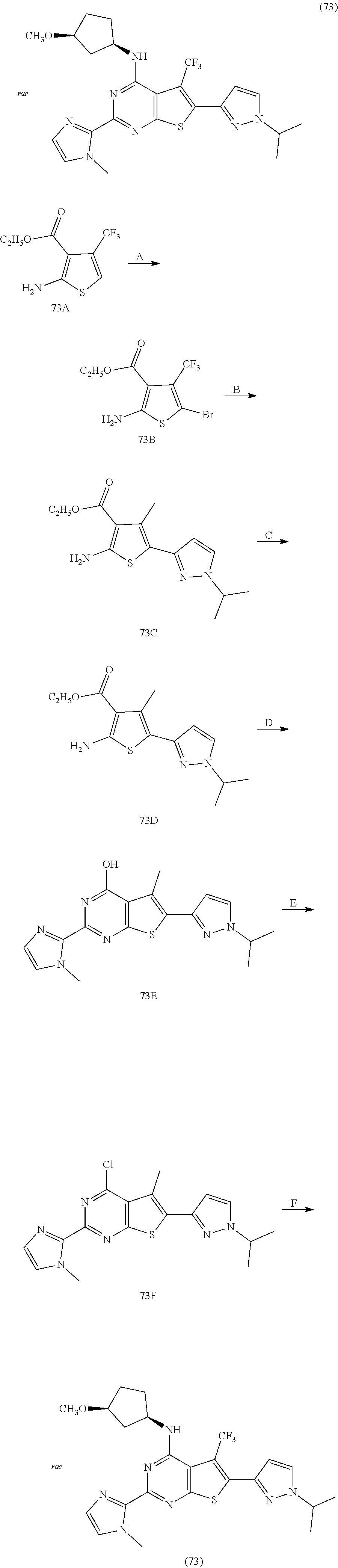

### Rac-6-(1-isopropyl-1H-pyrazol-3-yl)-N-((1R,3S)-3-methoxycyclopentyl)-2-(1-methyl-1H-imidazol-2-yl)-5-(trifluoromethyl)thieno[2,3-d]pyrimidin-4-amine (73)

### Step A: Ethyl 2-amino-5-bromo-4-(trifluoromethyl)thiophene-3-carboxylate 73B

N-Bromosuccinimide (4.46 g, 25.08 mmol) was added portionwise to the solution of ethyl 2-amino-4-(trifluoromethyl)thiophene-3-carboxylate 73A (5.0 g, 20.9 mmol) in DMF (100 mL) at 0° C. The reaction mixture was stirred at room temperature for 16 h, then poured in ice water. The formed precipitate was collected by filtration, washed by water (2*100 mL) and dried in vacuo. Ethyl 2-amino-5-bromo-4-(trifluoromethyl)thiophene-3-carboxylate 73B (6.1 g, 90.0% purity, 17.26 mmol, 82.6% yield) was obtained.

### Step B: Ethyl 2-amino-5-(1-isopropyl-1H-pyrazol-3-yl)-4-(trifluoromethyl)thiophene-3-carboxylate 73C

Ethyl 2-amino-5-bromo-4-(trifluoromethyl)thiophene-3-carboxylate 73B (6.1 g, 19.18 mmol), 1-(propan-2-yl)-3-(tetramethyl-1,3,2-dioxaborolan-2-yl)-1H-pyrazole (5.43 g, 23.01 mmol), cesium carbonate (12.5 g, 38.35 mmol) and Pd(dppf)Cl₂*CH₂Cl₂ (1.57 g, 1.92 mmol) were dissolved in degassed dioxane (80 mL) under Ar atmosphere. 3.8 mL of water was added via syringe. The reaction mixture was heated to 80° C. and stirred at this temperature for 16 h. The mixture was cooled to room temperature, filtered through celite and concentrated under reduced pressure. The residue was purified by column chromatography (SiO₂; Eluted by Hex:EtOAc 20:1 to 1:2). Ethyl 2-amino-5-(1-isopropyl-1H-pyrazol-3-yl)-4-(trifluoromethyl)thiophene-3-carboxylate 73C (2.5 g, 7.2 mmol, 37.5% yield) was obtained as light-yellow powder.

### Step C: 6-(1-Isopropyl-1H-pyrazol-3-yl)-2-(1-methyl-1H-imidazol-2-yl)-5-(trifluoromethyl)thieno[2,3-d]pyrimidin-4-ol 73D

Sodium methanolate (1.17 g, 21.59 mmol) was added portionwise to the solution of ethyl 2-amino-5-(1-isopropyl-1H-pyrazol-3-yl)-4-(trifluoromethyl)thiophene-3-carboxylate 73C (2.5 g, 7.2 mmol) and 1-methyl-1H-imidazole-2-carbonitrile X1 (770.83 mg, 7.2 mmol, 770.0 μl, 1.0 equiv) in MeOH (40 mL). The reaction mixture was heated to reflux and stirred at this temperature for 16 h. Then, the mixture was cooled to room temperature and neutralized by acetic acid (2.16 g, 35.98 mmol). The solution was concentrated, dissolved in 50 mL EtOAc, washed by brine (2*50 mL), dried over sodium sulphate and evaporated under reduced pressure. 6-(1-Isopropyl-1H-pyrazol-3-yl)-2-(1-methyl-1H-imidazol-2-yl)-5-(trifluoromethyl)thieno[2,3-d]pyrimidin-4-ol 73D (1.2 g, 50.0% purity, 1.47 mmol) was obtained and used in next step without further purification.

### Step D: 4-Chloro-6-(1-isopropyl-1H-pyrazol-3-yl)-2-(1-methyl-1H-imidazol-2-yl)-5-(trifluoromethyl)thieno[2,3-d]pyrimidine 73E

Ethyl bis(propan-2-yl)amine (949.23 mg, 7.34 mmol) was added dropwise to the solution of 6-(1-Isopropyl-1H-pyrazol-3-yl)-2-(1-methyl-1H-imidazol-2-yl)-5-(trifluoromethyl)thieno[2,3-d]pyrimidin-4-ol 73D (1.2 g, 2.94 mmol) in phosphoroyl trichloride (9.01 g, 58.76 mmol). The mixture was heated to 100° C. and stirred at this temperature for 16 h. The reaction mixture was concentrated in vacuo, poured in ice water (5 mL) and neutralized by K₂CO₃. The formed precipitate was collected by filtration, washed by water (2*5 mL) and dried in vacuo. 4-Chloro-6-(1-isopropyl-1H-pyrazol-3-yl)-2-(1-methyl-1H-imidazol-2-yl)-5-(trifluoromethyl)thieno[2,3-d]pyrimidine 73E (850.0 mg, 30.0% purity, 597.41 μmol) was obtained. Product was used in next steps without further purification. Step E: rac-6-(1-isopropyl-1H-pyrazol-3-yl)-N-((1R,3S)-3-methoxycyclopentyl)-2-(1-methyl-1H-imidazol-2-yl)-5-(trifluoromethyl)thieno[2,3-d]pyrimidin-4-amine (73). Ethylbis(propan-2-yl)amine (133.96 mg, 1.04 mmol) was added in one portion to the solution of 4-chloro-6-(1-isopropyl-1H-pyrazol-3-yl)-2-(1-methyl-1H-imidazol-2-yl)-5-(trifluoromethyl)thieno[2,3-d]pyrimidine 73E (370.0 mg, 866.82 μmol) and rac-(1S,3R)-3-methoxycyclopentan-1-amine hydrochloride (47.15 mg, 310.94 μmol) in DMF (1 mL). The mixture was heated to 100° C. and stirred at this temperature for 16 h. The mixture was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 mL/min (loading pump 4 mL/min MeOH)) to afford rac-6-(1-isopropyl-1H-pyrazol-3-yl)-N-((1R,3S)-3-methoxycyclopentyl)-2-(1-methyl-1H-imidazol-2-yl)-5-(trifluoromethyl)thieno[2,3-d]pyrimidin-4-amine (73). (43.0 mg, 85.05 μmol, 32.8% yield).

### Example 74

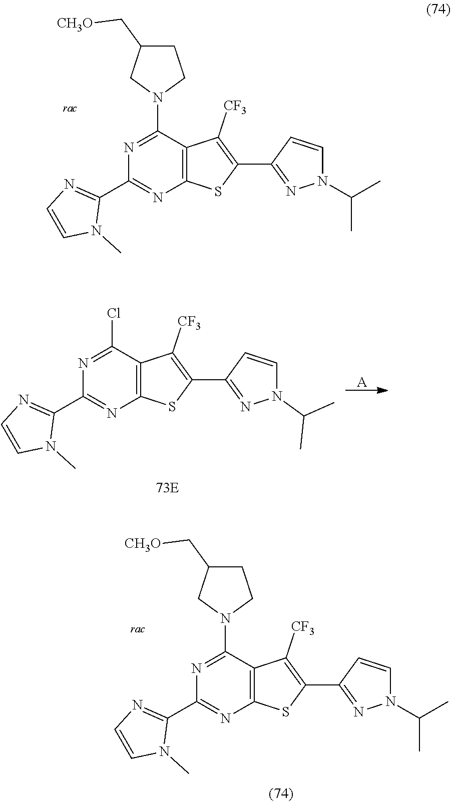

### Rac-6-(1-Isopropyl-1H-pyrazol-3-yl)-4-(3-(methoxymethyl)pyrrolidin-1-yl)-2-(1-methyl-1H-imidazol-2-yl)-5-(trifluoromethyl)thieno[2,3-d]pyrimidine (74)

Ethylbis(propan-2-yl)amine (245.42 mg, 1.9 mmol, 330.0 μl, 3.0 equiv) was added in one portion to the solution of 4-chloro-6-(1-isopropyl-1H-pyrazol-3-yl)-2-(1-methyl-1H-imidazol-2-yl)-5-(trifluoromethyl)thieno[2,3-d]pyrimidine 73E (prepared as described in Example 73, Step D) (900.0 mg, 2.11 mmol) and rac 3-(methoxymethyl)pyrrolidine (87.48 mg, 759.55 μmol) in DMF (1 mL). The resulting mixture was heated to 100° C. and stirred at this temperature for 16 h. The mixture was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 65-80% MeOH-water; flow 30 mL/min (loading pump 4 mL/min MeOH)) to afford rac-6-(1-isopropyl-1H-pyrazol-3-yl)-4-(3-(methoxymethyl)pyrrolidin-1-yl)-2-(1-methyl-1H-imidazol-2-yl)-5-(trifluoromethyl)thieno[2,3-d]pyrimidine (74) (70.0 mg, 138.46 μmol, 21.9% yield).

### Example 75

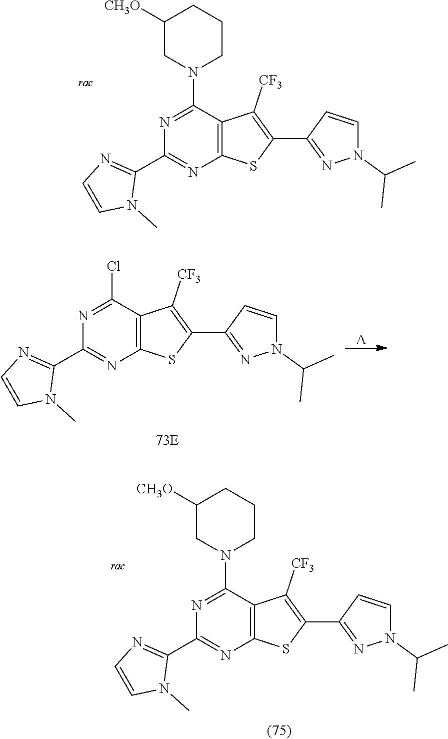

### Rac-6-(1-Isopropyl-1H-pyrazol-3-yl)-4-(3-methoxypiperidin-1-yl)-2-(1-methyl-1H-imidazol-2-yl)-5-(trifluoromethyl)thieno[2,3-d]pyrimidine (75)

Ethylbis(propan-2-yl)amine (83.54 mg, 646.41 μmol, 110.0 μl, 4.0 equiv) was added in one portion to the solution of 4-chloro-6-(1-isopropyl-1H-pyrazol-3-yl)-2-(1-methyl-1H-imidazol-2-yl)-5-(trifluoromethyl)thieno[2,3-d]pyrimidine 73E (prepared as described in Example 73, Step D) (230.0 mg, 538.84 μmol) and rac 3-methoxypiperidine hydrochloride (29.41 mg, 193.92 μmol) in DMF (1 ml). The mixture was heated to 100° C. and stirred at this temperature for 16 h. The obtained mixture was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 ml/min (loading pump 4 ml/min MeOH)) to afford rac-6-(1-isopropyl-1H-pyrazol-3-yl)-4-(3-methoxypiperidin-1-yl)-2-(1-methyl-1H-imidazol-2-yl)-5-(trifluoromethyl)thieno[2,3-d]pyrimidine (75) (60.0 mg, 118.68 μmol, 73.4% yield).

### Example 76

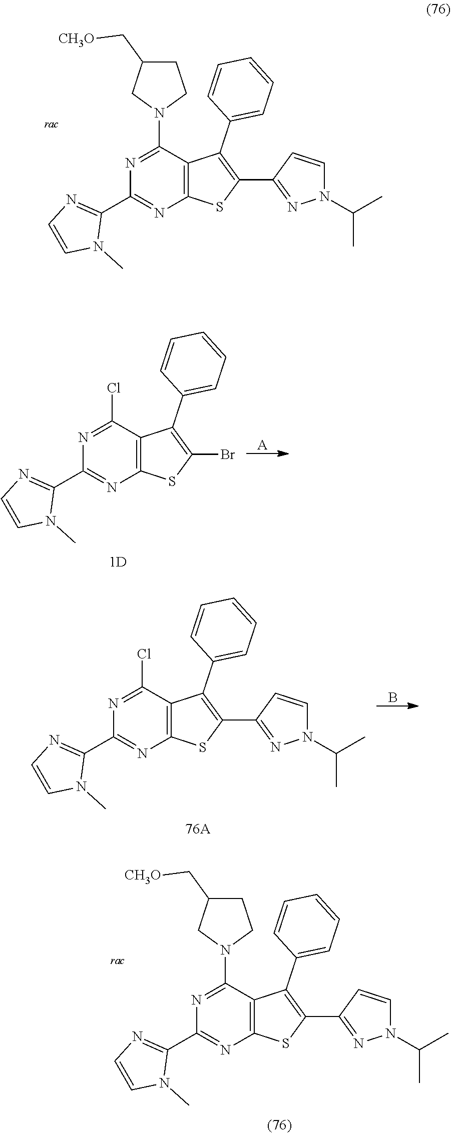

### Rac-6-(1-isopropyl-1H-pyrazol-3-yl)-4-(3-(methoxymethyl)pyrrolidin-1-yl)-2-(1-methyl-1H-imidazol-2-yl)-5-phenylthieno[2,3-d]pyrimidine (76)

### Step A: 4-Chloro-6-(1-isopropyl-1H-pyrazol-3-yl)-2-(1-methyl-1H-imidazol-2-yl)-5-phenylthieno[2,3-d]pyrimidine 76A

6-Bromo-4-chloro-2-(1-methyl-1H-imidazol-2-yl)-5-phenylthieno[2,3-d]pyrimidine 1D (prepared as described in Example 1, Step C) (900.0 mg, 2.22 mmol), 3-(methoxymethyl)pyrrolidine (383.39 mg, 3.33 mmol) and ethylbis(propan-2-yl)amine (860.44 mg, 6.66 mmol, 1.16 ml, 3.0 equiv) were mixed in DMSO (5 mL). The resulting mixture was stirred at 100° C. for 12 hours and then allowed to cool to room temperature. The mixture was diluted with water (20 mL) and extracted several times with EtOAc (3*25 mL). The combined extracts were washed with brine (25 mL), dried over anhydrous sodium sulfate, and filtered. The filtrate was concentrated under reduced pressure. The residue was subjected to HPLC purification (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 mL/min (loading pump 4 mL/min MeOH)) to afford 4-chloro-6-(1-isopropyl-1H-pyrazol-3-yl)-2-(1-methyl-1H-imidazol-2-yl)-5-phenylthieno[2,3-d]pyrimidine 76A (600.0 mg, 91.0% purity, 1.13 mmol, 50.8% yield).

### Step B: rac-6-(1-Isopropyl-1H-pyrazol-3-yl)-4-(3-(methoxymethyl)pyrrolidin-1-yl)-2-(1-methyl-1H-imidazol-2-yl)-5-phenylthieno[2,3-d]pyrimidine (76)

4-Chloro-6-(1-isopropyl-1H-pyrazol-3-yl)-2-(1-methyl-1H-imidazol-2-yl)-5-phenylthieno[2,3-d]pyrimidine 76A (605.23 mg, 1.26 mmol), 1-(propan-2-yl)-3-(tetramethyl-1,3,2-dioxaborolan-2-yl)-1H-pyrazole (405.51 mg, 1.72 mmol), cesium carbonate (932.61 mg, 2.86 mmol) and Pd(dppf)Cl₂*CH₂Cl₂ (116.87 mg, 143.12 μmol) were dissolved in degassed dioxane (0.4 mL) under an Ar atmosphere. 0.018 mL of water was added via syringe. The reaction mixture was heated to 80° C. and stirred at this temperature for 16 h. The mixture was cooled to room temperature, filtered through celite and concentrated under reduced pressure. The residue was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 mL/min (loading pump 4 mL/min MeOH) to obtain rac-6-(1-isopropyl-1H-pyrazol-3-yl)-4-(3-(methoxymethyl)pyrrolidin-1-yl)-2-(1-methyl-1H-imidazol-2-yl)-5-phenylthieno[2,3-d]pyrimidine (76) (180.0 mg, 350.43 μmol, 28% yield).

### Example 77

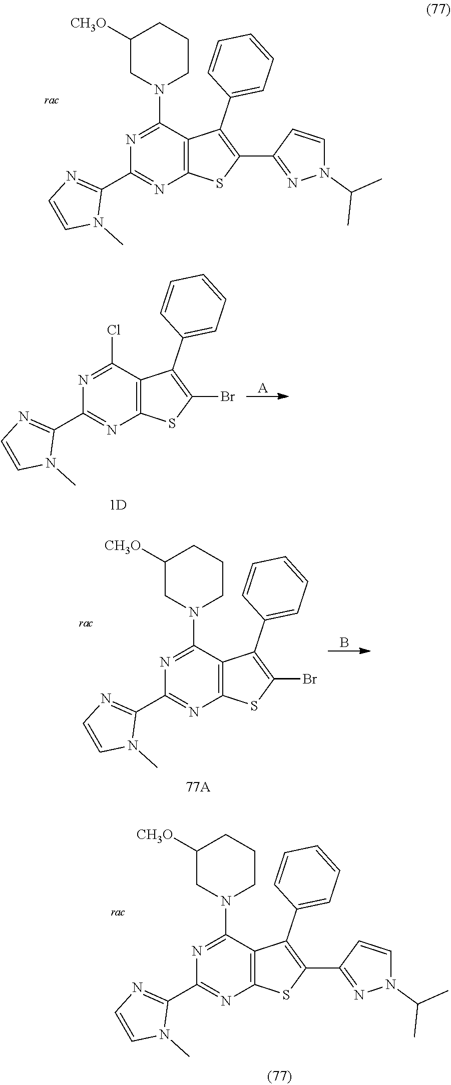

### Rac-6-(1-isopropyl-1H-pyrazol-3-yl)-4-(3-methoxypiperidin-1-yl)-2-(1-methyl-1H-imidazol-2-yl)-5-phenylthieno[2,3-d]pyrimidine (77)

### Step A: rac-6-Bromo-4-(3-methoxypiperidin-1-yl)-2-(1-methyl-1H-imidazol-2-yl)-5-phenylthieno[2,3-d]pyrimidine 77A

6-Bromo-4-chloro-2-(1-methyl-1H-imidazol-2-yl)-5-phenylthieno[2,3-d]pyrimidine 1D (prepared as described in Example 1, Step C) (500.83 mg, 1.23 mmol), racemic 3-methoxypiperidine hydrochloride (280.79 mg, 1.85 mmol) and ethylbis(propan-2-yl)amine (638.12 mg, 4.94 mmol, 860.0 μL, 4.0 equiv) were mixed in DMSO (5 mL). The resulting reaction mixture is stirred at 100° C. for 12 hours and then allowed to cool to room temperature. The mixture is diluted with water (20 mL) and extracted several times with EtOAc (3*25 mL). The combined extracts were washed with brine (25 mL), dried over anhydrous sodium sulfate, and filtered. The filtrate was concentrated under reduced pressure. The residue was purified using HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 mL/min (loading pump 4 mL/min MeOH)) to afford rac-bromo-4-(3-methoxypiperidin-1-yl)-2-(1-methyl-1H-imidazol-2-yl)-5-phenylthieno[2,3-d]pyrimidine 77A (430.0 mg, 887.67 μmol, 71.9% yield).

### Step B: rac-6-(1-Isopropyl-1H-pyrazol-3-yl)-4-(3-methoxypiperidin-1-yl)-2-(1-methyl-1H-imidazol-2-yl)-5-phenylthieno[2,3-d]pyrimidine (77)

rac-Bromo-4-(3-methoxypiperidin-1-yl)-2-(1-methyl-1H-imidazol-2-yl)-5-phenylthieno[2,3-d]pyrimidine 77A (430.0 mg, 1.03 mmol), 1-(propan-2-yl)-3-(tetramethyl-1,3,2-dioxaborolan-2-yl)-1H-pyrazole (290.55 mg, 1.23 mmol), cesium carbonate (668.22 mg, 2.05 mmol) and Pd(dppf)Cl₂*CH₂Cl₂ (83.74 mg, 102.54 μmol) were dissolved in degassed dioxane (0.4 mL) under an Ar atmosphere. 0.018 mL of water was added via syringe. The reaction mixture was heated to 80° C. and stirred at this temperature for 16 h. The mixture was cooled to room temperature, filtered through celite and concentrated in vacuo. The residue was purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min 70-85% MeOH-water; flow 30 mL/min (loading pump 4 mL/min MeOH)) to afford rac-6-(1-isopropyl-1H-pyrazol-3-yl)-4-(3-methoxypiperidin-1-yl)-2-(1-methyl-1H-imidazol-2-yl)-5-phenylthieno[2,3-d]pyrimidine (77) (121.8 mg, 237.12 μmol, 26.5% yield).

### Example 78

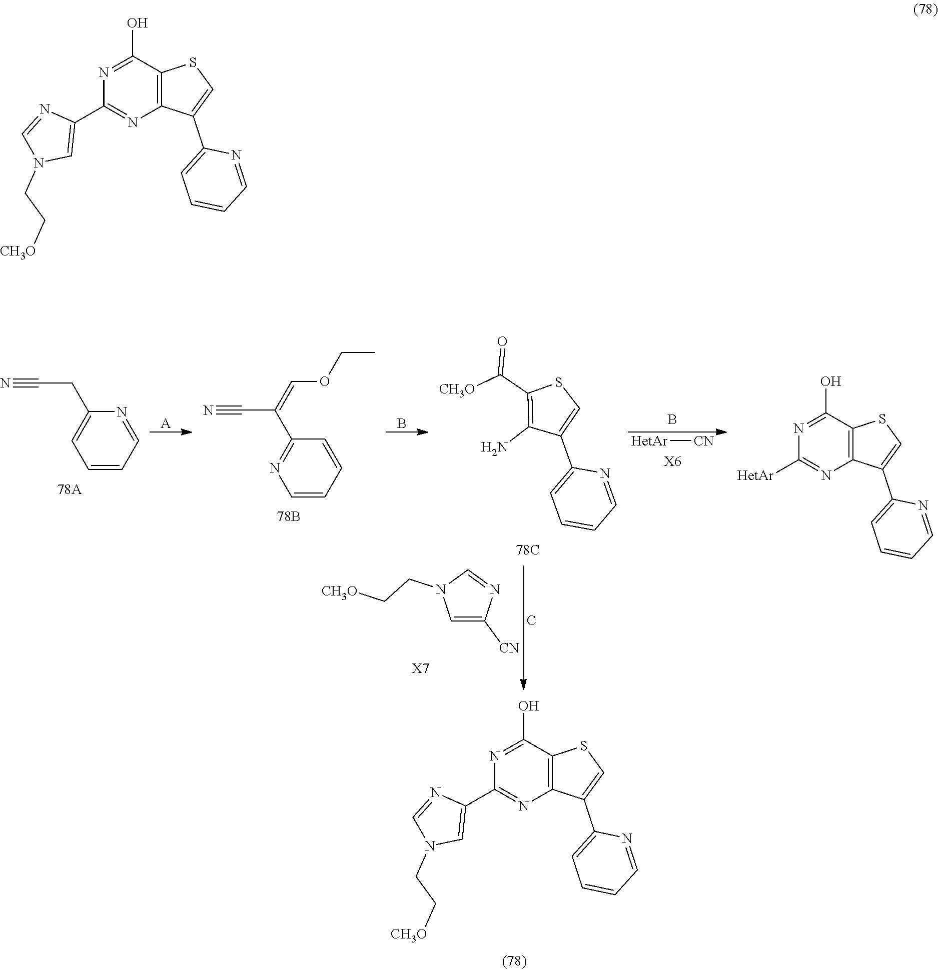

### 2-(1-(2-Methoxyethyl)-1H-imidazol-4-yl)-7-(pyridin-2-yl)thieno[3,2-d]pyrimidin-4-ol (78)

### Step A: (E)-3-Ethoxy-2-(pyridin-2-yl)acrylonitrile 78B

2-(Pyridin-2-yl)acetonitrile 78A (32.00 g, 1 equiv) and triethyl orthoformate (40 g, 1 equiv) was added to acetic anhydride (55.3 g, 2 equiv) at room temperature. The resulting reaction mixture was heated at 100° C. for 3 h, cooled to room temperature, diluted with water (500 mL), and extracted with ethyl acetate (100 mL×3). The combined organic layers were washed with brine, dried over anhydrous Na₂SO₄, and chromatographed on silica gel yielding (E)-3-ethoxy-2-(pyridin-2-yl)acrylonitrile 78B as a brown solid (16.4 g, 35%).

### Step B: Methyl 3-amino-4-(pyridin-2-yl)thiophene-2-carboxylate 78C

Methyl mercaptoacetate (7.2 g, 1.1 equiv) was dissolved in dry THE (200 mL) and cooled to 0° C. DBU (14.1 g, 1.5 equiv) was added thereto portion wise followed by slow addition of (E)-3-ethoxy-2-(pyridin-2-yl)acrylonitrile 78B (10.7 g, 1 equiv). The mixture was stirred at room temperature overnight, the solvent was evaporated and the residue was chromatographed on silica gel yielding methyl 3-amino-4-(pyridin-2-yl)thiophene-2-carboxylate 78C as a brownish solid (6.2 g, 43%).

### Step C: 2-(1-(2-Methoxyethyl)-1H-imidazol-4-yl)-7-(pyridin-2-yl)thieno[3,2-d]pyrimidin-4-ol (78)

General Procedure A: Sodium hydride (2.5 eq) (60% in mineral oil) was added portionwise (during 3 min) to the solution of methyl 3-amino-4-(pyridin-2-yl)thiophene-2-carboxylate 78C (300.0-500.0 mg) and the correct corresponding nitrile HetArCN X6 (1 eq) in dioxane (6 mL). The reaction mixture was heated to reflux and stirred at this temperature for 16 h. Then the mixture was cooled to the room temperature, poured in ice water and neutralized by acetic acid to pH 6-4. The formed participate was collected by filtration, washed by water (2*5 mL) and purified by HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-8 min 50-75% MeOH-water; flow 30 mL/min (loading pump 4 mL/min MeOH)). Products of cyclization were obtained with good yields (4-35%). Using 1-(2-methoxyethyl)-1H-imidazole-4-carbonitrile X7 as the HetArCN nitrile produced 2-(1-(2-methoxyethyl)-1H-imidazol-4-yl)-7-(pyridin-2-yl)thieno[3,2-d]pyrimidin-4-ol (78) (19.1 mg, 4.3% yield). General Procedure B: Sodium hydride (60% dispertion in mineral oil; 2.0 equiv) was added portionwise (during 3 min) to the solution of methyl 3-amino-4-(pyridin-2-yl)thiophene-2-carboxylate 78C (0.2 g, 8 mmol, 1.0 equiv) and corresponding HetArCN nitrile X7 (1.0 equiv) in dioxane (10.0 mL). The reaction mixture was heated under reflux for 16 hours. Then, the resulting mixture was allowed to cool to room temperature, poured in ice and water and neutralized with acetic acid. The formed precipitate was collected by filtration, washed with water (2*10.0 mL) and dried in vacuo. The obtained product was subjected to HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min, 0-60% MeOH—H₂O flow 30 mL/min (loading pump 4 mL/min MeOH)) to afford pure product.

### Examples 79-82

Following the procedure described in Example 78, Step C, using General Procedure A, methyl 3-amino-4-(pyri din-2-yl)thiophene-2-carboxylate 78C (300.0-500.0 mg) was treated with the appropriate corresponding corresponding nitrile HetArCN X6 (1 eq) to produce the Example compounds (79)-(82) shown in Table E.

TABLE E

| | | | Amount |
|---|---|---|---|
| Example | Structure | Name | (Yield) |
| 79 | 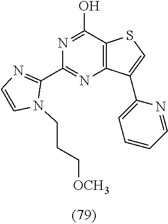 [SMILES: COCCCn1ccnc1-c1nc(O)c2scc(-c3ccccn3)c2n1] | 2-(1-(3- Methoxypropyl)-1H- imidazol-2-yl)-7- (pyridin-2- yl)thieno[3,2- d]pyrimidin-4-ol | 117.0 mg (24.8%) |
| 80 | 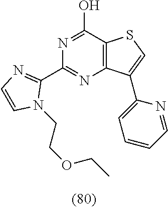 [SMILES: CCOCCn1ccnc1-c1nc(O)c2scc(-c3ccccn3)c2n1] | 2-(1-(2-Ethoxyethyl)- 1H-imidazol-2-yl)-7- (pyridin-2- yl)thieno[3,2- d]pyrimidin-4-ol | 186.2 mg (27.9%) |
| 81 | 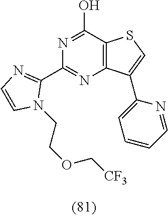 [SMILES: Oc1nc(-c2nccn2CCOCC(F)(F)F)nc2c(-c3ccccn3)csc12] | 7-(Pyridin-2-yl)-2-(1- (2-(2,2,2- trifluoroethoxy)ethyl)- 1H-imidazol-2- yl)thieno[3,2- d]pyrimidin-4-ol | 123.5 mg (21.4%) |
| 82 | 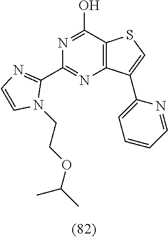 [SMILES: CC(C)OCCn1ccnc1-c1nc(O)c2scc(-c3ccccn3)c2n1] | 2-(1-(2- Isopropoxyethyl)-1H- imidazol-2-yl)-7- (pyridin-2- yl)thieno[3,2- d]pyrimidin-4-ol | 221.0 mg (34.6%) |

### Examples 83-87

Following the procedure described in Example 78, Step C, using General Procedure B, methyl 3-amino-4-(pyridin-2-yl)thiophene-2-carboxylate 78C (0.2 g, 8 mmol, 1.0 equiv) was treated with the appropriate corresponding nitrile HetArCN X6 (1.0 equiv) in dioxane (10.0 mL) to produce the Example compounds (83)-(87) shown in Table F.

TABLE F

| | | | Amount |
|---|---|---|---|
| Example | Structure | Name | (Yield) |
| 83 | 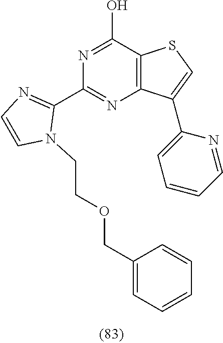 [SMILES: Oc1nc(-c2nccn2CCOCc2ccccc2)nc2c(-c3ccccn3)csc12] | 2-(1-(2-(Benzyloxy)ethyl)- 1H-imidazol-2-yl)-7- (pyridin-2-yl)thieno[3,2- d]pyrimidin-4-ol | 13.0 mg (3.8%) |
| 84 | 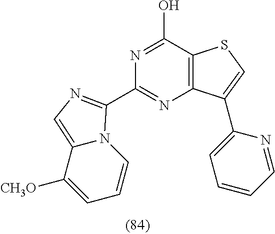 | 2-(8-Methoxyimidazo[1,5- a]pyridin-3-yl)-7-(pyridin- 2-yl)thieno[3,2- d]pyrimidin-4-ol | 7.0 mg (2.2%) |
| 85 | 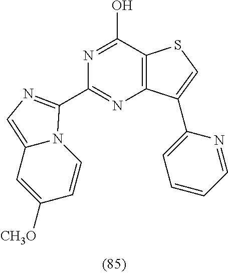 | 2-(7-Methoxyimidazo[1,5- a]pyridin-3-yl)-7-(pyridin- 2-yl)thieno[3,2- d]pyrimidin-4-ol | 6.8 mg (2.1%) |
| 86 | 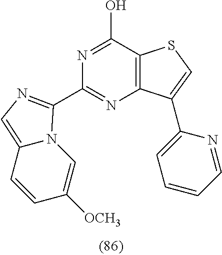 [SMILES: COc1ccc2cnc(-c3nc(O)c4scc(-c5ccccn5)c4n3)n2c1] | 2-(6-Methoxyimidazo[1,5- a]pyridin-3-yl)-7-(pyridin- 2-yl)thieno[3,2- d]pyrimidin-4-ol | 19.0 mg (5.8%) |
| 87 | 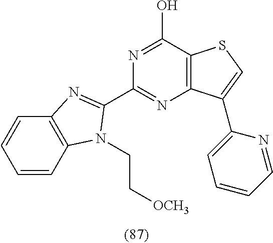 [SMILES: COCCn1c(-c2nc(O)c3scc(-c4ccccn4)c3n2)nc2ccccc21] | 2-(1-(2-Methoxyethyl)-1H- benzo[d]imidazol-2-yl)-7- (pyridin-2-yl)thieno[3,2- d]pyrimidin-4-ol | 21.0 mg (6.0%) |

### Example 88

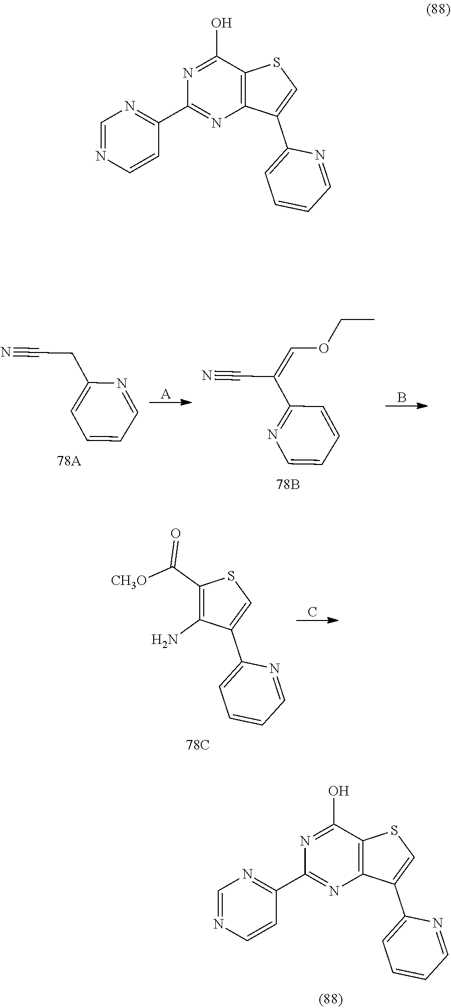

### 7-(Pyridin-2-yl)-2-(pyrimidin-4-yl)thieno[3,2-d]pyrimidin-4-ol (88)

### Step A: (E)-3-Ethoxy-2-(pyridin-2-yl)acrylonitrile 78B

A mixture of 2-(pyridin-2-yl)acetonitrile 78A (25.0 g, 211.63 mmol), triethoxymethane (47.05 g, 317.45 mmol, 52.86 ml, 1.5 equiv) and acetic anhydride (194.45 g, 1.9 mol) was refluxed for 4 hours. Then it was evaporated and purified by flash chromatography (SiO₂, Hexane-EtOAc as a solvent mixture) to give (E)-3-ethoxy-2-(pyridin-2-yl)acrylonitrile 78B (36.3 g, 3.82% purity, 7.96 mmol, 3.8% yield).

### Step B: Methyl 3-amino-4-(pyridin-2-yl)thiophene-2-carboxylate 78C

To the stirred solution of methyl 2-sulfanylacetate (25.44 g, 239.65 mmol, 21.43 ml, 1.15 equiv) in dry THE (300 mL) sodium methanolate (20.26 g, 375.11 mmol) was added at 0° C. after 5 min to the resulted mixture a solution of (E)-3-ethoxy-2-(pyridin-2-yl)acrylonitrile 78B (36.3 g, 3.82% purity, 208.39 mmol) in dry THF (100 mL) was added dropwise. This mixture was heated at 65° C. overnight. Then the solvent was evaporated, 200 mL of water was added and extracted with EtOAc. The organic extracts were dried over Na₂SO₄, and concentrated in vacuo. Methyl 3-amino-4-(pyridin-2-yl)thiophene-2-carboxylate 78C (3.5 g, 66.0% purity, 9.86 mmol, 4.7% yield) was obtained after column chromatography.

### Step C: 7-(Pyridin-2-yl)-2-(pyrimidin-4-yl)thieno[3,2-d]pyrimidin-4-ol (88)

Methyl 3-amino-4-(pyridin-2-yl)thiophene-2-carboxylate 78C (150.0 mg, 640.28 μmol) and pyrimidine-4-carbonitrile (67.37 mg, 641.0 μmol) were dissolved in DMF (20 mL) and sodium hydride (46.15 mg, 1.92 mmol) was added at 0° C. The reaction mixture was heated at 80° C. overnight. The solution of NH₄Cl (15 mL) was added dropwise to reaction mixture after cooling to 0° C., then extracted with EtOAc (3×30 mL). The organic extracts were dried over Na₂SO₄, concentrated in vacuo and purified by HPLC (2-10 min; 40-65% water/MeCN; Flow rate 30 mL/min; loading pump MeCN 4 mL/min; column SunFire 19*100 mm) to give 7-(Pyridin-2-yl)-2-(pyrimidin-4-yl)thieno[3,2-d]pyrimidin-4-ol (88) (10.1 mg, 95.0% purity, 31.22 μmol, 4.9% yield).

### Example 89

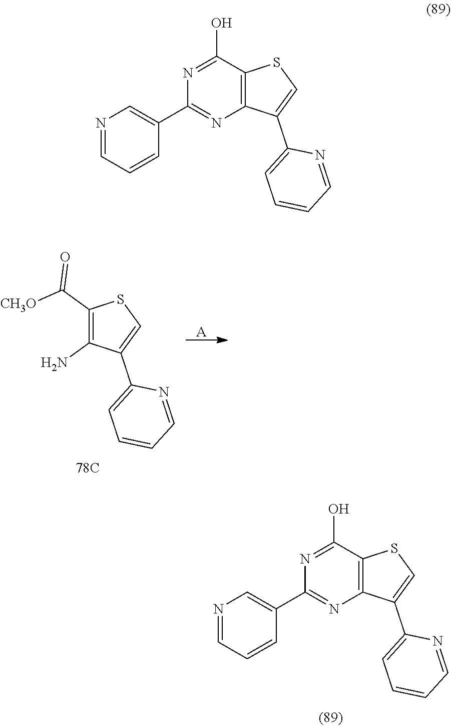

### 7-(Pyridin-2-yl)-2-(pyridin-3-yl)thieno[3,2-d]pyrimidin-4-ol (89)

Methyl 3-amino-4-(pyridin-2-yl)thiophene-2-carboxylate 78C (prepared as described in Example 88, Step B) (149.89 mg, 639.81 μmol) and pyridine-3-carbonitrile (66.61 mg, 639.81 μmol) were dissolved in DMF (20 mL) and sodium hydride (76.77 mg, 3.2 mmol) was added at 0° C. The reaction mixture was heated at 80° C. overnight. Solution of NH₄Cl (15 mL) was added dropwise to reaction mixture after cooling to 0° C., then extracted with EtOAc (3×30 mL). The organic extracts were dried over Na₂SO₄, concentrated in vacuo and purified by HPLC (2-10 min; 40-65% water/MeCN; Flow rate 30 mL/min; loading pump MeCN 4 mL/min; column SunFire 19*100 mm) to give 7-(pyridin-2-yl)-2-(pyridin-3-yl)thieno[3,2-d]pyrimidin-4-ol (89) (5.9 mg, 95.0% purity, 18.3 μmol, 2.9% yield).

### Example 90

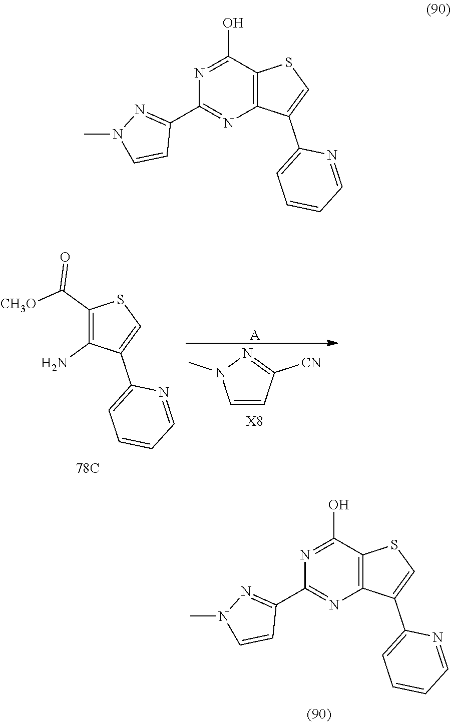

### 2-(1-Methyl-1H-pyrazol-3-yl)-7-(pyridin-2-yl)thieno[3,2-d]pyrimidin-4-ol (90)

Methyl 3-amino-4-(pyridin-2-yl)thiophene-2-carboxylate 78C (synthesized as described in Example 88, Step B) (149.95 mg, 640.06 μmol) and 1-methyl-1H-pyrazole-3-carbonitrile X8 (68.56 mg, 640.06 μmol) were dissolved in DMF (20 mL) and sodium hydride (102.4 mg, 4.27 mmol) was added at 0° C. Reaction mixture was heated at 80° C. overnight. Solution of NH₄Cl (15 mL) was added dropwise to reaction mixture after cooling to 0° C., then extracted with EtOAc (3×30 mL). The organic extracts were dried over Na₂SO₄, concentrated in vacuo and purified by HPLC (2-10 min 40-65% water-MeCN; flow 30 mL/min (loading pump MeCN 4 mL/min); column SunFire 19×100 mm, 5 um) to give 2-(1-methyl-1H-pyrazol-3-yl)-7-(pyridin-2-yl)thieno[3,2-d]pyrimidin-4-ol (90) (17.5 mg, 95.0% purity, 53.74 μmol, 8.4% yield).

### Example 91

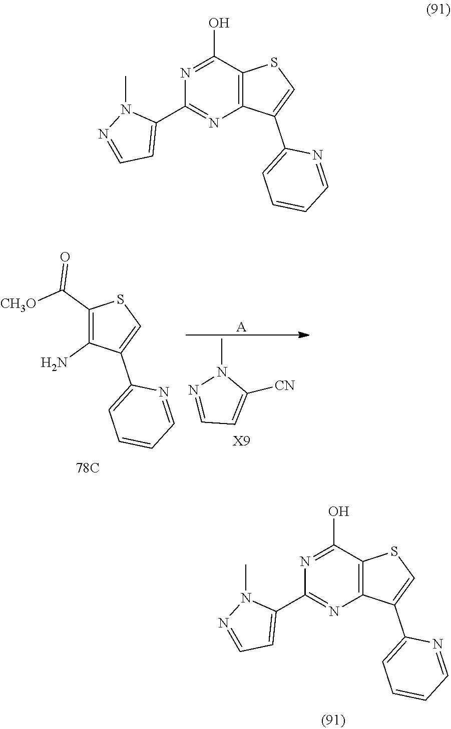

### 2-(1-Methyl-1H-pyrazol-5-yl)-7-(pyridin-2-yl)thieno[3,2-d]pyrimidin-4-ol (91)

Methyl 3-amino-4-(pyridin-2-yl)thiophene-2-carboxylate 78C (synthesized as described in Example 88, Step B) (149.95 mg, 640.06 μmol) and 1-methyl-1I-pyrazole-5-carbonitrile X9 (68.56 mg, 640.06 μmol) were dissolved in DMF (20 mL) and sodium hydride (102.4 mg, 4.27 mmol) was added at 0° C. Reaction mixture was heated at 80° C. overnight. Solution of NH₄Cl (15 mL) was added dropwise to reaction mixture after cooling to 0° C., then extracted with EtOAc (3×30 mL). The organic extracts were dried over Na₂SO₄, concentrated in vacuo and purified by HPLC (2-10 min 34-45% water-MeCN; flow 30 mL/min (loading pump MeCN 4 mL/min); column SunFire 19×100 mm, 5 um) to give 2-(1-methyl-1H-pyrazol-5-yl)-7-(pyridin-2-yl)thieno[3,2-d]pyrimidin-4-ol (91) (7.5 mg, 95.0% purity, 23.03 μmol, 3.6% yield).

### Example 92

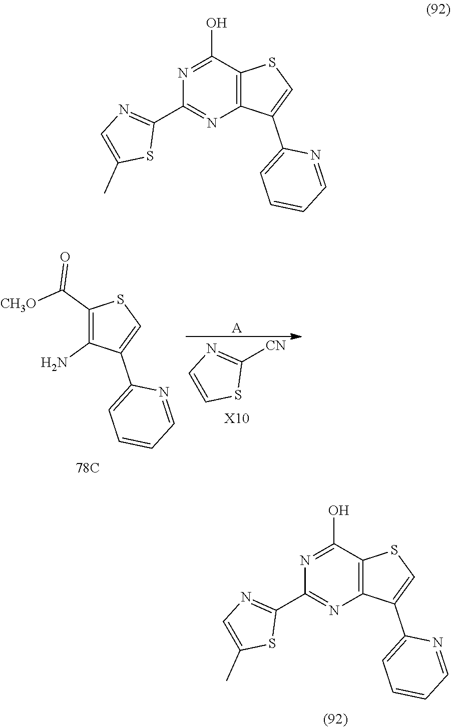

### 2-(5-Methylthiazol-2-yl)-7-(pyridin-2-yl)thieno[3,2-d]pyrimidin-4-ol (92)

Methyl 3-amino-4-(pyridin-2-yl)thiophene-2-carboxylate 78C (synthesized as described in Example 88, Step B) (200.26 mg, 854.79 μmol) and thiazole-2-carbonitrile X10 (106.13 mg, 854.79 μmol) were dissolved in DMF (20 mL) and sodium hydride (136.75 mg, 5.7 mmol) was added at 0° C. Reaction mixture was heated at 80° C. overnight. Solution of NH₄Cl (15 mL) was added dropwise to reaction mixture after cooling to 0° C., then extracted with EtOAc (3×30 mL). The organic extracts were dried over Na₂SO₄, concentrated in vacuo and purified by HPLC (2-10 min 60-75% water-methanol; flow 30 mL/min (loading pump 4 mL/min methanol), Column Sun Fire C18 100×19 mm, 5 um) to give 2-(5-methylthiazol-2-yl)-7-(pyridin-2-yl)thieno[3,2-d]pyrimidin-4-ol (92). (34.0 mg, 95.0% purity, 98.96 μmol, 11.6% yield).

### Example 93

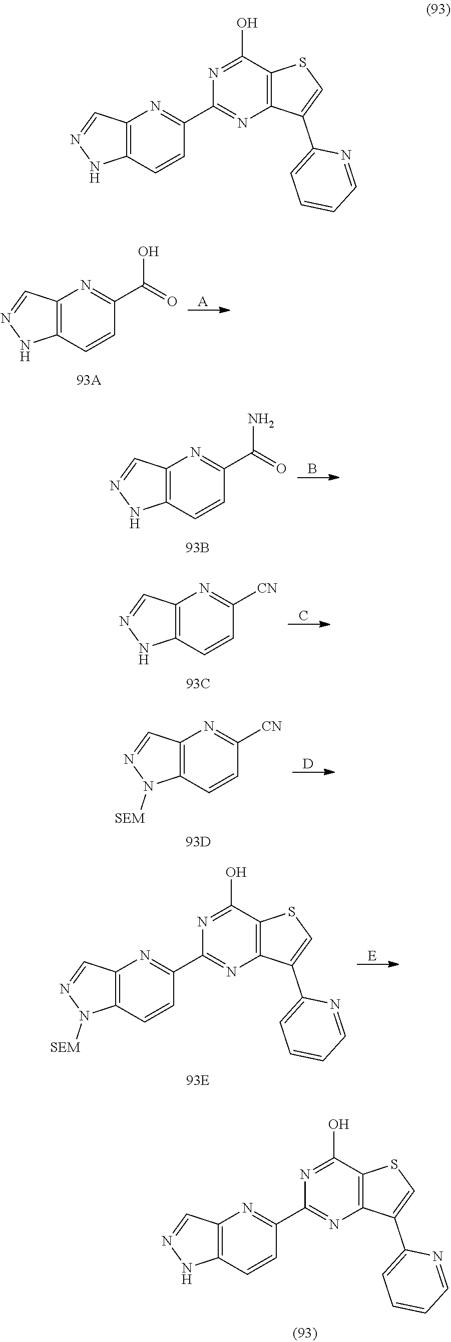

### 2-(1H-Pyrazolo[4,3-b]pyridine-5-yl)-7-(pyridine-2-yl)thieno[3,2-d]pyrimidin-4-ol (93)

### Step A: 1H-Pyrazolo[4,3-b]pyridine-5-carboxamide 93B

To a solution of 1H-pyrazolo[4,3-b]pyridine-5-carboxylic acid 93A (5.0 g, 30.64 mmol) in DMF (50 mL), 1-methyl-1H-imidazole (10.06 g, 122.57 mmol, 9.77 ml, 4.0 equiv), ammonium chloride (3.28 g, 61.28 mmol) and chloro-N,N,N,N-tetramethylformamidinium hexafluorophosphate (10.32 g, 36.77 mmol) were added. The reaction mixture was stirred at room temperature overnight. Water was added (50 mL) and precipitate was formed. The mixture was filtered and washed with water. The precipitate was dried on air to give 1H-pyrazolo[4,3-b]pyridine-5-carboxamide 93B (1.17 g, 98.0% purity, 7.07 mmol, 23.1% yield).

### Step B: 1H-Pyrazolo[4,3-b]pyridine-5-carbonitrile 93C

To a solution of 1H-pyrazolo[4,3-b]pyridine-5-carboxamide 93B (1.17 g, 7.22 mmol) and triethylamine (2.34 g, 23.11 mmol, 3.22 ml, 3.2 equiv) in THE (25 mL), trifluoroacetyl 2,2,2-trifluoroacetate (2.43 g, 11.55 mmol, 1.62 mL, 1.6 equiv) was added dropwise at 0° C. The reaction mixture was stirred at room temperature overnight. Then, the mixture was concentrated under reduced pressure. Ethyl acetate was added (30 mL) and washed with water (3×10 mL), The organic extracts were dried over Na₂SO₄ and concentrated in vacuo to give 1H-pyrazolo[4,3-b]pyridine-5-carbonitrile 93C (650.0 mg, 98.53% purity, 4.44 mmol, 61.5% yield).

### Step C: 1-((2-(Trimethylsilyl)ethoxy)methyl)-1H-pyrazolo[4,3-b]pyridine-5-carbonitrile 93D

To a solution of 1H-pyrazolo[4,3-b]pyridine-5-carbonitrile 93C (200.66 mg, 1.39 mmol) in DMF (20 mL), sodium hydride (83.52 mg, 3.48 mmol) was added at 0° C. portionwise. The resulting mixture was stirred for 30 min following by the addition of [2-(chloromethoxy)ethyl]trimethylsilane (348.54 mg, 2.09 mmol, 370.0 μL, 1.5 equiv). The reaction mixture was stirred at room temperature overnight. Then, saturated solution of ammonium chloride was added at 0° C. (10 mL), extracted with EtOAc (3×15 mL). Combined EtOAc was washed with water (5×10 mL), dried over Na₂SO₄ and concentrated in vacuo to give 1-((2-(trimethylsilyl)ethoxy)methyl)-1H-pyrazolo[4,3-b]pyridine-5-carbonitrile 93D (400.0 mg, 90.0% purity, 1.31 mmol, 94.2% yield).

### Step D: 7-(Pyridin-2-yl)-2-(1-((2-(trimethylsilyl)ethoxy)methyl)-1H-pyrazolo[4,3-b]pyridin-5-yl)thieno[3,2-d]pyrimidin-4-ol 93E

1-((2-(Trimethylsilyl)ethoxy)methyl)-1H-pyrazolo[4,3-b]pyridine-5-carbonitrile 93D (199.77 mg, 728.03 μmol) and methyl 3-amino-4-(pyridin-2-yl)thiophene-2-carboxylate (170.56 mg, 728.03 μmol) were dissolved in DMF (20 mL) and sodium hydride (87.36 mg, 3.64 mmol) was added at 0° C. portionwise. The reaction mixture was heated at 80° C. overnight. Then, the solution of NH₄Cl (15 mL) was added dropwise to reaction mixture after cooling to 0° C., then extracted with EtOAc (3×30 mL). The organic extracts were dried over Na₂S04, concentrated in vacuo to give 7-(pyridin-2-yl)-2-(1-((2-(trimethylsilyl)ethoxy)methyl)-1H-pyrazolo[4,3-b]pyridin-5-yl)thieno[3,2-d]pyrimidin-4-ol 93E (400.0 mg, 33.43% purity, 280.56 μmol, 38.5% yield).

### Step E: 2-(1H-Pyrazolo[4,3-b]pyridin-5-yl)-7-(pyridin-2-yl)thieno[3,2-d]pyrimidin-4-ol (93)

To a solution of 7-(pyridin-2-yl)-2-(1-((2-(trimethylsilyl)ethoxy)methyl)-1H-pyrazolo[4,3-b]pyridin-5-yl)thieno[3,2-d]pyrimidin-4-ol 93E (400.0 mg, 839.23 μmol) in DCM (2 mL), 2,2,2-trifluoroacetic acid (191.59 mg, 1.68 mmol) was added. The reaction mixture was stirred at room temperature for 2 days. The mixture was concentrated under reduce pressure and purified by HPLC (2-10 min; 30-45% water/MeOH+NH₃; Flow rate 30 mL/min; loading pump MeOH+NH₃ 4 mL/min; column SunFire 19*100 mm) to afford 2-(1H-pyrazolo[4,3-b]pyridin-5-yl)-7-(pyridin-2-yl)thieno[3,2-d]pyrimidin-4-ol (93). (4.2 mg, 95.0% purity, 11.52 μmol, 1.4% yield).

### Example 94

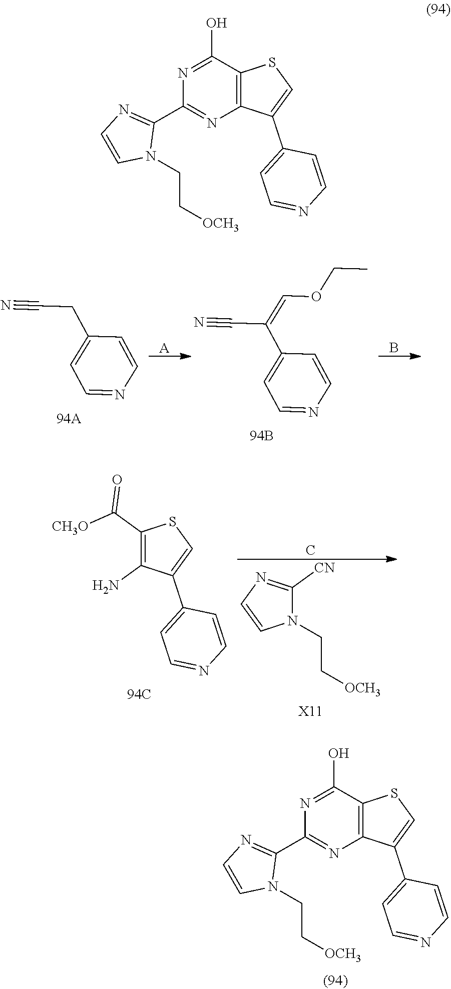

### 2-(1-(2-Methoxyethyl)-1H-imidazol-2-yl)-7-(pyridin-4-yl)thieno[3,2-d]pyrimidin-4-ol (94)

### Step A: (E)-3-Ethoxy-2-(pyridin-4-yl)acrylonitrile 94B

A mixture of 2-(pyridin-4-yl)acetonitrile 94A (25.0 g, 211.63 mmol), triethoxymethane (47.05 g, 317.45 mmol, 52.86 ml, 1.5 equiv) and acetic anhydride (194.45 g, 1.9 mol) was refluxed for 4 hours. Then it was evaporated and purified by flash chromatography (SiO₂, Hexane-EtOAc as a mobile phase) to give (E)-3-ethoxy-2-(pyridin-4-yl)acrylonitrile 94B (36.3 g, 3.82% purity, 7.96 mmol, 3.8% yield).

### Step B: Methyl 3-amino-4-(pyridin-4-yl)thiophene-2-carboxylate 94C

To the stirred solution of methyl 2-sulfanylacetate (25.44 g, 239.65 mmol, 21.43 ml, 1.15 equiv) in dry THE (300 mL) sodium methanolate (20.26 g, 375.11 mmol) was added at 0° C. The resulting mixture was stirred for 5 min following by the dropwise addition of a solution of (E)-3-ethoxy-2-(pyridin-4-yl)acrylonitrile 94B (36.3 g, 3.82% purity, 208.39 mmol) in dry THE (100 mL). This mixture was heated at 65° C. overnight. Then the solvent was evaporated in vacuo, 200 mL of water was added and extracted with EtOAc. The organic extracts were dried over Na₂SO₄, and concentrated in vacuo. Methyl 3-amino-4-(pyridin-4-yl)thiophene-2-carboxylate 94C (3.5 g, 66.0% purity, 9.86 mmol, 4.7% yield) was obtained after column chromatography (SiO₂, Hexane-EtOAc as a mobile phase).

### Step C: 2-(1-(2-Methoxyethyl)-1H-imidazol-2-yl)-7-(pyridin-4-yl)thieno[3,2-d]pyrimidin-4-ol (94)

Methyl 3-amino-4-(pyridin-4-yl)thiophene-2-carboxylate 94C (169.71 mg, 724.4 μmol) and 1-(2-methoxyethyl)-1H-imidazole-2-carbonitrile (109.5 mg, 724.4 μmol) were dissolved in DMF (20 mL) and sodium hydride (115.89 mg, 4.83 mmol) was added at 0° C. Reaction mixture was heated at 80° C. overnight. Solution of NH₄Cl (15 mL) was added dropwise to reaction mixture after cooling to 0° C., then extracted with EtOAc (3×30 mL). The organic extracts were dried over Na₂SO₄, concentrated in vacuo and purified by HPLC (2-10 min 0-45% water-MeOH-0.1% NH₄OH; flow 30 mL/min (loading pump MeOH 4 mL/min); column SunFire 19×100 mm, 5 um) to give 2-(1-(2-methoxyethyl)-1H-imidazol-2-yl)-7-(pyridin-4-yl)thieno[3,2-d]pyrimidin-4-ol (94) (12.4 mg, 95.0% purity, 33.33 μmol, 4.6% yield).

### Example 95

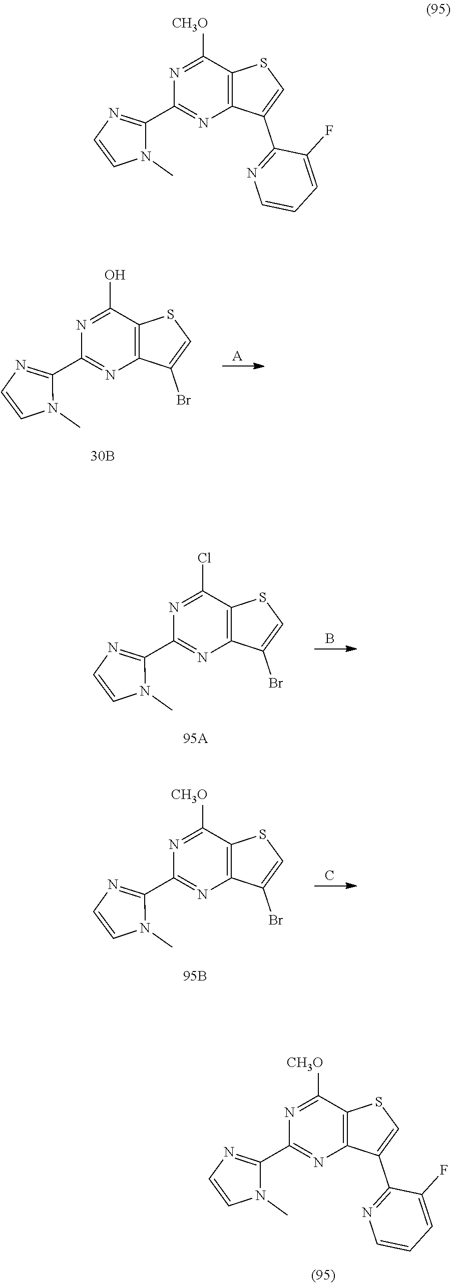

### 7-(3-Fluoropyridin-2-yl)-4-methoxy-2-(1-methyl-1H-imidazol-2-yl)thieno[3,2-d]pyrimidine (95)

### Step A: 7-Bromo-4-chloro-2-(1-methyl-1H-imidazol-2-yl)thieno[3,2-d]pyrimidine 95A

To a suspension of 7-bromo-2-(1-methyl-1H-imidazol-2-yl)thieno[3,2-d]pyrimidin-4-ol 30B (prepared as described in Example 30, Step A) (300.26 mg, 964.96 μmol) in phosphoryl trichloride (3.11 g, 20.26 mmol) triethylamine (99.6 mg, 984.26 μmol) was slowly added at room temperature. The resulting mixture was stirred for 8 h at 60° C. After cooling, the excess of phosphoryl chloride was removed under reduced pressure. The residue was mixed with chloroform (10 mL) and sodium bicarbonate aqueous solution (10 ml). The mixture was extracted with chloroform (10 mL), and the organic phase was separated, dried over Na₂SO₄, filtered and evaporated in vacuo to obtain 7-bromo-4-chloro-2-(1-methyl-1H-imidazol-2-yl)thieno[3,2-d]pyrimidine 95A (300.0 mg, 90.0% purity, 814.19 μmol, 84.4% yield).

### Step B: 7-Bromo-4-methoxy-2-(1-methyl-1H-imidazol-2-yl)thieno[3,2-d]pyrimidine 95B

Sodium methanolate (73.31 mg, 1.36 mmol) was added to suspension of 7-bromo-4-chloro-2-(1-methyl-1H-imidazol-2-yl)thieno[3,2-d]pyrimidine 95A (300.0 mg, 904.64 μmol) in MeOH (3 mL). The reaction mixture was stirred overnight at room temperature. After the completion of the reaction, the solvent was removed under reduced pressure. The crude product was purified by column chromatography (SiO₂, Hexane/EtOAc (10/1-1/1) as a solvent mixture) to afford 7-bromo-4-methoxy-2-(1-methyl-1H-imidazol-2-yl)thieno[3,2-d]pyrimidine 95B (100.0 mg, 90.0% purity, 275.06 μmol, 30.4% yield) as yellow solid.

### Step C: 7-(3-Fluoropyridin-2-yl)-4-methoxy-2-(1-methyl-1H-imidazol-2-yl)thieno[3,2-d]pyrimidine (95)

7-Bromo-4-methoxy-2-(1-methyl-1H-imidazol-2-yl)thieno[3,2-d]pyrimidine 95B (100.0 mg, 305.62 μmol) and 2-methyl-6-(tributylstannyl)pyridine (128.55 mg, 336.36 μmol) were mixed in DMSO (3 mL) and stirred under an Ar atmosphere for 5 min followed by the addition of Pd(PPh₃)₄ (35.46 mg, 30.58 μmol). The reaction mixture was heated under reflux overnight. The mixture was extracted with ethyl acetate (10 ml*2) and brine (10 mL). The organic phase was washed with saturated aqueous solution of NaCl (10 ml), dried over MgSO₄ and concentrated in vacuo. The crude material was purified via HPLC (Agilent 1260 Infinity systems equipped with DAD and mass-detector; Waters Sunfire C18 OBD Prep Column, 100 A, 5 μm, 19 mm*100 mm with SunFire C18 Prep Guard Cartridge, 100 A, 10 μm, 19 mm*10 mm; 2-10 min, 0-60% MeCN—H₂O+TFA flow 30 ml/min (loading pump 4 ml/min H₂O)) to give 7-(3-fluoropyridin-2-yl)-4-methoxy-2-(1-methyl-1H-imidazol-2-yl)thieno[3,2-d]pyrimidine (95) (3.0 mg, 97.0% purity, 8.47 μmol, 2.8% yield).

### Example 96

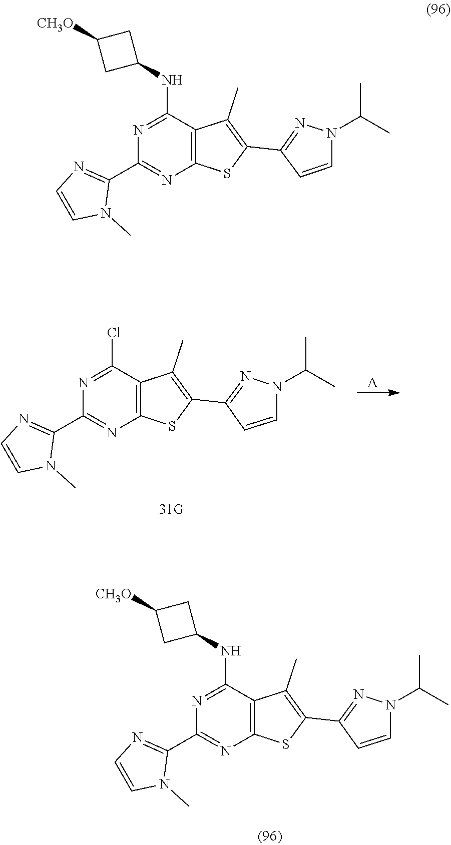

### 6-(1-isopropyl-1H-pyrazol-3-yl)-N-((1s,3s)-3-methoxycyclobutyl)-5-methyl-2-(1-methyl-1H-imidazol-2-yl)thieno[2,3-d]pyrimidin-4-amine (96)

### Step A: 6-(1-isopropyl-1H-pyrazol-3-yl)-N-((1s,3s)-3-methoxycyclobutyl)-5-methyl-2-(1-methyl-1H-imidazol-2-yl)thieno[2,3-d]pyrimidin-4-amine (96)

4-Chloro-6-(1-isopropyl-1H-pyrazol-3-yl)-5-methyl-2-(1-methyl-1H-imidazol-2-yl)thieno[2,3-d]pyrimidine 31G (142.31 mg, 381.66 μmol) (prepared as described in Example 31, Step F) was dissolved in DMSO (3 mL) and N,N-diisopropylethylamine (148.4 mg, 1.15 mmol, 200.0 μL) with corresponding amine (1s,3s)-3-methoxycyclobutan-1-amine hydrochloride (105.04 mg, 763.32 μmol) was added at room temperature. The mixture was heated at 100° C. overnight, cooled and purified by HPLC. 6-(1-Isopropyl-1H-pyrazol-3-yl)-N-((1s,3s)-3-methoxycyclobutyl)-5-methyl-2-(1-methyl-1H-imidazol-2-yl)thieno[2,3-d]pyrimidin-4-amine (96) (12.1 mg, 95.0% purity, 26.27 μmol, 6.9% yield) was obtained as yellow gum after HPLC.

### Purification and Analytical Procedures:

The purification of compounds disclosed herein was performed using HPLC (H₂O—MeOH; Agilent 1260 Infinity systems equipped with DAD and mass-detectors. Waters Sunfire C18 OBD Prep Column, 100A, 5 μm, 19 mm×100 mm with SunFire C18 Prep Guard Cartridge, 100 Å, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated under the N2 flow upon heating to 80° C. On the basis of post-chromatography LCMS analysis fractions were united. Solid fractions were dissolved in 0.5 mL MeOH and transferred into a pre-weighted marked vials. Obtained solutions were again evaporated under the N2 flow upon heating to 80° C. After drying, products were finally characterized by LCMS and ¹H NMR.

NMR Instrument specifications: Bruker AVANCE DRX 500, Varian UNITY plus 400.

LC/MS Instrument specifications: Agilent 1100 Series LC/MSD system with DAD\ELSD and Agilent LC\MSD VL (G1956A), SL (G1956B) mass-spectrometer. Agilent 1200 Series LC/MSD system with DAD\ELSD and Agilent LC\MSD SL (G6130A), SL (G6140A) mass-spectrometer. All the LC/MS data were obtained using positive/negative mode switching. Column Zorbax SB-C18 1.8 μm 4.6×15 mm Rapid Resolution cartridge (PN 821975-932) Mobile phase A-acetonitrile, 0.1% formic acid, B-water (0.1% formic acid) Flow rate 3 ml/min Gradient 0 min-100% B, 0.01 min-100% B, 1.5 min-0% B, 1.8 min-0% B, 1.81 min-100% B. Injection volume 1 μl. Ionization mode atmospheric pressure chemical ionization (APCI). Scan range m/z 80-1000.

Table 3 below provides the both the calculated and observed mass spectral data for each of the above-noted exemplified compounds, and also provides the calculated Log P (using ChemDraw Professional version 20.0.0.38).

TABLE 3
Mass Spectral Data and Calculated LogP

| | | M + H | M + H | LogP |
|---|---|---|---|---|
| Compound | MW | (calculated) | (observed) | (calculated) |
| 1 | 472 | 472.2 | 472.2 | 3.28 |
| 2 | 472 | 472.2 | 472.2 | 2.64 |
| 3 | 486 | 486.2 | 486.2 | 3.09 |
| 4 | 514 | 514.2 | 514.4 | 4.17 |
| 5 | 514 | 514.2 | 514.4 | 4.17 |
| 6 | 530 | 530.2 | 530.1 | 3.36 |
| 7 | 516 | 516.2 | 516.0 | 2.91 |
| 8 | 373 | 374.1 | 374.2 | 2.87 |
| 9 | 359 | 360.1 | 360.2 | 2.42 |
| 10 | 329 | 330.1 | 330.2 | 3.03 |
| 11 | 292 | 293.1 | 293.2 | 1.60 |
| 12 | 362 | 363.1 | 363.2 | 1.67 |
| 13 | 334 | 335.1 | 335.2 | 2.49 |
| 14 | 350 | 351.1 | 351.1 | 1.68 |
| 15 | 368 | 369.1 | 369.2 | 3.50 |
| 16 | 369 | 370.1 | 370.2 | 2.88 |
| 17 | 401 | 401.1 | 401.2 | 1.37 |
| 18 | 272 | 273.0 | 273.0 | 1.66 |
| 19 | 326 | 327.1 | 327.0 | 2.45 |
| 20 | 314 | 315.0 | 315.2 | 2.55 |
| 21 | 330 | 331.0 | 331.2 | 1.74 |
| 22 | 342 | 343.3 | 343.5 | 1.73 |
| 23 | 348 | 349.0 | 349.0 | 3.56 |
| 24 | 349 | 350.0 | 350.0 | 2.94 |
| 25 | 346 | 347.1 | 347.2 | 2.39 |
| 26 | 320 | 321.1 | 321.0 | 3.14 |
| 27 | 324 | 325.1 | 325.0 | 3.67 |
| 30 | 327 | 328.1 | 328.2 | 4.00 |
| 31 | 452 | 452.3 | 452.2 | 3.57 |
| 32 | 452 | 452.3 | 452.2 | 3.24 |
| 33 | 452 | 452.2 | 452.2 | 2.97 |
| 34 | 438 | 438.2 | 438.0 | 2.97 |
| 35 | 412 | 412.2 | 412.2 | 2.97 |
| 36 | 426 | 426.2 | 426.2 | 3.22 |
| 37 | 424 | 424.2 | 424.2 | 2.59 |
| 38 | 452 | 452.3 | 452.2 | 2.70 |
| 39 | 468 | 468.2 | 468.2 | 2.43 |
| 40 | 452 | 452.2 | 452.2 | 3.46 |
| 41 | 468 | 468.2 | 468.2 | 2.94 |
| 42 | 482 | 482.2 | 482.2 | 3.67 |
| 43 | 482 | 482.2 | 482.2 | 2.96 |
| 44 | 482 | 482.2 | 482.0 | 2.34 |
| 45 | 468 | 468.2 | 468.2 | 2.34 |
| 46 | 450 | 450.2 | 450.2 | 2.34 |
| 47 | 452 | 452.2 | 452.2 | 2.96 |
| 48 | 452 | 452.2 | 452.2 | 3.39 |
| 49 | 452 | 452.2 | 452.2 | 3.02 |
| 50 | 452 | 452.2 | 452.2 | 3.02 |
| 51 | 451 | 451.2 | 451.2 | 3.67 |
| 52 | 451 | 451.2 | 451.2 | 3.67 |
| 53 | 448 | 448.2 | 448.2 | 1.91 |
| 54 | 466 | 466.2 | 466.2 | 1.91 |
| 55 | 466 | 466.2 | 466.2 | 2.84 |
| 56 | 465 | 465.2 | 465.0 | 3.19 |
| 57 | 434 | 434.2 | 434.2 | 3.19 |
| 58 | 462 | 462.2 | 462.2 | 2.01 |
| 59 | 434 | 434.2 | 434.2 | 2.55 |
| 60 | 465 | 465.2 | 465.1 | 2.25 |
| 61 | 452 | 452.2 | 452.2 | 1.91 |
| 62 | 480 | 480.2 | 480.3 | 1.86 |
| 63 | 466 | 466.2 | 466.2 | 3.07 |
| 64 | 496 | 460.2 | 460.2 | 4.12 |
| 65 | 497 | 461.2 | 461.1 | 3.45 |
| 66 | 462 | 462.2 | 462.2 | 4.59 |
| 67 | 461 | 461.2 | 461.2 | 4.68 |
| 68 | 462 | 462.2 | 462.2 | 4.08 |
| 69 | 449 | 449.2 | 449.2 | 3.97 |
| 70 | 449 | 449.2 | 449.2 | 3.88 |
| 71 | 449 | 449.2 | 449.4 | 3.40 |
| 72 | 477 | 477.2 | 477.2 | 4.10 |
| 73 | 506 | 506.2 | 506.2 | 3.89 |
| 74 | 506 | 506.2 | 506.2 | 4.55 |
| 75 | 506 | 506.2 | 506.2 | 3.41 |
| 76 | 514 | 514.2 | 514.2 | 3.89 |
| 77 | 514 | 514.2 | 514.2 | 4.11 |
| 78 | 353 | 354.1 | 354.0 | 4.66 |
| 79 | 367 | 368.1 | 368.2 | 4.88 |
| 80 | 367 | 368.1 | 368.0 | 2.62 |
| 81 | 421 | 422.1 | 422.0 | 3.04 |
| 82 | 382 | 382.1 | 382.0 | 3.27 |
| 83 | 430 | 430.1 | 430.0 | 3.91 |
| 84 | 375 | 376.1 | 376.0 | 3.59 |
| 85 | 375 | 376.1 | 376.2 | 4.66 |
| 86 | 375 | 376.1 | 376.0 | 2.08 |
| 87 | 404 | 404.1 | 404.0 | 2.08 |
| 88 | 307 | 308.1 | 308.0 | 2.08 |
| 89 | 306 | 307.1 | 307.0 | 4.58 |
| 90 | 309 | 310.1 | 310.0 | 2.92 |
| 91 | 310 | 310.1 | 310.0 | 3.09 |
| 92 | 326 | 327.0 | 327.0 | 2.43 |
| 93 | 346 | 347.1 | 347.0 | 2.21 |
| 94 | 353 | 354.1 | 354.0 | 3.28 |
| 95 | 341 | 342.1 | 342.0 | 3.29 |
| 96 | 438 | 438.2 | 438.2 | 2.52 |

### Kinetic Solubility Assay:

Equipment: Water purification system Millipore Milli-Q Gradient A10 (Millipore, France); Thermomixer R Block, 1.5 ml (Eppendorf, Germany; Cat #5355); Matrix Multichannel Electronic Pipette 2-125 μL, 5-250 μL, 15-1250 μL (Thermo Scientific, USA; Cat ##2011, 2012, 2004); SpectraMax Plus Microplate Reader (Molecular Devices, USA; Product #02196); Multi-Well Plate Vacuum Manifold (Pall Corporation, USA; Product #5014); and Vacuum pump (Millipore, USA; Model #XX5500000)

Analytical System: The measurements were performed using SpectraMax Plus reader in UV-Vis mode. Acquisition and analysis of the data were performed using SoftMax Pro v.5.4 (Molecular Devices) and Excel 2010 data analysis software.

Methods: Briefly, using a 20 mM stock solution of the compound in 100% DMSO dilutions were prepared to a theoretical concentration of 400 μM in duplicates in phosphate-buffered saline pH 7.4 (138 mM NaCl, 2.7 mM KCl, 10 mM K-phosphate) with 2% final DMSO. The experimental compound dilutions in PBS were further allowed to equilibrate at 25° C. on a thermostatic shaker for two hours and then filtered through HTS filter plates using a vacuum manifold. The filtrates of test compounds were diluted 2-fold with acetonitrile with 2% DMSO before measuring.

In parallel, compound dilutions in acetonitrile/PBS (1:1) were prepared to theoretical concentrations of 0 μM (blank), 10 μM, 25 μM, 50 μM, 100 μM, and 200 μM with 2% final DMSO to generate calibration curves. Ondansetron was used as reference compound to control proper assay performance. 200 μL of each sample was transferred to 96-well plate and measured in 200-550 nm range with 5 nm step.

The concentrations of compounds in PBS filtrate are calculated using a dedicated Microsoft Excel calculation script. Proper absorbance wavelengths for calculations are selected for each compound manually based on absorbance maximums (absolute absorbance unit values for the minimum and maximum concentration points within 0-3 OD range). Each of the final datasets is additionally visually evaluated by the operator and goodness of fit (R2) is calculated for each calibration curve. The effective range of this assay is approximately 2-400 μM and the compounds returning values close to the upper limit of the range may have higher actual solubility (e.g. 5′-deoxy-5-fluorouridine). This method is not suitable for liquid (at 25° C.) substances (were not present among the tested compounds). Table 3A provides the kinetic solubility for some of the exemplified compounds.

TABLE 3A
Kinetic Solubility

| | Kinetic Solubility |
|---|---|
| | (μM) (pH 7.4 PBS + |
| Compound | 2% DMSO) |
| 4 | 5.0 |
| 33 | 49.0 |
| 34 | >400 |
| 36 | 268.0 |
| 38 | >400 |
| 40 | 82.0 |
| 44 | 76.0 |
| 63 | 18.0 |
| 66 | 4.0 |
| 69 | 79.0 |

### BIOLOGICAL EXAMPLES

### Biological Example 1

### Ras GTP Binding Domain Inhibition Assay

The following method was developed as specific assay for the following proteins: KRas wild type, KRas Q61H mutant, KRas G12C mutant, KRas G12D mutant, Rac-1, and Rho-A.

Buffer-I:

25 mM Tris-HCl, pH 7.4

27 mM KCl

137 mM NaCl

1 mM MgCl₂

1 mM DTT

Buffer-II:

50 mM Tris-HCl, pH 7.0

1 mM MgCl₂

1 mM DTT

The small GTPases proteins: KRas wild type. KRas Q61H mutant, KRas G12C mutant, KRas G12D mutants, Rac-1, and Rho-A were expressed as His-tagged proteins. In addition, the Guanosine nucleotide Exchange Factor (GEF) Sos protein (residues 556-1049) was expressed as a His-tagged protein. In cells, the guanine nucleotide exchange factor Sos protein promotes activation of Ras proteins by stimulating an exchange of GDP for GTP. The inclusion of Sos to the Ras GTP binding domain inhibition assay may be considered as an alternative representation of physiological cellular conditions for evaluating the inhibitory activity of some of the tested small molecules.

For the assay, all purified small GTPases proteins were diluted in Buffer-I or Buffer-II to a final concentration of 10-30 μg/mL. 200 μL of each diluted protein was added to a nickel-coated 96-well plate and incubated overnight at 4° C. Then the protein solution was discarded and 200 μL of Buffer-I or Buffer-II was added to each well in the presence of 1% DMSO. Compounds to be tested were added to the protein-coated wells at final concentration of 20 μM, and incubated for 3 hours at room temperature with and without 10-30 μg/mL of Sos added to the final hour of the incubation. When performing IC₅₀ measurements a serial dilution of all tested concentrations was added. Then Cy3-GTP or Cy5-GTP was added to each well to a final concentration of 100 nM. The labeled GTP was incubated for 45 minutes at room temperature. Following GTP incubation, wells were washed 3× in Buffer-I or Buffer-II and 200 μL of Buffer-I or Buffer-II was added to each well. Following washes, the amount of bound labeled-GTP was measured using a SpectraMax M3 (Molecular Devices).

Table 4 and Table 5 show % inhibition data, and Table 6 shows IC₅₀ measurements, for selected compounds tested in the screening assays described above.

TABLE 4
% Inhibition at 20 μM of K-Ras mutant and wild-type protein

| | KRas wild | KRas wild | KRas G12D | KRas G12D | KRas G12C | KRas G12C | KRas Q61H | KRas Q61H |
|---|---|---|---|---|---|---|---|---|
| | type: | type + SOS: | mutant: | mutant + SOS: | mutant: | mutant + SOS: | mutant: | mutant + SOS: |
| Compound | % inh. | % inh. | % inh. | % inh. | % inh. | % inh. | % inh. | % inh. |
| 1 | 0 | A | 0 | 0 | A | A | A | A |
| 2 | D | D | C | C | D | D | C | D |
| 3 | C | D | D | D | D | D | D | D |
| 4 | C | D | C | C | C | D | C | D |
| 5 | C | D | A | C | C | D | B | D |
| 6 | D | D | D | D | D | D | D | D |
| 7 | D | D | C | D | D | D | D | D |
| 8 | D | D | D | D | D | D | D | D |
| 9 | D | D | C | D | D | D | D | D |
| 10 | C | D | C | C | D | D | C | D |
| 11 | 0 | A | A | 0 | A | A | 0 | 0 |
| 12 | 0 | A | A | 0 | A | A | A | 0 |
| 13 | 0 | A | A | 0 | 0 | 0 | 0 | 0 |
| 14 | 0 | 0 | A | A | 0 | 0 | 0 | A |
| 15 | A | 0 | 0 | 0 | A | 0 | A | 0 |
| 16 | A | 0 | A | A | A | A | A | 0 |
| 17 | A | A | 0 | 0 | 0 | 0 | A | 0 |
| 18 | B | A | C | B | B | B | B | B |
| 19 | A | A | B | A | A | A | A | A |
| 20 | C | A | B | B | C | B | B | B |
| 21 | B | A | B | B | B | B | B | B |
| 22 | A | A | A | 0 | A | 0 | A | A |
| 23 | A | A | A | A | A | 0 | 0 | A |
| 24 | A | A | A | 0 | A | A | A | 0 |
| 25 | 0 | 0 | 0 | 0 | 0 | 0 | 0 | 0 |
| 26 | D | C | D | D | D | D | D | D |
| 27 | D | D | C | D | C | D | C | D |
| 30 | D | D | C | C | C | D | C | D |
| 31 | B | D | B | D | C | D | B | D |
| 32 | D | D | B | D | C | D | C | D |
| 33 | D | D | C | D | D | D | D | D |
| 34 | D | D | D | D | D | D | D | D |
| 35 | D | D | D | D | D | D | D | D |
| 36 | D | D | C | D | D | D | D | D |
| 37 | D | D | D | D | D | D | D | D |
| 38 | D | D | C | D | C | D | D | D |
| 39 | D | C | C | D | C | C | C | D |
| 40 | D | D | B | B | C | D | C | D |
| 41 | C | C | 0 | B | C | C | C | B |
| 42 | C | D | D | C | D | D | D | D |
| 43 | B | D | D | D | A | D | A | D |
| 44 | D | D | C | D | D | D | D | D |
| 45 | D | D | D | D | C | D | D | D |
| 46 | D | C | B | D | C | C | D | D |
| 47 | D | D | C | C | C | D | C | D |
| 48 | D | C | B | C | C | C | C | C |
| 49 | C | D | 0 | B | A | C | C | C |
| 50 | C | C | 0 | A | B | C | B | C |
| 51 | C | C | B | C | C | B | C | C |
| 52 | C | C | C | C | C | B | C | C |
| 53 | B | C | A | B | B | B | B | B |
| 54 | D | D | C | D | D | D | C | D |
| 55 | A | A | C | B | B | B | A | B |
| 56 | D | D | B | D | D | D | C | D |
| 57 | B | B | A | B | B | B | B | B |
| 58 | D | D | B | C | D | D | C | C |
| 59 | B | D | N/T | N/T | C | D | C | D |
| 60 | C | C | N/T | N/T | B | C | C | C |
| 61 | D | D | N/T | N/T | C | D | D | D |
| 62 | C | C | B | C | N/T | C | D | C |
| 63 | C | B | C | C | B | B | C | B |
| 64 | 0 | D | N/T | N/T | B | D | B | D |
| 65 | B | C | B | C | B | D | B | C |
| 66 | C | C | D | C | B | B | C | B |
| 67 | B | C | 0 | C | B | C | C | C |
| 68 | D | D | D | D | D | C | D | C |
| 69 | C | C | C | C | C | C | D | D |
| 70 | B | B | B | B | C | B | B | B |
| 71 | B | B | 0 | A | B | B | B | C |
| 72 | A | 0 | C | 0 | 0 | A | 0 | 0 |
| 73 | A | D | N/T | N/T | A | D | C | C |
| 74 | B | D | N/T | N/T | A | C | C | C |
| 75 | B | C | N/T | N/T | B | C | C | B |
| 76 | C | C | C | B | A | 0 | B | C |
| 77 | B | B | C | B | B | 0 | A | B |
| 78 | D | C | D | C | C | C | D | C |
| 79 | D | C | C | C | C | C | C | C |
| 80 | D | C | D | D | C | C | D | C |
| 81 | D | D | C | D | C | C | C | C |
| 82 | C | C | C | C | C | C | C | C |
| 83 | C | B | C | C | A | C | B | C |
| 84 | B | A | B | B | B | A | B | B |
| 85 | C | A | C | B | B | A | C | B |
| 86 | C | B | C | C | B | B | B | C |
| 87 | B | A | C | B | A | A | A | B |
| 88 | B | A | C | B | A | B | B | B |
| 89 | 0 | 0 | 0 | 0 | 0 | 0 | A | A |
| 90 | 0 | 0 | B | A | A | A | A | B |
| 91 | 0 | 0 | A | A | 0 | A | A | B |
| 92 | C | C | D | C | C | C | D | C |
| 93 | B | 0 | B | A | B | A | A | A |
| 94 | C | C | D | D | C | C | C | C |
| 95 | D | D | D | D | D | D | D | D |
| 96 | 0 | A | 0 | A | 0 | 0 | 0 | 0 |

0 = 0% inhibition, A = 1-25% inhibition, B = 26-50% inhibition, C = 51-75% inhibition, D = 76-100% inhibition, N/T = Not tested.

TABLE 5
% Inhibition at 20 μM of KRas G12D mutant, Rac-1 and Rho-A protein

| | KRas G12D | KRas G12D | | | | |
|---|---|---|---|---|---|---|
| | mutant: | mutant + SOS: | Rac-1: | Rac-1 + SOS: | Rho-A: | Rho-A + SOS: |
| Compound | % inh. | % inh. | % inh. | % inh. | % inh. | % inh. |
| 1 | 0 | 0 | 0 | A | A | 0 |
| 2 | C | C | C | C | D | D |
| 3 | D | D | D | D | C | D |
| 4 | C | C | A | B | B | B |
| 5 | A | C | B | B | C | C |
| 6 | D | D | D | D | D | D |
| 7 | C | D | D | D | D | D |
| 8 | D | D | D | D | D | D |
| 9 | C | D | D | D | D | D |
| 10 | C | C | D | D | D | D |
| 11 | A | 0 | 0 | 0 | A | A |
| 12 | A | 0 | 0 | A | A | A |
| 13 | A | 0 | A | 0 | A | A |
| 14 | A | A | 0 | 0 | A | A |
| 15 | 0 | 0 | 0 | 0 | 0 | 0 |
| 16 | A | A | A | 0 | 0 | A |
| 17 | 0 | 0 | 0 | A | B | 0 |
| 18 | C | B | B | B | B | B |
| 19 | B | A | A | A | B | B |
| 20 | B | B | B | B | B | B |
| 21 | B | B | B | B | C | B |
| 22 | A | 0 | A | A | A | A |
| 23 | A | A | A | A | A | A |
| 24 | A | 0 | 0 | A | A | A |
| 25 | 0 | 0 | 0 | 0 | 0 | 0 |
| 26 | D | D | D | D | D | D |
| 27 | C | D | D | D | D | D |
| 30 | C | C | C | C | D | D |
| 31 | B | D | N/T | B | C | C |
| 32 | B | D | B | B | C | C |
| 33 | C | D | C | C | D | D |
| 34 | D | D | D | D | D | D |
| 35 | D | D | D | D | D | D |
| 36 | C | D | D | D | B | D |
| 37 | D | D | D | D | D | D |
| 38 | C | D | D | D | D | D |
| 39 | C | D | C | C | D | D |
| 40 | B | B | C | C | D | D |
| 41 | 0 | B | D | D | C | D |
| 42 | D | C | C | C | C | D |
| 43 | D | D | B | C | D | D |
| 44 | C | D | D | D | D | D |
| 45 | D | D | D | D | D | D |
| 46 | B | D | D | D | D | D |
| 47 | C | C | C | C | D | D |
| 48 | B | C | B | B | C | D |
| 49 | 0 | B | B | N/T | C | N/T |
| 50 | 0 | A | 0 | N/T | D | N/T |
| 51 | B | C | C | N/T | D | N/T |
| 52 | C | C | C | N/T | D | N/T |
| 53 | A | B | A | N/T | D | N/T |
| 54 | C | D | B | N/T | C | N/T |
| 55 | C | B | 0 | N/T | C | N/T |
| 56 | B | D | D | N/T | D | N/T |
| 57 | A | B | 0 | N/T | C | N/T |
| 58 | B | C | B | N/T | D | N/T |
| 59 | N/T | N/T | C | N/T | C | N/T |
| 60 | N/T | N/T | C | N/T | D | N/T |
| 61 | N/T | N/T | B | N/T | C | N/T |
| 62 | B | C | A | N/T | D | N/T |
| 63 | C | C | B | N/T | C | N/T |
| 64 | N/T | N/T | B | N/T | B | N/T |
| 65 | B | C | B | N/T | C | N/T |
| 66 | D | C | C | N/T | D | N/T |
| 67 | 0 | C | A | N/T | B | N/T |
| 68 | D | D | D | N/T | D | N/T |
| 69 | C | C | A | B | C | C |
| 70 | B | B | B | B | C | C |
| 71 | 0 | A | 0 | N/T | D | N/T |
| 72 | C | 0 | 0 | N/T | A | N/T |
| 73 | N/T | N/T | A | N/T | C | N/T |
| 74 | N/T | N/T | A | N/T | C | N/T |
| 75 | N/T | N/T | A | N/T | C | N/T |
| 76 | C | B | A | N/T | B | N/T |
| 77 | C | B | A | N/T | B | N/T |
| 78 | D | C | C | N/T | C | N/T |
| 79 | C | C | C | N/T | D | N/T |
| 80 | D | D | D | N/T | C | N/T |
| 81 | C | D | D | N/T | D | N/T |
| 82 | C | C | D | N/T | C | N/T |
| 83 | C | C | D | N/T | C | N/T |
| 84 | B | B | B | N/T | C | N/T |
| 85 | C | B | C | N/T | C | N/T |
| 86 | C | C | C | N/T | C | N/T |
| 87 | C | B | C | N/T | C | N/T |
| 88 | C | B | C | N/T | C | N/T |
| 89 | 0 | 0 | 0 | N/T | A | N/T |
| 90 | B | A | B | N/T | C | N/T |
| 91 | A | A | 0 | N/T | 0 | N/T |
| 92 | D | C | D | N/T | D | N/T |
| 93 | B | A | A | N/T | C | N/T |
| 94 | D | D | D | N/T | D | N/T |
| 95 | D | D | D | D | D | D |
| 96 | 0 | A | A | A | 0 | 0 |

0 = 0% inhibition, A = 1-25% inhibition, B = 26-50% inhibition, C = 51-75% inhibition,
D = 76-100% inhibition, N/T = Not tested.

TABLE 6
KRas 061H mutant IC₅₀ (μM) values

| | KRas Q61H |
|---|---|
| | mutant + SOS: |
| Compound | IC50 (μM) |
| 1 | I |
| 2 | K |
| 3 | J |
| 4 | K |
| 5 | K |
| 6 | K |
| 7 | K |
| 8 | K |
| 9 | J |
| 10 | J |
| 11 | I |
| 12 | I |
| 13 | I |
| 14 | I |
| 15 | I |
| 16 | I |
| 17 | I |
| 18 | I |
| 19 | I |
| 20 | I |
| 21 | I |
| 22 | I |
| 23 | I |
| 24 | I |
| 25 | N/T |
| 26 | J |
| 27 | K |
| 30 | K |
| 31 | K |
| 32 | K |
| 96 | I |

I = >10 uM IC₅₀,
J = 5-10 uM IC₅₀,
K = 1-4.99 uM IC₅₀,
L = <1 uM IC₅₀,
N/T = Not tested

### Biological Example 2

### Protocols for Cell-Based Phosphorylation Assays

### Protocol for Western ERK1/2 Cell-Based Phosphorylation Assay

Cell lines: Human tumor-derived pancreatic cancer cell line Panc-1 was purchased from American Type Culture Collection and grown in complete DMEM-High Glucose, supplemented with penicillin (100 U/mL), streptomycin (100 μg/mL), and 10% heat-inactivated FBS at 37° C. in a humidified incubator with 5% CO₂.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 6 hours incubation at 37° C. For IC₅₀ value determination, serial dilutions of compounds were added to cells under the same conditions. Next, cells were stimulated with 1.5 ng/ml EGF for 15 minutes then cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of ERK1/2 was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of ERK1/2 were assessed by incubating overnight at 4° C. with anti-phospho-p44/42 (Thr202/Tyr204) antibody (Cell Signaling) followed by HRP-conjugated secondary antibody (Jackson Immunoresearch, West Grove, Pa.). Bands were incubated in Amersham ECL Prime Western Blotting Detection Reagent (GE Healthcare) and visualized using the ChemiDoc MP imaging system (Bio-Rad). Protocol for Western AKT cell-based phosphorylation assay

### Human Tumor-Derived Pancreatic Cancer Cell Line Panc-1 was Purchased from

American Type Culture Collection and grown in complete DMEM-High Glucose supplemented with penicillin (100 U/mL), streptomycin (100 μg/mL), and 10% heat-inactivated FBS at 37° C. in a humidified incubator with 5% CO₂.

Method: Cells were plated at 350000 cells/well density in a 12-well plate, allowed 3 hours to adhere to the plate, then starved in the appropriate medium in the presence of 0.5% FBS overnight. The small molecules to be tested were added to the cells in the final concentration of 10 μM in the presence of 0.3% DMSO for 6 hours incubation at 37° C. Next, cells were stimulated with 1.5 ng/ml EGF for 15 minutes then cells were lysed with lysis buffer containing 1% Triton X-100, EDTA, and Halt™ Protease & Phosphatase Inhibitor Cocktail (Thermo Scientific). Protein concentration was assessed by BCA protein assay (Thermo Scientific). Phosphorylation level of Akt was determined by western blot.

Western blot protocol: Equal amounts of protein (15-50 μg) were separated by SDS-PAGE and transferred to nitrocellulose membranes (Invitrogen by Thermo Fisher Scientific). The membrane was stained with Ponceau S Stain (Boston BioProducts) to verify uniform protein loading. Membranes were blocked with 10% milk and phosphorylation levels of Akt were assessed by incubating overnight at 4° C. with anti-phospho-Akt (Ser473) antibody (Cell Signaling) followed by HRP-conjugated secondary antibody (Jackson Immunoresearch, West Grove, Pa.). Bands were incubated in Amersham ECL Prime Western Blotting Detection Reagent (GE Healthcare) and visualized using the ChemiDoc MP imaging system (Bio-Rad).

Table 7 shows inhibition data for selected compounds tested in one or more of the cellular assays described above.

TABLE 7
% Inhibition of ERK1/2 phosphorylation (at 10 μM)
and % Inhibition of AKT phosphorylation
(at 10 μM) in PANC-1 pancreatic cancer cell line

| | % inh of | |
|---|---|---|
| | ERK1/2 | % inh of AKT |
| | phosphorylation | phosphorylation |
| Compound | in PANC-1 | in PANC-1 |
| 1 | 0 | 0 |
| 2 | C | 0 |
| 3 | A | 0 |
| 4 | D | D |
| 5 | D | D |
| 6 | D | 0 |
| 7 | D | 0 |
| 8 | 0 | 0 |
| 9 | 0 | 0 |
| 10 | A | 0 |
| 11 | 0 | 0 |
| 12 | 0 | 0 |
| 13 | 0 | 0 |
| 14 | 0 | 0 |
| 15 | 0 | 0 |
| 16 | 0 | 0 |
| 17 | A | 0 |
| 18 | 0 | 0 |
| 19 | 0 | 0 |
| 20 | 0 | 0 |
| 21 | 0 | 0 |
| 22 | 0 | 0 |
| 23 | 0 | 0 |
| 24 | 0 | 0 |
| 25 | 0 | 0 |
| 26 | 0 | 0 |
| 30 | 0 | 0 |
| 31 | D | 0 |
| 32 | D | 0 |
| 33 | D | 0 |
| 34 | D | 0 |
| 35 | 0 | 0 |
| 36 | C | 0 |
| 37 | 0 | 0 |
| 38 | D | B |
| 39 | 0 | <B> |
| 40 | D | D |
| 41 | 0 | 0 |
| 42 | B | <D> |
| 43 | B | <D> |
| 44 | D | <A> |
| 45 | B | <D> |
| 46 | 0 | A |
| 47 | D | 0 |
| 48 | D | 0 |
| 49 | D | D |
| 50 | D | D |
| 51 | 0 | 0 |
| 52 | 0 | 0 |
| 53 | 0 | 0 |
| 54 | D | <A> |
| 55 | D | <A> |
| 56 | 0 | 0 |
| 57 | A | 0 |
| 58 | A | 0 |
| 59 | 0 | 0 |
| 60 | 0 | 0 |
| 61 | D | 0 |
| 62 | D | B |
| 63 | D | C |
| 64 | D | B |
| 65 | D | B |
| 66 | 0 | B |
| 67 | 0 | <A> |
| 68 | C | <A> |
| 69 | D | D |
| 70 | 0 | 0 |
| 71 | D | D |
| 72 | D | B |
| 73 | D | A |
| 74 | D | 0 |
| 75 | D | 0 |
| 76 | D | 0 |
| 77 | B | <A> |
| 78 | 0 | 0 |
| 79 | 0 | <B> |
| 80 | A | <D> |
| 81 | B | <D> |
| 82 | A | <D> |
| 83 | B | <D> |
| 84 | 0 | A |
| 85 | 0 | 0 |
| 86 | 0 | B |
| 87 | A | <A> |
| 88 | <B> | <B> |
| 89 | A | 0 |
| 90 | 0 | 0 |
| 91 | 0 | 0 |
| 92 | 0 | 0 |
| 94 | 0 | 0 |
| 95 | 0 | 0 |
| 96 | B | 0 |

0 = 0% inhibition,
A = 1-25% inhibition,
B = 26-50% inhibition,
C = 51-75% inhibition,
D = 76-100% inhibition.
Values in brackets “< >” are understood to be activation %, rather than inhibition.

### Biological Example 3

### Protocol for Cell Proliferation Assay

Cell lines: Human tumor-derived cell lines were purchased from American Type Culture Collection. Human tumor-derived pancreatic cancer cell line MIA-PACA2 (having a KRAS G12C mutation) and Panc-1 (having a KRAS G12D mutation), and Human non-small cell lung cancer (NSCLC) cell line A549 (having a KRAS G12S mutation) were grown in complete DMEM-High Glucose. Human colon cancer cell line SW-620 (having a KRAS G12V mutation) was grown in RPMI. All cell lines were supplemented with penicillin (100 U/mL), streptomycin (100 μg/mL), and 10% heat-inactivated FBS at 37° C. in a humidified incubator with 5% CO₂.

Method: Cells were plated at 2000 to 5000 cells/well density in 96-wells plate and cultured overnight. Small molecules to be tested were added to the cells in the final concentration of 10 μM in the presence of 0.3% DMSO and 10% FBS, and incubated for 2-4 days at 37° C. in a humidified incubator with 5% CO₂. For IC₅₀ value determination, serial dilutions of compounds were added to cells under the same conditions.

Assay: At the end of the incubation period, cell viability was measured using the CellTiter 96® Aqueous One Solution Cell Proliferation Assay according to manufacturer specifications (Promega, Madison, Wis.). The percentage of small molecules inhibition of cellular proliferation was calculated from raw data.

Table 8 shows inhibition data for selected compounds tested in the MIA-PaCa-2 pancreatic cancer cell line using the cellular assay conditions described above.

TABLE 8
% Inhibition of cell proliferation at 10 μM in MIA-PaCa-2
pancreatic cancer cell line with select IC₅₀
(μM) values in the MIA PaCa-2 pancreatic cancer cell line

| | % inh cell proliferation | IC₅₀ (μM) cell proliferation |
|---|---|---|
| Compound | (10 μM) in MIA PaCa-2 | in MIA PaCa-2 |
| 1 | 0 | I |
| 2 | C | K |
| 3 | D | K |
| 4 | D | L |
| 5 | D | L |
| 6 | D | K |
| 7 | D | K |
| 8 | B | I |
| 9 | A | I |
| 10 | B | I |
| 11 | A | I |
| 12 | A | I |
| 13 | 0 | I |
| 14 | 0 | I |
| 15 | A | I |
| 16 | A | I |
| 17 | A | I |
| 18 | 0 | I |
| 19 | 0 | I |
| 20 | A | I |
| 21 | A | I |
| 22 | A | I |
| 23 | 0 | I |
| 24 | A | I |
| 25 | A | I |
| 26 | C | J |
| 27 | D | K |
| 30 | B | I |
| 31 | D | L |
| 32 | D | L |
| 33 | D | K |
| 34 | D | K |
| 35 | D | J |
| 36 | D | L |
| 37 | C | J |
| 38 | D | K |
| 39 | D | J |
| 40 | D | L |
| 41 | C | J |
| 42 | C | J |
| 43 | C | J |
| 44 | D | K |
| 45 | B | J |
| 46 | A | I |
| 47 | D | L |
| 48 | D | L |
| 49 | D | J |
| 50 | D | A |
| 51 | 0 | I |
| 52 | A | I |
| 53 | D | J |
| 54 | D | L |
| 55 | D | L |
| 56 | A | I |
| 57 | C | J |
| 58 | D | J |
| 59 | C | J |
| 60 | A | I |
| 61 | D | L |
| 62 | D | L |
| 63 | D | L |
| 64 | D | J |
| 65 | D | J |
| 66 | D | L |
| 67 | D | J |
| 68 | D | K |
| 69 | D | J |
| 70 | D | J |
| 71 | D | J |
| 72 | C | J |
| 73 | D | K |
| 74 | D | L |
| 75 | D | J |
| 76 | D | L |
| 77 | D | J |
| 78 | C | J |
| 79 | D | K |
| 80 | D | K |
| 81 | D | K |
| 82 | D | K |
| 83 | D | J |
| 84 | C | J |
| 85 | C | J |
| 86 | C | J |
| 87 | D | J |
| 88 | B | I |
| 89 | B | I |
| 90 | A | I |
| 91 | 0 | I |
| 92 | 0 | I |
| 94 | C | J |
| 95 | 0 | I |
| 96 | A | I |
| AMG510 (sotorasib) | N/T | L |

0 = 0% inhibition,
A = 1-25% inhibition,
B = 26-50% inhibition,
C = 51-75% inhibition,
D = 76-100% inhibition;
I = >10 μM IC₅₀,
J = 5-10 μM IC₅₀,
K = 1-4.99 μM IC₅₀,
L = < 1 μM IC₅₀;
N/T-Not tested

Table 9 shows inhibition data (IC50 (μM) values-micromolar units) for selected compounds tested in the MIA-PaCa-2 pancreatic cancer cell line, the PANC-1 pancreatic cancer cell line, the A-549 NSCLC cell line, and the SW-620 colorectal cancer cell line using the cellular assay conditions described above.

TABLE 9
% Inhibition of cell proliferation-IC₅₀ (μM) values in the MIA-PaCa-2
pancreatic cancer cell line, the PANC-1 pancreatic cancer cell line, the
A-549 NSCLC cell line, and the SW-620 colorectal cancer cell line

| | IC₅₀ (μM) cell | IC₅₀ (μM) cell | IC₅₀ (μM) cell | IC₅₀ (μM) cell |
|---|---|---|---|---|
| | proliferation in | proliferation in | proliferation in | proliferation in |
| Compound | MIA PaCa-2 | PANC-1 | A549 | SW-620 |
| 4 | M | M | L | L |
| 38 | K | K | K | K |
| 63 | M | L | L | M |
| 87 | I | M | N/T | M |
| AMG510 (sotorasib) | M | I | I | I |

I = >3 μM IC₅₀,
J = 1-3 μM IC₅₀,
K = <1-0.75 μM IC₅₀,
L = <0.75-0.40 μM IC₅₀,
M = <0.40 μM IC₅₀;
N/T-Not tested.

### EXEMPLARY EMBODIMENTS

Embodiment 1. A compound of Formula IA:

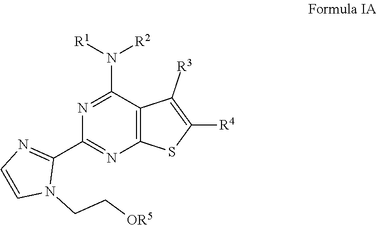

or a pharmaceutically acceptable derivative thereof, wherein:

—NR¹R² is

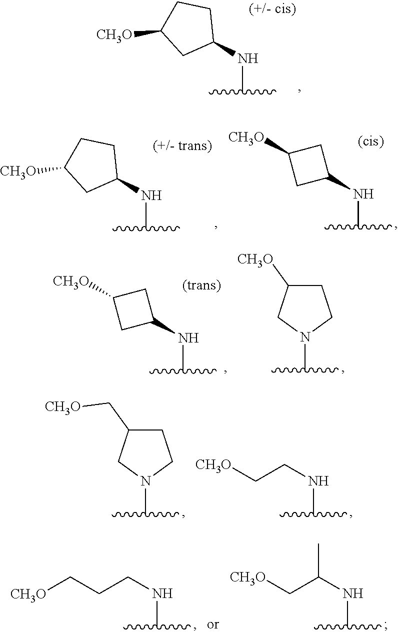

R³ is hydrogen, —CH₃, —CF₃, or phenyl;

R⁴ is hydrogen,

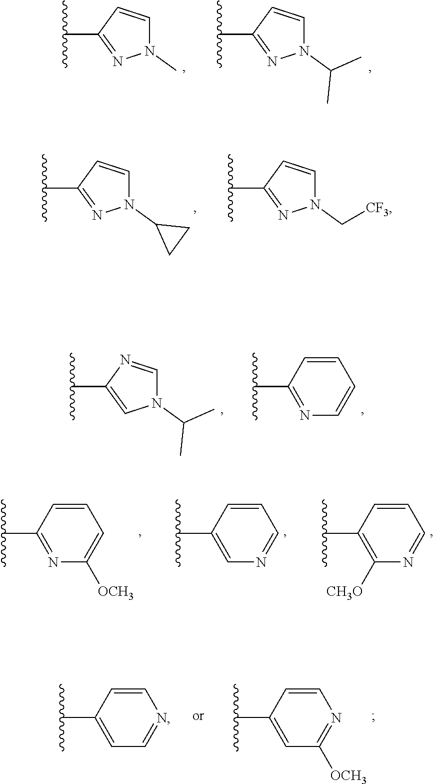

and

R⁵ is —CH₃ or —CH₂CF₃.

Embodiment 2. The compound or pharmaceutically acceptable derivative of embodiment 1, wherein —NR¹R² is:

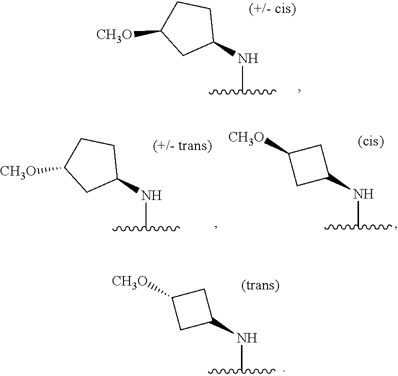

Embodiment 3. The compound or pharmaceutically acceptable derivative of embodiment 1 or 2, wherein R³ is hydrogen.

Embodiment 4. The compound or pharmaceutically acceptable derivative of embodiment 1 or 2, wherein R³ is —CH₃.

Embodiment 5. The compound or pharmaceutically acceptable derivative of embodiment 1 or 2, wherein R³ is phenyl.

Embodiment 6. The compound or pharmaceutically acceptable derivative of embodiment 1, wherein:

—NR¹R² is

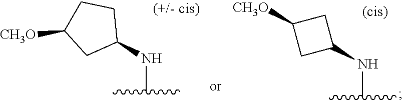

R³ is hydrogen or phenyl;

R⁴ is hydrogen or

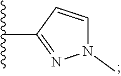 [SMILES: *c1ccn(C)n1]

and

R⁵ is —CH₃.

Embodiment 7. The compound or pharmaceutically acceptable derivative of embodiment 6, wherein R³ is hydrogen.

Embodiment 8. The compound or pharmaceutically acceptable derivative of embodiment 6, wherein R³ is phenyl.

Embodiment 9. The compound or pharmaceutically acceptable derivative of any one of embodiments 1-8, wherein R⁴ is hydrogen.

Embodiment 10. The compound or pharmaceutically acceptable derivative of any one of embodiments 1-8, wherein R⁴ is:

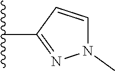 [SMILES: *c1ccn(C)n1]

Embodiment 11. The compound or pharmaceutically acceptable derivative of any one of embodiments 1-10, wherein the compound of Formula IA is:

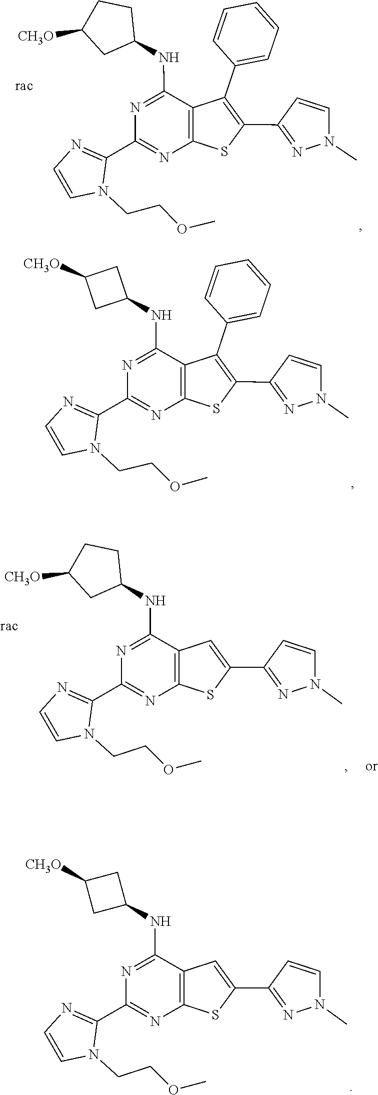

Embodiment 12. A compound of Formula IB:

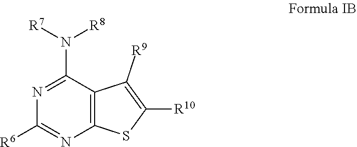

or a pharmaceutically acceptable derivative thereof, wherein:

R⁶ is

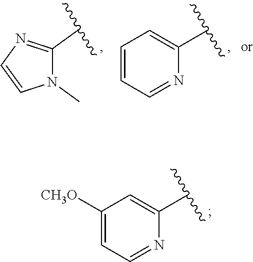

—NR⁷R⁸ is

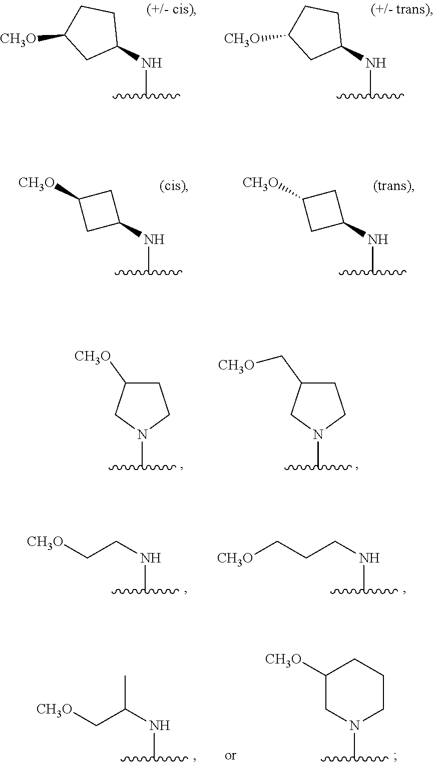

R⁹ is hydrogen, —CH₃, —CF₃, or phenyl; and

R¹⁰ is

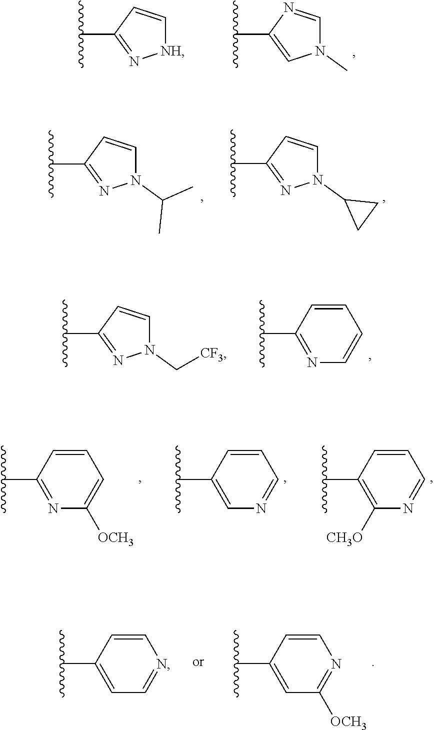

Embodiment 13. The compound or pharmaceutically acceptable derivative of embodiment 12, wherein R⁶ is:

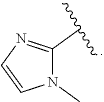 [SMILES: *c1nccn1C]

Embodiment 14. The compound or pharmaceutically acceptable derivative of embodiment 12, wherein R⁶ is:

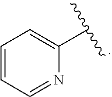 [SMILES: *c1ccccn1]

Embodiment 15. The compound or pharmaceutically acceptable derivative of embodiment 12, wherein R⁶ is:

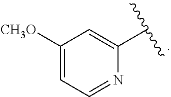

Embodiment 16. The compound or pharmaceutically acceptable derivative of any one of embodiments 12-15, wherein —NR⁷R⁸ is:

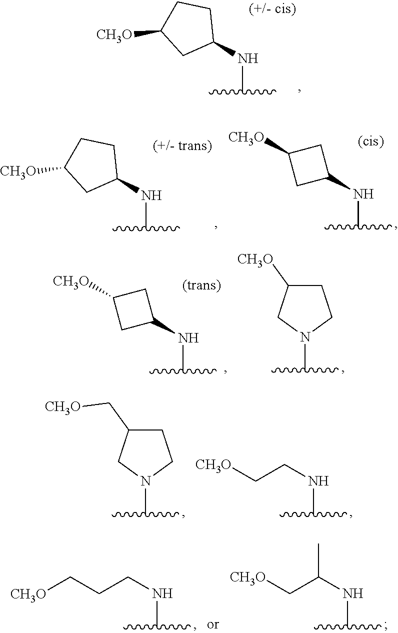

Embodiment 17. The compound or pharmaceutically acceptable derivative of any one of embodiments 12-15, wherein —NR⁷R⁸ is:

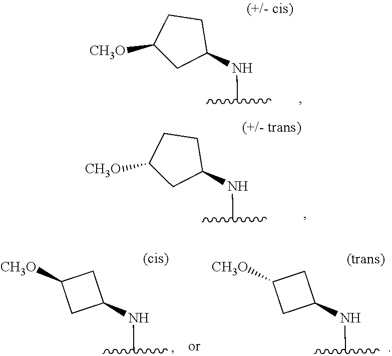

Embodiment 18. The compound or pharmaceutically acceptable derivative of any one of embodiments 12-15, wherein —NR⁷R⁸ is:

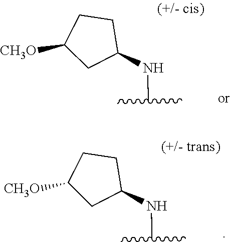

Embodiment 19. The compound or pharmaceutically acceptable derivative of any one of embodiments 12-15, wherein —NR⁷R⁸ is:

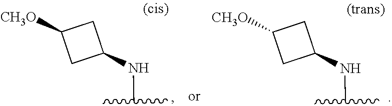

Embodiment 20. The compound or pharmaceutically acceptable derivative of any one of embodiments 12-15, wherein —NR⁷R⁸ is:

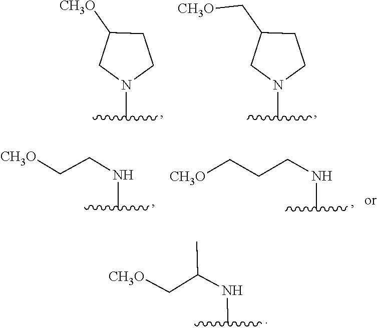

Embodiment 21. The compound or pharmaceutically acceptable derivative of any one of embodiments 12-15, wherein —NR⁷R⁸ is:

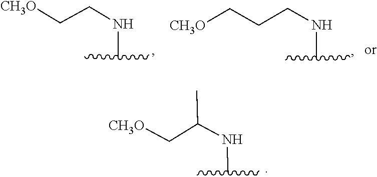

Embodiment 22. The compound or pharmaceutically acceptable derivative of any one of embodiments 12-15, wherein —NR⁷R⁸ is: CH₃O CH₃O CH₃O

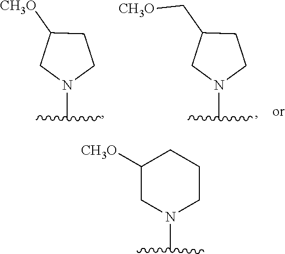

Embodiment 23. The compound or pharmaceutically acceptable derivative of any one of embodiments 12-15, wherein —NR⁷R⁸ is:

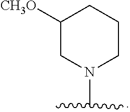

Embodiment 24. The compound or pharmaceutically acceptable derivative of any one of embodiments 12-23, wherein R⁹ is hydrogen.

Embodiment 25. The compound or pharmaceutically acceptable derivative of any one of embodiments 12-23, wherein R⁹ is —CH₃.

Embodiment 26. The compound or pharmaceutically acceptable derivative of any one of embodiments 12-23, wherein R⁹ is phenyl.

Embodiment 27. The compound or pharmaceutically acceptable derivative of any one of embodiments 12-26, wherein R¹⁰ is:

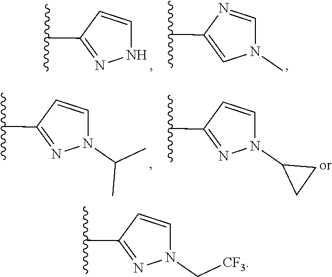

Embodiment 28. The compound or pharmaceutically acceptable derivative of any one of embodiments 12-26, wherein R¹⁰ is:

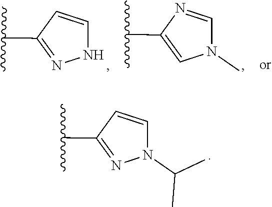

Embodiment 29. The compound or pharmaceutically acceptable derivative of any one of embodiments 12-26, wherein R¹⁰ is:

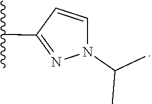 [SMILES: *c1ccn(C(C)C)n1]

Embodiment 30. The compound or pharmaceutically acceptable derivative of any one of embodiments 12-26 wherein R¹⁰ is:

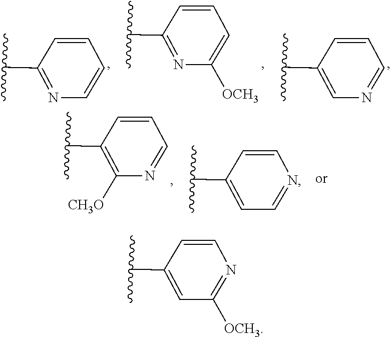

Embodiment 31. The compound or pharmaceutically acceptable derivative of any one of embodiments 12-30, wherein the compound of Formula IB is:

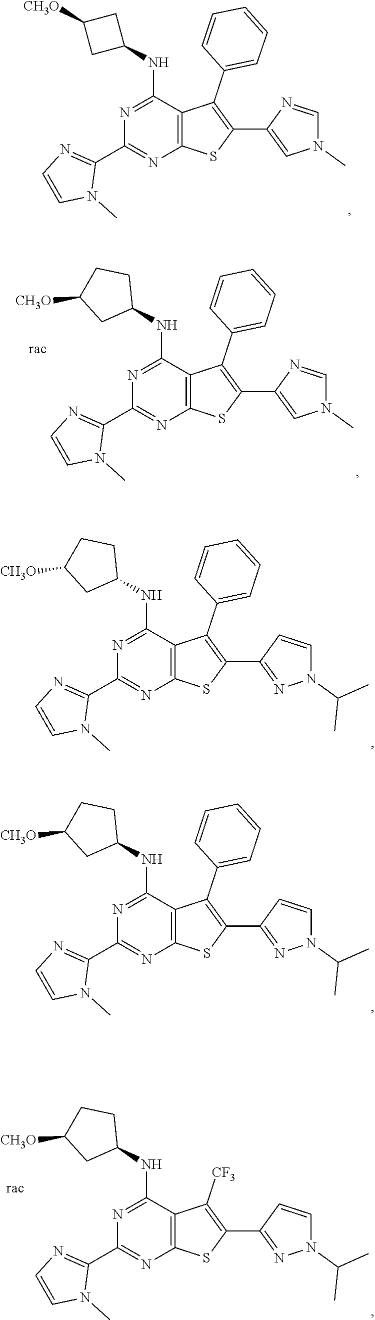

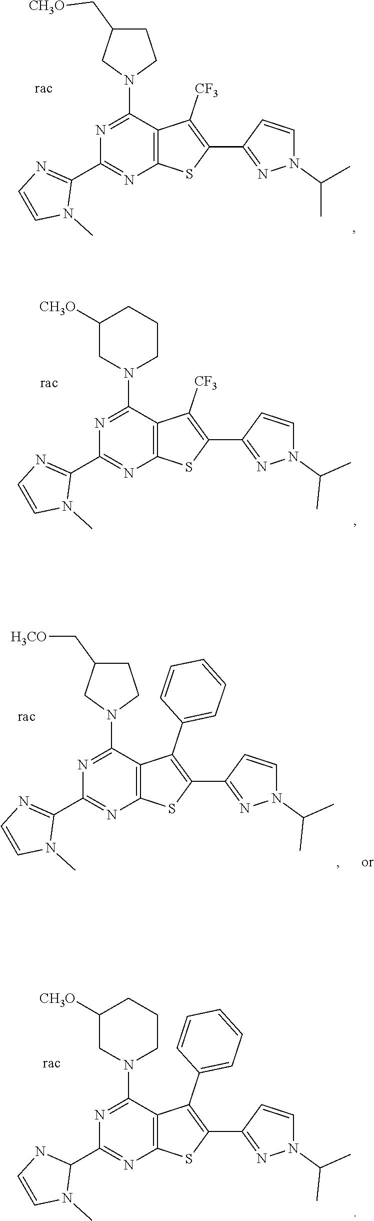

Embodiment 32. The compound or pharmaceutically acceptable derivative of any one of embodiments 12-31, wherein the compound of Formula IB is:

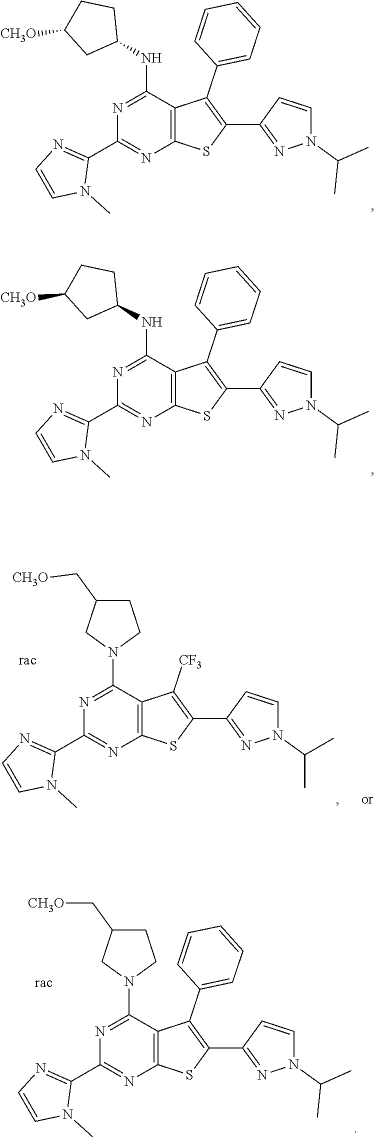

Embodiment 33. The compound or pharmaceutically acceptable derivative of any one of embodiments 12-32, wherein the compound of Formula IB is:

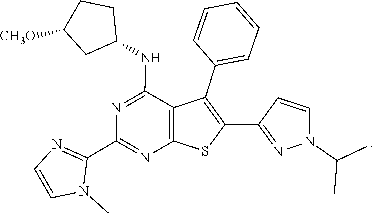

Embodiment 34. The compound or pharmaceutically acceptable derivative of embodiment 12, wherein:

R⁶ is

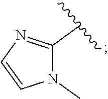 [SMILES: *c1nccn1C]

—NR⁷R⁸ is

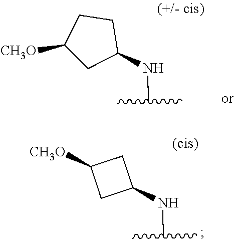

R⁹ is phenyl; and

R¹⁰ is

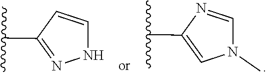

Embodiment 35. The compound or pharmaceutically acceptable derivative of any one of embodiments 12-30 or embodiment 34, wherein —NR⁷R⁸ is:

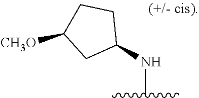

Embodiment 36. The compound or pharmaceutically acceptable derivative of any one of embodiments 12-30 or embodiment 34, wherein —NR⁷R⁸ is:

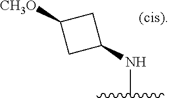

Embodiment 37. The compound or pharmaceutically acceptable derivative of any one of embodiments 12-30 or embodiments 34-36, wherein R¹⁰ is:

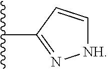 [SMILES: *c1cc[nH]n1]

Embodiment 38. The compound or pharmaceutically acceptable derivative of any one of embodiments 12-30 or embodiments 34-36, wherein R¹⁰ is:

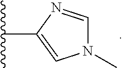 [SMILES: *c1cn(C)cn1]

Embodiment 39. The compound or pharmaceutically acceptable derivative of any one of embodiments 12-31 or embodiments 34-38, wherein the compound of Formula IB is:

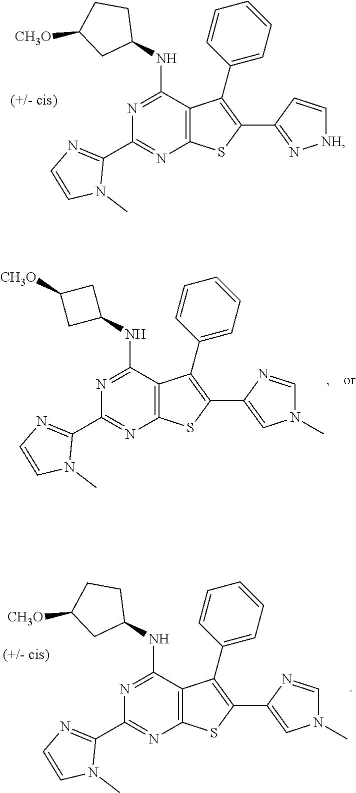

Embodiment 40. A compound of Formula IC:

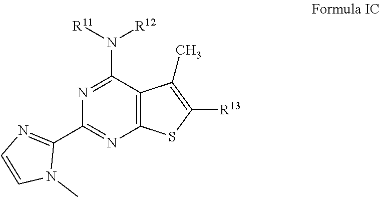

or a pharmaceutically acceptable derivative thereof, wherein:

—NR¹¹R¹² is

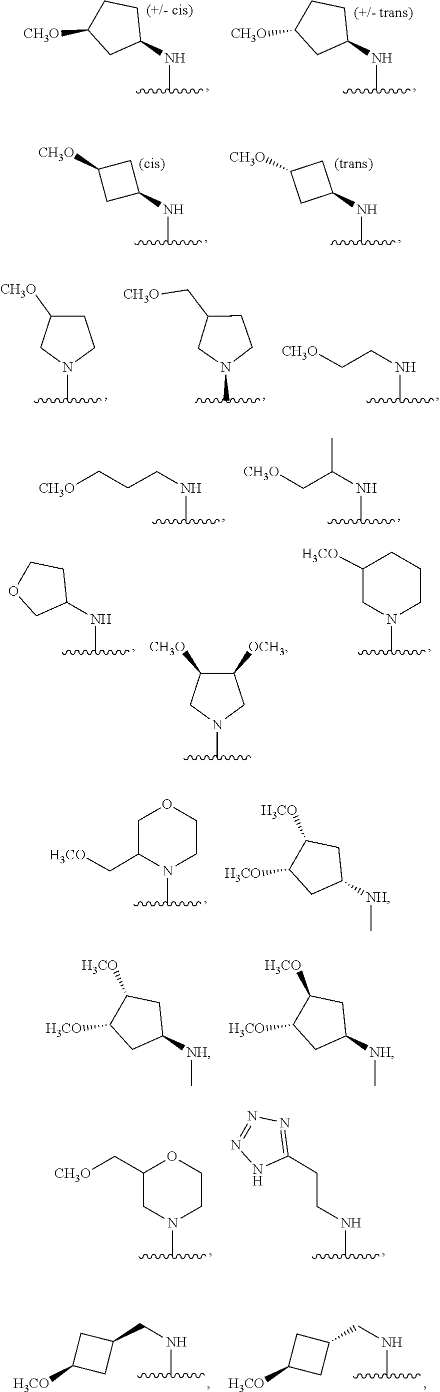

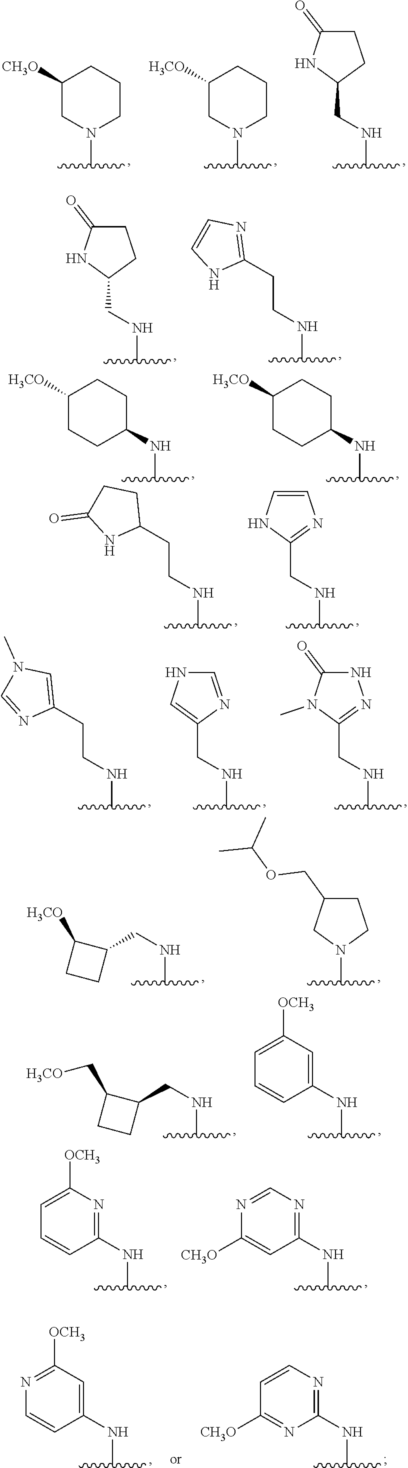

and

R¹³ is

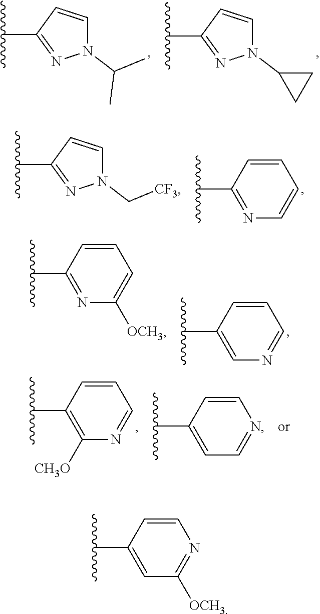

Embodiment 41. The compound or pharmaceutically acceptable derivative of embodiment 40, wherein R¹³ is:

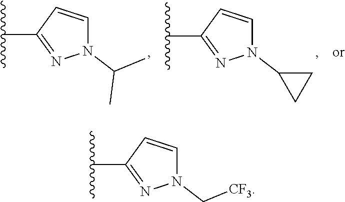

Embodiment 42. The compound or pharmaceutically acceptable derivative of embodiment 40, wherein R¹³ is:

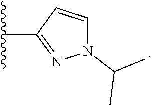 [SMILES: *c1ccn(C(C)C)n1]

Embodiment 43. The compound or pharmaceutically acceptable derivative of embodiment 40, wherein R¹³ is:

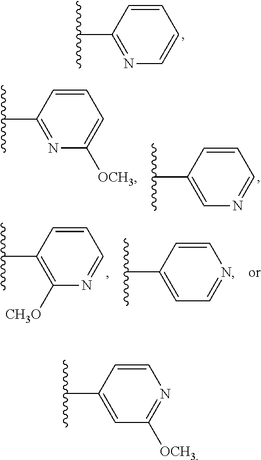

Embodiment 44. The compound or pharmaceutically acceptable derivative of any one of embodiments 40-43, wherein —NR¹¹R¹² is:

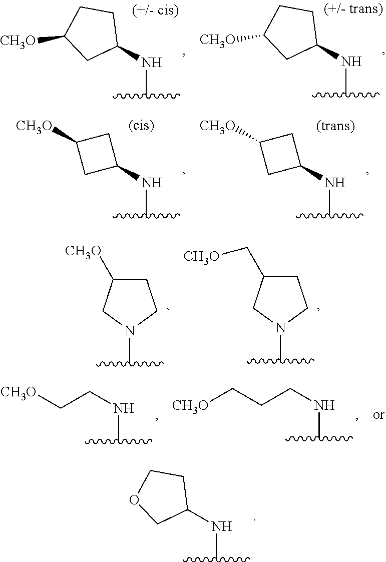

Embodiment 45. The compound or pharmaceutically acceptable derivative of any one of embodiments 40-44, wherein —NR¹¹R¹² is:

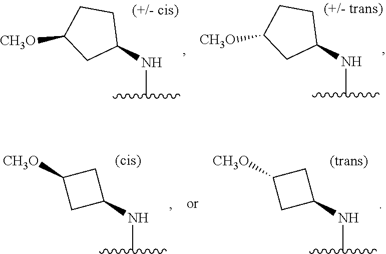

Embodiment 46. The compound or pharmaceutically acceptable derivative of any one of embodiments 40-44, wherein —NR¹¹R¹² is:

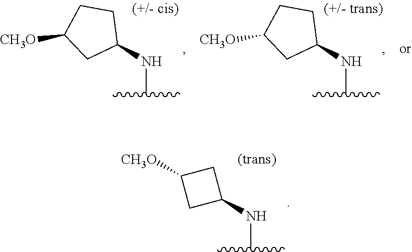

Embodiment 47. The compound or pharmaceutically acceptable derivative of any one of embodiments 40-44, wherein —NR¹¹R¹² is:

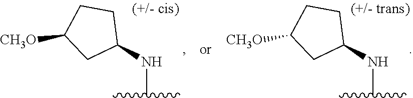

Embodiment 48. The compound or pharmaceutically acceptable derivative of any one of embodiments 40-44, wherein —NR¹¹R¹² is:

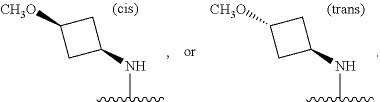

Embodiment 50. The compound or pharmaceutically acceptable derivative of any one of embodiments 40-44, wherein —NR¹¹R¹² is:

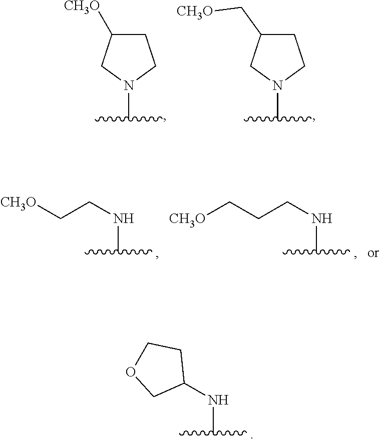

Embodiment 51. The compound or pharmaceutically acceptable derivative of any one of embodiments 40-43, wherein —NR¹¹R¹² is:

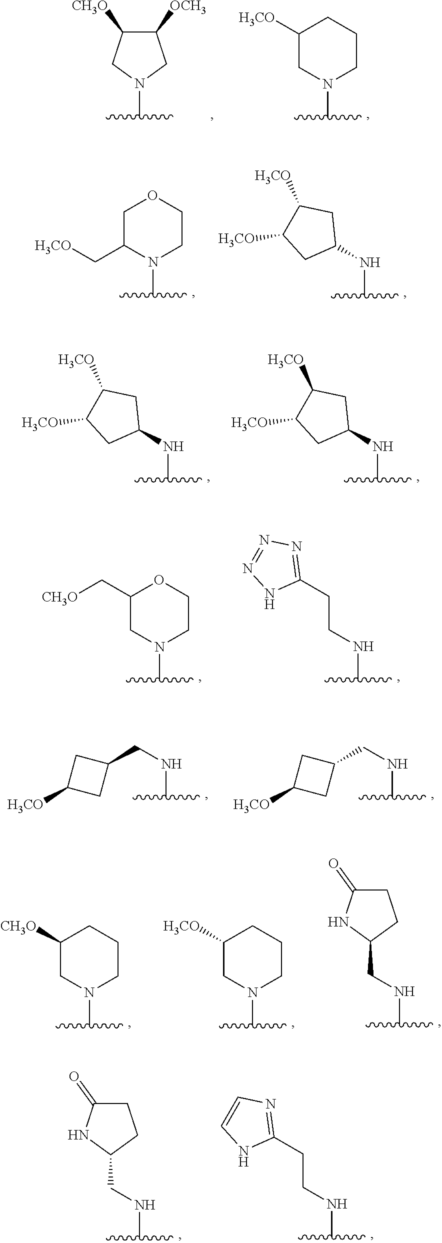

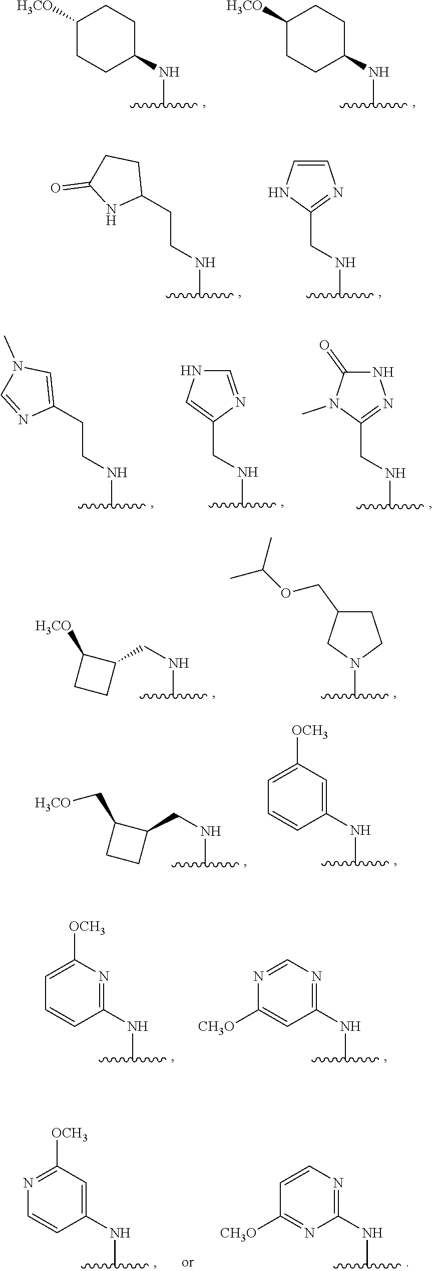

Embodiment 52. The compound or pharmaceutically acceptable derivative of any one of embodiments 40-43 or embodiment 51, wherein —NR¹¹R¹² is:

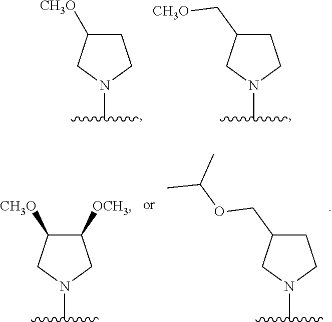

Embodiment 53. The compound or pharmaceutically acceptable derivative of any one of embodiments 40-43 or embodiment 51, wherein —NR¹¹R¹² is:

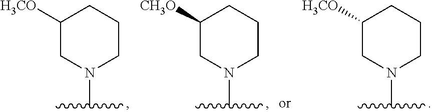

Embodiment 54. The compound or pharmaceutically acceptable derivative of any one of embodiments 40-43 or embodiment 51, wherein —NR¹¹R¹² is:

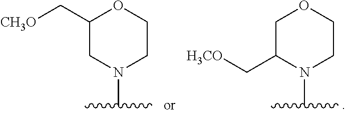

Embodiment 55. The compound or pharmaceutically acceptable derivative of any one of embodiments 40-43 or embodiment 51, wherein —NR¹¹R¹² is:

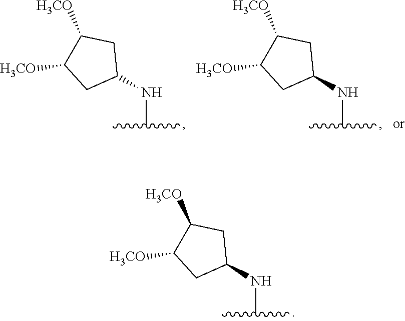

Embodiment 56. The compound or pharmaceutically acceptable derivative of any one of embodiments 40-43 or embodiment 51, wherein —NR¹¹R¹² is:

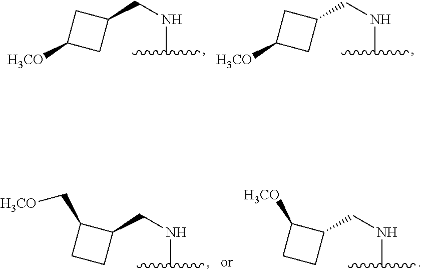 [SMILES: *NC[C@H]1C[C@@H](OC)C1 ; *NC[C@H]1C[C@H](OC)C1 ; *NC[C@H]1CC[C@H]1COC ; *NC[C@@H]1CC[C@H]1OC]

Embodiment 57. The compound or pharmaceutically acceptable derivative of any one of embodiments 40-43 or embodiment 51, wherein —NR¹¹R¹² is:

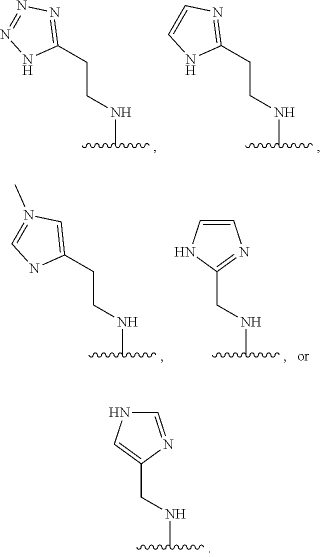

Embodiment 58. The compound or pharmaceutically acceptable derivative of any one of embodiments 40-43 or embodiment 51, wherein —NR¹¹R¹² is:

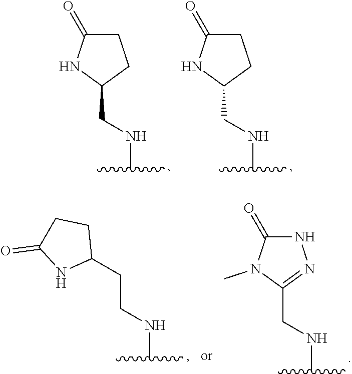

Embodiment 59. The compound or pharmaceutically acceptable derivative of any one of embodiments 40-43 or embodiment 51, wherein —NR¹¹R¹² is:

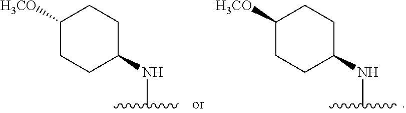 [SMILES: *N[C@H]1CC[C@H](OC)CC1 ; *N[C@H]1CC[C@@H](OC)CC1]

Embodiment 60. The compound or pharmaceutically acceptable derivative of any one of embodiments 40-43 or embodiment 51, wherein —NR¹¹R¹² is:

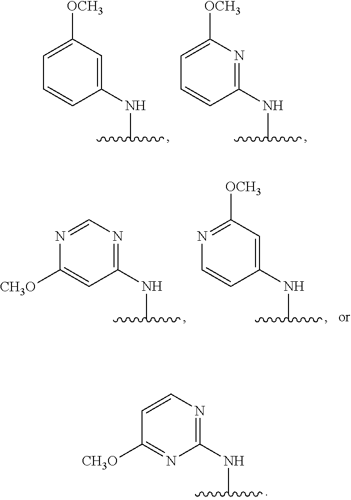

Embodiment 61. The compound or pharmaceutically acceptable derivative of any one of embodiments 40-60, wherein the compound of Formula IC is:

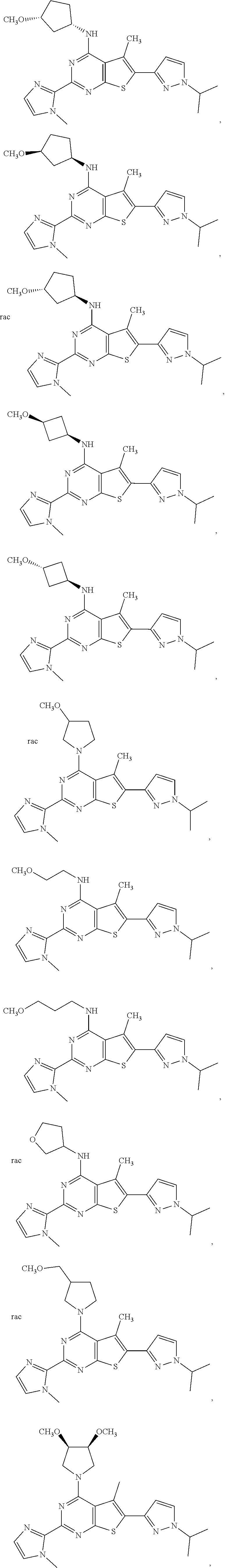

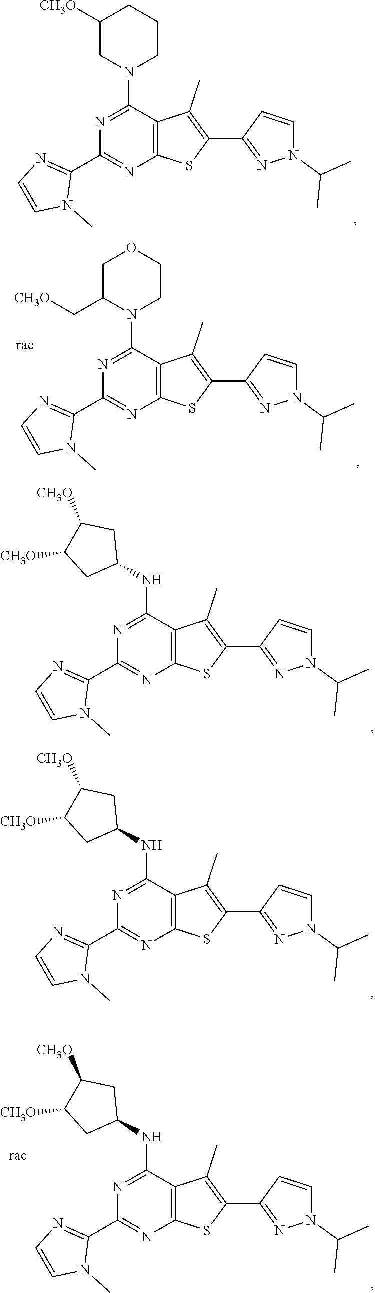

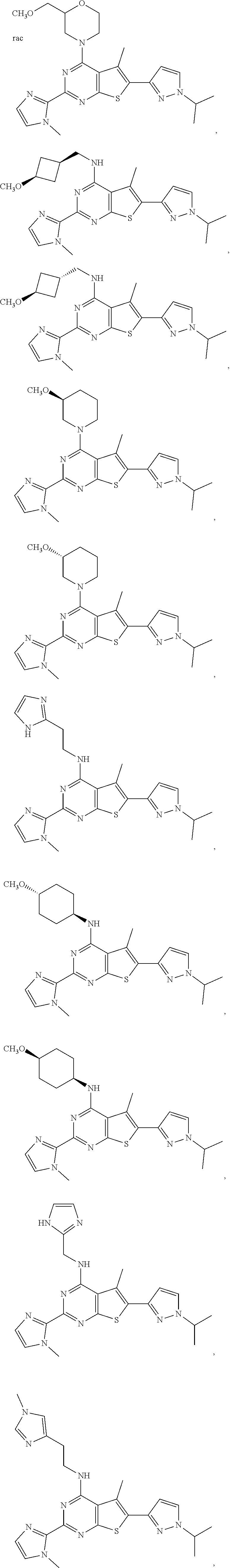

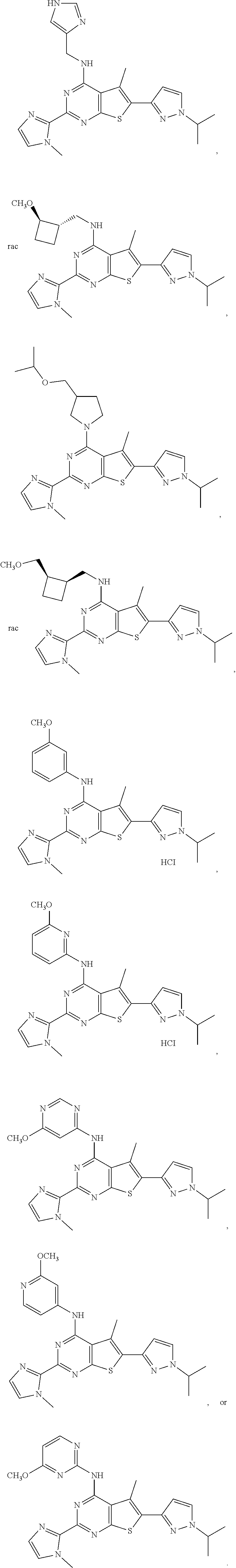

Embodiment 62. The compound or pharmaceutically acceptable derivative of any one of embodiments 40-61, wherein the compound of Formula 1C is:

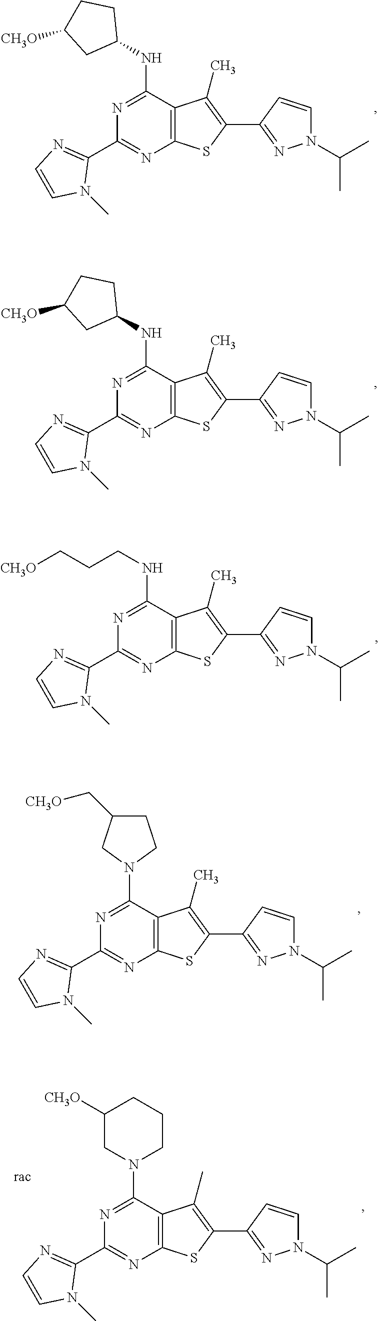

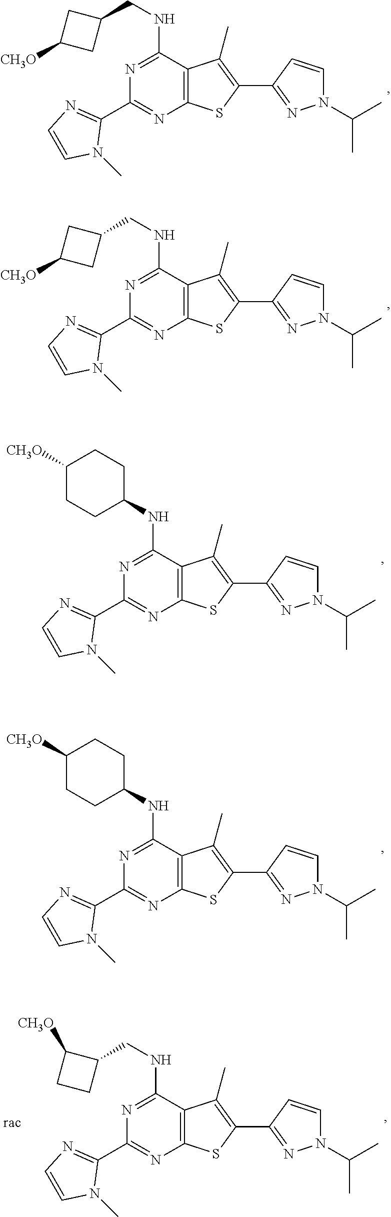

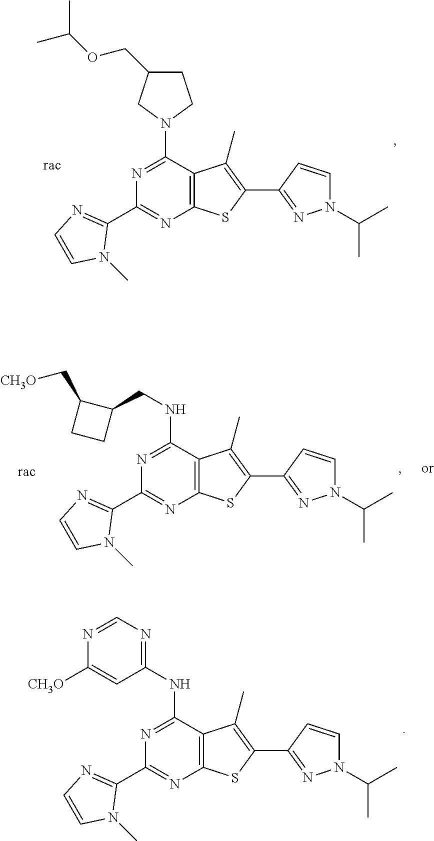

Embodiment 63. A compound of Formula ID:

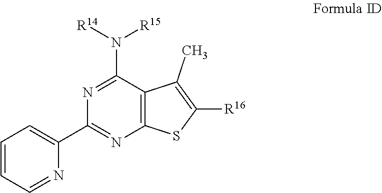

or a pharmaceutically acceptable derivative thereof, wherein:

—NR¹⁴R¹⁵ is

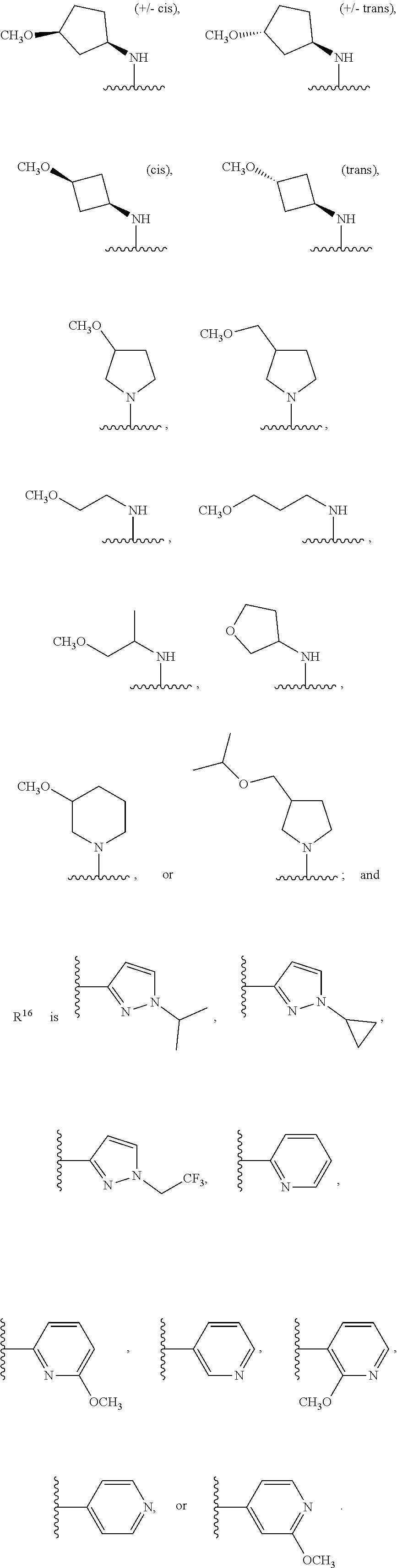

Embodiment 64. The compound of embodiment 63, wherein —NR¹⁴R¹⁵ is:

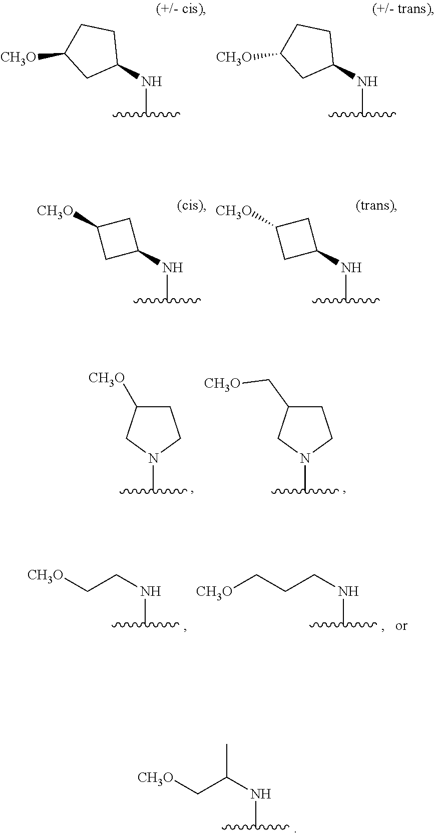

Embodiment 65. The compound or pharmaceutically acceptable derivative of embodiment 63, wherein —NR 4R¹⁵ is:

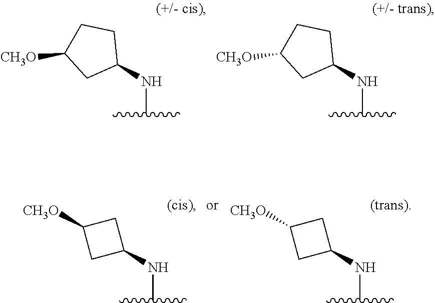

Embodiment 66. The compound or pharmaceutically acceptable derivative of embodiment 63, wherein —NR¹⁴R¹⁵ is:

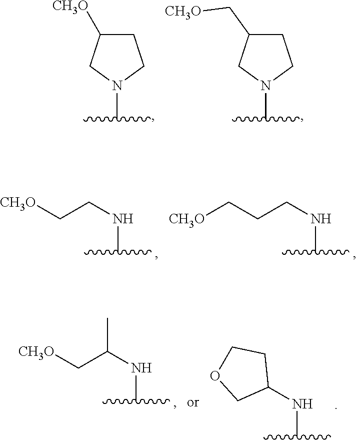

Embodiment 67. The compound of embodiment 63, wherein —NR¹⁴R¹⁵ is:

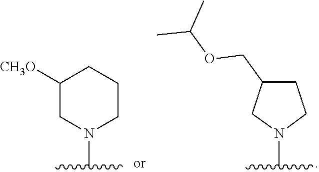

Embodiment 68. The compound or pharmaceutically acceptable derivative of any one of embodiments 63-67, wherein R¹⁶ is:

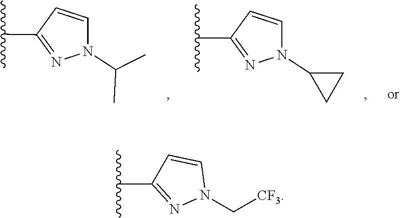

Embodiment 69. The compound or pharmaceutically acceptable derivative of any one of embodiments 63-67, wherein R¹⁶ is:

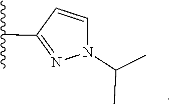 [SMILES: *c1ccn(C(C)C)n1]

Embodiment 70. The compound or pharmaceutically acceptable derivative of any one of embodiments 63-67, wherein R¹⁶ is:

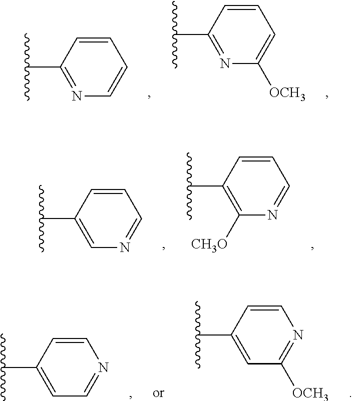

Embodiment 71. The compound or pharmaceutically acceptable derivative of embodiment 63, wherein:

—NR¹⁴R¹⁵ is

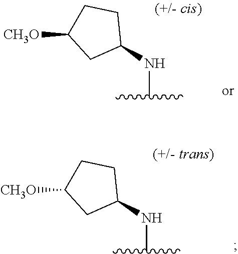

and

R¹⁶ is

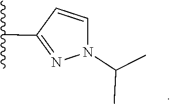 [SMILES: *c1ccn(C(C)C)n1]

Embodiment 72. The compound or pharmaceutically acceptable derivative of any one of embodiments 63-71, wherein —NR¹⁴R¹⁵ is

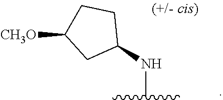

Embodiment 73. The compound or pharmaceutically acceptable derivative of any one of embodiments 64-72, wherein the compound of Formula ID is:

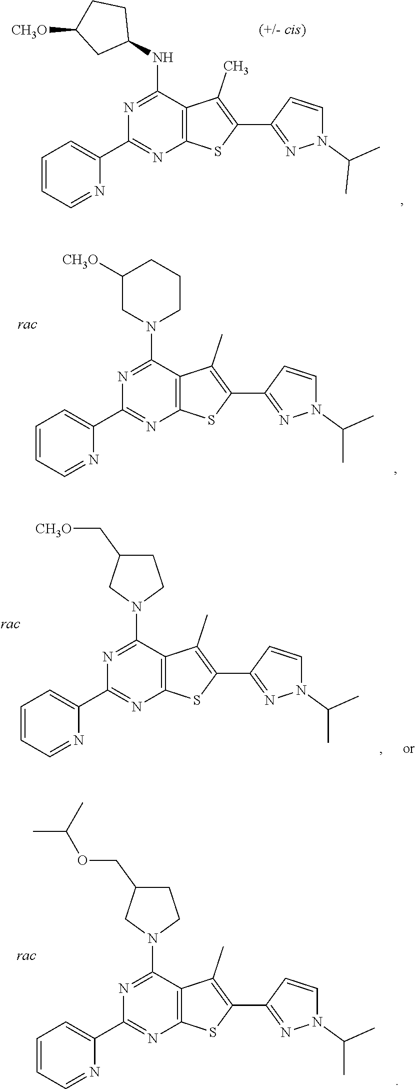

Embodiment 74. A compound of Formula IE:

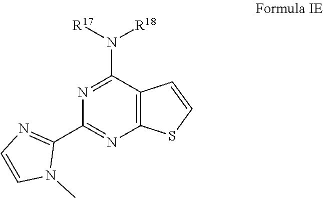

or a pharmaceutically acceptable derivative thereof, wherein:

—NR¹⁷R¹⁸ is

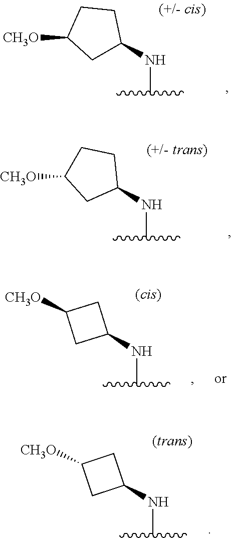

Embodiment 75. The compound or pharmaceutically acceptable derivative of embodiment 74, wherein —NR¹⁷R¹⁸ is:

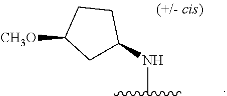

Embodiment 76. The compound or pharmaceutically acceptable derivative of embodiment 74, wherein —NR¹⁷R¹⁸ is:

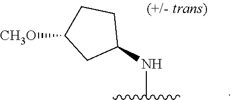

Embodiment 77. The compound or pharmaceutically acceptable derivative of embodiment 74, wherein —NR¹⁷R¹⁸ is:

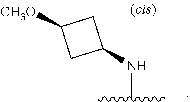

Embodiment 78. The compound or pharmaceutically acceptable derivative of embodiment 74, wherein —NR¹⁷R¹⁸ is:

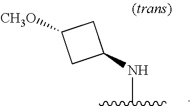

Embodiment 79. The compound or pharmaceutically acceptable derivative of embodiment 74, wherein the compound of Formula IE is:

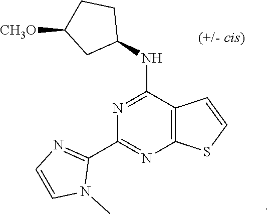

Embodiment 80. A compound of Formula IF:

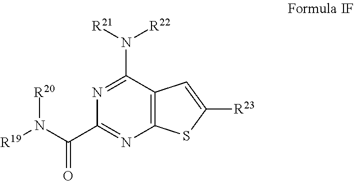

or a pharmaceutically acceptable derivative thereof, wherein:

—NR¹⁹R²⁰ is —NH₂,

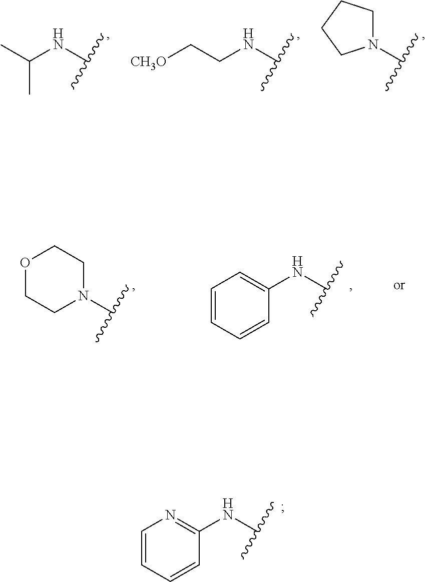

—NR²¹R²² is

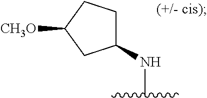

and

R²³ is hydrogen or

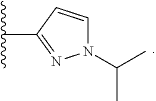 [SMILES: *c1ccn(C(C)C)n1]

Embodiment 81. The compound or pharmaceutically acceptable derivative of embodiment 80, wherein R²³ is hydrogen.

Embodiment 82. The compound or pharmaceutically acceptable derivative of embodiment 80, wherein R²³ is:

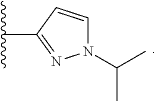 [SMILES: *c1ccn(C(C)C)n1]

Embodiment 83. The compound or pharmaceutically acceptable derivative of any one of embodiments 80-82, wherein the compound of Formula IF is:

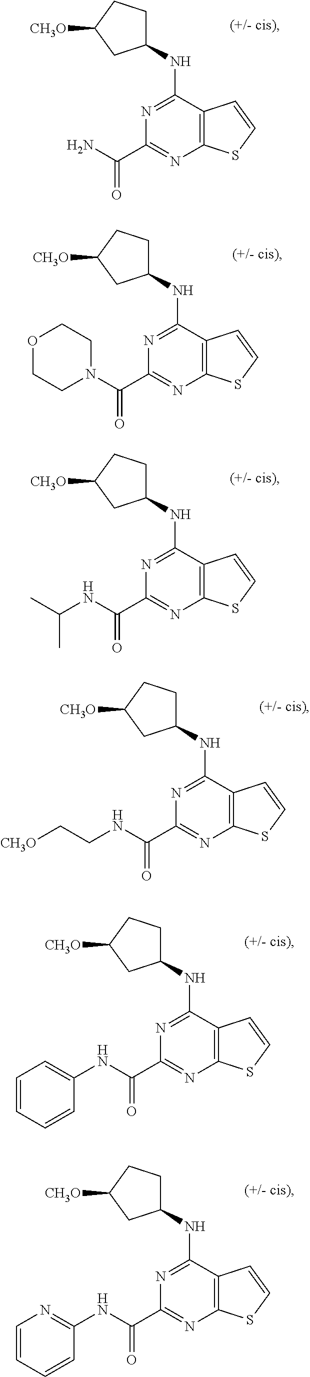

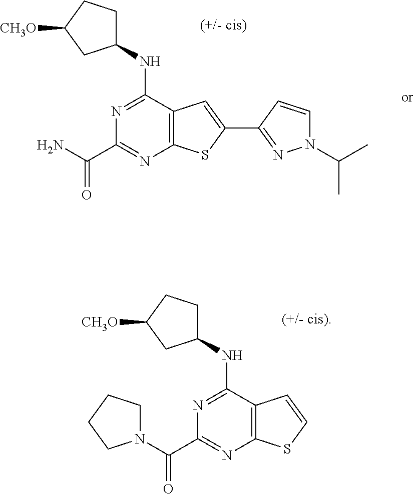

Embodiment 84. A compound of Formula IIA:

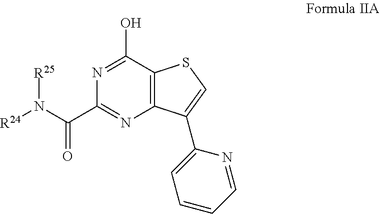

or a pharmaceutically acceptable derivative thereof, wherein:

—NR²⁴R⁵ is —NH₂,

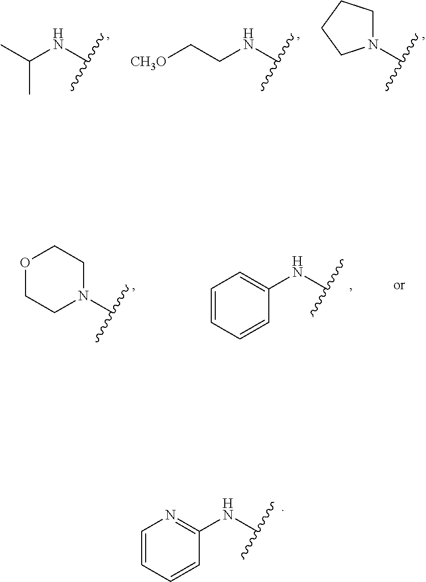

Embodiment 85. The compound or pharmaceutically acceptable derivative of embodiment 84, wherein the compound of Formula IIA is:

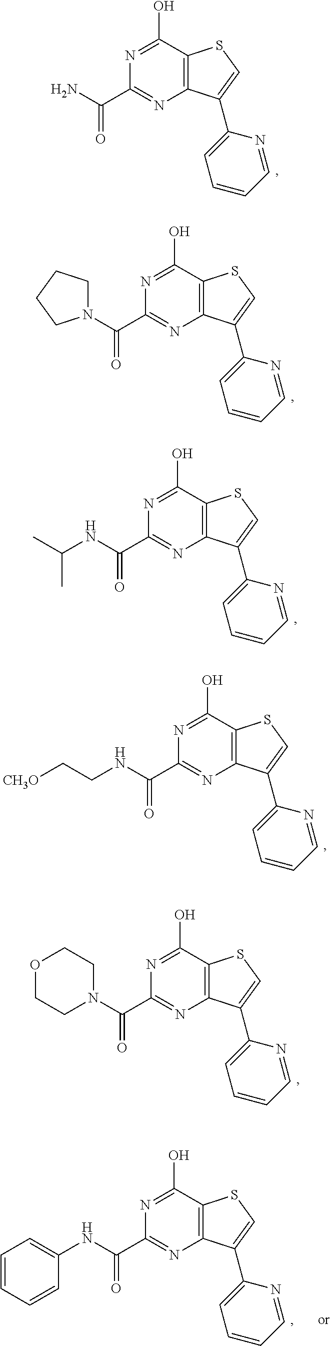

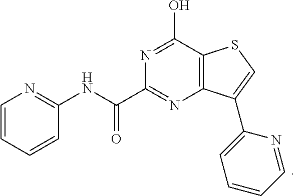 [SMILES: O=C(Nc1ccccn1)c1nc(O)c2scc(-c3ccccn3)c2n1]

Embodiment 86. A compound of Formula IIB:

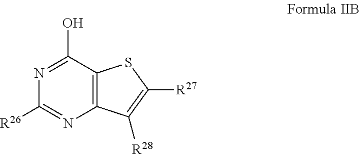

or a pharmaceutically acceptable derivative thereof, wherein:

R²⁶ is

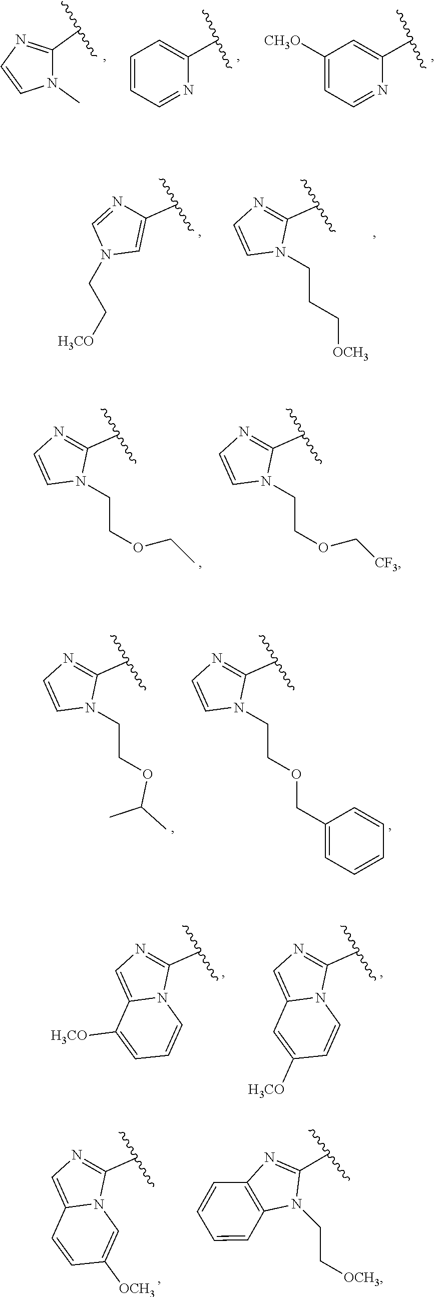

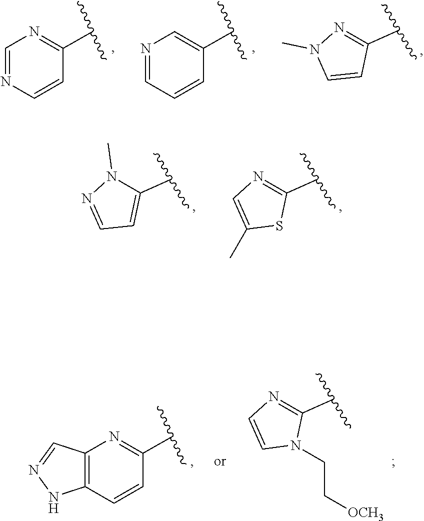

R²⁷ is hydrogen, —CH₃, or —CF₃; and

R²⁸ is

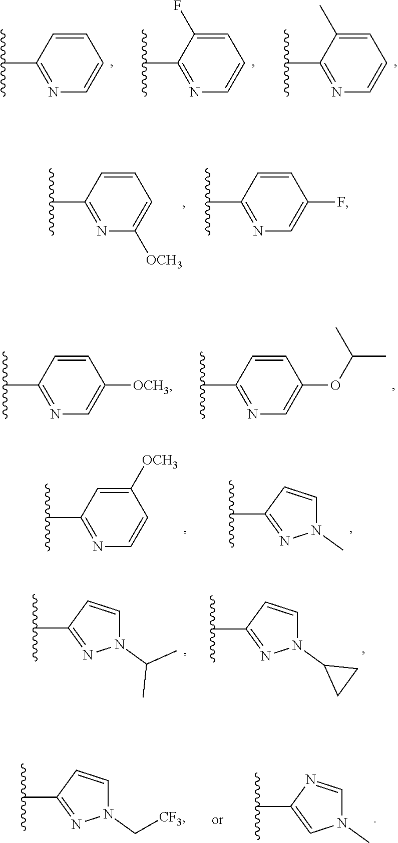

Embodiment 87. The compound or pharmaceutically acceptable derivative of embodiment 86, wherein R²⁶ is:

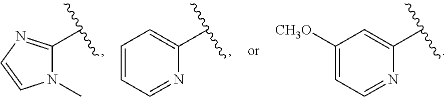

Embodiment 88. The compound or pharmaceutically acceptable derivative of embodiment 86, wherein R²⁶ is:

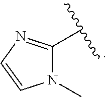 [SMILES: *c1nccn1C]

Embodiment 89. The compound or pharmaceutically acceptable derivative of embodiment 86, wherein R²⁶ is:

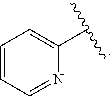 [SMILES: *c1ccccn1]

Embodiment 90. The compound or pharmaceutically acceptable derivative of embodiment 86, wherein R²⁶ is:

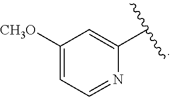

Embodiment 91. The compound or pharmaceutically acceptable derivative of embodiment 86, wherein R²⁶ is:

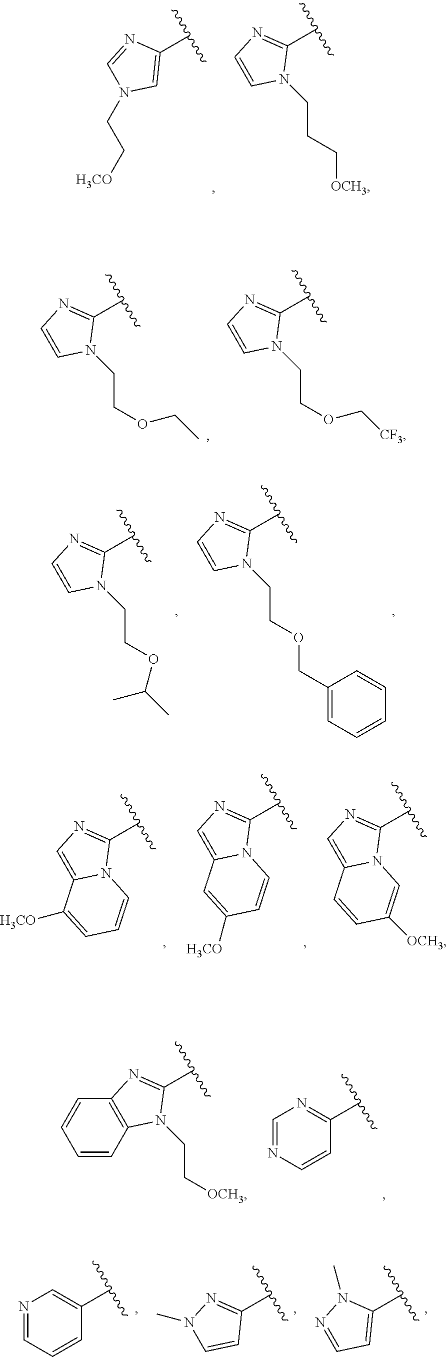

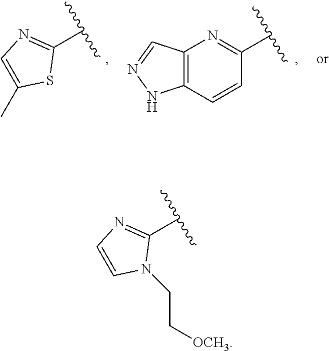

Embodiment 92. The compound or pharmaceutically acceptable derivative of embodiment 86, wherein R²⁶ is:

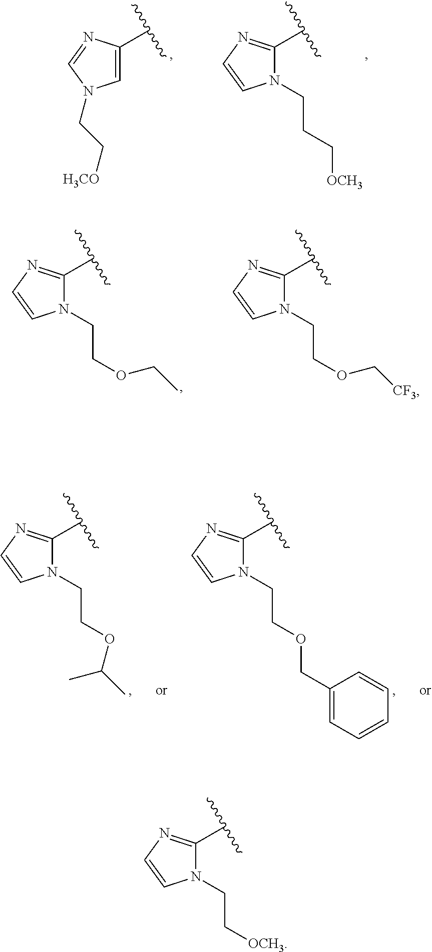

Embodiment 93. The compound or pharmaceutically acceptable derivative of embodiment 86, wherein R²⁶ is:

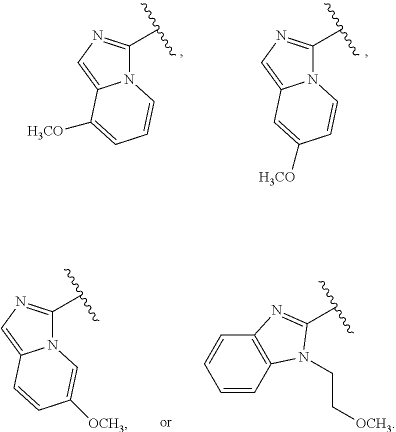 [SMILES: *c1ncc2c(OC)cccn12 ; *c1ncc2cc(OC)ccn12 ; *c1ncc2ccc(OC)cn12 ; "or" ; *c1nc2ccccc2n1CCOC]

Embodiment 94. The compound or pharmaceutically acceptable derivative of embodiment 86, wherein R²⁶ is:

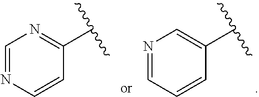 [SMILES: *c1ccncn1 ; *c1cccnc1]

Embodiment 95. The compound or pharmaceutically acceptable derivative of embodiment 86, wherein R²⁶ is:

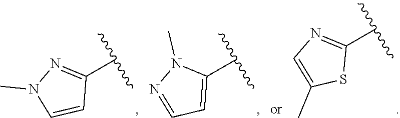

Embodiment 96. The compound or pharmaceutically acceptable derivative of embodiment 86, wherein R²⁶ is:

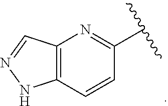 [SMILES: *c1ccc2[nH]ncc2n1]

Embodiment 97. The compound or pharmaceutically acceptable derivative of any one of embodiments 86-96, wherein R²⁷ is hydrogen.

Embodiment 98. The compound or pharmaceutically acceptable derivative of any one of embodiments 86-96, wherein R²⁷ is —CH₃.

Embodiment 99. The compound or pharmaceutically acceptable derivative of any one of embodiments 86-96, wherein R²⁷ is —CF₃.

Embodiment 100. The compound or pharmaceutically acceptable derivative of any one of any one of embodiments 86-98, wherein R²⁸ is:

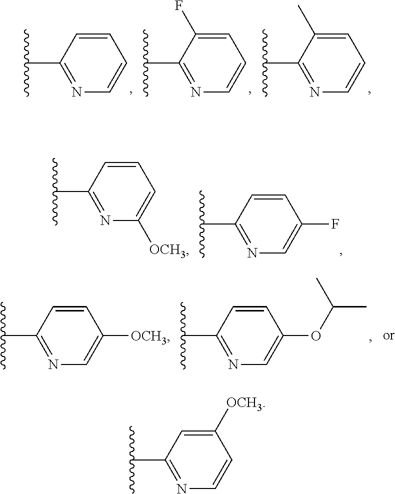

Embodiment 101. The compound or pharmaceutically acceptable derivative of any one of embodiments 86-98, wherein R²⁸ is:

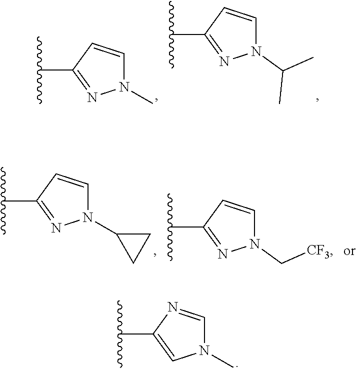

Embodiment 102. The compound or pharmaceutically acceptable derivative of embodiment 86, wherein:

R²⁶ is

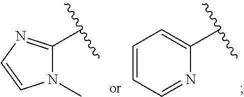

R²⁷ is hydrogen; and

R²⁸ is

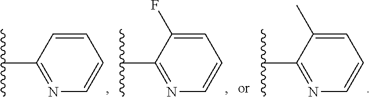

Embodiment 103. The compound or pharmaceutically acceptable derivative of embodiment 102, wherein R²⁶ is:

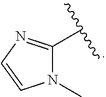 [SMILES: *c1nccn1C]

Embodiment 104. The compound or pharmaceutically acceptable derivative of embodiment 102, wherein R²⁶ is:

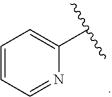 [SMILES: *c1ccccn1]

Embodiment 105. The compound or pharmaceutically acceptable derivative of any one of embodiments 102-104, wherein R²⁸ is:

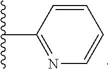 [SMILES: *c1ccccn1]

Embodiment 106. The compound or pharmaceutically acceptable derivative of any one of embodiments 102-104, wherein R²⁸ is:

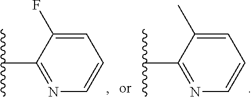

Embodiment 107. The compound or pharmaceutically acceptable derivative of any one of embodiments 86-106, with the proviso that when:

R²⁶ is

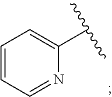 [SMILES: *c1ccccn1]

and

R²⁷ is hydrogen; then

R²⁸ is not

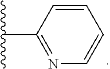 [SMILES: *c1ccccn1]

Embodiment 108. The compound or pharmaceutically acceptable derivative of any one of embodiments 86-107, wherein the compound of Formula IIB is:

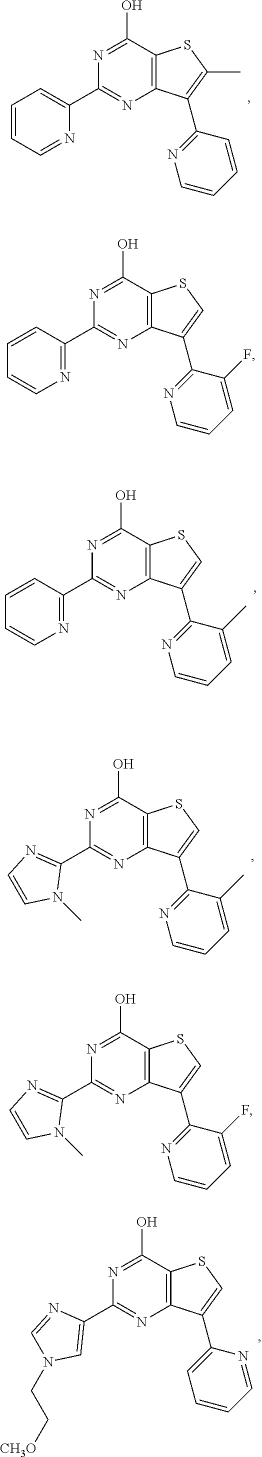

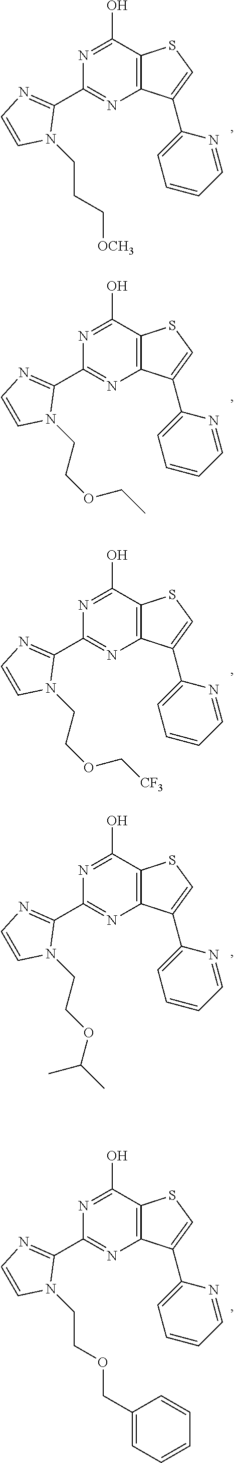

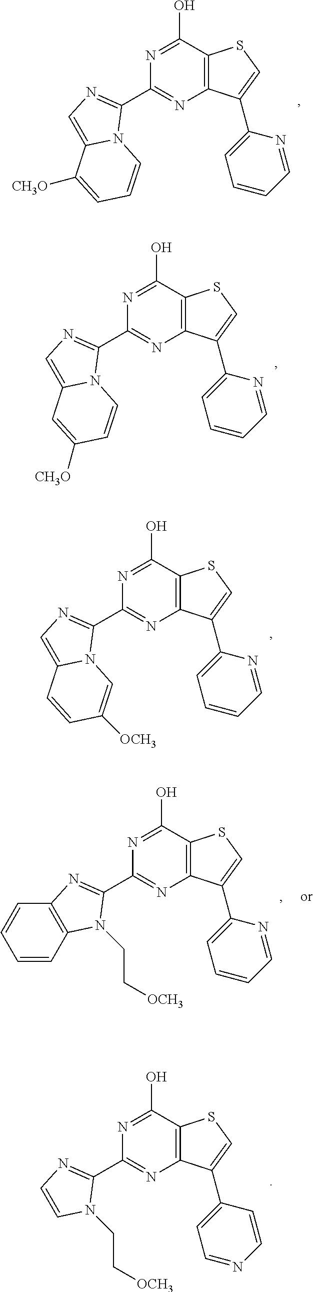

Embodiment 109. The compound or pharmaceutically acceptable derivative of any one of embodiments 86-107, wherein the compound of Formula IIB is:

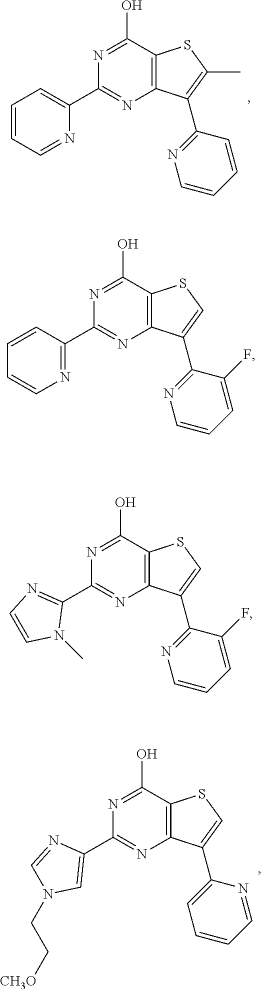

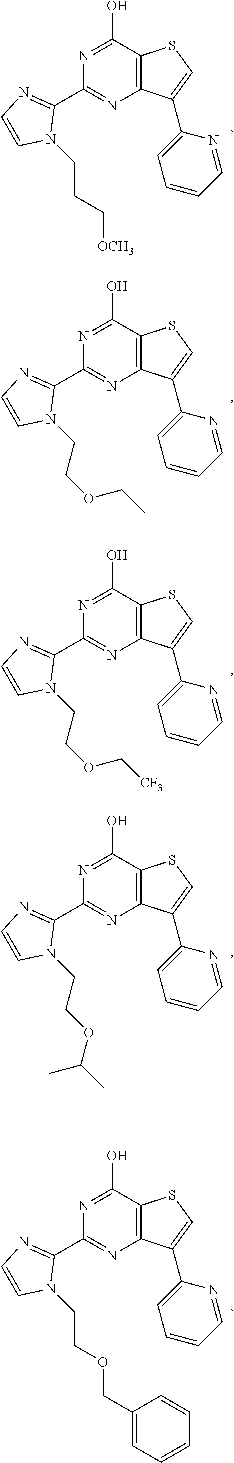

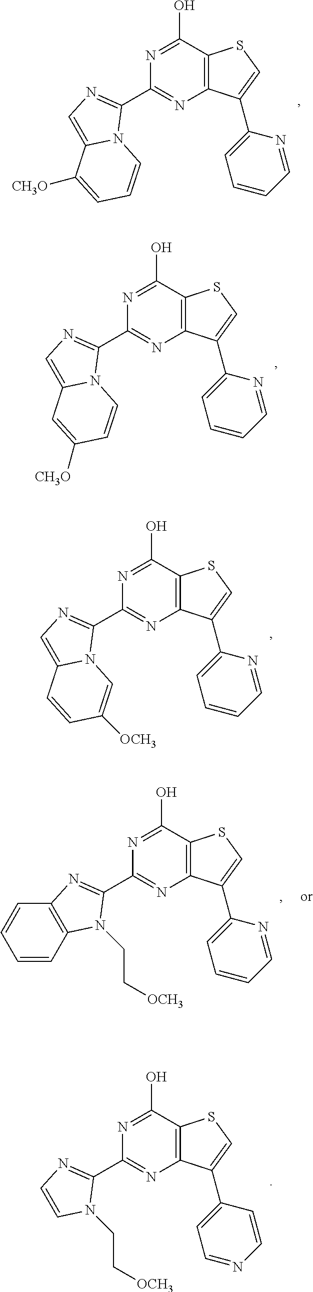

Embodiment 110. The compound or pharmaceutically acceptable derivative of any one of embodiments 86-107, wherein the compound of Formula IIB is:

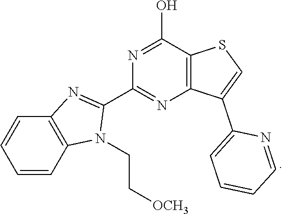 [SMILES: COCCn1c(-c2nc(O)c3scc(-c4ccccn4)c3n2)nc2ccccc21]

Embodiment 111. A compound or a pharmaceutically acceptable derivative thereof, wherein the compound is:

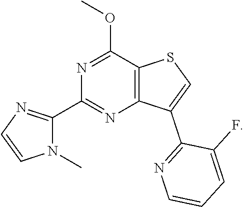 [SMILES: COc1nc(-c2nccn2C)nc2c(-c3ncccc3F)csc12]

Embodiment 112. A compound of selected from the group consisting of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, and 95, or a pharmaceutically acceptable derivative thereof.

Embodiment 113. A compound of selected from the group consisting of compounds 1, 2, 3, 4, 5, 6, 7, 8, 9, 10, 11, 12, 13, 14, 15, 16, 17, 18, 19, 20, 21, 22, 23, 24, 25, 26, and 27, or a pharmaceutically acceptable derivative thereof.

Embodiment 114. A compound of selected from the group consisting of compounds 28, 29, 30, 31, 32, 33, 34, 35, 36, 37, 38, and 39 or a pharmaceutically acceptable derivative thereof.

Embodiment 115. A compound which binds to the GTP binding domain of one or more members of the Ras superfamily and inhibits the one or more members of the Ras superfamily with an IC₅₀ value of less than 10 micromolar, wherein the compound is the compound or pharmaceutically acceptable derivative of any one of embodiments 1-114.

Embodiment 116. The compound of embodiment 115, wherein one or more members of the Ras superfamily is Ras.

Embodiment 117. The compound of embodiment 115, wherein one or more members of the Ras superfamily is Rho.

Embodiment 118. The compound of embodiment 115, wherein one or more members of the Ras superfamily is Rac.

Embodiment 119. The compound of embodiment 116, wherein the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

Embodiment 120. The compound of embodiment 119, wherein the Ras is HRAS, KRAS, NRAS, or a mutant thereof.

Embodiment 121. The compound of embodiment 120, wherein the Ras is HRAS or a mutant thereof.

Embodiment 122. The compound of embodiment 120, wherein the Ras is KRAS or a mutant thereof.

Embodiment 123. The compound of embodiment 120, wherein the Ras is NRAS or a mutant thereof.

Embodiment 124. The compound of embodiment 117, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

Embodiment 125. The compound of embodiment 117, wherein the Rho is Rac.

Embodiment 126. The compound of embodiment 118 or 125, wherein the Rac is RAC1; RAC2; RAC3; RHOG, or a mutant thereof.

Embodiment 127. The compound or pharmaceutically acceptable derivative of any one of embodiments 1-126, wherein the pharmaceutically acceptable derivative of the compound is a pharmaceutically acceptable salt of said compound.

Embodiment 128. A method of inhibiting the function of one or more members of the Ras superfamily, comprising administering to a subject a compound which inhibits the one or more members of the Ras superfamily with an IC₅₀ value of less than 10 μM, wherein the compound is the compound or pharmaceutically acceptable derivative of any one of embodiments 1-126 or the compound is the compound or pharmaceutically acceptable salt of embodiment 127.

Embodiment 129. The method of embodiment 128, wherein one or more members of the Ras superfamily is Ras.

Embodiment 130. The method of embodiment 128, wherein one or more members of the Ras superfamily is Rho.

Embodiment 131. The method of embodiment 128, wherein one or more members of the Ras superfamily is Rac.

Embodiment 132. The method of embodiment 128, wherein the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

Embodiment 133. The method of embodiment 132, wherein the Ras is HRAS, KRAS, NRAS or a mutant thereof

Embodiment 134. The method of embodiment 132, wherein the Ras is HRAS or a mutant thereof.

Embodiment 135. The method of embodiment 132, wherein the Ras is KRAS or a mutant thereof.

Embodiment 136. The method of embodiment 132, wherein the Ras is NRAS or a mutant thereof.

Embodiment 137. The method of embodiment 130, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

Embodiment 138. The method of embodiment 137, wherein the Rho is Rac.

Embodiment 139. The method of embodiment 131 or 138, wherein the Rac is RAC1; RAC2; RAC3; RHOG, or a mutant thereof.

Embodiment 140. The method of embodiment 128, wherein the inhibiting the function of one or more members of the Ras superfamily is a treatment, prevention or amelioration of one or more symptoms of cancer.

Embodiment 141. The method of any of embodiments 129 or 132-136, wherein the inhibiting the function of Ras is a treatment, prevention or amelioration of one or more symptoms of cancer.

Embodiment 142. The method of any of embodiments 130 or 137-138, wherein the inhibiting the function of Rho is a treatment, prevention or amelioration of one or more symptoms of cancer.

Embodiment 143. The method of any of embodiments 131 or 138-139, wherein the inhibiting the function of Rac is a treatment, prevention or amelioration of one or more symptoms of cancer.

Embodiment 144. The method of any of embodiments 140-143, wherein the cancer is a solid tumor.

Embodiment 145. The method of embodiment 144, wherein the solid tumor is hepatocellular carcinoma, prostate cancer, pancreatic cancer, lung cancer, breast cancer, ovarian cancer, colon cancer, small intestine cancer, biliary tract cancer, endometrium cancer, skin cancer (melanoma), cervix cancer, urinary tract cancer, or glioblastoma.

Embodiment 146. The method of embodiment 145, wherein the solid tumor is pancreatic cancer.

Embodiment 147. The method of embodiment 145, wherein the solid tumor is colon cancer.

Embodiment 148. The method of embodiment 145, wherein the solid tumor is small intestine cancer.

Embodiment 149. The method of embodiment 145, wherein the solid tumor is biliary tract cancer.

Embodiment 150. The method of embodiment 145, wherein the solid tumor is endometrium cancer.

Embodiment 151. The method of embodiment 145, wherein the solid tumor is lung cancer.

Embodiment 152. The method of embodiment 145, wherein the solid tumor is breast cancer.

Embodiment 153. The method of embodiment 145, wherein the solid tumor is skin cancer.

Embodiment 154. The method of embodiment 145, wherein the solid tumor is cervix cancer.

Embodiment 155. The method of embodiment 145, wherein the solid tumor is urinary tract cancer.

Embodiment 156. The method of any of embodiments 140-143, wherein the cancer is a blood borne tumor.

Embodiment 157. The method of embodiment 156, wherein the blood borne tumor is a leukemia.

Embodiment 158. The method of embodiment 156, wherein the blood borne tumor is chronic lymphocytic leukemia (CLL), chronic myelocytic leukemia (CML), acute lymphoblastic leukemia (ALL), acute myeloid leukemia (AML), or acute myeloblastic leukemia (AML).

Embodiment 159. The method of any one of embodiments 156-158, wherein the blood borne tumor is metastatic.

Embodiment 160. The method of embodiment 128, wherein the inhibiting the function of one or more members of the Ras superfamily is a treatment, prevention or amelioration of one or more symptoms of an inflammatory disease.

Embodiment 161. The method of any of embodiments 129 or 132-136, wherein inhibiting the function of Ras is a treatment, prevention or amelioration of one or more symptoms of an inflammatory disease.

Embodiment 162. The method of any of embodiments 130 or 137-138, wherein the inhibiting the function of Rho is a treatment, prevention or amelioration of one or more symptoms of inflammatory disease.

Embodiment 163. The method of any of embodiments 131 or 138-139, wherein the inhibiting the function of Rac is a treatment, prevention or amelioration of one or more symptoms of inflammatory disease.

Embodiment 164. The method of any of embodiments 160-163, wherein the inflammatory disease is gastritis, schistosomiasis, cholangitis, chronic cholecystitis, pelvic inflammatory disease, chronic cervicitis, osteomyelitis, inflammatory bowel disease, reflux esophagitis, Barrett's esophagus, bladder inflammation (cystitis), asbestosis, silicosis, gingivitis, lichen planus, pancreatitis, protease mutation, lichen sclerosis, slaladenitis, bronchitis, Sjogren syndrome or Hashimoto's thyroiditis.

Embodiment 165. The method of any of embodiments 160-163, wherein the inflammatory disease is Alzheimer's disease (AD), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus, erythematous (SLE), nephritis, Parkinson's disease, ulcerative colitis.

Embodiment 166. The method of embodiment 165, wherein the inflammatory disease is Alzheimer's disease (AD).

Embodiment 167. The method of embodiment 165, wherein the inflammatory disease is ankylosing spondylitis.

Embodiment 168. The method of embodiment 165, wherein the inflammatory disease is arthritis (osteoarthritis, rheumatoid arthritis (RA), psoriatic arthritis).

Embodiment 169. The method of embodiment 165, wherein the inflammatory disease is asthma.

Embodiment 170. The method of embodiment 165, wherein the inflammatory disease is atherosclerosis.

Embodiment 171. The method of embodiment 165, wherein the inflammatory disease is Crohn's disease.

Embodiment 172. The method of embodiment 165, wherein the inflammatory disease is colitis.

Embodiment 173. The method of embodiment 165, wherein the inflammatory disease is dermatitis.

Embodiment 174. The method of embodiment 165, wherein the inflammatory disease is diverticulitis.

Embodiment 175. The method of embodiment 165, wherein the inflammatory disease is fibromyalgia.

Embodiment 176. The method of embodiment 165, wherein the inflammatory disease is hepatitis.

Embodiment 177. The method of embodiment 165, wherein the inflammatory disease is irritable bowel syndrome (IBS).

Embodiment 178. The method of embodiment 165, wherein the inflammatory disease is systemic lupus.

Embodiment 179. The method of embodiment 165, wherein the inflammatory disease is erythematous (SLE).

Embodiment 180. The method of embodiment 165, wherein the inflammatory disease is nephritis.

Embodiment 181. The method of embodiment 165, wherein the inflammatory disease is Parkinson's disease.

Embodiment 182. The method of embodiment 165, wherein the inflammatory disease is ulcerative colitis.

Embodiment 183. The method of embodiment 128, wherein the inhibiting the function of one or more members of the Ras superfamily is a treatment, prevention or amelioration of one or more symptoms of a rasopathy.

Embodiment 184. The method of any of embodiments 129 or 132-136, wherein the inhibiting the function of Ras is a treatment for a rasopathy.

Embodiment 185. The method of any of embodiments 130 or 137-138, wherein the inhibiting the function of Rho is a treatment for a rasopathy.

Embodiment 186. The method of any of embodiments 131 or 138-139, wherein the inhibiting the function of Rac is a treatment for a rasopathy.

Embodiment 187. The method of any of embodiments 183-186, wherein the rasopathy is neurofibromatosis type 1, Noonan's syndrome or Costello syndrome.

Embodiment 188. The method of any of embodiments 129 or 132-136, wherein the inhibiting the function of Ras is a treatment for Ras-associated autoimmune leukoproliferative disorder.

Embodiment 189. The method of embodiment 128, wherein the inhibiting the function of one or more members of the Ras superfamily is a treatment, prevention or amelioration of one or more symptoms of a fibrotic disease.

Embodiment 190. The method of any of embodiments 129 or 132-136, wherein the inhibiting the function of Ras is a treatment, prevention or amelioration of one or more symptoms of a fibrotic disease.

Embodiment 191. The method of any of embodiments 130 or 137-138, wherein the inhibiting the function of Rho is a treatment, prevention or amelioration of one or more symptoms of a fibrotic disease.

Embodiment 192. The method of any of embodiments 130 or 137-138, wherein the inhibiting the function of Rac is a treatment, prevention or amelioration of one or more symptoms of a fibrotic disease.

Embodiment 193. The method of any one of embodiments 140, 160, 183, or 189, wherein one or more members of the Ras superfamily is Ras.

Embodiment 194. The method of any one of embodiments 140, 160, 183, or 189, wherein one or more members of the Ras superfamily is Rho.

Embodiment 195. The method of any one of embodiments 140, 160, 183, or 189, wherein one or more members of the Ras superfamily is Rac.

Embodiment 196. A pharmaceutical composition, comprising the compound or pharmaceutically acceptable derivative of any one of embodiments 1-126, and a pharmaceutically acceptable carrier.

Embodiment 197. The pharmaceutical composition of embodiment 196, wherein the pharmaceutical composition comprises a therapeutic amount of said compound or pharmaceutically acceptable derivative thereof.

Embodiment 198. A pharmaceutical composition, comprising the compound or pharmaceutically acceptable salt of embodiment 127, and a pharmaceutically acceptable carrier.

Embodiment 199. The pharmaceutical composition of embodiment 198, wherein the pharmaceutical composition comprises a therapeutic amount of said compound or pharmaceutically acceptable salt thereof.

Embodiment 200. A method of inhibiting the function of one or more members of the Ras superfamily, comprising administering to a subject the pharmaceutical composition of any one of embodiments 196-199.

Embodiment 201. A method of inhibiting the function of one or more members of the Ras superfamily, comprising administering to a subject the compound or pharmaceutically acceptable derivative of any one of embodiments 1-126.

This disclosure is not to be limited in scope by the embodiments disclosed in the examples which are intended as single illustrations of individual aspects, and any methods which are functionally equivalent are within the scope of this disclosure. Indeed, various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound of Formula IA: 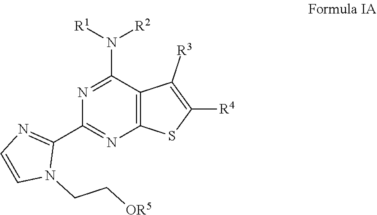
or a pharmaceutically acceptable derivative thereof, wherein:
—NR¹R² is 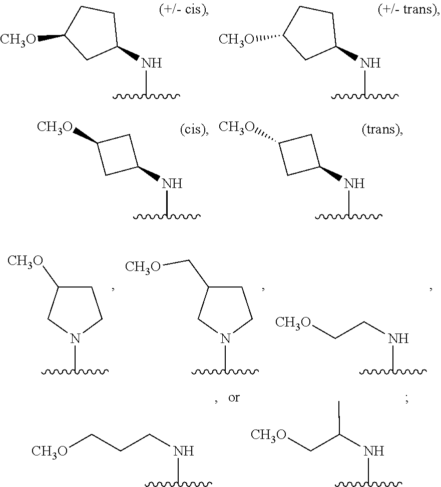
R³ is hydrogen, —CH₃, —CF₃, or phenyl;
R⁴ is hydrogen, 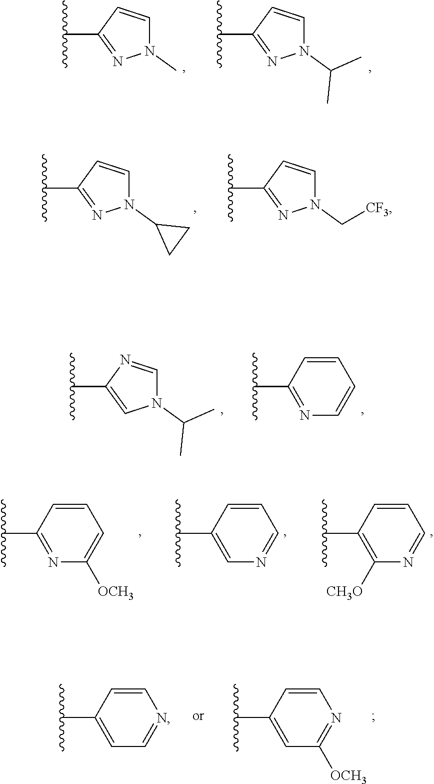
and
R⁵ is —CH₃ or —CH₂CF₃.

2. The compound or pharmaceutically acceptable derivative of claim 1, wherein —NR¹R² is:

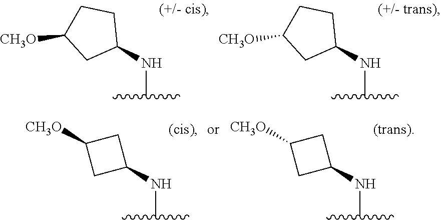

3. The compound or pharmaceutically acceptable derivative of claim 1 or 2, wherein R³ is hydrogen.

4. The compound or pharmaceutically acceptable derivative of claim 1 or 2, wherein R³ is —CH₃.

5. The compound or pharmaceutically acceptable derivative of claim 1 or 2, wherein R³ is phenyl.

6. The compound or pharmaceutically acceptable derivative of claim 1, wherein:
—NR¹R² is 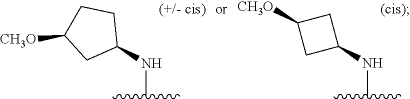
R³ is hydrogen or phenyl;
R⁴ is hydrogen or 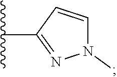
and
R⁵ is —CH₃.

7. The compound or pharmaceutically acceptable derivative of claim 6, wherein R³ is hydrogen.

8. The compound or pharmaceutically acceptable derivative of claim 6, wherein R³ is phenyl.

9. The compound or pharmaceutically acceptable derivative of any one of claims 1-8, wherein R⁴ is hydrogen.

10. The compound or pharmaceutically acceptable derivative of any one of claims 1-8, wherein R⁴ is:

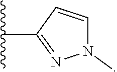

11. The compound or pharmaceutically acceptable derivative of any one of claims 1-10, wherein the compound of Formula IA is:

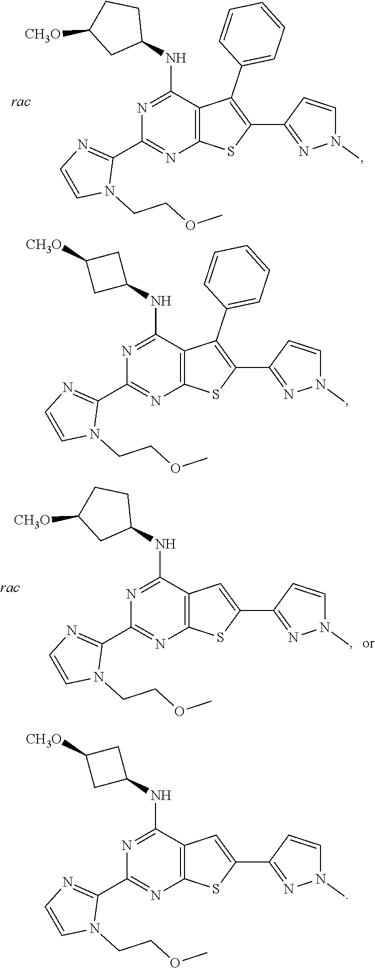

12. A compound of Formula IB: 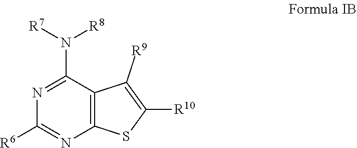
or a pharmaceutically acceptable derivative thereof, wherein:
R⁶ is 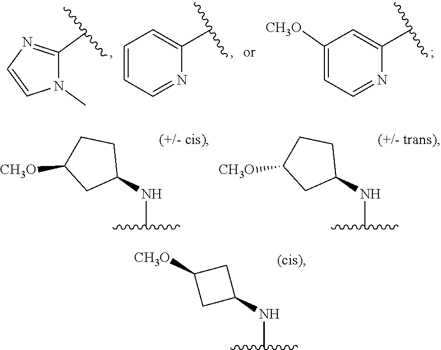
—NR⁷R⁸ is 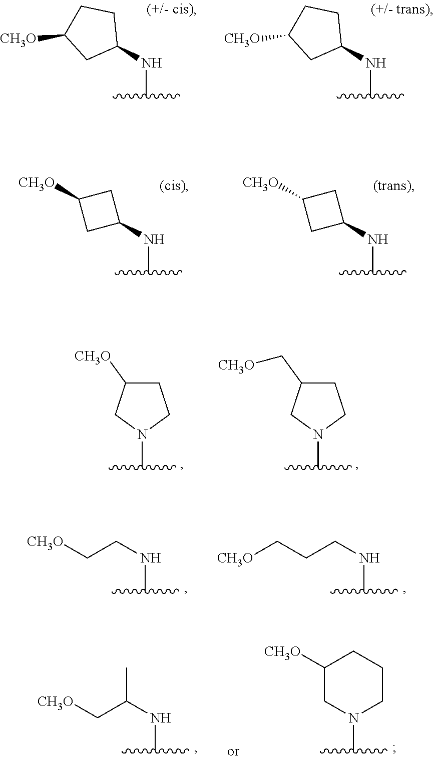
R⁹ is hydrogen, —CH₃, —CF₃, or phenyl; and
R¹⁰ is

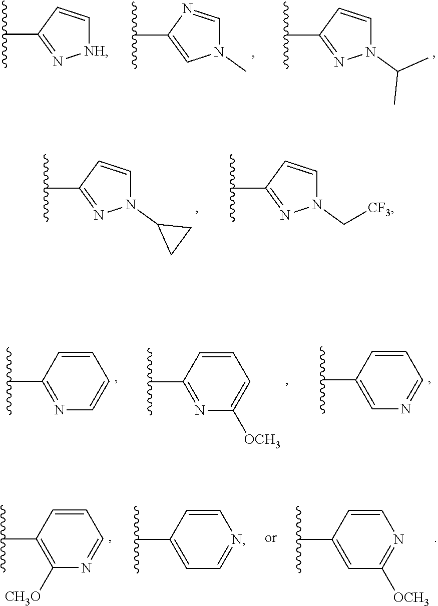

13. The compound or pharmaceutically acceptable derivative of claim 12, wherein R⁶ is:

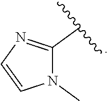 [SMILES: *c1nccn1C]

14. The compound or pharmaceutically acceptable derivative of claim 12, wherein R⁶ is:

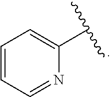 [SMILES: *c1ccccn1]

15. The compound or pharmaceutically acceptable derivative of claim 12, wherein R⁶ is:

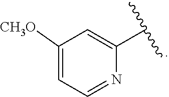

16. The compound or pharmaceutically acceptable derivative of any one of claims 12-15, wherein —NR⁷R⁸ is:

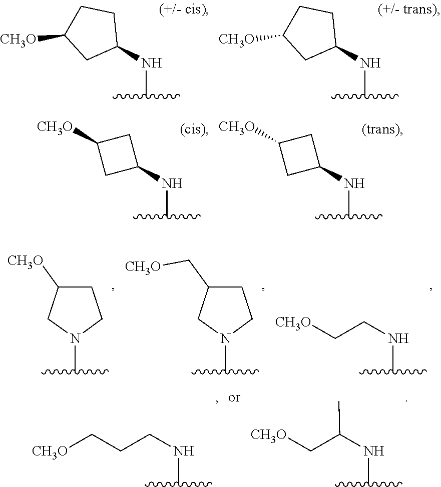

17. The compound or pharmaceutically acceptable derivative of any one of claims 12-15, wherein —NR⁷R⁸ is:

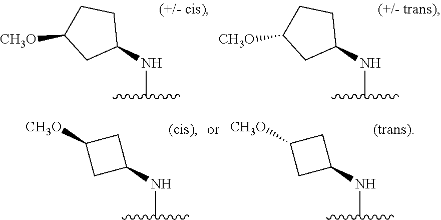

18. The compound or pharmaceutically acceptable derivative of any one of claims 12-15, wherein —NR⁷R⁸ is:

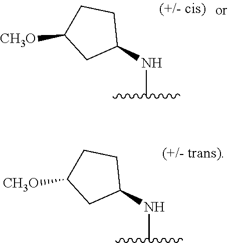

19. The compound or pharmaceutically acceptable derivative of any one of claims 12-15, wherein —NR⁷R⁸ is:

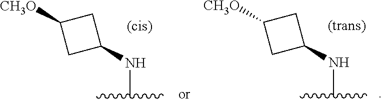

20. The compound or pharmaceutically acceptable derivative of any one of claims 12-15, wherein —NR⁷R⁸ is:

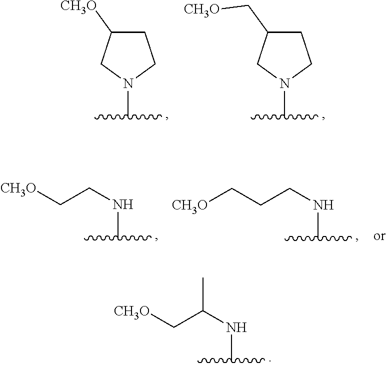

21. The compound or pharmaceutically acceptable derivative of any one of claims 12-15, wherein —NR⁷R⁸ is:

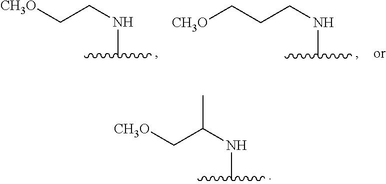

22. The compound or pharmaceutically acceptable derivative of any one of claims 12-15, wherein —NR⁷R⁸ is:

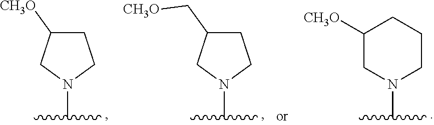

23. The compound or pharmaceutically acceptable derivative of any one of claims 12-15, wherein —NR⁷R⁸ is:

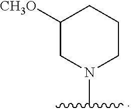

24. The compound or pharmaceutically acceptable derivative of any one of claims 12-23, wherein R⁹ is hydrogen.

25. The compound or pharmaceutically acceptable derivative of any one of claims 12-23, wherein R⁹ is —CH₃.

26. The compound or pharmaceutically acceptable derivative of any one of claims 12-23, wherein R⁹ is phenyl.

27. The compound or pharmaceutically acceptable derivative of any one of claims 12-26, wherein R¹⁰ is:

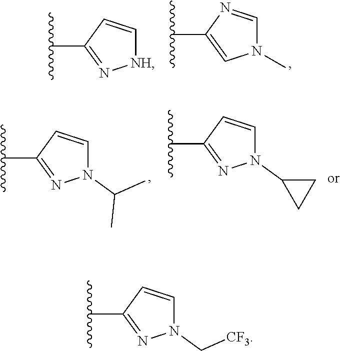

28. The compound or pharmaceutically acceptable derivative of any one of claims 12-26, wherein R¹⁰ is:

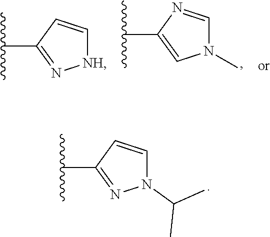

29. The compound or pharmaceutically acceptable derivative of any one of claims 12-26, wherein R¹⁰ is:

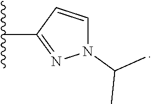 [SMILES: *c1ccn(C(C)C)n1]

30. The compound or pharmaceutically acceptable derivative of any one of claims 12-26, wherein R¹⁰ is:

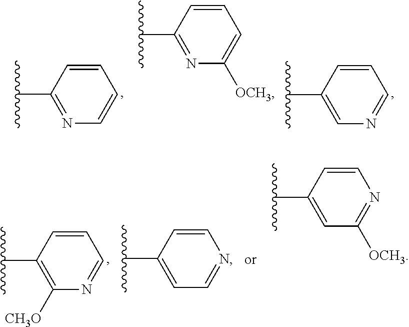

31. The compound or pharmaceutically acceptable derivative of any one of claims 12-30, wherein the compound of Formula IB is:

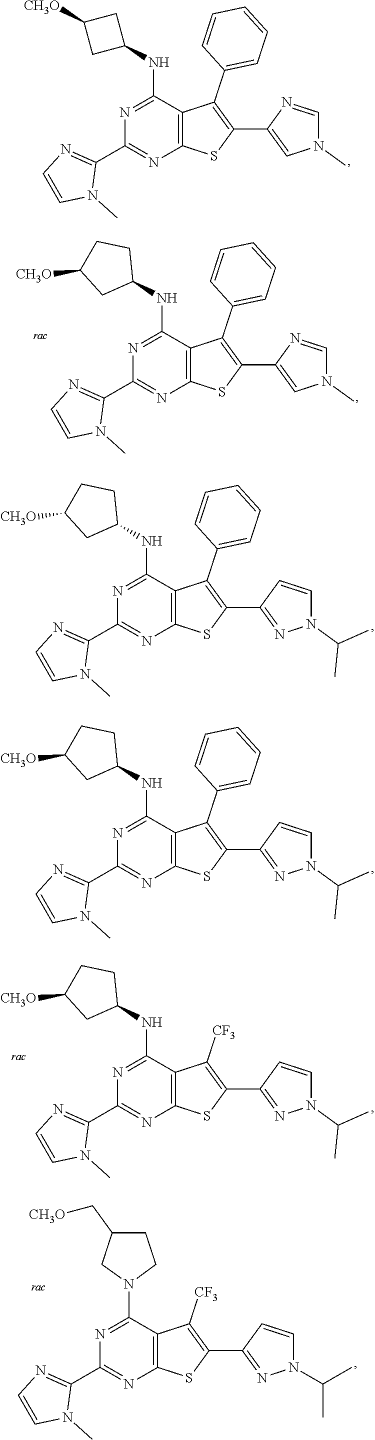

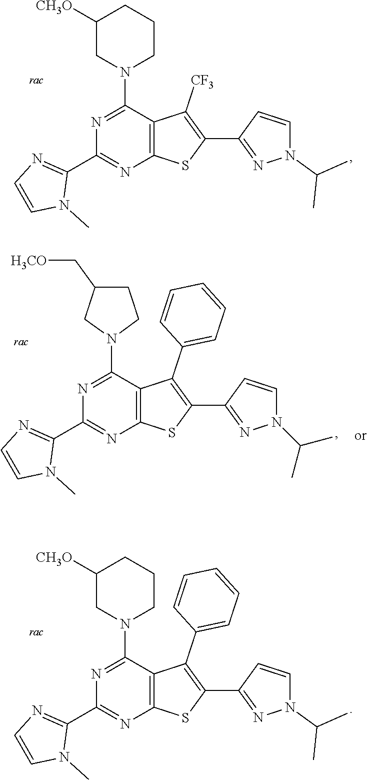

32. The compound or pharmaceutically acceptable derivative of any one of claims 12-31, wherein the compound of Formula IB is:

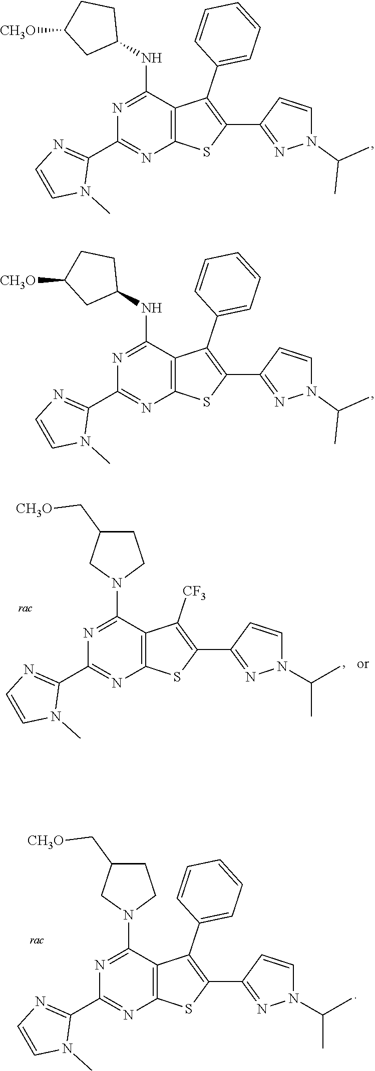

33. The compound or pharmaceutically acceptable derivative of any one of claims 12-32, wherein the compound of Formula IB is:

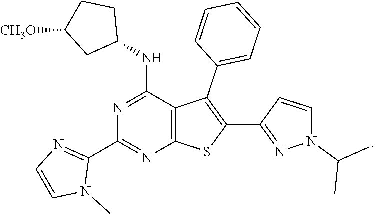

34. The compound or pharmaceutically acceptable derivative of claim 12, wherein:
R⁶ is 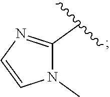 [SMILES: *c1nccn1C]
—NR⁷R⁸ is or 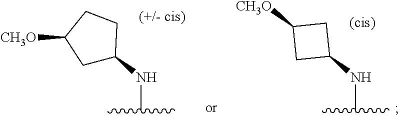
R⁹ is phenyl; and
R¹⁰ is

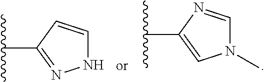

35. The compound or pharmaceutically acceptable derivative of any one of claims 12-30 or claim 34, wherein —NR⁷R⁸ is:

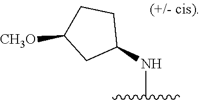

36. The compound or pharmaceutically acceptable derivative of any one of claims 12-30 or claim 34, wherein —NR⁷R⁸ is:

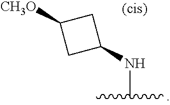

37. The compound or pharmaceutically acceptable derivative of any one of claims 12-30 or claims 34-36, wherein R¹⁰ is:

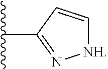 [SMILES: *c1cc[nH]n1]

38. The compound or pharmaceutically acceptable derivative of any one of claims 12-30 or claims 34-36, wherein R¹⁰ is:

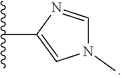 [SMILES: *c1cn(C)cn1]

39. The compound or pharmaceutically acceptable derivative of any one of claims 12-31 or claims 34-38, wherein the compound of Formula IB is:

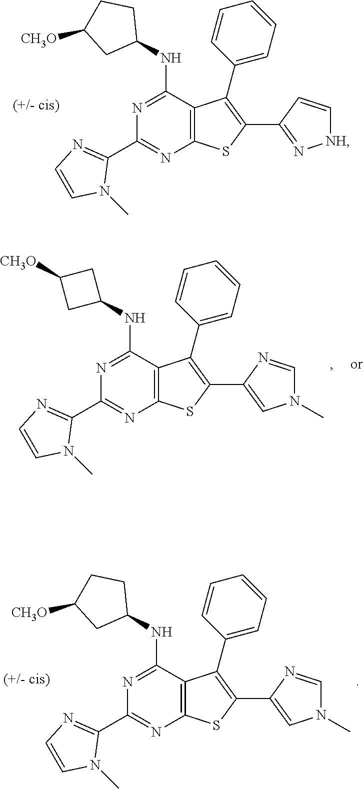

40. A compound of Formula IC: 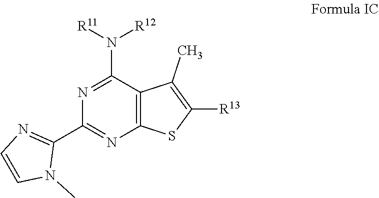
or a pharmaceutically acceptable derivative thereof, wherein:
—NR¹¹R¹² is 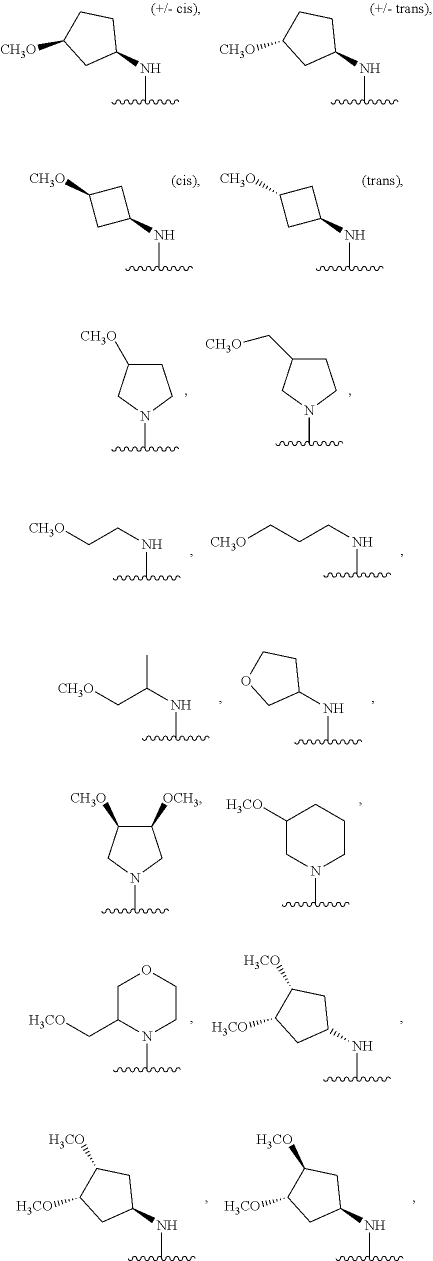 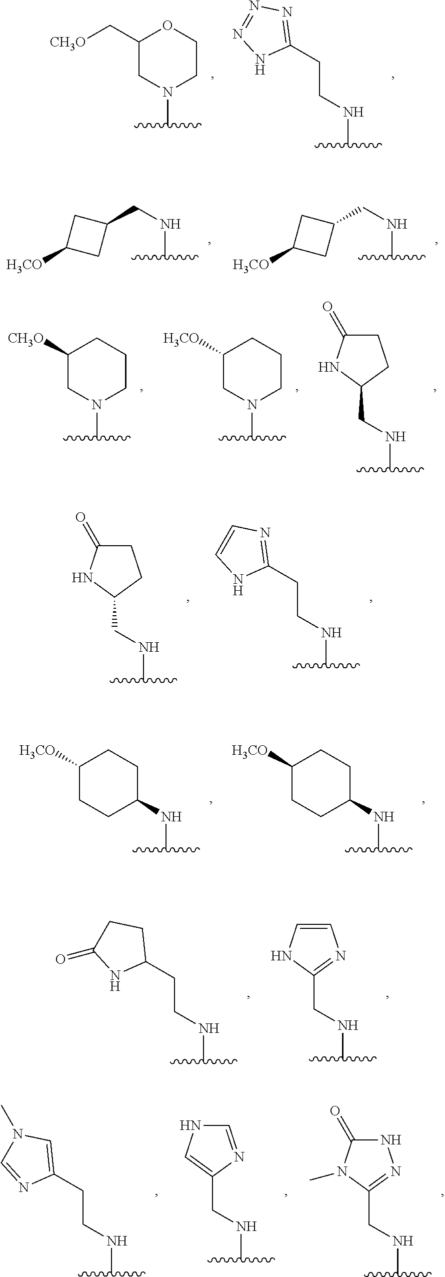 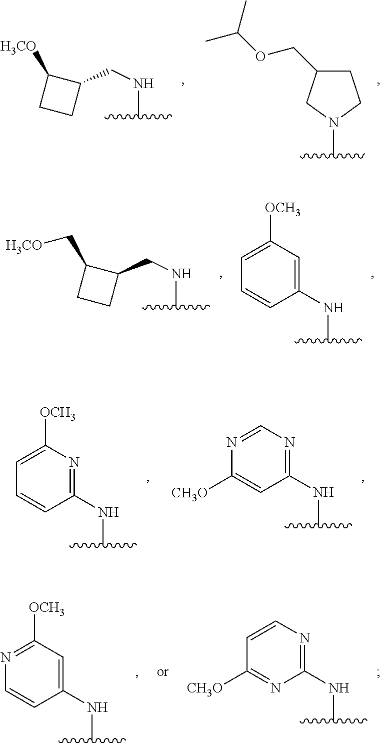
and
R¹³ is

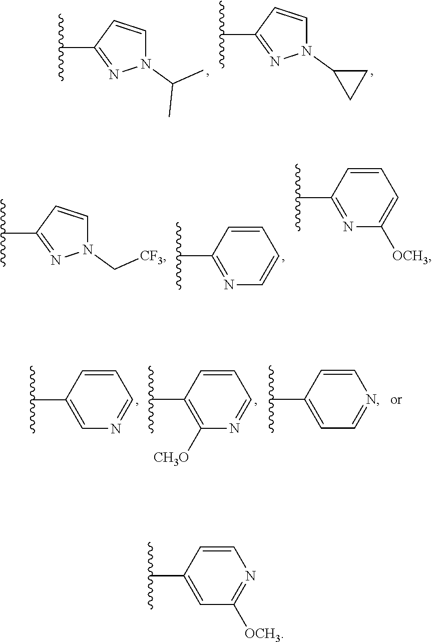

41. The compound or pharmaceutically acceptable derivative of claim 40, wherein R¹³ is:

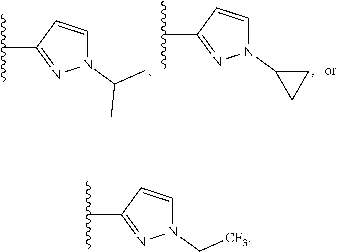

42. The compound or pharmaceutically acceptable derivative of claim 40, wherein R¹³ is:

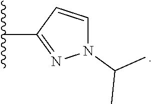 [SMILES: *c1ccn(C(C)C)n1]

43. The compound or pharmaceutically acceptable derivative of claim 40, wherein R¹³ is:

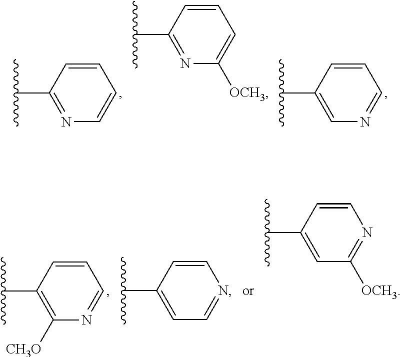

44. The compound or pharmaceutically acceptable derivative of any one of claims 40-43, wherein —NR¹¹R¹² is:

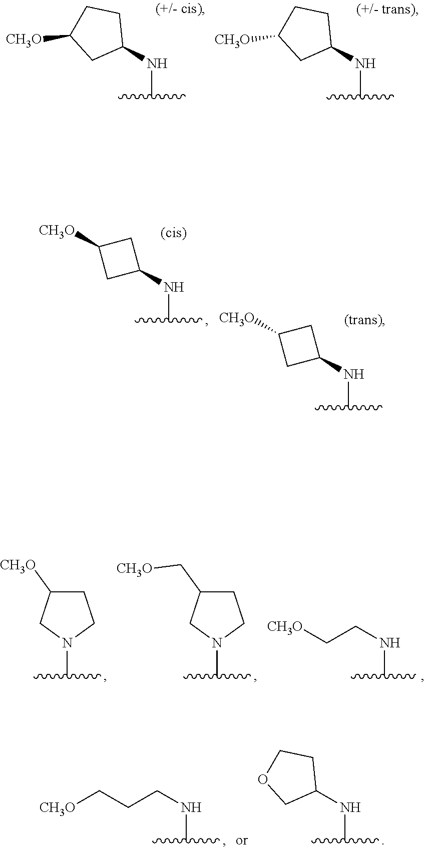

45. The compound or pharmaceutically acceptable derivative of any one of claims 40-44, wherein —NR¹¹R¹² is:

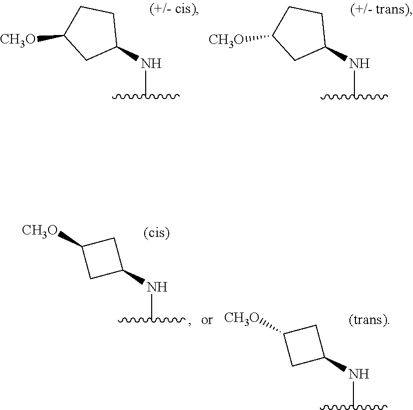

46. The compound or pharmaceutically acceptable derivative of any one of claims 40-44, wherein —NR¹¹R¹² is:

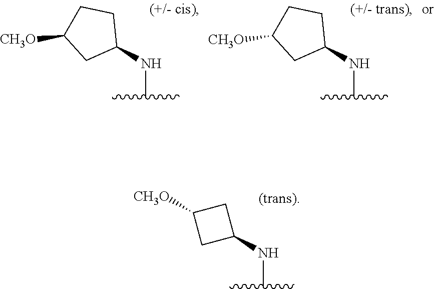

47. The compound or pharmaceutically acceptable derivative of any one of claims 40-44, wherein —NR¹¹R¹² is:

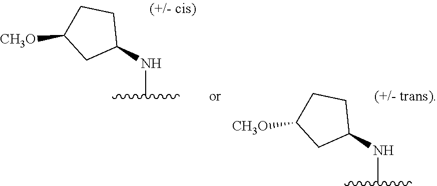

48. The compound or pharmaceutically acceptable derivative of any one of claims 40-44, wherein —NR¹¹R¹² is:

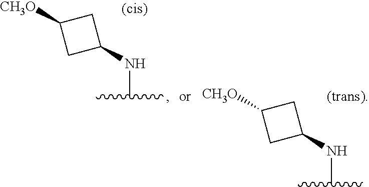

50. The compound or pharmaceutically acceptable derivative of any one of claims 40-44, wherein —NR¹¹R¹² is:

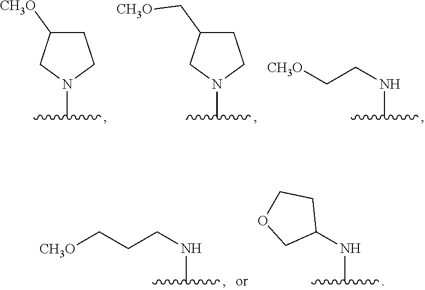

51. The compound or pharmaceutically acceptable derivative of any one of claims 40-43, wherein —NR¹¹R¹² is:

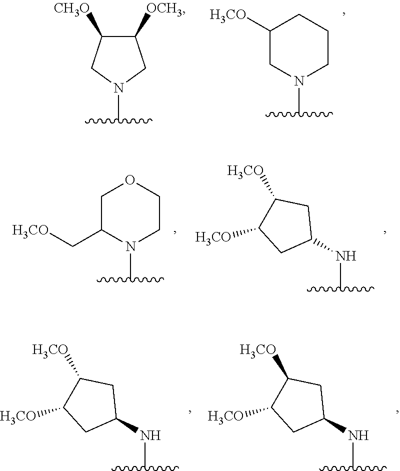

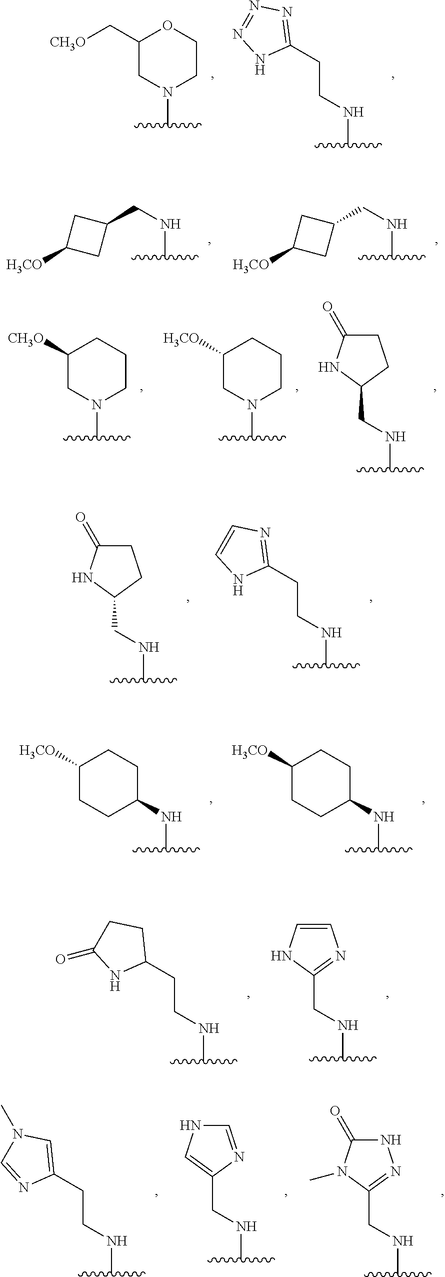

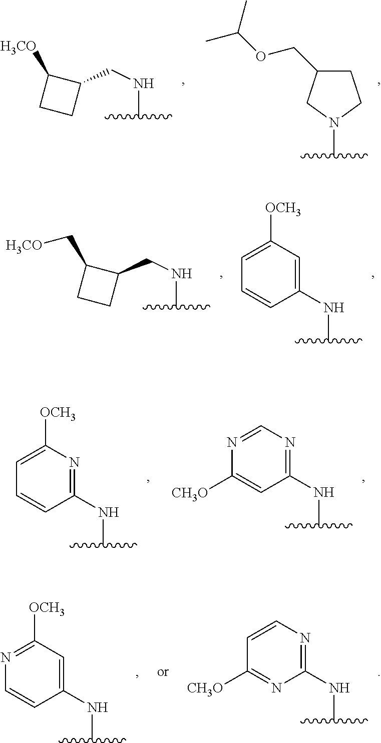

52. The compound or pharmaceutically acceptable derivative of any one of claims 40-43 or claim 51, wherein —NR¹¹R¹² is:

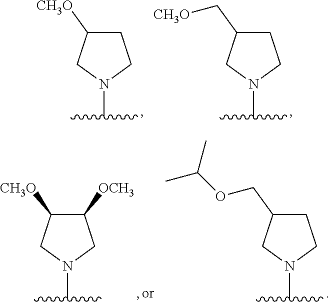

53. The compound or pharmaceutically acceptable derivative of any one of claims 40-43 or claim 51, wherein —NR¹¹R¹² is:

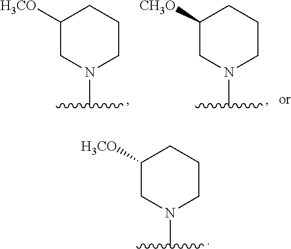

54. The compound or pharmaceutically acceptable derivative of any one of claims 40-43 or claim 51, wherein —NR¹¹R¹² is:

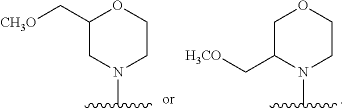

55. The compound or pharmaceutically acceptable derivative of any one of claims 40-43 or claim 51, wherein —NR¹¹R¹² is:

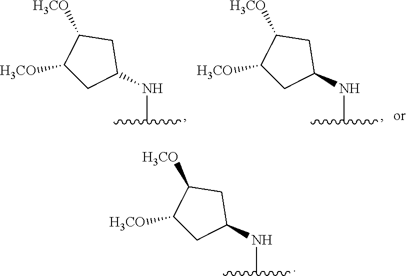

56. The compound or pharmaceutically acceptable derivative of any one of claims 40-43 or claim 51, wherein —NR¹¹R¹² is:

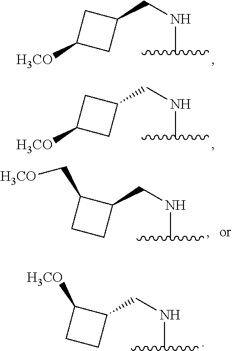

57. The compound or pharmaceutically acceptable derivative of any one of claims 40-43 or claim 51, wherein —NR¹¹R¹² is:

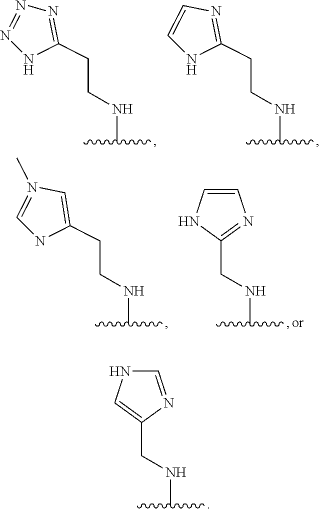

58. The compound or pharmaceutically acceptable derivative of any one of claims 40-43 or claim 51, wherein —NR¹¹R¹² is:

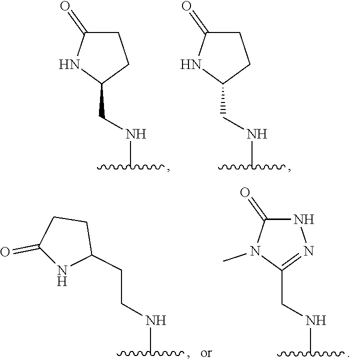

59. The compound or pharmaceutically acceptable derivative of any one of claims 40-43 or claim 51, wherein —NR¹¹R¹² is:

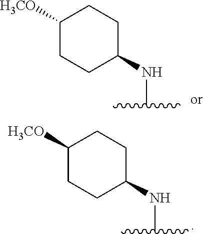 [SMILES: *N[C@H]1CC[C@H](OC)CC1 ; *N[C@H]1CC[C@@H](OC)CC1]

60. The compound or pharmaceutically acceptable derivative of any one of claims 40-43 or claim 51, wherein —NR¹¹R¹² is:

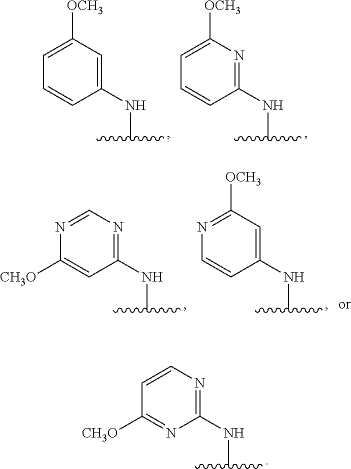

61. The compound or pharmaceutically acceptable derivative of any one of claims 40-60, wherein the compound of Formula IC is:

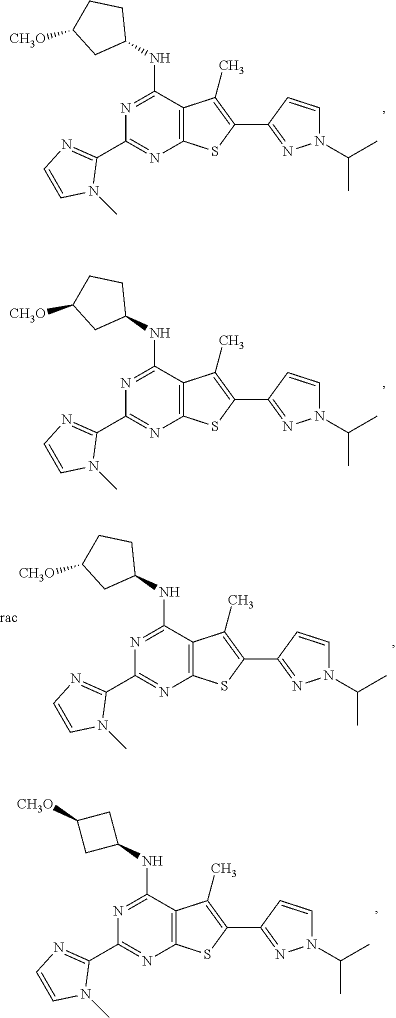

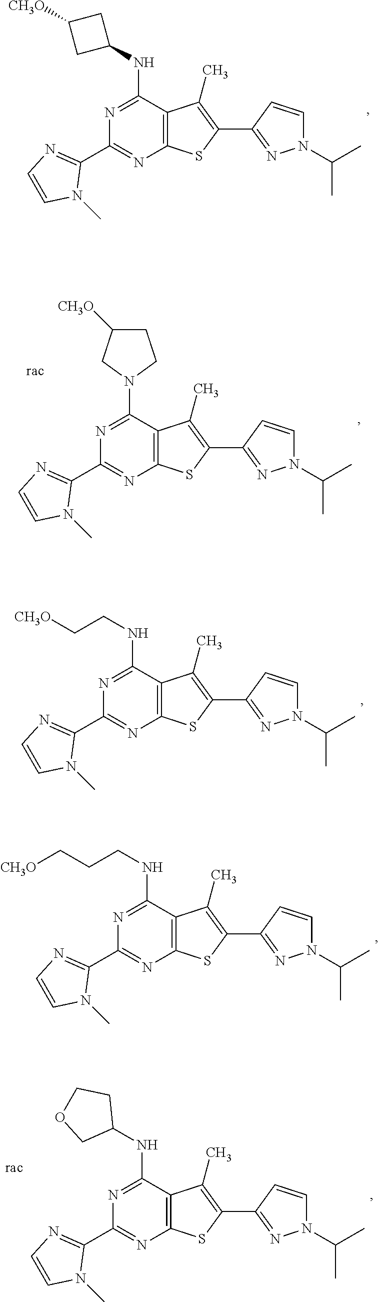

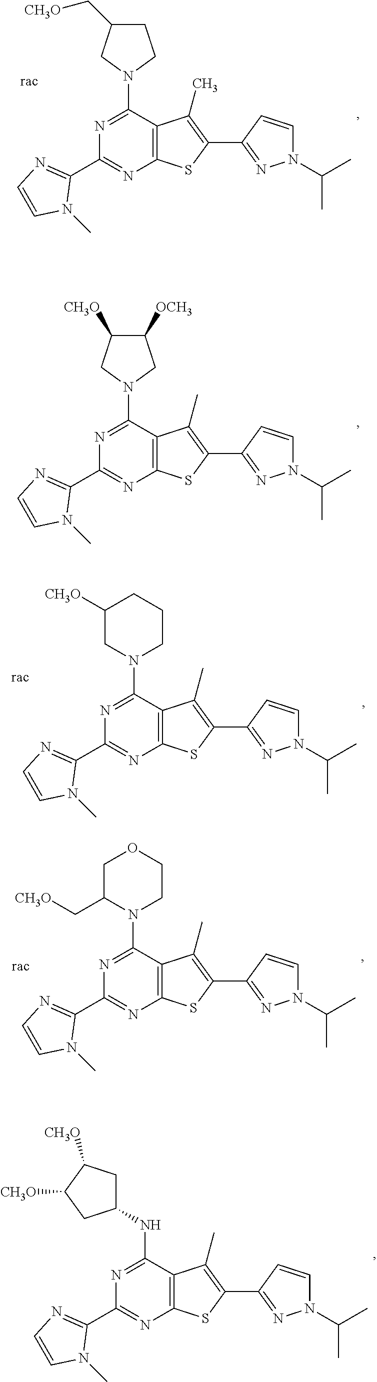

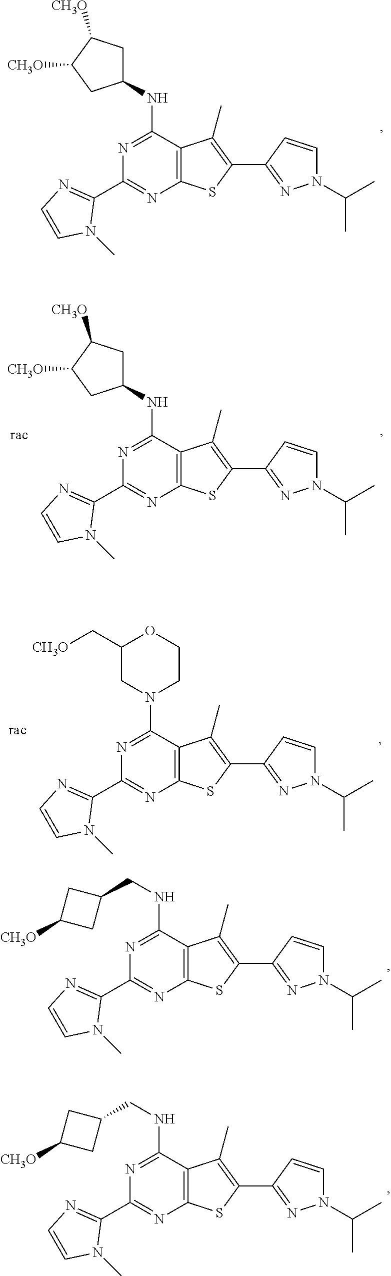

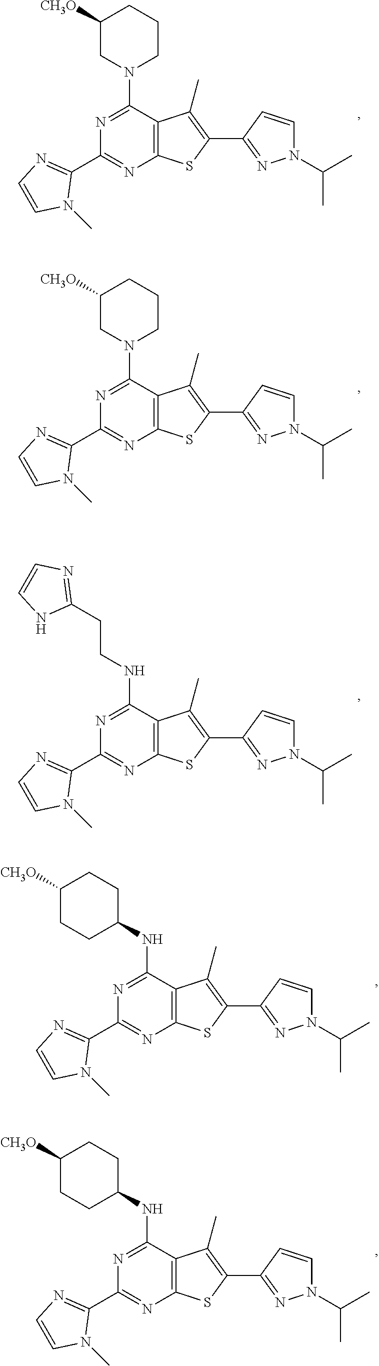

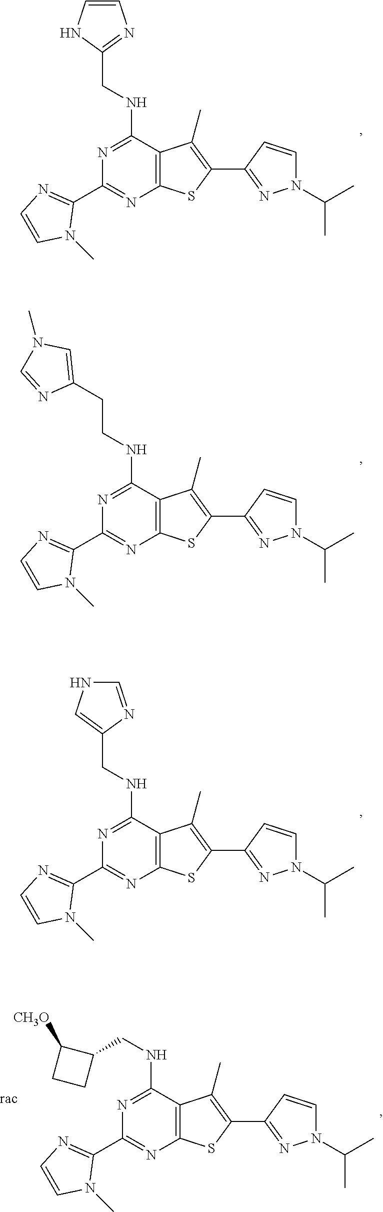

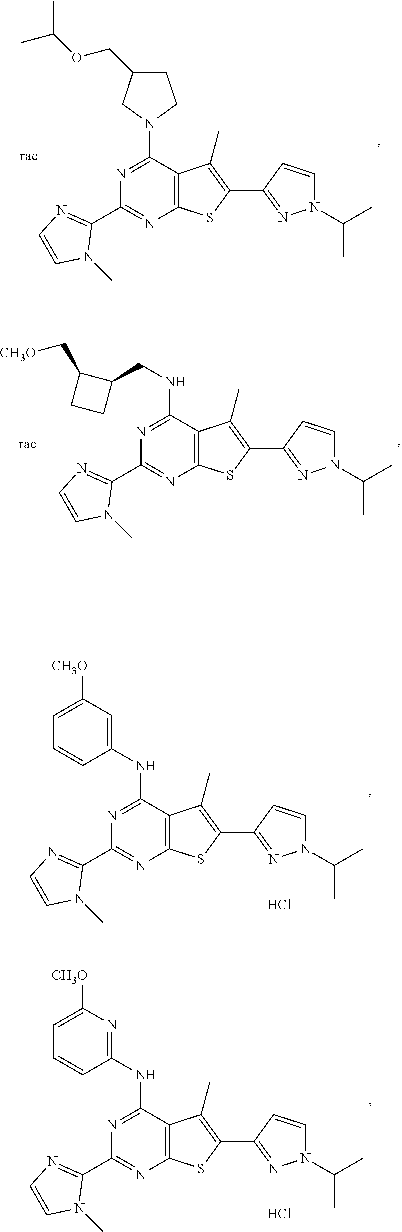

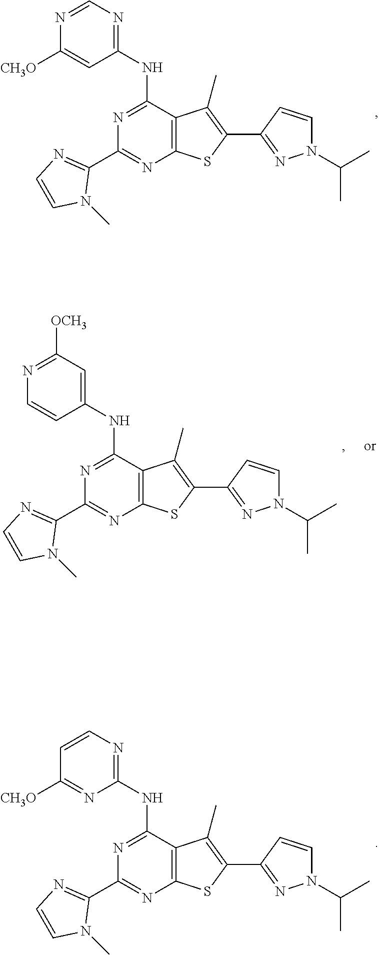

62. The compound or pharmaceutically acceptable derivative of any one of claims 40-61, wherein the compound of Formula 1C is:

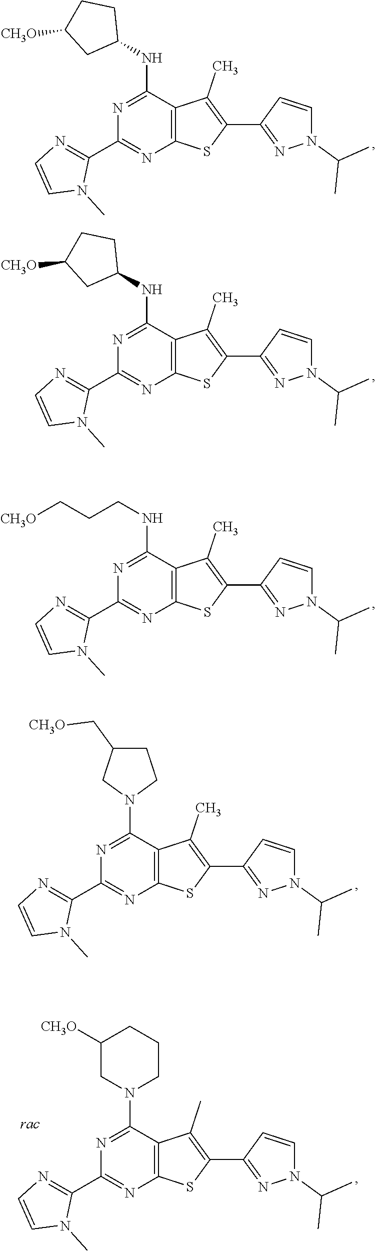

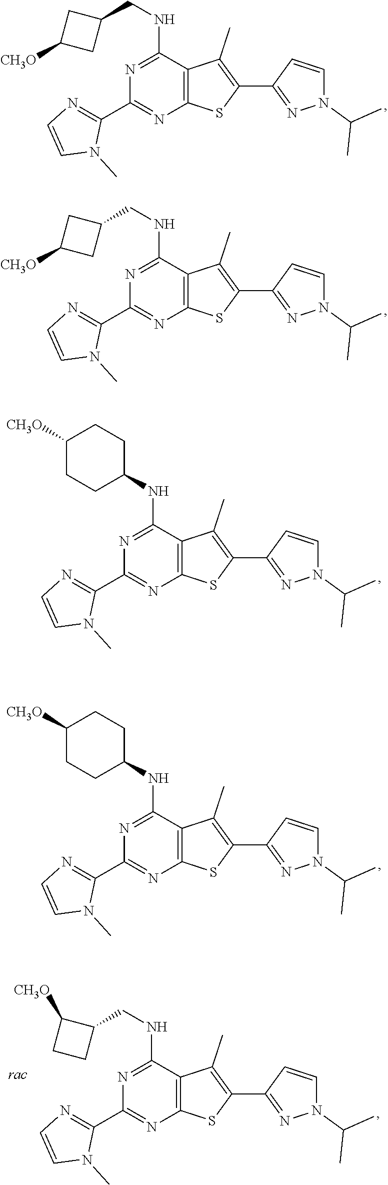

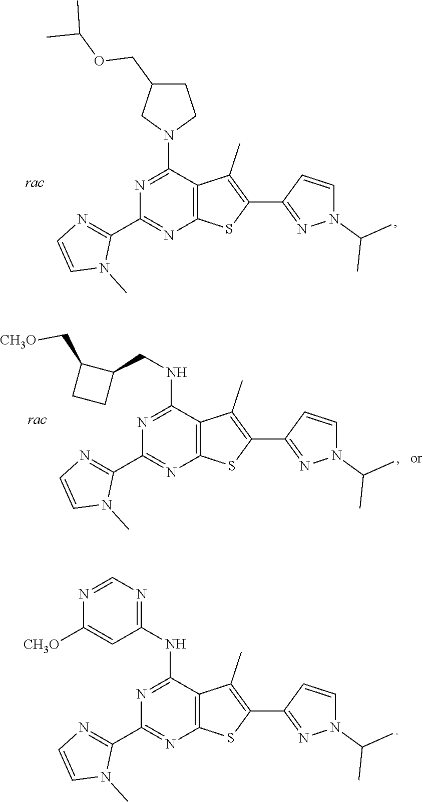

63. A compound of Formula ID: 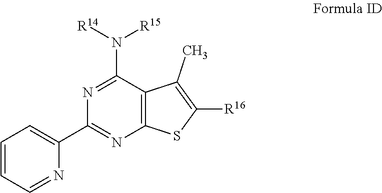
or a pharmaceutically acceptable derivative thereof, wherein:
—NR¹⁴R¹⁵ is 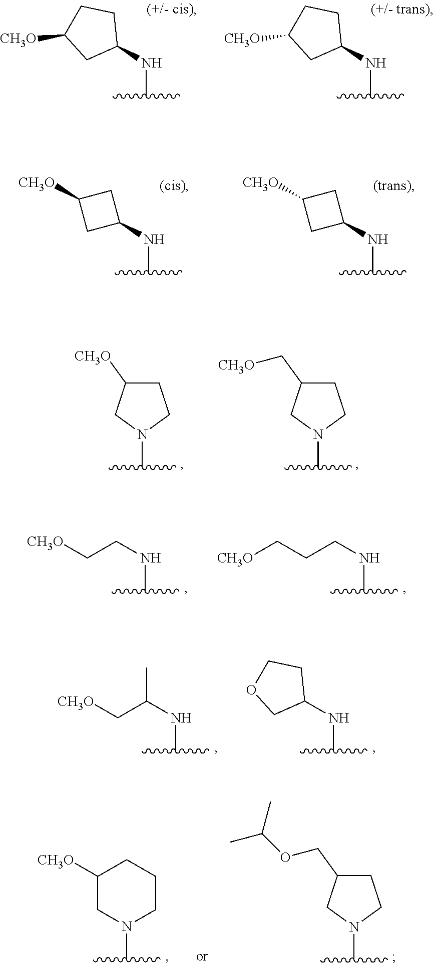
and
R¹⁶ is

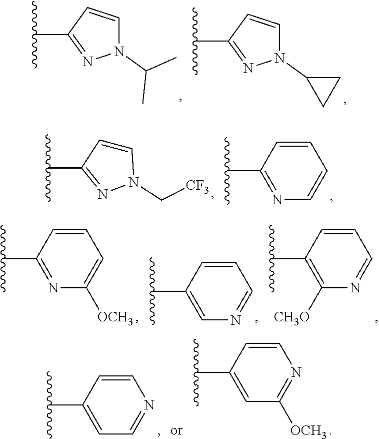

64. The compound of claim 63, wherein —NR¹⁴R¹⁵ is:

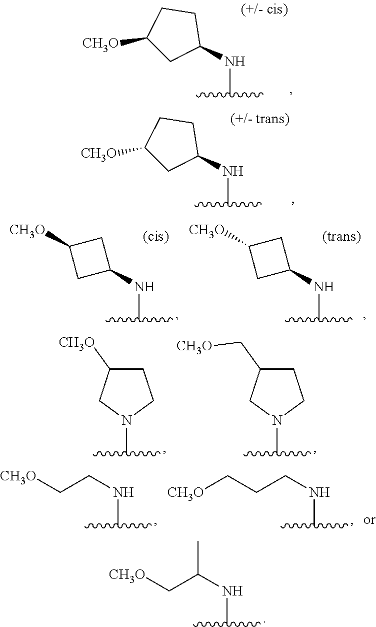

65. The compound or pharmaceutically acceptable derivative of claim 63, wherein —NR¹⁴R¹⁵ is:

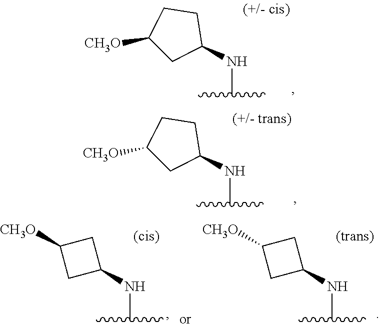

66. The compound or pharmaceutically acceptable derivative of claim 63, wherein —NR¹⁴R¹⁵ is:

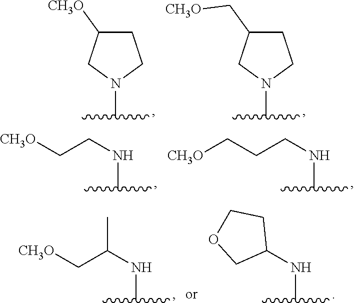

67. The compound of claim 63, wherein —NR¹⁴R¹⁵ is:

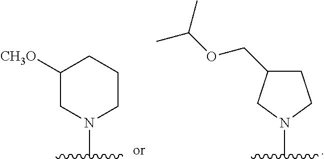

68. The compound or pharmaceutically acceptable derivative of any one of claims 63-67, wherein R¹⁶ is:

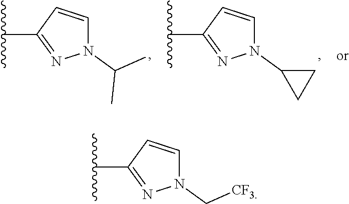

69. The compound or pharmaceutically acceptable derivative of any one of claims 63-67, wherein R¹⁶ is:

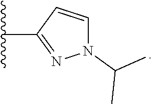 [SMILES: *c1ccn(C(C)C)n1]

70. The compound or pharmaceutically acceptable derivative of any one of claims 63-67, wherein R¹⁶ is:

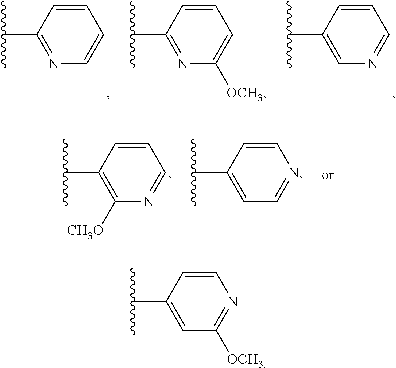

71. The compound or pharmaceutically acceptable derivative of claim 63, wherein:
—NR¹⁴R¹⁵ is 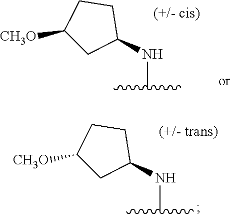
and
R¹⁶ is

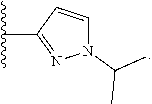 [SMILES: *c1ccn(C(C)C)n1]

72. The compound or pharmaceutically acceptable derivative of any one of claims 63-71, wherein —NR¹⁴R¹⁵ is

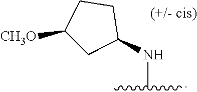

73. The compound or pharmaceutically acceptable derivative of any one of claims 64-72, wherein the compound of Formula ID is:

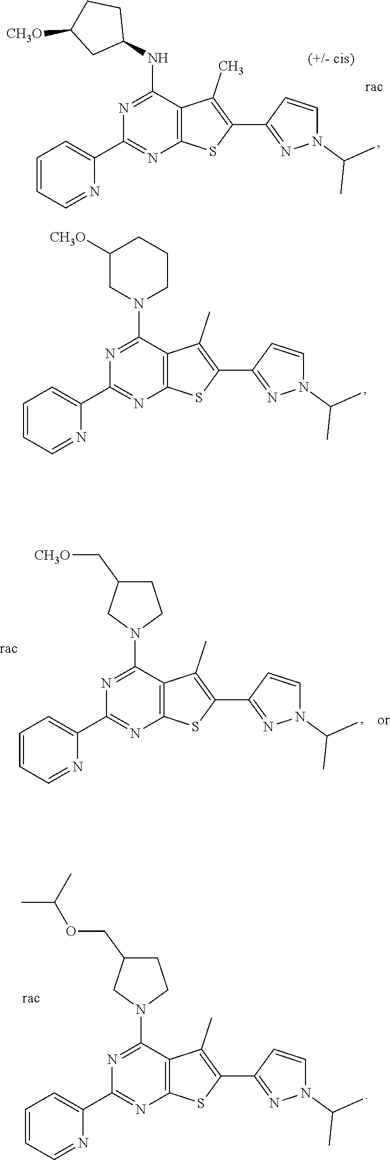

74. A compound of Formula IE: 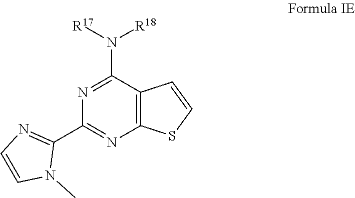
or a pharmaceutically acceptable derivative thereof, wherein:
—NR¹⁷R¹⁸ is

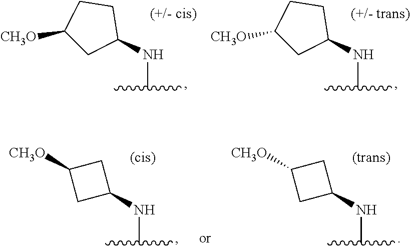

75. The compound or pharmaceutically acceptable derivative of claim 74, wherein —NR¹⁷R¹⁸ is:

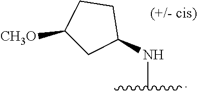

76. The compound or pharmaceutically acceptable derivative of claim 74, wherein —NR¹⁷R¹⁸ is:

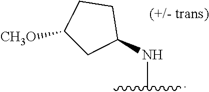

77. The compound or pharmaceutically acceptable derivative of claim 74, wherein —NR¹⁷R¹⁸ is:

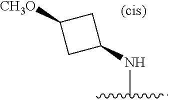

78. The compound or pharmaceutically acceptable derivative of claim 74, wherein —NR¹⁷R¹⁸ is:

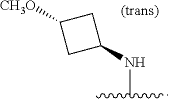

79. The compound or pharmaceutically acceptable derivative of claim 74, wherein the compound of Formula IE is:

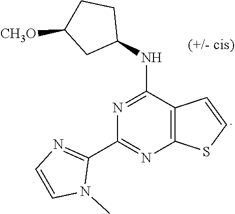

80. A compound of Formula IF: 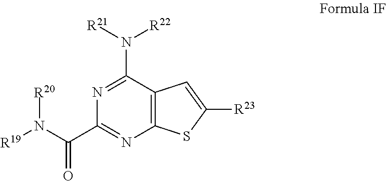
or a pharmaceutically acceptable derivative thereof, wherein:
—N¹⁹R²⁰ is —NH₂, 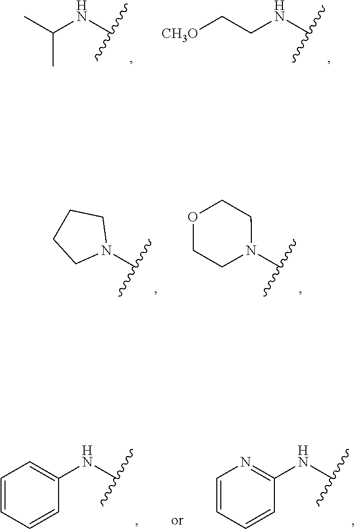
—NR²¹R²² is 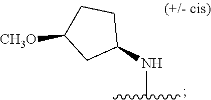
and
R²³ is hydrogen or

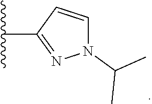 [SMILES: *c1ccn(C(C)C)n1]

81. The compound or pharmaceutically acceptable derivative of claim 80, wherein R²³ is hydrogen.

82. The compound or pharmaceutically acceptable derivative of claim 80, wherein R²³ is:

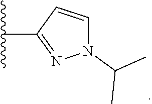 [SMILES: *c1ccn(C(C)C)n1]

83. The compound or pharmaceutically acceptable derivative of any one of claims 80-82, wherein the compound of Formula IF is:

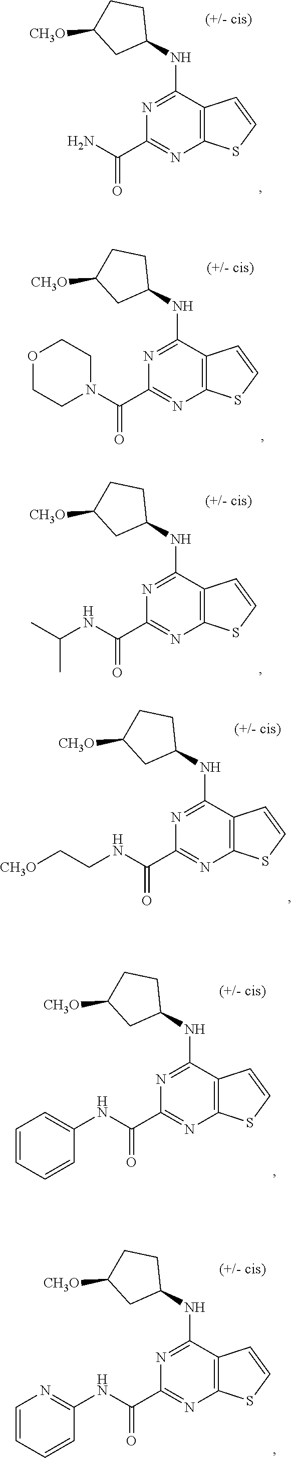

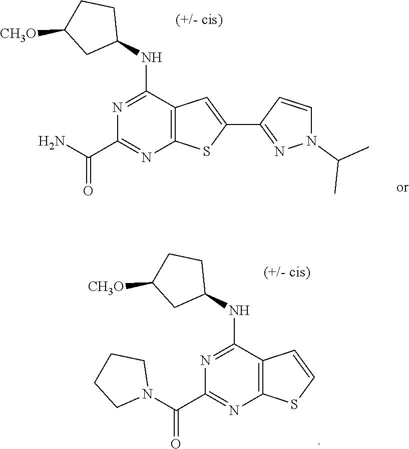

84. A compound of Formula IIA: 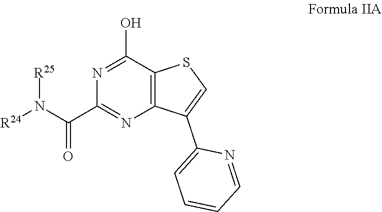
or a pharmaceutically acceptable derivative thereof, wherein:
—NR²⁴R²⁵ is —NH₂,

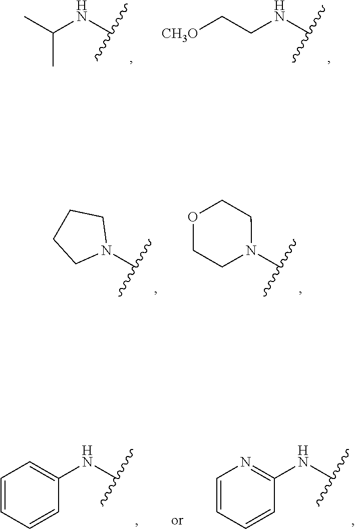

85. The compound or pharmaceutically acceptable derivative of claim 84, wherein the compound of Formula IIA is:

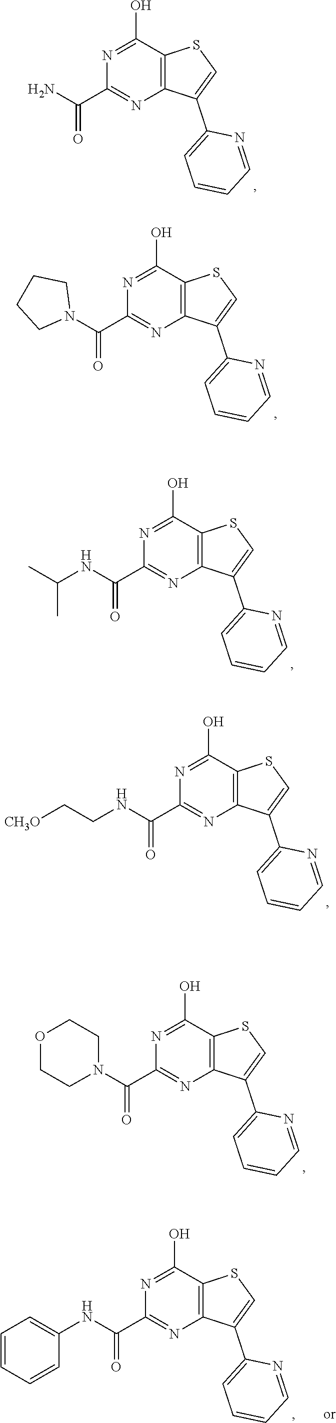

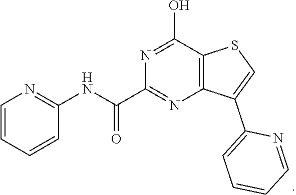 [SMILES: O=C(Nc1ccccn1)c1nc(O)c2scc(-c3ccccn3)c2n1]

86. A compound of Formula IIB: 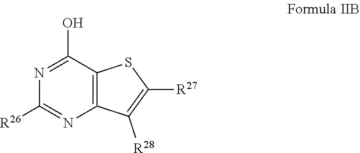
or a pharmaceutically acceptable derivative thereof, wherein:
R²⁶ is 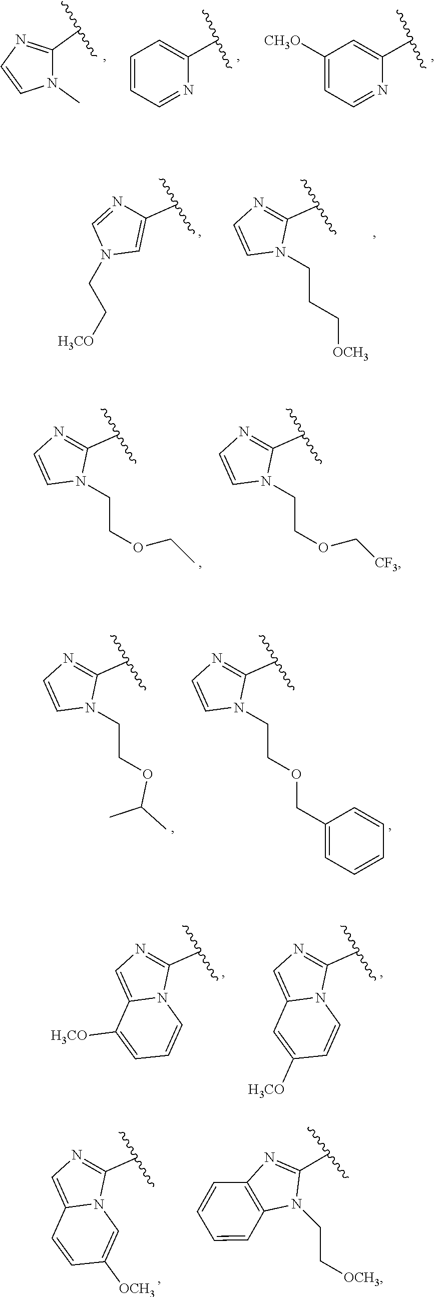 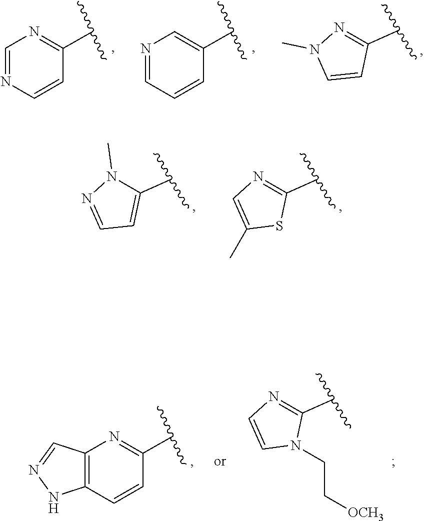
R²⁷ is hydrogen —CH₃, or —CF₃; and
R²⁸ is

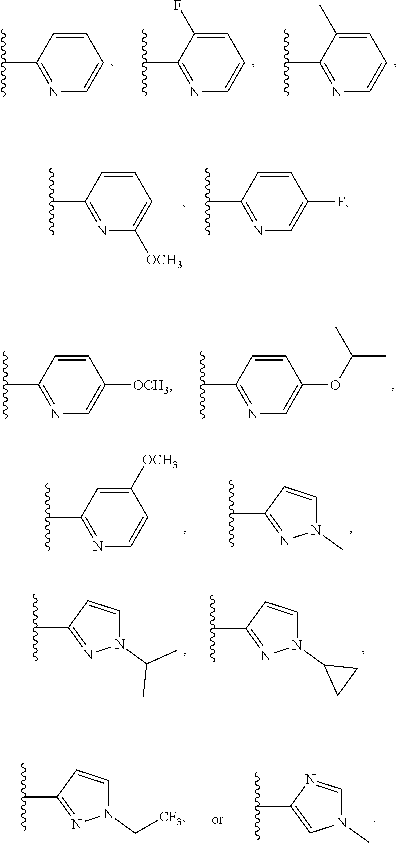

87. The compound or pharmaceutically acceptable derivative of claim 86, wherein R²⁶ is:

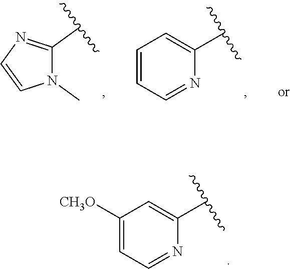

88. The compound or pharmaceutically acceptable derivative of claim 86, wherein R²⁶ is:

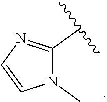 [SMILES: *c1nccn1C]

89. The compound or pharmaceutically acceptable derivative of claim 86, wherein R²⁶ is:

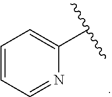 [SMILES: *c1ccccn1]

90. The compound or pharmaceutically acceptable derivative of claim 86, wherein R²⁶ is:

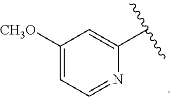

91. The compound or pharmaceutically acceptable derivative of claim 86, wherein R²⁶ is:

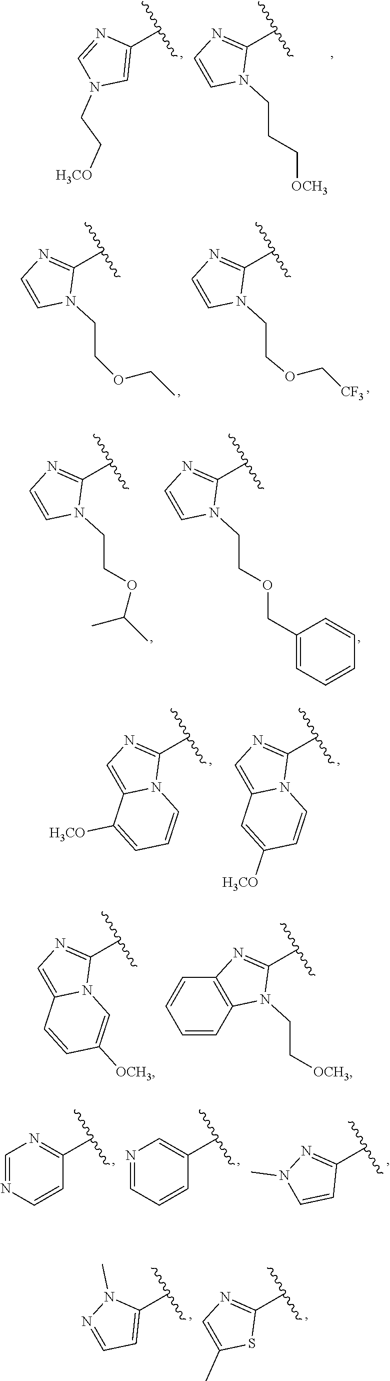

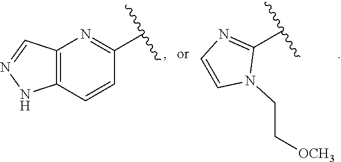 [SMILES: *c1ccc2[nH]ncc2n1 ; *c1nccn1CCOC]

92. The compound or pharmaceutically acceptable derivative of claim 86, wherein R²⁶ is:

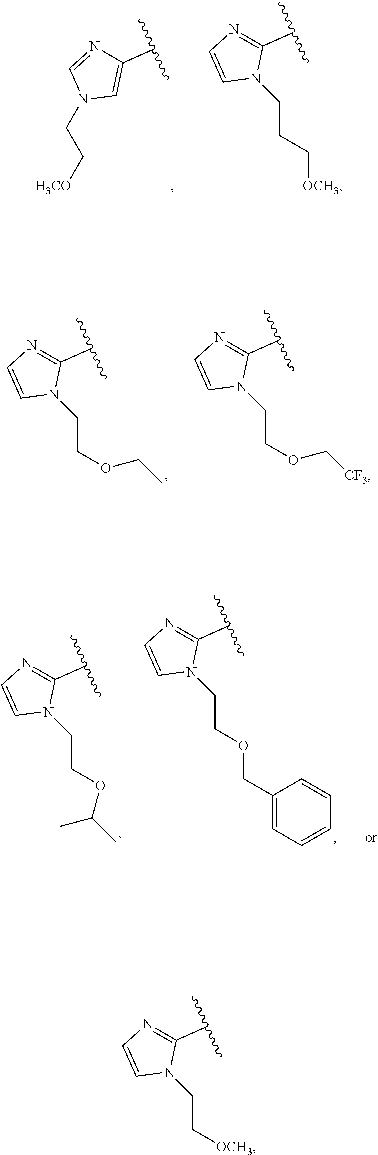

93. The compound or pharmaceutically acceptable derivative of claim 86, wherein R²⁶ is:

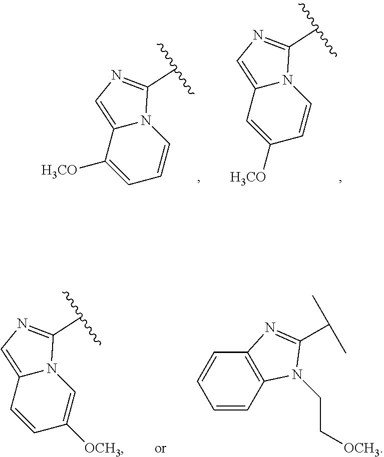 [SMILES: *c1ncc2c(OC)cccn12 ; *c1ncc2cc(OC)ccn12 ; *c1ncc2ccc(OC)cn12 ; COCCn1c(C(C)C)nc2ccccc21]

94. The compound or pharmaceutically acceptable derivative of claim 86, wherein R²⁶ is:

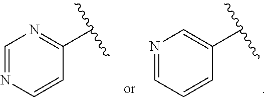 [SMILES: *c1ccncn1 ; *c1cccnc1]

95. The compound or pharmaceutically acceptable derivative of claim 86, wherein R²⁶ is:

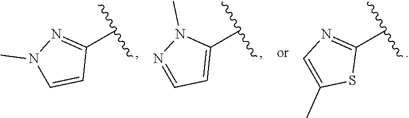

96. The compound or pharmaceutically acceptable derivative of claim 86, wherein R²⁶ is:

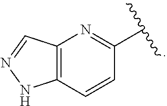 [SMILES: *c1ccc2[nH]ncc2n1]

97. The compound or pharmaceutically acceptable derivative of any one of claims 86-96, wherein R²⁷ is hydrogen.

98. The compound or pharmaceutically acceptable derivative of any one of claims 86-96, wherein R²⁷ is —CH₃.

99. The compound or pharmaceutically acceptable derivative of any one of claims 86-96, wherein R²⁷ is —CF₃.

100. The compound or pharmaceutically acceptable derivative of any one of any one of claims 86-98, wherein R²⁸ is:

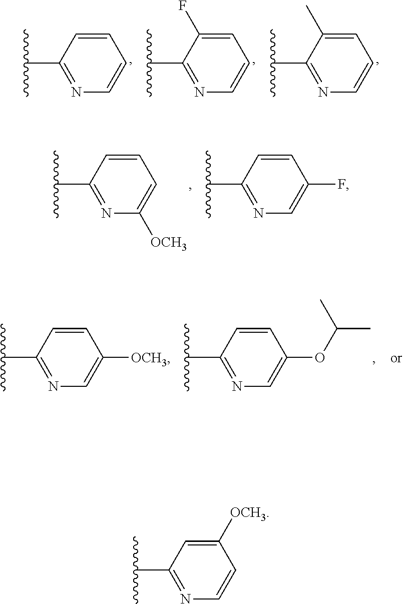

101. The compound or pharmaceutically acceptable derivative of any one of claims 86-98, wherein R²⁸ is:

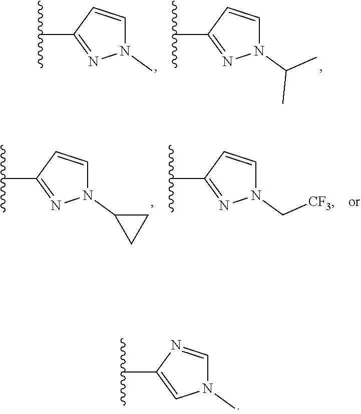

102. The compound or pharmaceutically acceptable derivative of claim 86, wherein:
R²⁶ is 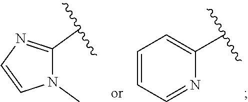
R²⁷ is hydrogen; and
R²⁸ is

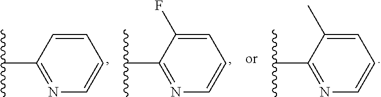

103. The compound or pharmaceutically acceptable derivative of claim 102, wherein R²⁶ is:

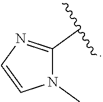 [SMILES: *c1nccn1C]

104. The compound or pharmaceutically acceptable derivative of claim 102, wherein R²⁶ is:

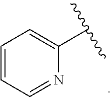 [SMILES: *c1ccccn1]

105. The compound or pharmaceutically acceptable derivative of any one of claims 102-104, wherein R²⁸ is:

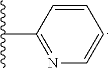 [SMILES: *c1ccccn1]

106. The compound or pharmaceutically acceptable derivative of any one of claims 102-104, wherein R²⁸ is:

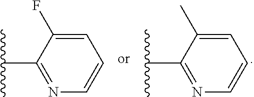

107. The compound or pharmaceutically acceptable derivative of any one of claims 86-106, with the proviso that when:
R²⁶ is 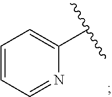 [SMILES: *c1ccccn1]
and
R²⁷ is hydrogen; then
R²⁸ is not

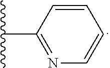 [SMILES: *c1ccccn1]

108. The compound or pharmaceutically acceptable derivative of any one of claims 86-107, wherein the compound of Formula IIB is:

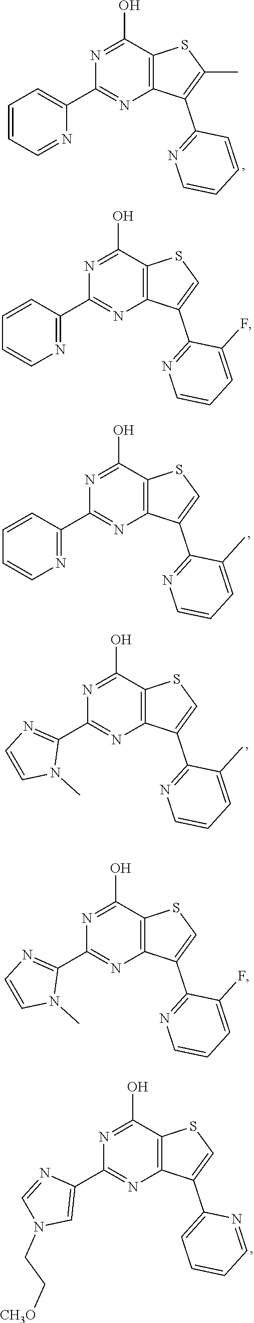

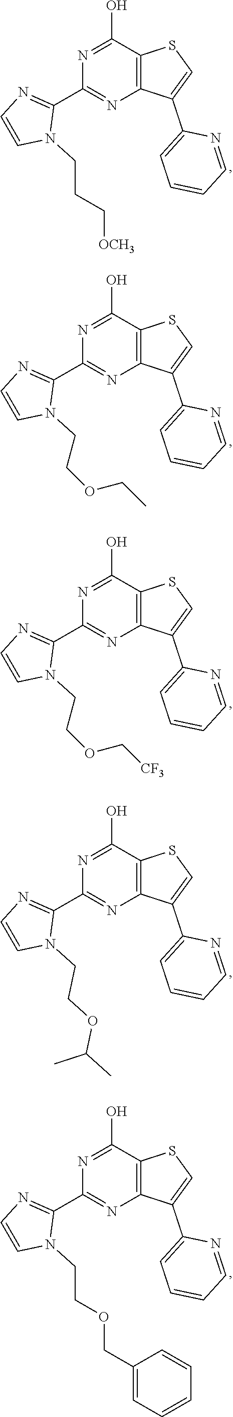

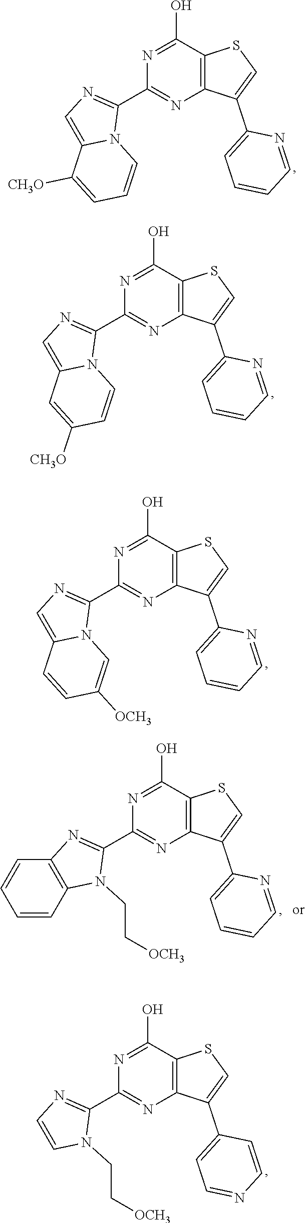

109. The compound or pharmaceutically acceptable derivative of any one of claims 86-107, wherein the compound of Formula IIB is:

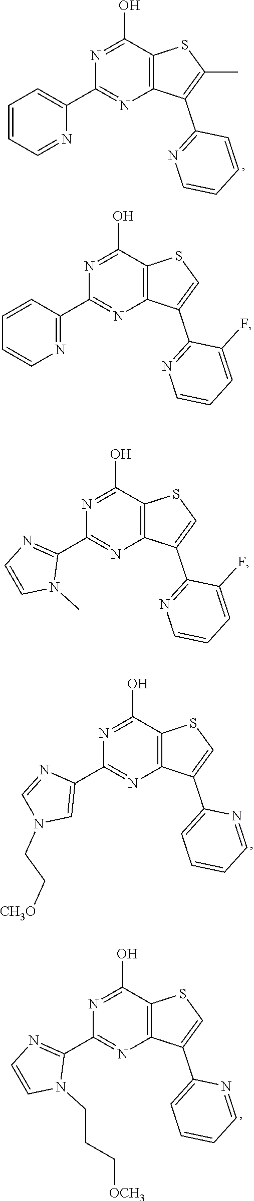

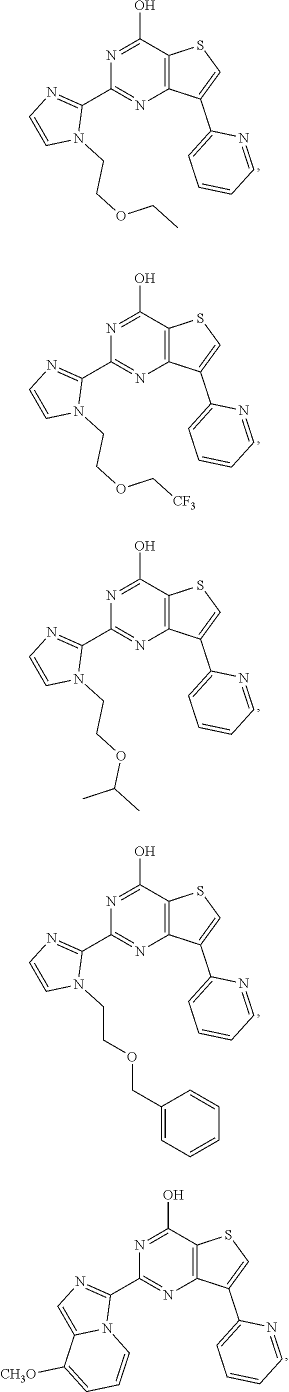

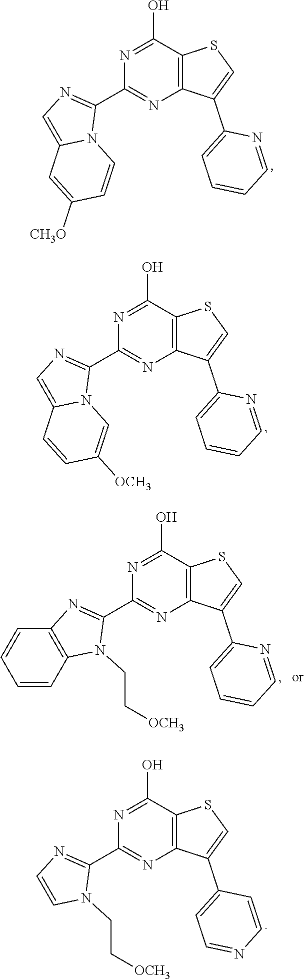

110. The compound or pharmaceutically acceptable derivative of any one of claims 86-107, wherein the compound of Formula IIB is:

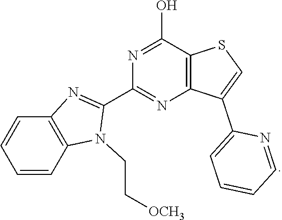 [SMILES: COCCn1c(-c2nc(O)c3scc(-c4ccccn4)c3n2)nc2ccccc21]

111. A compound or a pharmaceutically acceptable derivative thereof, wherein the compound is:

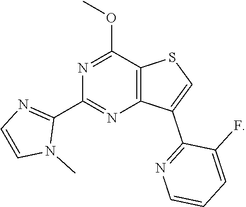 [SMILES: COc1nc(-c2nccn2C)nc2c(-c3ncccc3F)csc12]

112. A compound of selected from the group consisting of compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, and 95, or a pharmaceutically acceptable derivative thereof.

113. A compound of selected from the group consisting of compounds 1, 2, 3, 4, 5, 6, 7, 8, 9, 10, 11, 12, 13, 14, 15, 16, 17, 18, 19, 20, 21, 22, 23, 24, 25, 26, and 27, or a pharmaceutically acceptable derivative thereof.

114. A compound of selected from the group consisting of compounds 28, 29, 30, 31, 32, 33, 34, 35, 36, 37, 38, and 39 or a pharmaceutically acceptable derivative thereof.

115. A compound which binds to the GTP binding domain of one or more members of the Ras superfamily and inhibits the one or more members of the Ras superfamily with an IC₅₀ value of less than 10 micromolar, wherein the compound is the compound or pharmaceutically acceptable derivative of any one of claims 1-114.

116. The compound of claim 115, wherein one or more members of the Ras superfamily is Ras.

117. The compound of claim 115, wherein one or more members of the Ras superfamily is Rho.

118. The compound of claim 115, wherein one or more members of the Ras superfamily is Rac.

119. The compound of claim 116, wherein the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

120. The compound of claim 119, wherein the Ras is HRAS, KRAS, NRAS, or a mutant thereof.

121. The compound of claim 120, wherein the Ras is HRAS or a mutant thereof.

122. The compound of claim 120, wherein the Ras is KRAS or a mutant thereof.

123. The compound of claim 120, wherein the Ras is NRAS or a mutant thereof.

124. The compound of claim 117, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

125. The compound of claim 117, wherein the Rho is Rac.

126. The compound of claim 118 or 125, wherein the Rac is RAC1; RAC2; RAC3; RHOG, or a mutant thereof.

127. The compound or pharmaceutically acceptable derivative of any one of claims 1-126, wherein the pharmaceutically acceptable derivative of the compound is a pharmaceutically acceptable salt of said compound.

128. A method of inhibiting the function of one or more members of the Ras superfamily, comprising administering to a subject a compound which inhibits the one or more members of the Ras superfamily with an IC₅₀ value of less than 10 μM, wherein the compound is the compound or pharmaceutically acceptable derivative of any one of claims 1-126 or the compound is the compound or pharmaceutically acceptable salt of claim 127.

129. The method of claim 128, wherein one or more members of the Ras superfamily is Ras.

130. The method of claim 128, wherein one or more members of the Ras superfamily is Rho.

131. The method of claim 128, wherein one or more members of the Ras superfamily is Rac.

132. The method of claim 128, wherein the Ras is DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; or RRAS2.

133. The method of claim 132, wherein the Ras is HRAS, KRAS, NRAS or a mutant thereof.

134. The method of claim 132, wherein the Ras is HRAS or a mutant thereof.

135. The method of claim 132, wherein the Ras is KRAS or a mutant thereof.

136. The method of claim 132, wherein the Ras is NRAS or a mutant thereof.

137. The method of claim 130, wherein the Rho is RHOA; RHOB; RHOBTB1; RHOBTB2; RHOBTB3; RHOC; RHOD; RHOF; RHOG; RHOH; RHOJ; RHOQ; RHOU; RHOV; RND1; RND2; RND3; RAC1; RAC2; RAC3; CDC42, or a mutant thereof.

138. The method of claim 137, wherein the Rho is Rac.

139. The method of claim 131 or 138, wherein the Rac is RAC1; RAC2; RAC3; RHOG, or a mutant thereof.

140. The method of claim 128, wherein the inhibiting the function of one or more members of the Ras superfamily is a treatment, prevention or amelioration of one or more symptoms of cancer.

141. The method of any of claim 129 or 132-136, wherein the inhibiting the function of Ras is a treatment, prevention or amelioration of one or more symptoms of cancer.

142. The method of any of claim 130 or 137-138, wherein the inhibiting the function of Rho is a treatment, prevention or amelioration of one or more symptoms of cancer.

143. The method of any of claim 131 or 138-139, wherein the inhibiting the function of Rac is a treatment, prevention or amelioration of one or more symptoms of cancer.

144. The method of any of claims 140-143, wherein the cancer is a solid tumor.

145. The method of claim 144, wherein the solid tumor is hepatocellular carcinoma, prostate cancer, pancreatic cancer, lung cancer, breast cancer, ovarian cancer, colon cancer, small intestine cancer, biliary tract cancer, endometrium cancer, skin cancer (melanoma), cervix cancer, urinary tract cancer, or glioblastoma.

146. The method of claim 145, wherein the solid tumor is pancreatic cancer.

147. The method of claim 145, wherein the solid tumor is colon cancer.

148. The method of claim 145, wherein the solid tumor is small intestine cancer.

149. The method of claim 145, wherein the solid tumor is biliary tract cancer.

150. The method of claim 145, wherein the solid tumor is endometrium cancer.

151. The method of claim 145, wherein the solid tumor is lung cancer.

152. The method of claim 145, wherein the solid tumor is breast cancer.

153. The method of claim 145, wherein the solid tumor is skin cancer.

154. The method of claim 145, wherein the solid tumor is cervix cancer.

155. The method of claim 145, wherein the solid tumor is urinary tract cancer.

156. The method of any of claims 140-143, wherein the cancer is a blood borne tumor.

157. The method of claim 156, wherein the blood borne tumor is a leukemia.

158. The method of claim 156, wherein the blood borne tumor is chronic lymphocytic leukemia (CLL), chronic myelocytic leukemia (CML), acute lymphoblastic leukemia (ALL), acute myeloid leukemia (AML), or acute myeloblastic leukemia (AML).

159. The method of any one of claims 156-158, wherein the blood borne tumor is metastatic.

160. The method of claim 128, wherein the inhibiting the function of one or more members of the Ras superfamily is a treatment, prevention or amelioration of one or more symptoms of an inflammatory disease.

161. The method of any of claim 129 or 132-136, wherein inhibiting the function of Ras is a treatment, prevention or amelioration of one or more symptoms of an inflammatory disease.

162. The method of any of claim 130 or 137-138, wherein the inhibiting the function of Rho is a treatment, prevention or amelioration of one or more symptoms of inflammatory disease.

163. The method of any of claim 131 or 138-139, wherein the inhibiting the function of Rac is a treatment, prevention or amelioration of one or more symptoms of inflammatory disease.

164. The method of any of claims 160-163, wherein the inflammatory disease is gastritis, schistosomiasis, cholangitis, chronic cholecystitis, pelvic inflammatory disease, chronic cervicitis, osteomyelitis, inflammatory bowel disease, reflux esophagitis, Barrett's esophagus, bladder inflammation (cystitis), asbestosis, silicosis, gingivitis, lichen planus, pancreatitis, protease mutation, lichen sclerosis, slaladenitis, bronchitis, Sjogren syndrome or Hashimoto's thyroiditis.

165. The method of any of claims 160-163, wherein the inflammatory disease is Alzheimer's disease (AD), ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus, erythematous (SLE), nephritis, Parkinson's disease, ulcerative colitis.

166. The method of claim 165, wherein the inflammatory disease is Alzheimer's disease (AD).

167. The method of claim 165, wherein the inflammatory disease is ankylosing spondylitis.

168. The method of claim 165, wherein the inflammatory disease is arthritis (osteoarthritis, rheumatoid arthritis (RA), psoriatic arthritis).

169. The method of claim 165, wherein the inflammatory disease is asthma.

170. The method of claim 165, wherein the inflammatory disease is atherosclerosis.

171. The method of claim 165, wherein the inflammatory disease is Crohn's disease.

172. The method of claim 165, wherein the inflammatory disease is colitis.

173. The method of claim 165, wherein the inflammatory disease is dermatitis.

174. The method of claim 165, wherein the inflammatory disease is diverticulitis.

175. The method of claim 165, wherein the inflammatory disease is fibromyalgia.

176. The method of claim 165, wherein the inflammatory disease is hepatitis.

177. The method of claim 165, wherein the inflammatory disease is irritable bowel syndrome (IBS).

178. The method of claim 165, wherein the inflammatory disease is systemic lupus.

179. The method of claim 165, wherein the inflammatory disease is erythematous (SLE).

180. The method of claim 165, wherein the inflammatory disease is nephritis.

181. The method of claim 165, wherein the inflammatory disease is Parkinson's disease.

182. The method of claim 165, wherein the inflammatory disease is ulcerative colitis.

183. The method of claim 128, wherein the inhibiting the function of one or more members of the Ras superfamily is a treatment, prevention or amelioration of one or more symptoms of a rasopathy.

184. The method of any of claim 129 or 132-136, wherein the inhibiting the function of Ras is a treatment for a rasopathy.

185. The method of any of claim 130 or 137-138, wherein the inhibiting the function of Rho is a treatment for a rasopathy.

186. The method of any of claim 131 or 138-139, wherein the inhibiting the function of Rac is a treatment for a rasopathy.

187. The method of any of claims 183-186, wherein the rasopathy is neurofibromatosis type 1, Noonan's syndrome or Costello syndrome.

188. The method of any of claim 129 or 132-136, wherein the inhibiting the function of Ras is a treatment for Ras-associated autoimmune leukoproliferative disorder.

189. The method of claim 128, wherein the inhibiting the function of one or more members of the Ras superfamily is a treatment, prevention or amelioration of one or more symptoms of a fibrotic disease.

190. The method of any of claim 129 or 132-136, wherein the inhibiting the function of Ras is a treatment, prevention or amelioration of one or more symptoms of a fibrotic disease.

191. The method of any of claim 130 or 137-138, wherein the inhibiting the function of Rho is a treatment, prevention or amelioration of one or more symptoms of a fibrotic disease.

192. The method of any of claim 130 or 137-138, wherein the inhibiting the function of Rac is a treatment, prevention or amelioration of one or more symptoms of a fibrotic disease.

193. The method of any one of claims 140, 160, 183, or 189, wherein one or more members of the Ras superfamily is Ras.

194. The method of any one of claims 140, 160, 183, or 189, wherein one or more members of the Ras superfamily is Rho.

195. The method of any one of claims 140, 160, 183, or 189, wherein one or more members of the Ras superfamily is Rac.

196. A pharmaceutical composition, comprising the compound or pharmaceutically acceptable derivative of any one of claims 1-126, and a pharmaceutically acceptable carrier.

197. The pharmaceutical composition of claim 196, wherein the pharmaceutical composition comprises a therapeutic amount of said compound or pharmaceutically acceptable derivative thereof.

198. A pharmaceutical composition, comprising the compound or pharmaceutically acceptable salt of claim 127, and a pharmaceutically acceptable carrier.

199. The pharmaceutical composition of claim 198, wherein the pharmaceutical composition comprises a therapeutic amount of said compound or pharmaceutically acceptable salt thereof.

200. A method of inhibiting the function of one or more members of the Ras superfamily, comprising administering to a subject the pharmaceutical composition of any one of claims 196-199.

201. A method of inhibiting the function of one or more members of the Ras superfamily, comprising administering to a subject the compound or pharmaceutically acceptable derivative of any one of claims 1-126.
